(19) United States
(12) Patent Application Publication
(10) Pub. No.: US 2022/0396565 A1
(43) Pub. Date: Dec. 15, 2022

(54) NOVEL HETEROCYCLIC COMPOUNDS

(71) Applicant: Hoffmann-La Roche Inc., Little Falls, NJ (US)

(72) Inventors: Zhanling CHENG, Shanghai (CN); Xingchun HAN, Shanghai (CN); Christian KRAMER, Lörrach (DE); Christian LERNER, Bottmingen (CH); Yongqiang LIU, Shanghai (CN); Matthias NETTEKOVEN, Grenzach-Wyhlen (DE); Philippe PFLIEGER, Schwoben (FR); Bernd PUELLMANN, Aesch (CH); Jianhua WANG, Shanghai (CN); Lisha WANG, Riehen (CH); Min WANG, Shanghai (CN); Yongguang WANG, Shanghai (CN); Song YANG, Shanghai (CN); Chengang ZHOU, Shanghai (CN)

(73) Assignee: Hoffmann-La Roche Inc., Little Falls, NJ (US)

(21) Appl. No.: 17/869,755

(22) Filed: Jul. 20, 2022

Related U.S. Application Data
(63) Continuation of application No. PCT/EP2021/051095, filed on Jan. 20, 2021.

(30) Foreign Application Priority Data
Jan. 22, 2020 (CN) PCT/CN2020/073830

(51) Int. Cl. C07D 403/12; C07D 233/90; C07D 403/14; C07D 487/10; C07D 401/14

(52) U.S. Cl. CPC C07D 403/12; C07D 233/90; C07D 403/14; C07D 487/10; C07D 401/14

(57) ABSTRACT

Provided are novel heterocyclic compounds having the general formula (I), and pharmaceutically acceptable salts thereof, wherein R¹ to R⁴, m, n, and p are as described herein.

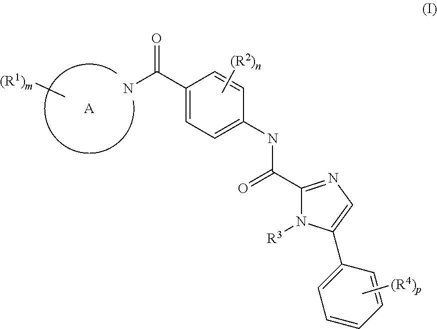

Further provided are pharmaceutical compositions including the compounds, processes of manufacturing the compounds and methods of using the compounds as medicaments, in particular methods of using the compounds as antibiotics for the treatment or prevention of bacterial infections and resulting diseases.

### CROSS-REFERENCE TO RELATED APPLICATIONS

This application is a Continuation application of International Patent Application No. PCT/EP2021/051095, filed on Jan. 20, 2021, which claims benefit of priority to International Patent Application No. PCT/CN2020/073830, filed on Jan. 22, 2020, all of which are incorporated herein by reference in their entirety.

### BACKGROUND

Certain embodiments of the present invention relates to novel heterocyclic compounds which exhibit antibacterial properties. Certain embodiments of the invention also relates to methods of using the compounds for the treatment or prevention of bacterial infections and resulting diseases, in particular for the treatment or prevention of infections with Acinetobacter baumannii and resulting diseases.

Acinetobacter baumannii is a Gram-negative, aerobic, nonfermenting bacterium recognized over the last decades as an emergining pathogen with very limited treatment options.

A. baumannii is considered to be a serious threat by the US Centers for Disease Control and Prevention and belongs to the so called ‘ESKAPE’ pathogens (Enterococcus faecium, Staphylococcus aureus, Klebsiella pneumoniae, Acinetobacter baumannii, Pseudomonas aeruginosa and Enterobacter species & E. coli) that currently cause the majority of nosocomial infections and effectively “escape” the activity of antimicrobial agents.

A. baumannii is most often encountered in intensive care units and surgical wards, where extensive antibiotic use has enabled selection for resistance against all known antimicrobials and where it causes infections that include bacteremia, pneumonia, meningitis, urinary tract infection, and wound infection.

A. baumannii has an exceptional ability to upregulate and acquire resistance determinants and shows an environmental persistance that allows its survival and spread in the nosocomial setting, making this organism a frequent cause of outbreaks of infection and an endemic, health care-associated pathogen.

Due to increasing antibiotic resistance to most if not all available therapeutic options, Multi-Drug Resistant (MDR) A. baumanniii infections, especially those caused by Carbapenem resistant A. baumannii, are extremely difficult or even impossible to treat with high mortality rate as well as increased morbidity and length of stay in intensive care unit.

Acinetobacter baumannii has been defined and still remains “a prime example of a mismatch between unmet medical needs and the current antimicrobial research and development pipeline” according to the Antimicrobial Availability Task Force (AATF) of the Infectious Diseases Society of America (IDSA). Thus, there is a high demand and need to identify compounds suitable for the treatment of diseases and infections caused by Acinetobacter baumannii.

The present invention provides novel compounds which exhibit activity against drug-susceptible as well as drug-resistant strains of Acinetobacter baumannii.

### SUMMARY OF THE DISCLOSURE

In a first aspect, the present invention provides compounds of formula (I)

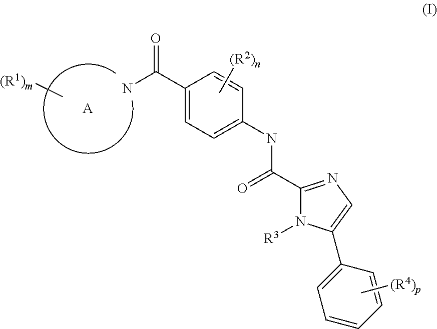

or pharmaceutically acceptable salts thereof, wherein R¹ to R⁴, m, n, and p are as defined herein.

In one aspect, the present invention provides a process of manufacturing the compounds of formula (I) described herein, wherein said process is as described in any one of Schemes 1 to 5 herein.

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a further aspect, the present invention provides a compound of formula (I) as described herein, or a pharmaceutically acceptable salt thereof, for use in the treatment or prevention of infections and resulting diseases caused by Gram-negative bacteria.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

### DETAILED DESCRIPTION OF THE DISCLOSURE

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The following definitions are provided to facilitate understanding of certain terms used frequently herein and are not meant to limit the scope of the present disclosure. All references referred to herein are incorporated by reference in their entirety.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkynyl” denotes a monovalent linear or branched hydrocarbon group of 2 to 6 carbon atoms with at least one carbon-carbon triple bond (“C₂-C₆-alkynyl”). In particular embodiments, alkynyl has 2 to 4 carbon atoms with at least one triple bond. Examples of alkynyl include ethynyl, propynyl, n-butynyl or isobutynyl. Preferred alkynyl is propynyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, and spiro[2.3]hexan-5-yl.

The term “aminoalkoxy” refers to an alkoxy group, wherein at least one of the hydrogen atoms of the alkoxy group has been replaced by an amino group. Preferably, “aminoalkoxy” refers to an alkoxy group wherein 1, 2 or 3 hydrogen atoms of the alkoxy group have been replaced by an amino group. Preferred, yet non-limiting examples of aminoalkoxy are aminomethoxy and 1-aminoethoxy.

The term “aminoalkoxyalkoxy” refers to an alkoxy group, wherein at least one of the hydrogen atoms of the alkoxy group has been replaced by an aminoalkoxy group. Preferably, “aminoalkoxyalkoxy” refers to an alkoxy group wherein 1, 2 or 3 hydrogen atoms of the alkoxy group have been replaced by an aminoalkoxy group. Most preferably, “aminoalkoxyalkoxy” refers to an alkoxy group wherein 1 hydrogen atom of the alkoxy group has been replaced by an aminoalkoxy group.

The term “heterocyclyl” refers to a saturated or partly unsaturated mono- or bicyclic, preferably monocyclic ring system of 3 to 10 ring atoms, preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidin-3-yl; azetidin-2-yl; oxetan-3-yl; oxetan-2-yl; piperidyl; piperazinyl; pyrrolidinyl; 2-oxopyrrolidin-1-yl; 2-oxopyrrolidin-3-yl; 5-oxopyrrolidin-2-yl; 5-oxopyrrolidin-3-yl; 2-oxo-1-piperidyl; 2-oxo-3-piperidyl; 2-oxo-4-piperidyl; 6-oxo-2-piperidyl; 6-oxo-3-piperidyl; 1-piperidinyl; 2-piperidinyl; 3-piperidinyl; 4-piperidinyl; morpholino; morpholin-2-yl; morpholin-3-yl; pyrrolidinyl (e.g., pyrrolidin-3-yl); 3-azabicyclo[3.1.0]hexan-6-yl; 2,5-diazabicyclo[2.2.1]heptan-2-yl; 2-azaspiro[3.3]heptan-2-yl; 2,6-diazaspiro[3.3]heptan-2-yl; and 2,3,3a,4,6,6a-hexahydro-1H-pyrrolo[3,4-c]pyrrol-5-yl.

The term “aryl” refers to a monocyclic, bicyclic, or tricyclic carbocyclic ring system having a total of 6 to 10 ring members (“C₆-C₁₀-aryl”) and wherein at least one ring in the system is aromatic. A particularly preferred, yet non-limiting example of aryl is phenyl.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, thiazol-4-yl, and 1,2,4-oxadiazol-3-yl. Most preferably, “heteroaryl” refers to pyridyl and pyrimidinyl.

The term “heteroaryloxy” refers to a heteroaryl group, as previously defined, attached to the parent molecular moiety via an oxygen atom.

The term “hydroxy” refers to an —OH group.

The term “amino” refers to an —NH₂ group.

The term “cyano” refers to a —CN (nitrile) group.

The term “oxo” refers to a double bonded oxygen (═O).

The term “carbamoyl” refers to a —C(O)NH₂ group.

The term “carbamimidoyl” refers to a group

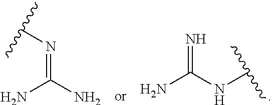 [SMILES: *N=C(N)N ; *NC(=N)N]

The term “carboxy” refers to a —C(O)OH group (i.e., a carboxyclic acid moiety).

The term “carbonyl” refers to a carbon radical having two of the four covalent bonds shared with an oxygen atom (C═O).

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Non-limiting examples of haloalkyl are fluoromethyl, difluoromethyl, trifluoromethyl, trifluoroethyl, 2-fluoroethyl, and 2,2-difluoroethyl. A particularly preferred, yet non-limiting example of haloalkyl is trifluoromethyl.

The term “cyanoalkyl” refers to an alkyl group, wherein at least one of the hydrogen atoms of the alkyl group has been replaced by cyano group. Preferably, “cyanoalkyl” refers to an alkyl group wherein 1, 2 or 3 hydrogen atoms of the alkyl group have been replaced by a cyano group. Most preferably, “cyanoalkyl” refers to an alkyl group wherein 1 hydrogen atom of the alkyl group has been replaced by a cyano group.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are fluoromethoxy (FCH₂O—), difluoromethoxy (F₂CHO—), and trifluoromethoxy (F₃CO—).

The term “cyanoalkoxy” refers to an alkoxy group, wherein at least one of the hydrogen atoms of the alkoxy group has been replaced by cyano group. Preferably, “cyanoalkoxy” refers to an alkoxy group wherein 1, 2 or 3 hydrogen atoms of the alkoxy group have been replaced by a cyano group. Most preferably, “cyanoalkoxy” refers to an alkoxy group wherein 1 hydrogen atom of the alkoxy group has been replaced by a cyano group.

The term “carbamoylalkoxy” refers to an alkoxy group, wherein at least one of the hydrogen atoms of the alkoxy group has been replaced by a carbamoyl group. Preferably, “carbamoylalkoxy” refers to an alkoxy group wherein 1, 2 or 3 hydrogen atoms of the alkoxy group have been replaced by a carbamoyl group. Most preferably, “carbamoylalkoxy” refers to an alkoxy group wherein 1 hydrogen atom of the alkoxy group has been replaced by a carbamoyl group.

The term “alkoxyalkoxy” refers to an alkoxy group, wherein at least one of the hydrogen atoms of the alkoxy group has been replaced by an alkoxy group, preferably methoxy. Preferably, “alkoxyalkoxy” refers to an alkoxy group wherein 1, 2 or 3 hydrogen atoms of the alkoxy group have been replaced by an alkoxy group, most preferably methoxy. A particularly preferred, yet non-limiting example of alkoxyalkoxy is 2-methoxyethoxy.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl, hydroxyethyl (e.g. 2-hydroxyethyl), and 3-hydroxy-3-methyl-butyl.

The term “aminoalkyl” refers to an alkyl group, wherein at least one of the hydrogen atoms of the alkyl group has been replaced by an amino group. Preferably, “aminoalkyl” refers to an alkyl group wherein 1, 2 or 3 hydrogen atoms, most preferably 1 hydrogen atom of the alkyl group have been replaced by an amino group. Preferred, yet non-limiting examples of aminoalkyl are aminomethyl, aminoethyl (e.g. 2-aminoethyl), and 3-amino-3-methyl-butyl.

The term “carboxyalkyl” refers to an alkyl group, wherein at least one of the hydrogen atoms of the alkyl group has been replaced by a carboxy group. Preferably, “carboxyalkyl” refers to an alkyl group wherein 1, 2 or 3 hydrogen atoms, most preferably 1 hydrogen atom of the alkyl group have been replaced by a carboxy group. Preferred, yet non-limiting examples of carboxyalkyl are carboxymethyl, carboxyethyl (e.g. 2-carboxyethyl), and 3-carboxy-3-methyl-butyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, fumarates, lactates (in particular derived from L-(+)-lactic acid), tartrates (in particular derived from L-(+)-tartaric acid) and trifluoroacetates.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “mammal” as used herein includes both humans and non-humans and includes but is not limited to humans, non-human primates, canines, felines, murines, bovines, equines, and porcines. In a particularly preferred embodiment, the term “mammal” refers to humans.

The term “nosocomial infection” refers to a hospital-acquired infection (HAI), which is an infection that is acquired in a hospital or other health care facility. To emphasize both hospital and nonhospital settings, it is sometimes instead called a health care-associated infection (HAI or HCAI). Such an infection can be acquired in hospitals, nursing homes, rehabilitation facilities, outpatient clinics, or other clinical settings.

### Compounds

In a first aspect, the present invention provides a compound of formula (I)

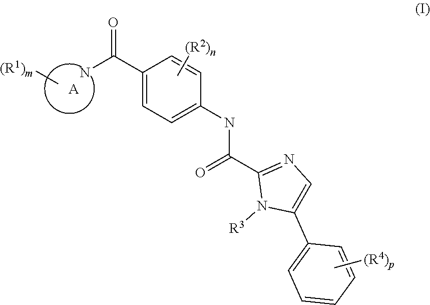

or a pharmaceutically acceptable salt thereof, wherein:
R¹ is, at each occurrence, independently selected from hydroxy, halogen, cyano, amino, C₁-C₆-alkoxy, halo-C₁-C₆-alkoxy, amino-C₁-C₆-alkoxy-, a group

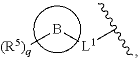

and a group C₁-C₆-alkyl-L²—; wherein C₁-C₆-alkyl is optionally substituted with 1-3 substituents selected from hydroxy, amino, halogen, cyano, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy;
R² is, at each occurrence, independently selected from halogen, cyano, C₁-C₆-alkyl, C₁-C₆-alkoxy, halo-C₁-C₆-alkyl, and halo-C₁-C₆-alkoxy;
R³ is selected from hydrogen, C₁-C₆-alkyl, and halo-C₁-C₆-alkyl;
R⁴ is, at each occurrence, independently selected from halogen, C₁-C₆-alkyl, C₁-C₆-alkoxy, cyano, halo-C₁-C₆-alkyl, cyano-C₁-C₆-alkyl, (C₁-C₆-alkyl)₂N—, halo-C₁-C₆-alkoxy, cyano-C₁-C₆-alkoxy, C₁-C₆-alkoxy-C₁-C₆-alkoxy-, (C₁-C₆-alkyl)₂N—C(O)—, and 5- to 14-membered heteroaryloxy; and
R⁵ is, at each occurrence, independently selected from amino, hydroxy, C₁-C₆-alkyl, amino-C₁-C₆-alkyl-, hydroxy-C₁-C₆-alkyl-, halo-C₁-C₆-alkyl-, C₁-C₆-alkoxy, halo-C₁-C₆-alkoxy, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen (fluoro), cyano, C₁-C₆-aminoalkyl-S(O)₂—, and a group

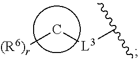

R⁶ is at each occurrence, independently selected from amino, hydroxy, C₁-C₆-alkyl, amino-C₁-C₆-alkyl-, hydroxy-C₁-C₆-alkyl-, halo-C₁-C₆-alkyl-, C₁-C₆-alkoxy, halo-C₁-C₆-alkoxy, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, cyano, and C₁-C₆-aminoalkyl-S(O)₂—;
A is 3- to 14-membered heterocyclyl;
B and C are independently selected from 3- to 14-membered heterocyclyl, C₃-C₁₀-cycloalkyl, 5- to 14-membered heteroaryl, and C₆-C₁₀-aryl;
L¹ and L³ are independently selected from a covalent bond, —O—, —NH—, —N(C₁-C₆-alkyl), —CH₂O—, —OCH₂—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—;
L² is selected from a covalent bond, carbonyl, —S(O)₂—, —NHC(O)—, —C(O)NH—, and —S(O)₂NH—;
m is 1, 2, 3, or 4;
n is 0, 1, 2, 3, or 4;
p is 0, 1, 2, 3, 4, or 5;
q is 0, 1, or 2;
r is 0 or 1; and
s is 0, 1, 2, 3, or 4.

In a preferred embodiment, the present invention provides a compound of formula (I) as described herein, or a pharmaceutically acceptable salt thereof, wherein m is 1.

In one embodiment, the present invention provides a compound of formula (I) as described herein, or a pharmaceutically acceptable salt thereof, wherein R¹ is selected from amino, amino-C₁-C₆-alkoxy-, a group

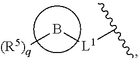

and a group C₁-C₆-alkyl-L²—; wherein: C₁-C₆-alkyl is substituted with 1-2 substituents selected from hydroxy, amino, cyano, C₁-C₆-alkyl-NH—, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy; and
wherein R⁵, q, B, L¹ and L² are as defined herein.

In a preferred embodiment, the present invention provides a compound of formula (I) as described herein, or a pharmaceutically acceptable salt thereof, wherein R¹ is a group

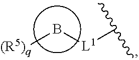

wherein R⁵, q, B, L¹ and L² are as defined herein.

In one embodiment, the present invention provides a compound of formula (I) as described herein, or a pharmaceutically acceptable salt thereof, wherein L¹ is selected from —CH₂O—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—.

In a preferred embodiment, the present invention provides a compound of formula (I) as described herein, or a pharmaceutically acceptable salt thereof, wherein L¹ is selected from carbonyl, —CH₂C(O)—, —CH₂NHC(O)—, and —NHC(O)—.

In a preferred embodiment, the present invention provides a compound of formula (I) as described herein, or a pharmaceutically acceptable salt thereof, wherein L² is selected from a covalent bond, carbonyl, —S(O)₂—, —NHC(O)—, —C(O)NH—, and —S(O)₂NH—.

In one embodiment, the present invention provides a compound of formula (I) as described herein, or a pharmaceutically acceptable salt thereof, wherein q is 0, 1, or 2.

In a preferred embodiment, the present invention provides a compound of formula (I) as described herein, or a pharmaceutically acceptable salt thereof, wherein q is 0 or 1.

In one embodiment, the present invention provides a compound of formula (I) as described herein, or a pharmaceutically acceptable salt thereof, wherein s is 0, 1, or 4.

In one embodiment, the present invention provides a compound of formula (I) as described herein, or a pharmaceutically acceptable salt thereof, wherein B is selected from 3- to 14-membered heterocyclyl, C₃-C₁₀-cycloalkyl, and 5- to 14-membered heteroaryl.

In a preferred embodiment, the present invention provides a compound of formula (I) as described herein, or a pharmaceutically acceptable salt thereof, wherein B is a 3- to 14-membered heterocyclyl.

In a particularly preferred embodiment, the present invention provides a compound of formula (I) as described herein, or a pharmaceutically acceptable salt thereof, wherein B is selected from azetidinyl, pyrrolidinyl, 3-azabicyclo[3.1.0]hexanyl, and piperidyl.

In one embodiment, the present invention provides a compound of formula (I) as described herein, or a pharmaceutically acceptable salt thereof, wherein R⁵ is, at each occurrence, independently selected from amino, hydroxy, C₁-C₆-alkyl, amino-C₁-C₆-alkyl-, hydroxy-C₁-C₆-alkyl-, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, aminoalkyl-S(O)₂—, and a group

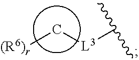

wherein R⁶, r, C and L³ are as defined herein.

In a preferred embodiment, the present invention provides a compound of formula (I) as described herein, or a pharmaceutically acceptable salt thereof, wherein R⁵ is, at each occurrence, independently selected from amino, hydroxy, and hydroxy-C₁-C₆-alkyl-.

In a particularly preferred embodiment, the present invention provides a compound of formula (I) as described herein, or a pharmaceutically acceptable salt thereof, wherein R⁵ is, at each occurrence, independently selected from amino, hydroxy, and hydroxymethyl.

In one embodiment, the present invention provides a compound of formula (I) as described herein, or a pharmaceutically acceptable salt thereof, wherein L³ is a covalent bond or carbonyl.

In one embodiment, the present invention provides a compound of formula (I) as described herein, or a pharmaceutically acceptable salt thereof, wherein r is 0 or 1.

In one embodiment, the present invention provides a compound of formula (I) as described herein, or a pharmaceutically acceptable salt thereof, wherein C is C₃-C₁₀-cycloalkyl or 3- to 14-membered heterocyclyl.

In one embodiment, the present invention provides a compound of formula (I) as described herein, or a pharmaceutically acceptable salt thereof, wherein R⁶ is hydroxy.

In one embodiment, the present invention provides a compound of formula (I) as described herein, or a pharmaceutically acceptable salt thereof, wherein:
m is 1; and
R¹ is selected from amino, amino-C₁-C₆-alkoxy-, a group

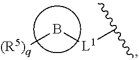

and a group C₁-C₆-alkyl-L²—; wherein:
   C₁-C₆-alkyl is substituted with 1-2 substituents selected from hydroxy, amino, cyano, C₁-C₆-alkyl-NH—, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy;
   L¹ is selected from —CH₂O—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—;
   L² is selected from a covalent bond, carbonyl, —S(O)₂—, —NHC(O)—, —C(O)NH—, and —S(O)₂NH—;
   q is 0, 1, or 2;
   s is 0, 1, or 4;
   B is selected from 3- to 14-membered heterocyclyl, C₃-C₁₀-cycloalkyl, and 5- to 14-membered heteroaryl; and
   R⁵ is, at each occurrence, independently selected from amino, hydroxy, C₁-C₆-alkyl, amino-C₁-C₆-alkyl-, hydroxy-C₁-C₆-alkyl-, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, aminoalkyl-S(O)₂—, and a group

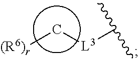

wherein:
   L³ is a covalent bond or carbonyl;
   r is 0 or 1;
   C is C₃-C₁₀-cycloalkyl or 3- to 14-membered heterocyclyl; and R⁶ is hydroxy.

In a preferred embodiment, the present invention provides a compound of formula (I) as described herein, or a pharmaceutically acceptable salt thereof, wherein:
m is 1; and
R¹ is a group

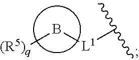

wherein:
   L¹ is selected from carbonyl, —CH₂C(O)—, —CH₂NHC(O)—, and —NHC(O)—;
   q is 0 or 1;
   B is 3- to 14-membered heterocyclyl; and
   R⁵ is selected from amino, hydroxy, and hydroxy-C₁-C₆-alkyl-.

In a particularly preferred embodiment, the present invention provides a compound of formula (I) as described herein, or a pharmaceutically acceptable salt thereof, wherein:
m is 1; and

R¹ is a group

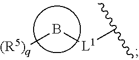

wherein:
   L¹ is selected from carbonyl, —CH₂C(O)—, —CH₂NHC(O)—, and —NHC(O)—;
   q is 0 or 1;
   B is selected from azetidinyl, pyrrolidinyl, 3-azabicyclo[3.1.0]hexanyl, and piperidyl; and
   R⁵ is selected from amino, hydroxy, and hydroxymethyl-.

In a preferred embodiment, the present invention provides a compound of formula (I) as described herein, or a pharmaceutically acceptable salt thereof, wherein n is 1 and R² is selected from halogen and C₁-C₆-alkyl.

In a particularly preferred embodiment, the present invention provides a compound of formula (I) as described herein, or a pharmaceutically acceptable salt thereof, wherein n is 1 and R² is selected from chloro and methyl.

In one embodiment, the present invention provides a compound of formula (I) as described herein, or a pharmaceutically acceptable salt thereof, wherein the compound of formula (I) is a compound of formula (II):

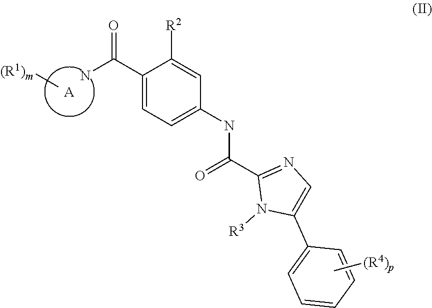

wherein R¹, R², R³, R⁴, m, and p are as defined herein.

In a preferred embodiment, the present invention provides a compound of formula (I) as described herein, or a pharmaceutically acceptable salt thereof, wherein R³ is C₁-C₆-alkyl.

In a particularly preferred embodiment, the present invention provides a compound of formula (I) as described herein, or a pharmaceutically acceptable salt thereof, wherein R³ is methyl.

In one embodiment, the present invention provides a compound of formula (I) as described herein, or a pharmaceutically acceptable salt thereof, wherein p is 1, 2, 3 or 4 and R⁴ is, at each occurrence, independently selected from halogen, C₁-C₆-alkyl, C₁-C₆-alkoxy, cyano, halo-C₁-C₆-alkyl, cyano-C₁-C₆-alkyl, (C₁-C₆-alkyl)₂N—, halo-C₁-C₆-alkoxy, cyano-C₁-C₆-alkoxy, C₁-C₆-alkoxy-C₁-C₆-alkoxy-, (C₁-C₆-alkyl)₂N—C(O)—, and heteroaryloxy.

In a preferred embodiment, the present invention provides a compound of formula (I) as described herein, or a pharmaceutically acceptable salt thereof, wherein p is 2 or 3 and R⁴ is, at each occurrence, independently selected from halogen, C₁-C₆-alkoxy, halo-C₁-C₆-alkoxy, and cyano-C₁-C₆-alkoxy.

In a particularly preferred embodiment, the present invention provides a compound of formula (I) as described herein, or a pharmaceutically acceptable salt thereof, wherein p is 2 or 3 and R⁴ is, at each occurrence, independently selected from fluoro, chloro, methoxy, FCH₂O—, F₂CHO— and CNCH₂O—.

In one embodiment, the present invention provides a compound of formula (I) as described herein, or a pharmaceutically acceptable salt thereof, wherein the compound of formula (I) is a compound of formula (III):

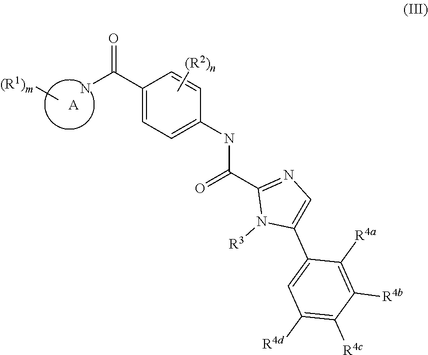

wherein:
R^{4a} is selected from hydrogen, halogen, C₁-C₆-alkyl, cyano, and halo-C₁-C₆-alkyl;
R^{4b} is selected from hydrogen, halogen, cyano, and C₁-C₆-alkoxy;
R^{4c} is selected from halogen, C₁-C₆-alkoxy, cyano-C₁-C₆-alkyl, cyano-C₁-C₆-alkoxy, (C₁-C₆-alkyl)₂N—, halo-C₁-C₆-alkoxy, C₁-C₆-alkoxy-C₁-C₆-alkoxy, (C₁-C₆-alkyl)₂N—C(O)—, and heteroaryloxy;
R^{4d} is selected from hydrogen and halogen; and
wherein R¹, R², R³, m, and n are as defined herein.

In a preferred embodiment, the present invention provides a compound of formula (III) as described herein, or a pharmaceutically acceptable salt thereof, wherein:
R^{4a} is halogen;
R^{4b} is selected from hydrogen and halogen;
R^{4c} is selected from C₁-C₆-alkoxy, cyano-C₁-C₆-alkoxy, and halo-C₁-C₆-alkoxy; and
R^{4d} is hydrogen.

In a particularly preferred embodiment, the present invention provides a compound of formula (III) as described herein, or a pharmaceutically acceptable salt thereof, wherein:
R^{4a} is selected from fluoro and chloro;
R^{4b} is selected from hydrogen, fluoro and chloro;
R^{4c} is selected from methoxy, FCH₂O—, F₂CHO— and CNCH₂O—; and
R^{4d} is hydrogen.

In one embodiment, the present invention provides a compound of formula (I) as described herein, or a pharmaceutically acceptable salt thereof, wherein:
m is 1; and
R¹ is selected from amino, amino-C₁-C₆-alkoxy-, a group

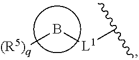

and a group C₁-C₆-alkyl-L²—; wherein:
   C₁-C₆-alkyl is substituted with 1-2 substituents selected from hydroxy, amino, cyano, C₁-C₆-alkyl-NH—, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy;
   L¹ is selected from —CH₂O—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—;
   L² is selected from a covalent bond, carbonyl, —S(O)₂—, —NHC(O)—, —C(O)NH—, and —S(O)₂NH—;
   q is 0, 1, or 2;
   s is 0, 1, or 4;
   B is selected from 3- to 14-membered heterocyclyl, C₃-C₁₀-cycloalkyl, and 5- to 14-membered heteroaryl; and
   R⁵ is, at each occurrence, independently selected from amino, hydroxy, C₁-C₆-alkyl, amino-C₁-C₆-alkyl-, hydroxy-C₁-C₆-alkyl-, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, aminoalkyl-S(O)₂—, and a group

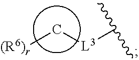

wherein:
   L³ is a covalent bond or carbonyl;
   r is 0 or 1;
   C is C₃-C₁₀-cycloalkyl or 3- to 14-membered heterocyclyl;
   R⁶ is hydroxy;
n is 1;
R² is selected from halogen and C₁-C₆-alkyl;
R³ is C₁-C₆-alkyl;
p is 1, 2, 3 or 4; and
R⁴ is, at each occurrence, independently selected from halogen, C₁-C₆-alkyl, C₁-C₆-alkoxy, cyano, halo-C₁-C₆-alkyl, cyano-C₁-C₆-alkyl, (C₁-C₆-alkyl)₂N—, halo-C₁-C₆-alkoxy, cyano-C₁-C₆-alkoxy, C₁-C₆-alkoxy-C₁-C₆-alkoxy-, (C₁-C₆-alkyl)₂N—C(O)—, and heteroaryloxy.

In a preferred embodiment, the present invention provides a compound of formula (I) as described herein, or a pharmaceutically acceptable salt thereof, wherein:
m is 1; and
R¹ is a group

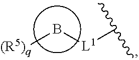

wherein:
   L¹ is selected from carbonyl, —CH₂C(O)—, —CH₂NHC(O)—, and —NHC(O)—;
   q is 0 or 1;
   B is 3- to 14-membered heterocyclyl; and
   R⁵ is selected from amino, hydroxy, and hydroxy-C₁-C₆-alkyl-
n is 1;
R² is selected from halogen and C₁-C₆-alkyl;
R³ is C₁-C₆-alkyl;
p is 2 or 3; and
R⁴ is, at each occurrence, independently selected from halogen, C₁-C₆-alkoxy, halo-C₁-C₆-alkoxy, and cyano-C₁-C₆-alkoxy.

In a particularly preferred embodiment, the present invention provides a compound of formula (I) as described herein, or a pharmaceutically acceptable salt thereof, wherein:
m is 1; and

R¹ is a group

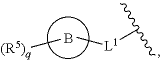

wherein:
   L¹ is selected from carbonyl, —CH₂C(O)—, —CH₂NHC(O)—, and —NHC(O)—;
   q is 0 or 1;
   B is selected from azetidinyl, pyrrolidinyl, 3-azabicyclo[3.1.0]hexanyl, and piperidyl; and
   R⁵ is selected from amino, hydroxy, and hydroxymethyl-;
n is 1;
R² is selected from chloro and methyl;
R³ is methyl;
p is 2 or 3; and
R⁴ is, at each occurrence, independently selected from fluoro, chloro, methoxy, FCH₂O—, F₂CHO— and CNCH₂O—.

In one embodiment, the present invention provides a compound of formula (I) as described herein, or a pharmaceutically acceptable salt thereof, wherein the compound of formula (I) is selected from:
N-[3-chloro-4-[4-[(3R)-pyrrolidine-3-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(3S)-pyrrolidine-3-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,4S)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-(piperidine-4-carbonyl)piperazine-1-carbonyl]phenyl]-5-[4-(cyanomethoxy)-2,3-difluoro-phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,3S)-3-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[3-(dimethylamino)propyl]piperazine-1-carbonyl]phenyl]-5-[4-(cyanomethoxy)-2,3-difluoro-phenyl]-1-methyl-imidazole-2-carboxamide;
1-[2-chloro-4-[[5-[4-(cyanomethoxy)-2,3-difluoro-phenyl]-1-methyl-imidazole-2-carbonyl]amino]benzoyl]-N-[2-(methylamino)ethyl]piperidine-4-carboxamide;
N-[4-[4-[(3R)-3-aminopyrrolidine-1-carbonyl]piperidine-1-carbonyl]-3-chloro-phenyl]-5-[4-(cyanomethoxy)-2,3-difluoro-phenyl]-1-methyl-imidazole-2-carboxamide;
N-[3-chloro-4-[4-(4-hydroxypiperidine-4-carbonyl)piperazine-1-carbonyl]phenyl]-5-[4-(cyanomethoxy)-2,3-difluoro-phenyl]-1-methyl-imidazole-2-carboxamide;
N-[4-[4-[2-(aminomethyl)morpholine-4-carbonyl]piperidine-1-carbonyl]-3-chloro-phenyl]-5-[4-(cyanomethoxy)-2,3-difluoro-phenyl]-1-methyl-imidazole-2-carboxamide;
N-[3-chloro-4-[4-[(3R)-3-(hydroxymethyl)piperazine-1-carbonyl]piperidine-1-carbonyl]phenyl]-5-[4-(cyanomethoxy)-2,3-difluoro-phenyl]-1-methyl-imidazole-2-carboxamide;
N-[3-chloro-4-[4-[2-(dimethylamino)ethyl]piperazine-1-carbonyl]phenyl]-5-(2,3-difluoro-4-methoxy-phenyl)-1-methyl-imidazole-2-carboxamide;
N-[4-[4-[(2S,4S)-4-aminopyrrolidine-2-carbonyl]piperazine-1-carbonyl]-3-chloro-phenyl]-5-[4-(cyanomethoxy)-2,3-difluoro-phenyl]-1-methyl-imidazole-2-carboxamide;
N-[3-chloro-4-[4-[(2S)-pyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[2-(dimethylamino)acetyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S)-piperidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(3S)-piperidine-3-carbonyl]piperazine-1-carbonyl]phenyl]-5-[4-(cyanomethoxy)-2,3-difluoro-phenyl]-1-methyl-imidazole-2-carboxamide;
N-[4-[4-(2-aminoethyl)piperazine-1-carbonyl]-3-chloro-phenyl]-5-[4-(cyanomethoxy)-2,3-difluoro-phenyl]-1-methyl-imidazole-2-carboxamide;
N-[3-chloro-4-[4-[2-(methylamino)acetyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2R)-pyrrolidine-2-carbonyl]piperazine-1-carbonyl]phenyl]-5-[4-(cyanomethoxy)-2,3-difluoro-phenyl]-1-methyl-imidazole-2-carboxamide;
N-(3-amino-2-hydroxy-propyl)-1-[2-chloro-4-[[5-[4-(cyanomethoxy)-2,3-difluoro-phenyl]-1-methyl-imidazole-2-carbonyl]amino]benzoyl]piperidine-4-carboxamide;
5-[4-(cyanomethoxy)-2,3-difluoro-phenyl]-1-methyl-N-[3-methyl-4-[4-[(3R)-pyrrolidine-3-carbonyl]piperazine-1-carbonyl]phenyl]imidazole-2-carboxamide;
N-[3-chloro-4-[4-[(3S)-3-(hydroxymethyl)piperazine-1-carbonyl]piperidine-1-carbonyl]phenyl]-5-[4-(cyanomethoxy)-2,3-difluoro-phenyl]-1-methyl-imidazole-2-carboxamide;
N-[3-chloro-4-[4-(1,1-dioxo-1,4-thiazinane-4-carbonyl)piperidine-1-carbonyl]phenyl]-5-[4-(cyanomethoxy)-2,3-difluoro-phenyl]-1-methyl-imidazole-2-carboxamide;
5-[4-(cyanomethoxy)-2,3-difluoro-phenyl]-1-methyl-N-[3-methyl-4-[4-[2-(methylamino)acetyl]piperazine-1-carbonyl]phenyl]imidazole-2-carboxamide;
N-[3-chloro-4-[4-(morpholine-4-carbonyl)piperidine-1-carbonyl]phenyl]-5-[4-(cyanomethoxy)-2,3-difluoro-phenyl]-1-methyl-imidazole-2-carboxamide;
5-[4-(cyanomethoxy)-2,3-difluoro-phenyl]-1-methyl-N-[3-methyl-4-[4-(morpholine-4-carbonyl)piperidine-1-carbonyl]phenyl]imidazole-2-carboxamide;
N-[3-chloro-4-[4-(2,6-diazaspiro[3.3]heptane-2-carbonyl)piperidine-1-carbonyl]phenyl]-5-[4-(cyanomethoxy)-2,3-difluoro-phenyl]-1-methyl-imidazole-2-carboxamide;
5-[4-(cyanomethoxy)-2,3-difluoro-phenyl]-N-[4-[4-(1,1-dioxo-1,4-thiazinane-4-carbonyl)piperidine-1-carbonyl]-3-methyl-phenyl]-1-methyl-imidazole-2-carboxamide;
N-[4-[4-[(2R)-2-(aminomethyl)morpholine-4-carbonyl]piperidine-1-carbonyl]-3-methyl-phenyl]-5-[4-(cyanomethoxy)-2,3-difluoro-phenyl]-1-methyl-imidazole-2-carboxamide;
N-[3-chloro-4-[4-(thiomorpholine-4-carbonyl)piperidine-1-carbonyl]phenyl]-5-[4-(cyanomethoxy)-2,3-difluoro-phenyl]-1-methyl-imidazole-2-carboxamide;
N-[4-[4-(3-aminoazetidine-1-carbonyl)piperidine-1-carbonyl]-3-methyl-phenyl]-5-[4-(cyanomethoxy)-2,3-difluoro-phenyl]-1-methyl-imidazole-2-carboxamide;
N-[4-[4-(aminomethyl)piperidine-1-carbonyl]-3-chloro-phenyl]-5-(2,3-difluoro-4-methoxy-phenyl)-1-methyl-imidazole-2-carboxamide;
N-[3-chloro-4-[4-(5-hydroxypiperidine-3-carbonyl)piperazine-1-carbonyl]phenyl]-5-[4-(cyanomethoxy)-2,3-difluoro-phenyl]-1-methyl-imidazole-2-carboxamide;
N-(2-aminoethyl)-4-[2-chloro-4-[[5-[4-(cyanomethoxy)-2,3-difluoro-phenyl]-1-methyl-imidazole-2-carbonyl]amino]benzoyl]piperazine-1-carboxamide;
N-[3-chloro-4-(4-piperazin-1-ylsulfonylpiperidine-1-carbonyl)phenyl]-5-(2,3-difluoro-4-methoxy-phenyl)-1-methyl-imidazole-2-carboxamide;
N-[3-chloro-4-[4-[2-[[(2S)-pyrrolidine-2-carbonyl]amino]ethyl]piperazine-1-carbonyl]phenyl]-5-[4-(cyanomethoxy)-2,3-difluoro-phenyl]-1-methyl-imidazole-2-carboxamide;
N-[4-[4-[(2S)-2-(aminomethyl)morpholine-4-carbonyl]piperidine-1-carbonyl]-3-methyl-phenyl]-5-[4-(cyanomethoxy)-2,3-difluoro-phenyl]-1-methyl-imidazole-2-carboxamide;
N-[3-chloro-4-[4-(4-piperidylsulfonyl)piperazine-1-carbonyl]phenyl]-5-(2,3-difluoro-4-methoxy-phenyl)-1-methyl-imidazole-2-carboxamide;
N-[4-[4-[2-[(2-amino-2-oxo-ethyl)amino]ethyl]piperazine-1-carbonyl]-3-chloro-phenyl]-5-[4-(cyanomethoxy)-2,3-difluoro-phenyl]-1-methyl-imidazole-2-carboxamide;
(3R)-1-[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]pyrrolidine-3-carboxylic acid;
1-[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]azetidine-3-carboxylic acid;
5-[3-chloro-4-(cyanomethoxy)-2-fluoro-phenyl]-N-[3-chloro-4-[4-(piperidine-4-carbonyl)piperazine-1-carbonyl]phenyl]-1-methyl-imidazole-2-carboxamide;
N-[3-chloro-4-[4-(piperidine-4-carbonyl)piperazine-1-carbonyl]phenyl]-5-(2,3-difluoro-4-methoxy-phenyl)-1-methyl-imidazole-2-carboxamide;
N-[3-chloro-4-[4-[2-(dimethylamino)ethyl]piperazine-1-carbonyl]phenyl]-5-(3-fluoro-4-methoxy-phenyl)-1-methyl-imidazole-2-carboxamide;
N-[3-chloro-4-[4-[rac-(1R,5S)-3-azabicyclo[3.1.0]hexane-6-carbonyl]piperazine-1-carbonyl]phenyl]-5-[2,3-difluoro-4-(fluoromethoxy)phenyl]-1-methyl-imidazole-2-carboxamide;
N-[4-[4-(2-aminoethyl)piperidine-1-carbonyl]-3-chloro-phenyl]-5-(2,3-difluoro-4-methoxy-phenyl)-1-methyl-imidazole-2-carboxamide;
N-[3-chloro-4-[4-[(3R)-pyrrolidine-3-carbonyl]piperazine-1-carbonyl]phenyl]-5-(2,3-difluoro-4-methoxy-phenyl)-1-methyl-imidazole-2-carboxamide;
N-[4-[4-(3-aminocyclobutanecarbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-[(3S)-pyrrolidine-3-carbonyl]piperazine-1-carbonyl]phenyl]-5-(2,3-difluoro-4-methoxy-phenyl)-1-methyl-imidazole-2-carboxamide;
N-[4-[4-(3-aminocyclobutanecarbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(3-hydroxypyrrolidine-3-carbonyl)piperazine-1-carbonyl]phenyl]-5-(2,3-difluoro-4-methoxy-phenyl)-1-methyl-imidazole-2-carboxamide;
N-[3-chloro-4-[4-[2-(4-piperidyl)acetyl]piperazine-1-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-[2,3-difluoro-4-(fluoromethoxy)phenyl]-1-methyl-imidazole-2-carboxamide;
N-[3-chloro-4-[4-(piperidine-4-carbonyl)piperazine-1-carbonyl]phenyl]-5-[4-(difluoromethoxy)-3-fluoro-phenyl]-1-methyl-imidazole-2-carboxamide;
N-[4-[4-[2-(azetidin-3-yl)acetyl]piperazine-1-carbonyl]-3-chloro-phenyl]-5-(2,3-difluoro-4-methoxy-phenyl)-1-methyl-imidazole-2-carboxamide;
5-[2-chloro-3-fluoro-4-(fluoromethoxy)phenyl]-N-[3-chloro-4-[4-[2-[(3S)-pyrrolidin-3-yl]acetyl]piperazine-1-carbonyl]phenyl]-1-methyl-imidazole-2-carboxamide;
1-[2-chloro-4-[[5-[2-chloro-3-fluoro-4-(fluoromethoxy)phenyl]-1-methyl-imidazole-2-carbonyl]amino]benzoyl]-N-[(3R,4R)-4-hydroxypyrrolidin-3-yl]piperidine-4-carboxamide;
5-[2-chloro-4-(difluoromethoxy)-3-fluoro-phenyl]-N-[3-chloro-4-[4-[2-[(3S)-pyrrolidin-3-yl]acetyl]piperazine-1-carbonyl]phenyl]-1-methyl-imidazole-2-carboxamide;
N-[3-chloro-4-[4-(4-hydroxypyrrolidine-3-carbonyl)piperazine-1-carbonyl]phenyl]-5-[2,3-difluoro-4-(fluoromethoxy)phenyl]-1-methyl-imidazole-2-carboxamide;
1-[2-chloro-4-[[5-[2,3-difluoro-4-(fluoromethoxy)phenyl]-1-methyl-imidazole-2-carbonyl]amino]benzoyl]-N-[[(3R)-pyrrolidin-3-yl]methyl]piperidine-4-carboxamide;
N-[4-[4-(2-aminoacetyl)piperazine-1-carbonyl]-3-chloro-phenyl]-5-(2,3-difluoro-4-methoxy-phenyl)-1-methyl-imidazole-2-carboxamide;
N-[4-[4-[(2S)-azetidine-2-carbonyl]piperazine-1-carbonyl]-3-chloro-phenyl]-5-(2,3-difluoro-4-methoxy-phenyl)-1-methyl-imidazole-2-carboxamide;
N-[3-chloro-4-[4-(2-pyrrolidin-1-ylacetyl)piperazine-1-carbonyl]phenyl]-5-(2,3-difluoro-4-methoxy-phenyl)-1-methyl-imidazole-2-carboxamide;
N-[3-chloro-4-[4-(2-pyrrolidin-3-ylacetyl)piperazine-1-carbonyl]phenyl]-5-(2,3-difluoro-4-methoxy-phenyl)-1-methyl-imidazole-2-carboxamide;
N-[3-chloro-4-[4-[2-[(2R)-pyrrolidin-2-yl]acetyl]piperazine-1-carbonyl]phenyl]-5-(2,3-difluoro-4-methoxy-phenyl)-1-methyl-imidazole-2-carboxamide;
N-[3-chloro-4-[4-[2-[(3R)-pyrrolidin-3-yl]acetyl]piperazine-1-carbonyl]phenyl]-5-(2,3-difluoro-4-methoxy-phenyl)-1-methyl-imidazole-2-carboxamide;
N-[3-chloro-4-[4-[2-[(2S)-pyrrolidin-2-yl]acetyl]piperazine-1-carbonyl]phenyl]-5-(2,3-difluoro-4-methoxy-phenyl)-1-methyl-imidazole-2-carboxamide;
N-[3-chloro-4-[4-[(3R)-pyrrolidine-3-carbonyl]piperazine-1-carbonyl]phenyl]-5-[2,3-difluoro-4-(fluoromethoxy)phenyl]-1-methyl-imidazole-2-carboxamide;
N-[3-chloro-4-[4-[2-(dimethylamino)acetyl]piperazine-1-carbonyl]phenyl]-5-[4-(difluoromethoxy)-2,3-difluoro-phenyl]-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[[(3R)-pyrrolidin-3-yl]methyl]piperidine-4-carboxamide;
1-[2-chloro-4-[[5-(2,3-difluoro-4-methoxy-phenyl)-1-methyl-imidazole-2-carbonyl]amino]benzoyl]-N-[[(3S)-pyrrolidin-3-yl]methyl]piperidine-4-carboxamide;
N-[3-chloro-4-[4-[2-(3-hydroxypyrrolidin-1-yl)acetyl]piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3-hydroxyazetidin-3-yl)methyl]piperidine-4-carboxamide;
N-[3-chloro-4-[4-[2-[(2S)-pyrrolidin-2-yl]acetyl]piperazine-1-carbonyl]phenyl]-5-[2,3-difluoro-4-(fluoromethoxy)phenyl]-1-methyl-imidazole-2-carboxamide;
N-[3-chloro-4-[4-[2-[(3S)-pyrrolidin-3-yl]acetyl]piperazine-1-carbonyl]phenyl]-5-[2,3-difluoro-4-(fluoromethoxy)phenyl]-1-methyl-imidazole-2-carboxamide;
N-[3-chloro-4-[4-[2-[(2S)-pyrrolidin-2-yl]acetyl]piperazine-1-carbonyl]phenyl]-5-[4-(difluoromethoxy)-2-fluoro-phenyl)-1-methyl-imidazole-2-carboxamide;
N-[3-chloro-4-[4-[2-(3-hydroxyazetidin-3-yl)acetyl]piperazine-1-carbonyl]phenyl]-5-[2,3-difluoro-4-(fluoromethoxy)phenyl]-1-methyl-imidazole-2-carboxamide;
N-[3-chloro-4-[4-[2-(4-hydroxy-4-piperidyl)acetyl]piperazine-1-carbonyl]phenyl]-5-(2,3-difluoro-4-methoxy-phenyl)-1-methyl-imidazole-2-carboxamide;
N-[3-chloro-4-[4-(4-hydroxypiperidine-4-carbonyl)piperazine-1-carbonyl]phenyl]-5-[2,3-difluoro-4-(fluoromethoxy)phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-5-[4-(difluoromethoxy)-2,3-difluoro-phenyl]-1-methyl-imidazole-2-carboxamide;
N-[4-[4-[1-(2-aminoethyl)piperidine-4-carbonyl]piperazine-1-carbonyl]-3-chloro-phenyl]-5-(2,3-difluoro-4-methoxy-phenyl)-1-methyl-imidazole-2-carboxamide;
N-[3-chloro-4-[4-[2-(4-hydroxy-4-piperidyl)acetyl]piperazine-1-carbonyl]phenyl]-5-[2,3-difluoro-4-(fluoromethoxy)phenyl]-1-methyl-imidazole-2-carboxamide;
N-[4-[4-(azetidine-3-carbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(pyrrolidin-3-ylmethyl)piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3S)-pyrrolidin-3-yl]piperidine-4-carboxamide;
1-[2-chloro-4-[[5-(2,3-difluoro-4-methoxy-phenyl)-1-methyl-imidazole-2-carbonyl]amino]benzoyl]-N-[rac-(1S,5R)-3-azabicyclo[3.1.0]hexan-6-yl]piperidine-4-carboxamide;
N-[3-chloro-4-[4-(piperidine-4-carbonyl)piperazine-1-carbonyl]phenyl]-5-[4-(dimethylamino)-2,3-difluoro-phenyl]-1-methyl-imidazole-2-carboxamide;
N-[3-chloro-4-[4-(4-hydroxypiperidine-4-carbonyl)piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3R,4R)-4-hydroxypyrrolidin-3-yl]piperidine-4-carboxamide;
5-(2-chloro-3-fluoro-4-methoxy-phenyl)-N-[3-chloro-4-[4-[2-[(3S)-pyrrolidin-3-yl]acetyl]piperazine-1-carbonyl]phenyl]-1-methyl-imidazole-2-carboxamide;
N-[3-chloro-4-[4-(piperidine-4-carbonyl)piperazine-1-carbonyl]phenyl]-1-methyl-5-(2,3,5-trifluoro-4-methoxy-phenyl)imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[rac-(1S,5R)-3-azabicyclo[3.1.0]hexan-6-yl]piperidine-4-carboxamide;
N-[4-[3-(2-aminoethoxy)azetidine-1-carbonyl]-3-chloro-phenyl]-5-(2,3-difluoro-4-methoxy-phenyl)-1-methyl-imidazole-2-carboxamide;
1-[2-chloro-4-[[5-[2-chloro-3-fluoro-4-(fluoromethoxy)phenyl]-1-methyl-imidazole-2-carbonyl]amino]benzoyl]-N-[[(3R)-pyrrolidin-3-yl]methyl]piperidine-4-carboxamide;
5-[2-chloro-4-(difluoromethoxy)-3-fluoro-phenyl]-N-[3-chloro-4-[4-(4-hydroxypiperidine-4-carbonyl)piperazine-1-carbonyl]phenyl]-1-methyl-imidazole-2-carboxamide;
N-[3-chloro-4-[4-(4-hydroxypiperidine-4-carbonyl)piperazine-1-carbonyl]phenyl]-5-(4-ethoxy-2,3-difluoro-phenyl)-1-methyl-imidazole-2-carboxamide;
N-[3-chloro-4-[4-(5-hydroxypiperidine-3-carbonyl)piperazine-1-carbonyl]phenyl]-5-[2,3-difluoro-4-(fluoromethoxy)phenyl]-1-methyl-imidazole-2-carboxamide;
5-[2-chloro-4-(difluoromethoxy)-3-fluoro-phenyl]-N-[3-chloro-4-[4-[2-(4-hydroxy-4-piperidyl)acetyl]piperazine-1-carbonyl]phenyl]-1-methyl-imidazole-2-carboxamide;
5-[2-chloro-3-fluoro-4-(fluoromethoxy)phenyl]-N-[3-chloro-4-[4-[2-(4-hydroxy-4-piperidyl)acetyl]piperazine-1-carbonyl]phenyl]-1-methyl-imidazole-2-carboxamide;
N-[4-[4-[1-(azetidine-3-carbonyl)piperidine-4-carbonyl]piperazine-1-carbonyl]-3-chloro-phenyl]-5-[4-(difluoromethoxy)-2,3-difluoro-phenyl]-1-methyl-imidazole-2-carboxamide;
N-[3-chloro-4-[4-[2-(dimethylamino)acetyl]piperazine-1-carbonyl]phenyl]-5-[4-(difluoromethoxy)-2-fluoro-phenyl]-1-methyl-imidazole-2-carboxamide;
N-[3-chloro-4-[4-[2-(dimethylamino)acetyl]piperazine-1-carbonyl]phenyl]-5-[4-(difluoromethoxy)-3-fluoro-phenyl]-1-methyl-imidazole-2-carboxamide;
N-(azetidin-2-ylmethyl)-1-[2-chloro-4-[[5-(2,3-difluoro-4-methoxy-phenyl)-1-methyl-imidazole-2-carbonyl]amino]benzoyl]piperidine-4-carboxamide;
N-[3-chloro-4-[4-[2-(3-hydroxyazetidin-3-yl)acetyl]piperazine-1-carbonyl]phenyl]-5-(2,3-difluoro-4-methoxy-phenyl)-1-methyl-imidazole-2-carboxamide;
N-[4-[4-[2-(azetidin-3-yl)acetyl]piperazine-1-carbonyl]-3-chloro-phenyl]-5-[4-(difluoromethoxy)-2-fluoro-phenyl]-1-methyl-imidazole-2-carboxamide;
N-[3-chloro-4-[4-[1-(4-hydroxypiperidine-4-carbonyl)piperidine-4-carbonyl]piperazine-1-carbonyl]phenyl]-5-[4-(difluoromethoxy)-2,3-difluoro-phenyl]-1-methyl-imidazole-2-carboxamide;
N-[3-chloro-4-[4-(piperidine-4-carbonyl)piperazine-1-carbonyl]phenyl]-5-[4-(difluoromethoxy)-2-fluoro-phenyl]-1-methyl-imidazole-2-carboxamide;
N-[3-chloro-4-[4-[2-[(2S)-pyrrolidin-2-yl]acetyl]piperazine-1-carbonyl]phenyl]-5-[4-(difluoromethoxy)-3-fluoro-phenyl]-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3R)-pyrrolidin-3-yl]piperidine-4-carboxamide;
1-[2-chloro-4-[[5-[2-chloro-3-fluoro-4-(fluoromethoxy)phenyl]-1-methyl-imidazole-2-carbonyl]amino]benzoyl]-N-[(3S)-pyrrolidin-3-yl]piperidine-4-carboxamide;
5-(2-chloro-3-fluoro-4-methoxy-phenyl)-N-[3-chloro-4-[4-[2-(4-hydroxy-4-piperidyl)acetyl]piperazine-1-carbonyl]phenyl]-1-methyl-imidazole-2-carboxamide;
N-[3-chloro-4-[4-[2-(4-hydroxy-4-piperidyl)acetyl]piperazine-1-carbonyl]phenyl]-5-(4-ethoxy-2,3-difluoro-phenyl)-1-methyl-imidazole-2-carboxamide;
N-[4-[3-[3-(aminomethyl)azetidine-1-carbonyl]azetidine-1-carbonyl]-3-chloro-phenyl]-5-(2,3-difluoro-4-methoxy-phenyl)-1-methyl-imidazole-2-carboxamide;
3-[[1-[2-chloro-4-[[5-(2,3-difluoro-4-methoxy-phenyl)-1-methyl-imidazole-2-carbonyl]amino]benzoyl]piperidine-4-carbonyl]amino]propanoic acid;
4-[[1-[2-chloro-4-[[5-(2,3-difluoro-4-methoxy-phenyl)-1-methyl-imidazole-2-carbonyl]amino]benzoyl]piperidine-4-carbonyl]amino]butanoic acid;
N-[3-chloro-4-[4-(piperidine-4-carbonyl)piperazine-1-carbonyl]phenyl]-5-[4-(dimethylcarbamoyl)-2,3-difluoro-phenyl]-1-methyl-imidazole-2-carboxamide;
N-[3-chloro-4-[4-[rac-(1R,5S)-3-azabicyclo[3.1.0]hexane-6-carbonyl]piperazine-1-carbonyl]phenyl]-5-(2,3-difluoro-4-methoxy-phenyl)-1-methyl-imidazole-2-carboxamide;
N-[4-[4-[3-(aminomethyl)cyclobutanecarbonyl]piperazine-1-carbonyl]-3-chloro-phenyl]-5-(2,3-difluoro-4-methoxy-phenyl)-1-methyl-imidazole-2-carboxamide;
N-[4-[4-(2-amino-2-oxo-ethyl)piperazine-1-carbonyl]-3-chloro-phenyl]-5-(2,3-difluoro-4-methoxy-phenyl)-1-methyl-imidazole-2-carboxamide;
N-[3-chloro-4-[rac-(3aR,6aS)-5-(piperidine-4-carbonyl)-1,3,3a,4,6,6a-hexahydropyrrolo[3,4-c]pyrrole-2-carbonyl]phenyl]-5-(2,3-difluoro-4-methoxy-phenyl)-1-methyl-imidazole-2-carboxamide;
N-[3-chloro-4-[4-[2-(3-hydroxyazetidin-1-yl)acetyl]piperazine-1-carbonyl]phenyl]-5-(2,3-difluoro-4-methoxy-phenyl)-1-methyl-imidazole-2-carboxamide;
N-[1-[2-chloro-4-[[5-(2,3-difluoro-4-methoxy-phenyl)-1-methyl-imidazole-2-carbonyl]amino]benzoyl]azetidin-3-yl]piperidine-4-carboxamide;
N-[3-chloro-4-[3-(piperazine-1-carbonyl)azetidine-1-carbonyl]phenyl]-5-(2,3-difluoro-4-methoxy-phenyl)-1-methyl-imidazole-2-carboxamide;
N-[3-chloro-4-[rac-(3aR,6aS)-5-[rac-(3R)-pyrrolidine-3-carbonyl]-1,3,3a,4,6,6a-hexahydropyrrolo[3,4-c]pyrrole-2-carbonyl]phenyl]-5-(2,3-difluoro-4-methoxy-phenyl)-1-methyl-imidazole-2-carboxamide;
N-[3-chloro-4-[3-[[2-(dimethyl amino)acetyl]amino]azetidine-1-carbonyl]phenyl]-5-(2,3-difluoro-4-methoxy-phenyl)-1-methyl-imidazole-2-carboxamide;
N-[3-chloro-4-[3-[rac-(3aR,6aS)-2,3,3a,4,6,6a-hexahydro-1H-pyrrolo[3,4-c]pyrrole-5-carbonyl]azetidine-1-carbonyl]phenyl]-5-(2,3-difluoro-4-methoxy-phenyl)-1-methyl-imidazole-2-carboxamide;
N-[3-chloro-4-[rac-(3aS,6aR)-2-[2-(dimethylamino)acetyl]-1,3,3a,4,6,6a-hexahydropyrrolo[3,4-c]pyrrole-5-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-1-methyl-5-(2,3,4-trifluorophenyl)imidazole-2-carboxamide;
N-[3-chloro-4-[4-(piperidine-4-carbonyl)piperazine-1-carbonyl]phenyl]-5-(3-cyano-4-methoxy-phenyl)-1-methyl-imidazole-2-carboxamide;
N-[3-chloro-4-[3-[rac-(3aR,6aR)-2,3,3a,4,6,6a-hexahydro-1H-pyrrolo[3,4-b]pyrrole-5-carbonyl]pyrrolidine-1-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-(3-cyano-2,4-difluoro-phenyl)-1-methyl-imidazole-2-carboxamide;
N-[3-chloro-4-[4-[2-[(3S)-pyrrolidin-3-yl]acetyl]piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3S,4S)-4-hydroxypyrrolidin-3-yl]piperidine-4-carboxamide;
N-[3-chloro-4-[4-[[2-(dimethylamino)acetyl]amino]piperidine-1-carbonyl]phenyl]-5-(2,3-difluoro-4-methoxy-phenyl)-1-methyl-imidazole-2-carboxamide;
1-[4-[[5-(2,3-difluoro-4-methoxy-phenyl)-1-methyl-imidazole-2-carbonyl]amino]-2-ethyl-benzoyl]-N-[3-(prop-2-ynylamino)propyl]piperidine-4-carboxamide;
5-(2,3-difluoro-4-methoxy-phenyl)-N-[4-[4-[4-[3-(dimethylamino)propyl]piperazine-1-carbonyl]piperidine-1-carbonyl]-3-ethyl-phenyl]-1-methyl-imidazole-2-carboxamide;
N-(2-aminoethyl)-1-[4-[[5-(2,3-difluoro-4-methoxy-phenyl)-1-methyl-imidazole-2-carbonyl]amino]-2-ethyl-benzoyl]piperidine-4-carboxamide;
1-[4-[[5-(2,3-difluoro-4-methoxy-phenyl)-1-methyl-imidazole-2-carbonyl]amino]-2-ethyl-benzoyl]-N-[2-[2-(dimethylamino)ethoxy]ethyl]piperidine-4-carboxamide;
5-[4-(difluoromethoxy)phenyl]-N-[4-[4-[2-(dimethyl amino)ethyl]piperazine-1-carbonyl]-3-ethyl-phenyl]-1-methyl-imidazole-2-carboxamide;
5-(2-chloro-4-methoxy-phenyl)-N-[4-[4-[2-(dimethyl amino)ethyl]piperazine-1-carbonyl]-3-ethyl-phenyl]-1-methyl-imidazole-2-carboxamide;
N-[4-[4-[2-(dimethyl amino)ethyl]piperazine-1-carbonyl]-3-ethyl-phenyl]-5-(3-fluoro-4-isopropoxy-phenyl)-1-methyl-imidazole-2-carboxamide;
N-[4-[4-[(3-cyclobutyl-1,2,4-oxadiazol-5-yl)methyl]piperidine-1-carbonyl]-3-ethyl-phenyl]-5-(2,3-difluoro-4-methoxy-phenyl)-1-methyl-imidazole-2-carboxamide;
4-[2-chloro-4-[[5-[4-(cyanomethoxy)-2,3-difluoro-phenyl]-1-methyl-imidazole-2-carbonyl]amino]benzoyl]-N-[(3S,4S)-4-hydroxypyrrolidin-3-yl]piperazine-1-carboxamide;
4-[2-chloro-4-[[5-[4-(cyanomethoxy)-2,3-difluoro-phenyl]-1-methyl-imidazole-2-carbonyl]amino]benzoyl]-N-[(3R,4R)-4-hydroxypyrrolidin-3-yl]piperazine-1-carboxamide;
4-[2-chloro-4-[[5-(2,3-difluoro-4-methoxy-phenyl)-1-methyl-imidazole-2-carbonyl]amino]benzoyl]-N-[(3S,4S)-4-hydroxypyrrolidin-3-yl]piperazine-1-carboxamide;
N-[3-chloro-4-[4-[(3S,4S)-3-hydroxypiperidine-4-carbonyl]piperazine-1-carbonyl]phenyl]-5-(2,3-difluoro-4-methoxy-phenyl)-1-methyl-imidazole-2-carboxamide;
N-[3-chloro-4-[4-[(2S,4S)-4-hydroxy-4-methyl-pyrrolidine-2-carbonyl]piperazine-1-carbonyl]phenyl]-5-(2,3-difluoro-4-methoxy-phenyl)-1-methyl-imidazole-2-carboxamide;
N-[3-chloro-4-[4-[(3S,4R)-3-hydroxypiperidine-4-carbonyl]piperazine-1-carbonyl]phenyl]-5-(2,3-difluoro-4-methoxy-phenyl)-1-methyl-imidazole-2-carboxamide;
4-[4-[[5-(2,3-difluoro-4-methoxy-phenyl)-1-methyl-imidazole-2-carbonyl]amino]-2-methyl-benzoyl]-N-[(3S,4S)-4-hydroxypyrrolidin-3-yl]piperazine-1-carboxamide;
N-[3-chloro-4-[4-[(2S,4S)-4-ethyl-4-hydroxy-pyrrolidine-2-carbonyl]piperazine-1-carbonyl]phenyl]-5-(2,3-difluoro-4-methoxy-phenyl)-1-methyl-imidazole-2-carboxamide;
5-[4-(cyanomethoxy)-2,3-difluoro-phenyl]-N-[4-[4-[(2S,4S)-4-ethyl-4-hydroxy-pyrrolidine-2-carbonyl]piperazine-1-carbonyl]-3-methyl-phenyl]-1-methyl-imidazole-2-carboxamide;
5-[4-(cyanomethoxy)-2,3-difluoro-phenyl]-N-[4-[4-[(2S,4S)-4-hydroxy-4-methyl-pyrrolidine-2-carbonyl]piperazine-1-carbonyl]-3-methyl-phenyl]-1-methyl-imidazole-2-carboxamide;
N-[3-chloro-4-[4-[(3R,4S)-3-hydroxypiperidine-4-carbonyl]piperazine-1-carbonyl]phenyl]-5-(2,3-difluoro-4-methoxy-phenyl)-1-methyl-imidazole-2-carboxamide;
5-(2,3-difluoro-4-methoxy-phenyl)-N-[4-[4-[(3R,4R)-3-hydroxypiperidine-4-carbonyl]piperazine-1-carbonyl]-3-methyl-phenyl]-1-methyl-imidazole-2-carboxamide;
N-[3-chloro-4-[4-[(3R,4R)-3-hydroxypiperidine-4-carbonyl]piperazine-1-carbonyl]phenyl]-5-(2,3-difluoro-4-methoxy-phenyl)-1-methyl-imidazole-2-carboxamide;
4-[2-chloro-4-[[5-(2,3-difluoro-4-methoxy-phenyl)-1-methyl-imidazole-2-carbonyl]amino]benzoyl]-N-[(3R,4R)-4-hydroxypyrrolidin-3-yl]piperazine-1-carboxamide;
N-[3-chloro-4-(piperazine-1-carbonyl)phenyl]-5-[4-(cyanomethoxy)-2,3-difluoro-phenyl]-1-methyl-imidazole-2-carboxamide;
N-[3-chloro-4-[4-[(3S,4R)-3-hydroxypiperidine-4-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-(4-guanidinobutanoyl)piperazine-1-carbonyl]phenyl]-5-(2,3-difluoro-4-methoxy-phenyl)-1-methyl-imidazole-2-carboxamide;
N-[4-[4-(3-aminopropanoyl)piperazine-1-carbonyl]-3-chloro-phenyl]-5-(2,3-difluoro-4-methoxy-phenyl)-1-methyl-imidazole-2-carboxamide;
N-[4-[4-(5-aminopentanoyl)piperazine-1-carbonyl]-3-chloro-phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-[4-(difluoromethoxy)-2,3-difluoro-phenyl]-1-methyl-imidazole-2-carboxamide;
N-[3-chloro-4-[4-(3-cyanopropanoyl)piperazine-1-carbonyl]phenyl]-5-(2,3-difluoro-4-methoxy-phenyl)-1-methyl-imidazole-2-carboxamide;
N-[4-[4-(3-aminopropanoylamino)piperidine-1-carbonyl]-3-chloro-phenyl]-5-(2,3-difluoro-4-methoxy-phenyl)-1-methyl-imidazole-2-carboxamide;
N-(azetidin-3-yl)-1-[2-chloro-4-[[5-(2,3-difluoro-4-methoxy-phenyl)-1-methyl-imidazole-2-carbonyl]amino]benzoyl]piperidine-4-carboxamide;
N-[4-[4-[(1S,3R)-3-aminocyclopentanecarbonyl]piperazine-1-carbonyl]-3-chloro-phenyl]-5-(2,3-difluoro-4-methoxy-phenyl)-1-methyl-imidazole-2-carboxamide;
N-[4-[4-(2-aminoethyl sulfonyl amino)piperidine-1-carbonyl]-3-chloro-phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-(4-piperidyl)piperidine-4-carboxamide;
1-[2-chloro-4-[[5-(2,3-difluoro-4-methoxy-phenyl)-1-methyl-imidazole-2-carbonyl]amino]benzoyl]-N-(4-pyridylmethyl)piperidine-4-carboxamide;
5-[2-chloro-4-(cyanomethoxy)-3-fluoro-phenyl]-N-[3-chloro-4-[4-[(3R)-pyrrolidine-3-carbonyl]piperazine-1-carbonyl]phenyl]-1-methyl-imidazole-2-carboxamide;
5-[2-chloro-4-(cyanomethoxy)-3-fluoro-phenyl]-N-[3-chloro-4-[4-[(3S)-pyrrolidine-3-carbonyl]piperazine-1-carbonyl]phenyl]-1-methyl-imidazole-2-carboxamide;
1-[2-chloro-4-[[5-[2,3-difluoro-4-(fluoromethoxy)phenyl]-1-methyl-imidazole-2-carbonyl]amino]benzoyl]-N-[(3S,4R)-4-fluoropyrrolidin-3-yl]piperidine-4-carboxamide;
5-[3-chloro-2-fluoro-4-(fluoromethoxy)phenyl]-N-[3-chloro-4-[4-[(2S,4R)-4-hydroxypyrrolidine-2-carbonyl]piperazine-1-carbonyl]phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3S,4R)-4-fluoropyrrolidin-3-yl]piperidine-4-carboxamide;
1-[2-chloro-4-[[5-[4-(difluoromethoxy)-2,3-difluoro-phenyl]-1-methyl-imidazole-2-carbonyl]amino]benzoyl]-N-[[(3S)-pyrrolidin-3-yl]methyl]piperidine-4-carboxamide;
N-[4-[4-(2-aminoethoxy)piperidine-1-carbonyl]-3-chloro-phenyl]-5-(2,3-difluoro-4-methoxy-phenyl)-1-methyl-imidazole-2-carboxamide;
N-[4-[4-(azetidin-3-ylmethoxy)piperidine-1-carbonyl]-3-chloro-phenyl]-5-(2,3-difluoro-4-methoxy-phenyl)-1-methyl-imidazole-2-carboxamide;
N-[4-[4-(4-aminobutanoyl)piperazine-1-carbonyl]-3-chloro-phenyl]-5-(2,3-difluoro-4-methoxy-phenyl)-1-methyl-imidazole-2-carboxamide;
N-[3-chloro-4-[4-[2-(dimethyl amino)acetyl]piperazine-1-carbonyl]phenyl]-5-(2,3-difluoro-4-methoxy-phenyl)-1-methyl-imidazole-2-carboxamide;
N-[(2S)-2-aminopropyl]-1-[2-chloro-4-[[5-(2,3-difluoro-4-methoxy-phenyl)-1-methyl-imidazole-2-carbonyl]amino]benzoyl]piperidine-4-carboxamide;
N-[4-[4-[1-(2-aminoethyl)piperidine-4-carbonyl]piperazine-1-carbonyl]-3-chloro-phenyl]-5-[4-(difluoromethoxy)-2,3-difluoro-phenyl]-1-methyl-imidazole-2-carboxamide;
N-[3-chloro-4-[4-[1-[2-(dimethylamino)acetyl]piperidine-4-carbonyl]piperazine-1-carbonyl]phenyl]-5-(2,3-difluoro-4-methoxy-phenyl)-1-methyl-imidazole-2-carboxamide;
5-(2-chloro-3-fluoro-4-methoxy-phenyl)-N-[4-[4-(4-hydroxypiperidine-4-carbonyl)piperazine-1-carbonyl]-3-methyl-phenyl]-1-methyl-imidazole-2-carboxamide;
N-[4-[4-(4-aminopiperidine-1-carbonyl)piperidine-1-carbonyl]-3-chloro-phenyl]-5-(2,3-difluoro-4-methoxy-phenyl)-1-methyl-imidazole-2-carboxamide;
N-[3-chloro-4-[4-[2-[(3S)-pyrrolidin-3-yl]acetyl]piperazine-1-carbonyl]phenyl]-5-[2-(difluoromethyl)-3-fluoro-4-methoxy-phenyl]-1-methyl-imidazole-2-carboxamide;
N-[3-chloro-4-[4-(pyrrolidin-3-ylsulfonyl amino)piperidine-1-carbonyl]phenyl]-5-(2,3-difluoro-4-methoxy-phenyl)-1-methyl-imidazole-2-carboxamide;
N-[4-[4-[(3S)-3-aminopyrrolidine-1-carbonyl]piperidine-1-carbonyl]-3-chloro-phenyl]-5-[4-(difluoromethoxy)-2,3-difluoro-phenyl]-1-methyl-imidazole-2-carboxamide;
5-[2-chloro-4-(cyanomethoxy)-3-fluoro-phenyl]-N-[3-chloro-4-[4-(piperidine-4-carbonyl)piperazine-1-carbonyl]phenyl]-1-methyl-imidazole-2-carboxamide;
N-[4-[4-[(3R)-3-aminopyrrolidine-1-carbonyl]piperidine-1-carbonyl]-3-chloro-phenyl]-5-[4-(difluoromethoxy)-2,3-difluoro-phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[[(3R)-pyrrolidin-3-yl]methyl]piperidine-4-carboxamide;
N-[3-chloro-4-[4-(4-piperidylsulfonylamino)piperidine-1-carbonyl]phenyl]-5-(2,3-difluoro-4-methoxy-phenyl)-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3S,4S)-4-hydroxypyrrolidin-3-yl]piperidine-4-carboxamide;
N-[3-chloro-4-[4-[3-(dimethylamino)azetidine-1-carbonyl]piperidine-1-carbonyl]phenyl]-5-[4-(difluoromethoxy)-2,3-difluoro-phenyl]-1-methyl-imidazole-2-carboxamide;
5-[2-chloro-4-(cyanomethoxy)-3-fluoro-phenyl]-N-[3-chloro-4-[4-(4-hydroxypiperidine-4-carbonyl)piperazine-1-carbonyl]phenyl]-1-methyl-imidazole-2-carboxamide;
N-[4-[4-(3-carbamoylpyrrolidine-1-carbonyl)piperidine-1-carbonyl]-3-chloro-phenyl]-5-[4-(difluoromethoxy)-2,3-difluoro-phenyl]-1-methyl-imidazole-2-carboxamide;
N-(azetidin-3-ylmethyl)-1-[2-chloro-4-[[5-(2,3-difluoro-4-methoxy-phenyl)-1-methyl-imidazole-2-carbonyl]amino]benzoyl]piperidine-4-carboxamide;
N-[4-[4-[1-(2-amino-2-oxo-ethyl)piperidine-4-carbonyl]piperazine-1-carbonyl]-3-chloro-phenyl]-5-[4-(difluoromethoxy)-2,3-difluoro-phenyl]-1-methyl-imidazole-2-carboxamide;
N-[4-[4-[1-(2-aminoethyl sulfonyl)piperidine-4-carbonyl]piperazine-1-carbonyl]-3-chloro-phenyl]-5-(2,3-difluoro-4-methoxy-phenyl)-1-methyl-imidazole-2-carboxamide;
N-[3-chloro-4-[4-[(2S,4S)-4-hydroxypyrrolidine-2-carbonyl]piperazine-1-carbonyl]phenyl]-5-(2,3-difluoro-4-methoxy-phenyl)-1-methyl-imidazole-2-carboxamide;
N-(azetidin-3-ylmethyl)-1-[2-chloro-4-[[5-[4-(difluoromethoxy)-2,3-difluoro-phenyl]-1-methyl-imidazole-2-carbonyl]amino]benzoyl]piperidine-4-carboxamide;
N-[3-chloro-4-(4-methylsulfonylpiperazine-1-carbonyl)phenyl]-5-(2,3-difluoro-4-methoxy-phenyl)-1-methyl-imidazole-2-carboxamide;
N-[3-chloro-4-[4-(methanesulfonamido)piperidine-1-carbonyl]phenyl]-5-(2,3-difluoro-4-methoxy-phenyl)-1-methyl-imidazole-2-carboxamide;
N-[4-[4-(2-aminoethyl sulfonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(2-oxoimidazolidine-4-carbonyl)piperazine-1-carbonyl]phenyl]-difluoro-4-methoxy-phenyl)-1-methyl-imidazole-2-carboxamide;
N-[4-[4-[3-(2-aminoethyl)azetidine-1-carbonyl]piperidine-1-carbonyl]-3-chloro-phenyl]-5-(2,3-difluoro-4-methoxy-phenyl)-1-methyl-imidazole-2-carboxamide;
N-[3-chloro-4-[4-[(2-pyrrolidin-3-ylacetyl)amino]piperidine-1-carbonyl]phenyl]-difluoro-4-methoxy-phenyl)-1-methyl-imidazole-2-carboxamide;
N-[3-chloro-4-[4-[2-[(3S)-pyrrolidin-3-yl]acetyl]piperazine-1-carbonyl]phenyl]-5-[3-fluoro-4-(fluoromethoxy)-2-methyl-phenyl]-1-methyl-imidazole-2-carboxamide;
5-[3-chloro-2-fluoro-4-(fluoromethoxy)phenyl]-N-[4-[4-[(2S,4R)-4-hydroxypyrrolidine-2-carbonyl]piperazine-1-carbonyl]-3-methyl-phenyl]-1-methyl-imidazole-2-carboxamide;
N-[4-[4-[(3S,4S)-3-amino-4-fluoro-pyrrolidine-1-carbonyl]piperidine-1-carbonyl]-3-chloro-phenyl]-5-(2,3-difluoro-4-methoxy-phenyl)-1-methyl-imidazole-2-carboxamide;
N-[3-chloro-4-(4-pyrrolidin-3-ylsulfonylpiperazine-1-carbonyl)phenyl]-5-(2,3-difluoro-4-methoxy-phenyl)-1-methyl-imidazole-2-carboxamide;
N-[4-[4-(3-aminobicyclo[1.1.1]pentane-1-carbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-[[(1-methyl-4-piperidyl)amino]carbamoyl]piperidine-1-carbonyl]phenyl]-difluoro-4-(fluoromethoxy)phenyl]-1-methyl-imidazole-2-carboxamide;
N-[3-chloro-4-[4-[2-[(3S)-pyrrolidin-3-yl]acetyl]piperazine-1-carbonyl]phenyl]-5-(2-cyano-3-fluoro-4-methoxy-phenyl)-1-methyl-imidazole-2-carboxamide;
N-[3-chloro-4-[4-[2-(5-oxopyrrolidin-3-yl)acetyl]piperazine-1-carbonyl]phenyl]-5-(2,3-difluoro-4-methoxy-phenyl)-1-methyl-imidazole-2-carboxamide;
N-[3-chloro-4-[4-[3-(dimethylamino)propanoylamino]piperidine-1-carbonyl]phenyl]-5-(2,3-difluoro-4-methoxy-phenyl)-1-methyl-imidazole-2-carboxamide;
5-[2-chloro-4-(cyanomethoxy)-3-fluoro-phenyl]-N-[3-chloro-4-[4-[2-(dimethyl amino)ethyl]piperazine-1-carbonyl]phenyl]-1-methyl-imidazole-2-carboxamide;
5-[3-chloro-4-(cyanomethoxy)phenyl]-N-[3-chloro-4-[4-[2-(dimethyl amino)ethyl]piperazine-1-carbonyl]phenyl]-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-(4-piperidylmethyl)piperidine-4-carboxamide;
N-[4-[4-[3-(aminomethyl)azetidine-1-carbonyl]piperidine-1-carbonyl]-3-chloro-phenyl]-5-(2,3-difluoro-4-methoxy-phenyl)-1-methyl-imidazole-2-carboxamide;
N-[3-chloro-4-[4-[2-(methylamino)acetyl]piperazine-1-carbonyl]phenyl]-5-(2,3-difluoro-4-methoxy-phenyl)-1-methyl-imidazole-2-carboxamide;
N-[4-[4-(3-aminoazetidine-1-carbonyl)piperidine-1-carbonyl]-3-chloro-phenyl]-5-(2,3-difluoro-4-methoxy-phenyl)-1-methyl-imidazole-2-carboxamide;
N-[3-chloro-4-[4-[[(2S,4R)-4-hydroxypyrrolidine-2-carbonyl]amino]piperidine-1-carbonyl]phenyl]-5-(2,3-difluoro-4-methoxy-phenyl)-1-methyl-imidazole-2-carboxamide;
N-[4-[4-[3-[2-(2-aminoethoxy)ethoxy]propanoyl]piperazine-1-carbonyl]-3-chloro-phenyl]-5-(2,3-difluoro-4-methoxy-phenyl)-1-methyl-imidazole-2-carboxamide;
N-[3-chloro-4-[4-[2-(5-oxopyrrolidin-2-yl)acetyl]piperazine-1-carbonyl]phenyl]-5-(2,3-difluoro-4-methoxy-phenyl)-1-methyl-imidazole-2-carboxamide;
N-[3-chloro-4-[4-(5-oxopyrrolidine-2-carbonyl)piperazine-1-carbonyl]phenyl]-5-(2,3-difluoro-4-methoxy-phenyl)-1-methyl-imidazole-2-carboxamide;
5-(2-chloro-3-fluoro-4-methoxy-phenyl)-N-[3-chloro-4-[4-[(2S,4S)-4-hydroxypyrrolidine-2-carbonyl]piperazine-1-carbonyl]phenyl]-1-methyl-imidazole-2-carboxamide;
N-[4-(4-aminopiperidine-1-carbonyl)-3-chloro-phenyl]-5-(2,3-difluoro-4-methoxy-phenyl)-1-methyl-imidazole-2-carboxamide;
N-[3-chloro-4-[4-[2-(methyl amino)ethyl]piperazine-1-carbonyl]phenyl]-5-(2,3-difluoro-4-methoxy-phenyl)-1-methyl-imidazole-2-carboxamide;
N-[3-chloro-4-[4-(6-oxopiperidine-3-carbonyl)piperazine-1-carbonyl]phenyl]-5-(2,3-difluoro-4-methoxy-phenyl)-1-methyl-imidazole-2-carboxamide;
N-[4-[4-(2-azaspiro[3.3]heptane-6-carbonyl)piperazine-1-carbonyl]-3-methyl-phenyl]-5-(2-chloro-3-fluoro-4-methoxy-phenyl)-1-methyl-imidazole-2-carboxamide;
5-(2-chloro-3-fluoro-4-methoxy-phenyl)-N-[3-chloro-4-[4-[(3R)-3-(hydroxymethyl)piperazine-1-carbonyl]piperidine-1-carbonyl]phenyl]-1-methyl-imidazole-2-carboxamide;
5-(2-chloro-3-fluoro-4-methoxy-phenyl)-N-[3-chloro-4-[4-[(2R,4R)-4-hydroxypyrrolidine-2-carbonyl]piperazine-1-carbonyl]phenyl]-1-methyl-imidazole-2-carboxamide;
N-[3-chloro-4-[6-[(3R)-3-(hydroxymethyl)piperazine-1-carbonyl]-2-azaspiro[3.3]heptane-2-carbonyl]phenyl]-5-(2,3-difluoro-4-methoxy-phenyl)-1-methyl-imidazole-2-carboxamide;
N-[3-chloro-4-[4-[3-[2-[[2-(dimethylamino)acetyl]amino]ethoxy]propanoyl]piperazine-1-carbonyl]phenyl]-5-(2,3-difluoro-4-methoxy-phenyl)-1-methyl-imidazole-2-carboxamide;
5-(2-chloro-3-fluoro-4-methoxy-phenyl)-1-methyl-N-[3-methyl-4-[4-(piperidine-4-carbonyl)piperazine-1-carbonyl]phenyl]imidazole-2-carboxamide;
N-[3-chloro-4-[3-[[rac-(3R)-pyrrolidine-3-carbonyl]amino]pyrrolidine-1-carbonyl]phenyl]-5-(2,3-difluoro-4-methoxy-phenyl)-1-methyl-imidazole-2-carboxamide;
N-[3-chloro-4-(2,6-diazaspiro[3.3]heptane-2-carbonyl)phenyl]-5-(2,3-difluoro-4-methoxy-phenyl)-1-methyl-imidazole-2-carboxamide;
N-[(1S)-2-amino-1-methyl-ethyl]-1-[2-chloro-4-[[5-(2,3-difluoro-4-methoxy-phenyl)-1-methyl-imidazole-2-carbonyl]amino]benzoyl]piperidine-4-carboxamide;
N-[3-chloro-4-[4-[(3R)-3-(hydroxymethyl)piperazine-1-carbonyl]piperidine-1-carbonyl]phenyl]-5-(2,3-difluoro-4-methoxy-phenyl)-1-methyl-imidazole-2-carboxamide;
N-[1-[2-chloro-4-[[5-(2,3-difluoro-4-methoxy-phenyl)-1-methyl-imidazole-2-carbonyl]amino]benzoyl]pyrrolidin-3-yl]piperidine-4-carboxamide;
N-[3-chloro-4-[4-[(3S)-3-(hydroxymethyl)piperazine-1-carbonyl]piperidine-1-carbonyl]phenyl]-5-(2,3-difluoro-4-methoxy-phenyl)-1-methyl-imidazole-2-carboxamide;
N-[3-chloro-4-[2-[(2S,4R)-4-hydroxypyrrolidine-2-carbonyl]-2,6-diazaspiro[3.3]heptane-6-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-(2-fluoro-3,4-dimethoxy-phenyl)-1-methyl-imidazole-2-carboxamide;
N-[4-[4-[5-[(3aS,4S,6aR)-2-oxo-1,3,3a,4,6,6a-hexahydrothieno[3,4-d]imidazol-4-yl]pentanoyl]piperazine-1-carbonyl]-3-chloro-phenyl]-5-(2,3-difluoro-4-methoxy-phenyl)-1-methyl-imidazole-2-carboxamide;
N-[3-chloro-4-[4-[2-(2,5-dioxoimidazolidin-4-yl)acetyl]piperazine-1-carbonyl]phenyl]-5-(2,3-difluoro-4-methoxy-phenyl)-1-methyl-imidazole-2-carboxamide;
N-[4-[3-[3-(2-aminoethoxy)propanoylamino]pyrrolidine-1-carbonyl]-3-chloro-phenyl]-5-(2,3-difluoro-4-methoxy-phenyl)-1-methyl-imidazole-2-carboxamide;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]acetic acid;
N-[3-chloro-4-[4-(piperidine-4-carbonyl)piperazine-1-carbonyl]phenyl]-5-[2,3-difluoro-4-(2-methoxyethoxy)phenyl]-1-methyl-imidazole-2-carboxamide;
N-[3-chloro-4-[4-[2-(dimethylamino)ethyl]piperazine-1-carbonyl]phenyl]-5-[2,3-difluoro-4-(2-pyridyloxy)phenyl]-1-methyl-imidazole-2-carboxamide;
N-[3-chloro-4-[4-[2-(dimethylamino)ethyl]piperazine-1-carbonyl]phenyl]-5-[2,3-difluoro-4-(4-pyridyloxy)phenyl]-1-methyl-imidazole-2-carboxamide;
N-[3-chloro-4-[4-(piperidine-4-carbonyl)piperazine-1-carbonyl]phenyl]-5-(3,4-difluoro-5-methoxy-phenyl)-1-methyl-imidazole-2-carboxamide;
N-[3-chloro-4-[4-[2-(dimethylamino)ethyl]piperazine-1-carbonyl]phenyl]-5-(2,3-difluoro-4-pyrimidin-2-yloxy-phenyl)-1-methyl-imidazole-2-carboxamide;
N-[3-chloro-4-[4-(piperidine-4-carbonyl)piperazine-1-carbonyl]phenyl]-5-[4-(cyanomethyl)-2-fluoro-phenyl]-1-methyl-imidazole-2-carboxamide;
N-[4-[4-(4-hydroxypiperidine-4-carbonyl)piperazine-1-carbonyl]-3-methyl-phenyl]-9-methoxy-6,7-dihydro-5H-imidazo[5,1-a][2]benzazepine-3-carboxamide;
N-[3-chloro-4-[4-[(2S,4R)-4-hydroxypyrrolidine-2-carbonyl]piperazine-1-carbonyl]phenyl]-5-(2,3-difluoro-4-methoxy-phenyl)-1-methyl-imidazole-2-carboxamide;
N-(azetidin-3-ylmethyl)-1-[2-chloro-4-[[5-[4-(cyanomethoxy)-2,3-difluoro-phenyl]-1-methyl-imidazole-2-carbonyl]amino]benzoyl]piperidine-4-carboxamide;
5-(2-chloro-3-fluoro-4-methoxy-phenyl)-N-[3-chloro-4-[4-(4-hydroxypiperidine-4-carbonyl)piperazine-1-carbonyl]phenyl]-1-methyl-imidazole-2-carboxamide;
N-[4-[4-[3-(2-aminoethoxy)propanoyl]piperazine-1-carbonyl]-3-chloro-phenyl]-5-(2,3-difluoro-4-methoxy-phenyl)-1-methyl-imidazole-2-carboxamide;
N-[4-[4-[1-(2-amino-2-oxo-ethyl)piperidine-4-carbonyl]piperazine-1-carbonyl]-3-chloro-phenyl]-5-[2,3-difluoro-4-(fluoromethoxy)phenyl]-1-methyl-imidazole-2-carboxamide;
N-[4-[4-[(3R)-3-aminopyrrolidine-1-carbonyl]piperazine-1-carbonyl]-3-chloro-phenyl]-5-[2,3-difluoro-4-(fluoromethoxy)phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3S)-pyrrolidin-3-yl]piperidine-4-carboxamide;
N-[3-chloro-4-[4-[2-[(2S)-pyrrolidin-2-yl]acetyl]piperazine-1-carbonyl]phenyl]-5-[4-(difluoromethoxy)-2,3-difluoro-phenyl]-1-methyl-imidazole-2-carboxamide;
N-[3-chloro-4-[4-[2-(dimethylamino)acetyl]piperazine-1-carbonyl]phenyl]-5-[2,3-difluoro-4-(fluoromethoxy)phenyl]-1-methyl-imidazole-2-carboxamide;
N-[3-chloro-4-[4-(piperidine-4-carbonyl)piperazine-1-carbonyl]phenyl]-5-[2-fluoro-4-(fluoromethoxy)phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-5-[2,3-difluoro-4-(fluoromethoxy)phenyl]-1-methyl-imidazole-2-carboxamide;
N-(2-aminoethyl)-1-[2-chloro-4-[[5-[2,3-difluoro-4-(fluoromethoxy)phenyl]-1-methyl-imidazole-2-carbonyl]amino]benzoyl]piperidine-4-carboxamide;
N-(2-aminoethyl)-1-[2-chloro-4-[[5-[4-(difluoromethoxy)-2,3-difluoro-phenyl]-1-methyl-imidazole-2-carbonyl]amino]benzoyl]piperidine-4-carboxamide;
1-[2-chloro-4-[[5-[2,3-difluoro-4-(fluoromethoxy)phenyl]-1-methyl-imidazole-2-carbonyl]amino]benzoyl]-N-(4-piperidyl)piperidine-4-carboxamide;
1-[2-chloro-4-[[5-[4-(difluoromethoxy)-2,3-difluoro-phenyl]-1-methyl-imidazole-2-carbonyl]amino]benzoyl]-N-[(3R)-pyrrolidin-3-yl]piperidine-4-carboxamide;
1-[2-chloro-4-[[5-[4-(difluoromethoxy)-2,3-difluoro-phenyl]-1-methyl-imidazole-2-carbonyl]amino]benzoyl]-N-(4-piperidyl)piperidine-4-carboxamide;
1-[2-chloro-4-[[5-[4-(difluoromethoxy)-2,3-difluoro-phenyl]-1-methyl-imidazole-2-carbonyl]amino]benzoyl]-N-[(3S,4S)-4-hydroxypyrrolidin-3-yl]piperidine-4-carboxamide;
N-[3-chloro-4-[4-[2-(4-hydroxy-4-piperidyl)acetyl]piperazine-1-carbonyl]phenyl]-5-[4-(difluoromethoxy)-2,3-difluoro-phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3R,4R)-4-hydroxypyrrolidin-3-yl]piperidine-4-carboxamide;
N-[3-chloro-4-[4-[2-(dimethyl amino)acetyl]piperazine-1-carbonyl]phenyl]-5-[2-fluoro-4-(fluoromethoxy)phenyl]-1-methyl-imidazole-2-carboxamide;
1-[2-chloro-4-[[5-[2,3-difluoro-4-(fluoromethoxy)phenyl]-1-methyl-imidazole-2-carbonyl]amino]benzoyl]-N-[(3R)-pyrrolidin-3-yl]piperidine-4-carboxamide;
N-[4-[3-[[3-(aminomethyl)cyclobutanecarbonyl]amino]azetidine-1-carbonyl]-3-chloro-phenyl]-5-(2,3-difluoro-4-methoxy-phenyl)-1-methyl-imidazole-2-carboxamide;
N-[4-[3-[(2-aminoacetyl)amino]azetidine-1-carbonyl]-3-chloro-phenyl]-5-(2,3-difluoro-4-methoxy-phenyl)-1-methyl-imidazole-2-carboxamide;
N-[4-[4-(3-carbamoyl cyclobutanecarbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(3-hydroxycyclobutanecarbonyl)piperazine-1-carbonyl]phenyl]-5-(2,3-difluoro-4-methoxy-phenyl)-1-methyl-imidazole-2-carboxamide;
N-[3-chloro-4-[4-(3-methoxypropanoyl)piperazine-1-carbonyl]phenyl]-5-(2,3-difluoro-4-methoxy-phenyl)-1-methyl-imidazole-2-carboxamide;
N-[4-[4-[3-(3-amino-3-oxo-propoxy)propanoyl]piperazine-1-carbonyl]-3-chloro-phenyl]-5-(2,3-difluoro-4-methoxy-phenyl)-1-methyl-imidazole-2-carboxamide; and
N-[4-[4-(5-amino-5-oxo-pentanoyl)piperazine-1-carbonyl]-3-chloro-phenyl]-5-(2,3-difluoro-4-methoxy-phenyl)-1-methyl-imidazole-2-carboxamide.

In a preferred embodiment, the present invention provides a compound of formula (I) as described herein, or a pharmaceutically acceptable salt thereof, wherein the compound of formula (I) is selected from:
N-[3-chloro-4-[4-[rac-(1R,5S)-3-azabicyclo[3.1.0]hexane-6-carbonyl]piperazine-1-carbonyl]phenyl]-5-(2,3-difluoro-4-methoxy-phenyl)-1-methyl-imidazole-2-carboxamide;
5-[3-chloro-2-fluoro-4-(fluoromethoxy)phenyl]-N-[3-chloro-4-[4-[(2S,4R)-4-hydroxypyrrolidine-2-carbonyl]piperazine-1-carbonyl]phenyl]-1-methyl-imidazole-2-carboxamide;
N-[3-chloro-4-[4-(piperidine-4-carbonyl)piperazine-1-carbonyl]phenyl]-5-[4-(difluoromethoxy)-2,3-difluoro-phenyl]-1-methyl-imidazole-2-carboxamide;
N-(azetidin-3-ylmethyl)-1-[2-chloro-4-[[5-[4-(difluoromethoxy)-2,3-difluoro-phenyl]-1-methyl-imidazole-2-carbonyl]amino]benzoyl]piperidine-4-carboxamide;
N-[3-chloro-4-[4-(2-pyrrolidin-3-ylacetyl)piperazine-1-carbonyl]phenyl]-5-(2,3-difluoro-4-methoxy-phenyl)-1-methyl-imidazole-2-carboxamide;
N-[4-[4-[2-(azetidin-3-yl)acetyl]piperazine-1-carbonyl]-3-chloro-phenyl]-5-(2,3-difluoro-4-methoxy-phenyl)-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3R,4R)-4-hydroxypyrrolidin-3-yl]piperidine-4-carboxamide;
4-[2-chloro-4-[[5-(2,3-difluoro-4-methoxy-phenyl)-1-methyl-imidazole-2-carbonyl]amino]benzoyl]-N-[(3R,4R)-4-hydroxypyrrolidin-3-yl]piperazine-1-carboxamide;
1-[2-chloro-4-[[5-[4-(difluoromethoxy)-2,3-difluoro-phenyl]-1-methyl-imidazole-2-carbonyl]amino]benzoyl]-N-[(3S,4S)-4-hydroxypyrrolidin-3-yl]piperidine-4-carboxamide;
N-[3-chloro-4-[4-[2-[(3S)-pyrrolidin-3-yl]acetyl]piperazine-1-carbonyl]phenyl]-5-(2,3-difluoro-4-methoxy-phenyl)-1-methyl-imidazole-2-carboxamide;
5-[3-chloro-2-fluoro-4-(fluoromethoxy)phenyl]-N-[4-[4-[(2S,4R)-4-hydroxypyrrolidine-2-carbonyl]piperazine-1-carbonyl]-3-methyl-phenyl]-1-methyl-imidazole-2-carboxamide;
N-[3-chloro-4-[4-[(2S,4S)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,3S)-3-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
4-[2-chloro-4-[[5-[4-(cyanomethoxy)-2,3-difluoro-phenyl]-1-methyl-imidazole-2-carbonyl]amino]benzoyl]-N-[(3S,4S)-4-hydroxypyrrolidin-3-yl]piperazine-1-carboxamide;
N-[3-chloro-4-[4-(4-hydroxypiperidine-4-carbonyl)piperazine-1-carbonyl]phenyl]-5-[4-(cyanomethoxy)-2,3-difluoro-phenyl]-1-methyl-imidazole-2-carboxamide;
N-[3-chloro-4-[4-(piperidine-4-carbonyl)piperazine-1-carbonyl]phenyl]-5-[4-(cyanomethoxy)-2,3-difluoro-phenyl]-1-methyl-imidazole-2-carboxamide;
N-[4-[4-[2-(azetidin-3-yl)acetyl]piperazine-1-carbonyl]-3-chloro-phenyl]-5-[4-(difluoromethoxy)-2-fluoro-phenyl]-1-methyl-imidazole-2-carboxamide;
N-(azetidin-3-ylmethyl)-1-[2-chloro-4-[[5-(2,3-difluoro-4-methoxy-phenyl)-1-methyl-imidazole-2-carbonyl]amino]benzoyl]piperidine-4-carboxamide;
N-[3-chloro-4-[4-[2-[(2S)-pyrrolidin-2-yl]acetyl]piperazine-1-carbonyl]phenyl]-difluoro-4-methoxy-phenyl)-1-methyl-imidazole-2-carboxamide;
N-[3-chloro-4-[4-[(3R)-3-(hydroxymethyl)piperazine-1-carbonyl]piperidine-1-carbonyl]phenyl]-5-[4-(cyanomethoxy)-2,3-difluoro-phenyl]-1-methyl-imidazole-2-carboxamide;
4-[2-chloro-4-[[5-[4-(cyanomethoxy)-2,3-difluoro-phenyl]-1-methyl-imidazole-2-carbonyl]amino]benzoyl]-N-[(3R,4R)-4-hydroxypyrrolidin-3-yl]piperazine-1-carboxamide;
N-[4-[4-[(2S,4S)-4-aminopyrrolidine-2-carbonyl]piperazine-1-carbonyl]-3-chloro-phenyl]-5-[4-(cyanomethoxy)-2,3-difluoro-phenyl]-1-methyl-imidazole-2-carboxamide;
4-[2-chloro-4-[[5-(2,3-difluoro-4-methoxy-phenyl)-1-methyl-imidazole-2-carbonyl]amino]benzoyl]-N-[(3S,4S)-4-hydroxypyrrolidin-3-yl]piperazine-1-carboxamide;
N-[3-chloro-4-[4-[(3S,4R)-3-hydroxypiperidine-4-carbonyl]piperazine-1-carbonyl]phenyl]-5-[4-(cyanomethoxy)-2,3-difluoro-phenyl]-1-methyl-imidazole-2-carboxamide;
N-(azetidin-3-ylmethyl)-1-[2-chloro-4-[[5-[4-(cyanomethoxy)-2,3-difluoro-phenyl]-1-methyl-imidazole-2-carbonyl]amino]benzoyl]piperidine-4-carboxamide;
N-[3-chloro-4-[4-[(2S,4R)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-5-(2,3-difluoro-4-methoxy-phenyl)-1-methyl-imidazole-2-carboxamide;
N-[3-chloro-4-[4-[(3S,4R)-3-hydroxypiperidine-4-carbonyl]piperazine-1-carbonyl]phenyl]-5-(2,3-difluoro-4-methoxy-phenyl)-1-methyl-imidazole-2-carboxamide;
4-[4-[[5-(2,3-difluoro-4-methoxy-phenyl)-1-methyl-imidazole-2-carbonyl]amino]-2-methyl-benzoyl]-N-[(3S,4S)-4-hydroxypyrrolidin-3-yl]piperazine-1-carboxamide; and
5-(2-chloro-3-fluoro-4-methoxy-phenyl)-N-[4-[4-(4-hydroxypiperidine-4-carbonyl)piperazine-1-carbonyl]-3-methyl-phenyl]-1-methyl-imidazole-2-carboxamide.

In one embodiment, the present invention provides pharmaceutically acceptable salts of the compounds of formula (I) as described herein, especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I) as described herein (i.e., as “free bases” or “free acids”, respectively).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N a N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the compounds of the invention are shown in the following schemes. The skills required for carrying out the reactions and purifications of the resulting products are known to those skilled in the art. The substituents and indices used in the following description of the processes have the significance given herein before unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 3rd Edition, Richard C. Larock. John Wiley & Sons, New York, N.Y. 2018). We find it convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux temperature. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

All substituents, in particular, R² to R⁴ are as defined above and in the claims, unless otherwise indicated. Furthermore, and unless explicitly otherwise stated, all reactions, reaction conditions, abbreviations and symbols have the meanings well known to a person of ordinary skill in organic chemistry.

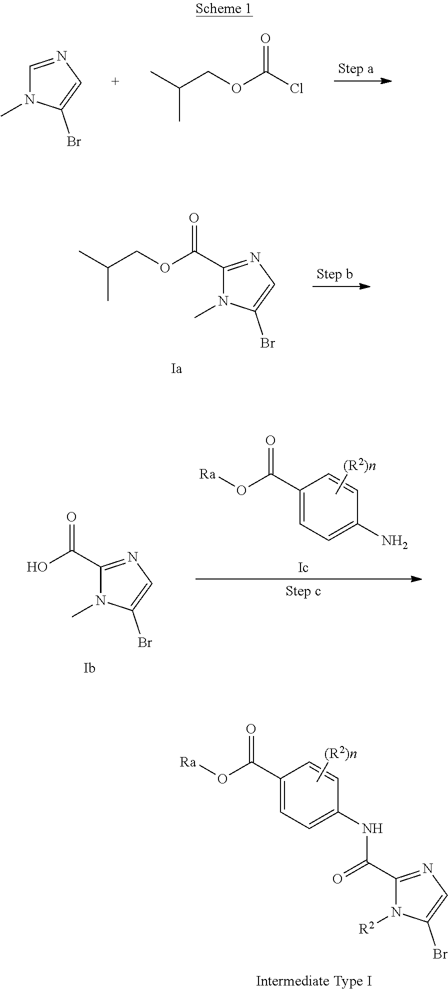

wherein Ra is alkyl, in particular Me, Et or iso-butyl.

Intermediates of Type I can be prepared according to Scheme 1. 5-bromo-1-methyl-imidazole can be acylated by isobutyl carbonochloridate under basic condition such as DIPEA in DCM to afford Ia (Step a). Acid Ib can be obtained by hydrolysis of Ia, using a suitable base and an appropriate solvent (e.g. K₂CO₃ in EtOH/water) at room temperature (Step b). Amide coupling of Ib and amine Ic with condensing agents, such as CDI, DCC, HATU, HBTU, T₃P in suitable solvent such as DMF, DMA or dioxane, optionally in the presence of a base, e.g. NEt3, DIPEA or DMAP affords Intermediates Type I (Step c).

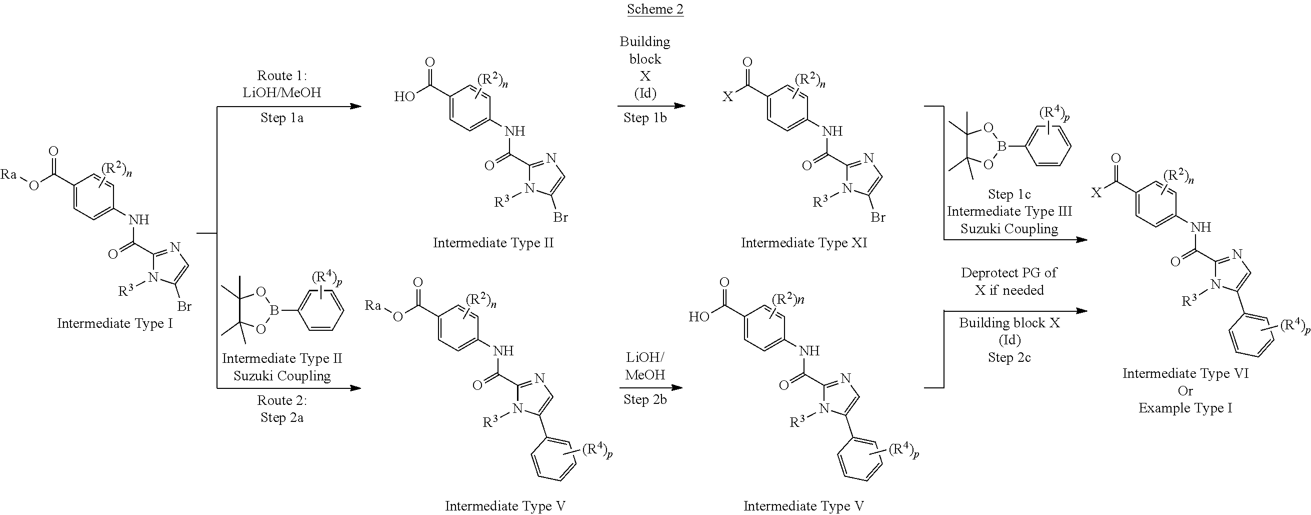

wherein Ra is alkyl, in particular Me, Et or iso-butyl.

Wherein “building block X” is a cyclic amine with or without PG, in which “PG” signifies a suitable protective group such as a Cbz or Boc protective group

Intermediate Type VI or Example Type I can be prepared according to route 1 in Scheme 2. Hydrolysis of the Intermediate Type I using a suitable base and an appropriate solvent (e.g. LiOH or NaOH in EtOH/water) at room temperature gives Intermediate Type II (Step 1a). Amide couplings of this intermediate with building block X with building block Y (Intermediate Type XIII), applying methods for example described under Scheme 3, Step 3b, followed by deprotection of the PG of Y (if needed) to give Intermediate Type IX or Example Type II, (Step 2a).

In route 3, the acid compound (Intermediate Type IV) can undergo amide coupling with building block X-Y (Intermediate Type VII), applying methods known in the art and for example described under Scheme 1, step C. Then followed by deprotection of the PG of X-Y (if needed) to give compound of formula Intermediate Type IX or Example Type II, applying methods known in the art and for example described under Scheme 2, step 1C, (Step 3a)

Compound of formula (Example Type III) can be prepared according to three routes outlined in Scheme 3.

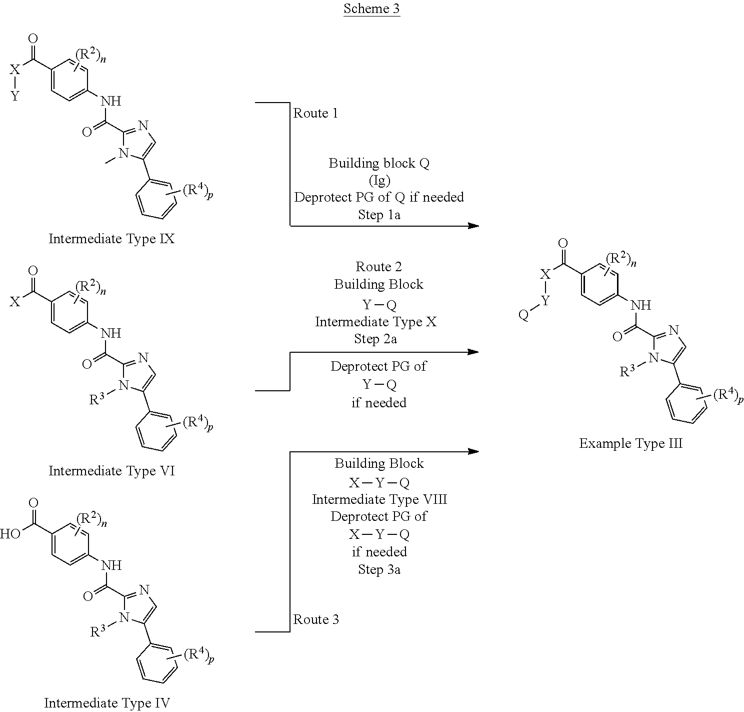

Wherein X is a cyclic amine with or without PG, in which “PG” signifies a suitable protective group such as a Cbz or Boc protective group

Wherein building block Q is an amine with or without PG, in which “PG” signifies a suitable protective group such as a Cbz or Boc protective group

Wherein building block Y-Q in Route 2 is an acid (Y)-amine (Q) compound with or without PG, in which “PG” signifies a suitable protective group such as a Cbz or Boc protective group.

Wherein building block X-Y-Q in Route 3 is an alkyl halide (Y) linked with two amines (X-cyclic amine and Q) compound with or without PG, in which “PG” signifies a suitable protective group such as a Cbz or Boc protective group.

In route 1, removal of the protective group (if needed) from Intermediate Type IX can be achieved by applying methods known in the art and for example described under Scheme 2, step 1C. Then amide coupling with building block Q (Ig) using methods for example described under Scheme 3, step 3b to give Example Type III (Step 1a).

In route 2, removal of the protective group from (if needed) Intermediate Type VI can be achieved by applying methods known in the art and for example described under Scheme 2, step 1C. Then amide coupling with building block Y-Q (Intermediate Type X) using methods for example described under Scheme 3, step 3b to give Example Type III, (Step 2a).

In route 3, the amide coupling between acid compound (Intermediate Type IV) and building block X-Y-Q (Intermediate Type VIII) can be achieved by using methods for example described under Scheme 3, step 3b. Then removal of the protective group (if needed) to give Example Type III, applying methods known in the art and for example described under Scheme 2, step 1C.

In one aspect, the present invention provides a process of manufacturing the compounds of formula (I) described herein, wherein said process is as described in any one of Schemes 1 to 5 above.

In a further aspect, the present invention provides a compound of formula (I) as described herein, or a pharmaceutically acceptable salt thereof, when manufactured according to the processes disclosed herein.

### Using the Compounds

As illustrated in the experimental section, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Acinetobacter species, most particularly by Acinetobacter baumannii.

The compounds of formula (I) and their pharmaceutically acceptable salts exhibit activity as antibiotics, particularly as antibiotics against Acinetobacter species, more particularly as antibiotics against Acinetobacter baumannii, most particularly as pathogen-specific antibiotics against Acinetobacter baumannii.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by Acinetobacter species, more particularly in the treatment and prevention of bacterial infections caused by Acinetobacter baumannii.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In one aspect, the present invention provides compounds of formula (I) or their pharmaceutically acceptable salts as described herein for use as therapeutically active substances.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a particular embodiment, said nosocomial infections and resulting diseases are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Gram-negative bacteria.

In a particular embodiment, said infections and resulting diseases caused by Gram-negative bacteria are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a particular embodiment, said infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, are selected from bacteremia, pneumonia, meningitis, urinary tract infection and wound infection, or a combination thereof.

In a further aspect, the present invention provides compounds of formula (I) or their pharmaceutically acceptable salts as defined above for use in the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides a method for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii, which method comprises administering a compound of formula (I) or a pharmaceutically acceptable salt thereof as defined above to a mammal.

In a further aspect, the present invention provides the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii. Such medicaments comprise compounds of formula (I) or their pharmaceutically acceptable salts as defined above.

### Pharmaceutical Compositions and Administration

In one aspect, the present invention provides pharmaceutical compositions comprising compounds of formula (I) or their pharmaceutically acceptable salts as defined above and one or more pharmaceutically acceptable excipients. Exemplary pharmaceutical compositions are described in Examples A-D.

In a further aspect, the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions or infusion solutions).

The compounds of formula (I) and their pharmaceutically acceptable salts can be processed with pharmaceutically inert, inorganic or organic excipients for the production of tablets, coated tablets, dragées and hard gelatin capsules. Lactose, corn starch or derivatives thereof, talc, stearic acid or its salts etc. can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with an antibiotic, in particular with an antibiotic for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including antibiotic agents. If the administration is not simultaneous, the compounds are administered in a close time proximity to each other. Furthermore, it does not matter if the compounds are administered in the same dosage form, e.g. one compound may be administered intravenously and another compound may be administered orally.

Typically, any agent that has antimicrobial activity may be co-administered. Particular examples of such agents are Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified e.g. in WO 2017072062 A1, WO 2019185572 A1 and WO 2019206853 A1, and Macrolides (erythromycin).

In one aspect, the present invention provides a pharmaceutical composition described herein, further comprising an additional therapeutic agent.

In one embodiment, said additional therapeutic agent is an antibiotic agent.

In one embodiment, said additional therapeutic agent is an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In one embodiment, said additional therapeutic agent is an antibiotic agent selected from Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified in WO 2017072062 A1, WO 2019185572 A1 and WO 2019206853 A1, and Macrolides (erythromycin).

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

Abbreviations used herein are as follows:
ACN or MeCN acetonitrile
BINAP 2,2′-Bis(diphenylphosphino)-1,1′-binaphthalene
CFU colony-forming, unit
d day
DCM dichloromethane
DIPEA N,N-diisopropylethylamine
EtOAc or EA ethyl acetate
FA formic acid
h(s) or hr(s) hour(s)
HATU: 1-[Bis(dimethylamino)methylene]-1H-1,2,3-triazolo[4,5-b]pyridinium 3-oxid hexafluorophosphate
HPLC: high performance liquid chromatography
HPLC-UV: high performance liquid chromatography with ultraviolet detector
IC50 half maximal inhibitory concentration
IC90 90% inhibitory concentration
PE petroleum ether
PdCl₂(DPPF) [1,1′-Bis(diphenylphosphino)ferrocene]dichloropalladium(II)
Pd₂(dba)₃ Tris(dibenzylideneacetone)dipalladium(0)
PG Protecting group
Precat precatalyst
prep-HPLC preparative high performance liquid chromatography
RBF Round bottom flask
rt room temperature
sat saturated
SEM 2-methoxyethyl(trimethyl)silane
FA Formic acid
TFA Trifluoroacetic Acid
wt weight
X-PHOS 2-Dicyclohexylphosphino-2′,4′,6′-triisopropylbiphenyl

### Intermediate Type I: 308

### Methyl 4-[(5-bromo-1-methyl-imidazole-2-carbonyl)amino]-2-chloro-benzoate

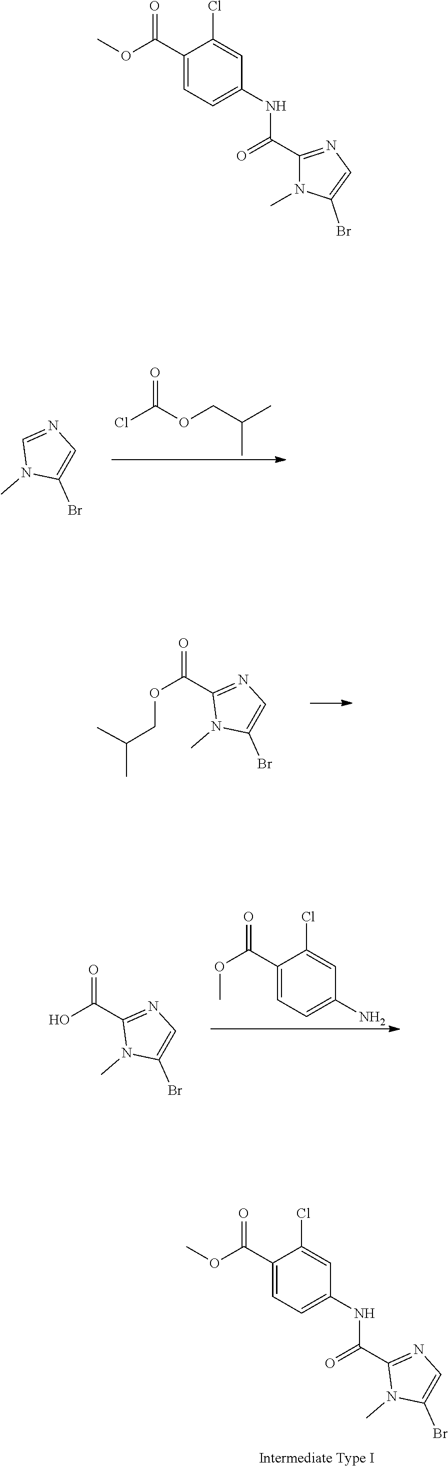

### Step 1: isobutyl 5-bromo-1-methyl-imidazole-2-carboxylate

To a solution of 5-bromo-1-methyl-imidazole (20 g, 124 mmol) and DIPEA (32.1 g, 43.4 mL, 248 mmol) in DCM (140 mL) at −70° C. was added slowly drop-wise within 30 min a solution of isobutyl carbonochloridate (22.1 g, 161 mmol) in DCM (60 mL). The mixture was stirred at −70° C. for 2 h. Then the mixture was slowly warmed to room temperature and stirred overnight. Then the solution was washed with water and concentrated in vacuo. The crude product was then purified by flash column chromatography to afford isobutyl 5-bromo-1-methyl-1H-imidazole-2-carboxylate (29 g, 89.4% yield) as a yellow oil. MS (ESI, m/z): 261.2 [M+H]⁺.

### Step 2: 5-bromo-1-methyl-imidazole-2-carboxylic acid

To a solution of isobutyl 5-bromo-1-methyl-1H-imidazole-2-carboxylate (29 g, 111 mmol) in MeOH (5 mL) and THF (120 mL) was added a solution of lithium hydroxide monohydrate (9.32 g, 222 mmol) in water (60 mL). The mixture was stirred at room temperature for 3 hrs. The organic solvent was removed under reduced pressure. 12 N HCl aqueous solution was added under stirring until pH 4-5. A white solid was filtered, washed with MeOH and dried to afford 5-bromo-1-methyl-imidazole-2-carboxylic acid (20.5 g, 90% yield) as a white solid. MS (ESI, m/z): 204.8 [M+H]⁺.

### Step 3: methyl 4-[(5-bromo-1-methyl-imidazole-2-carbonyl)amino]-2-chloro-benzoate

A mixture of 5-bromo-1-methyl-1H-imidazole-2-carboxylic acid (13 g, 63.4 mmol), methyl 2-chloro-4-(methylamino)benzoate (11.8 g, 63.4 mmol), HATU (24.1 g, 63.4 mmol) and DIPEA (24.6 g, 33.2 mL) in DMF (30 mL) was stirred at room temperature overnight. Then the mixture was poured into water. The water phase was extracted with DCM (3×50 mL). The combined organic phases were washed with water, dried over anhydrous Na₂SO₄ and concentrated in vacuo. The solids precipitated from the concentrated solution. The solids were collected, washed with MeOH and dried to afford methyl 4-[(5-bromo-1-methyl-imidazole-2-carbonyl)amino]-2-chloro-benzoate (18 g, 76% yield) as a light yellow solid. MS (ESI, m/z): 371.8 [M+H]⁺.

The following Type I Intermediates were prepared in analogy to intermediate 308.

| | | | ESI MS | |
|---|---|---|---|---|
| Int. | Name | Structure | [M + H]⁺ | Starting Material |
| 309 or 310 | methyl 4-[(5- bromo-1-methyl- imidazole-2- carbonyl)amino]-2- methyl-benzoate | 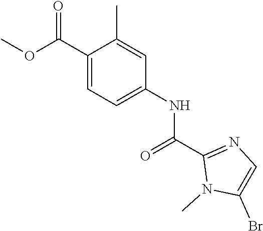 [SMILES: COC(=O)c1ccc(NC(=O)c2ncc(Br)n2C)cc1C] | 352.0 | 5-bromo-1-methyl- 1H-imidazole-2- carboxylic acid and methyl 4-amino-2- methylbenzoate |
| 311 | tert-butyl 4-[(5- bromo-1-methyl- imidazole-2- carbonyl)amino]-2- chloro-benzoate | 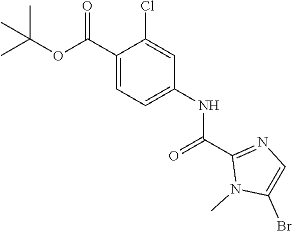 [SMILES: Cn1c(Br)cnc1C(=O)Nc1ccc(C(=O)OC(C)(C)C)c(Cl)c1] | 414.1 | 5-bromo-1-methyl- 1H-imidazole-2- carboxylic acid and tert-butyl 4-amino- 2-chloro-benzoate and |
| 312 | tert-butyl 4-(5- bromo-1-methyl-1H- imidazole-2- carboxamido)-2- methylbenzoate | 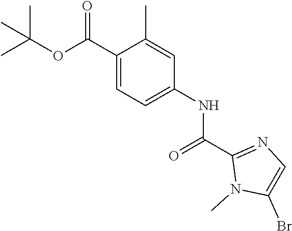 [SMILES: Cc1cc(NC(=O)c2ncc(Br)n2C)ccc1C(=O)OC(C)(C)C] | 394.1 | 2-methyl-4- nitrobenzoic acid and tert-butyl 4- amino-2-chloro- benzoate and |
| 1 | 4-(5-bromo-1- methyl-1H- imidazole-2- carboxamido)-2- ethylbenzoate | 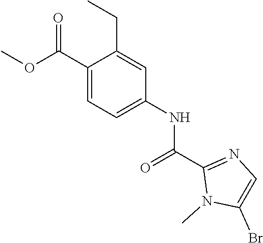 [SMILES: CCc1cc(NC(=O)c2ncc(Br)n2C)ccc1C(=O)OC] | 366.1 | 5-bromo-1-methyl- 1H-imidazole-2- carboxylic acid and methyl 4-amino-2- ethylbenzoate |

### Intermediate Type II: 313

### 4-[(5-Bromo-1-methyl-imidazole-2-carbonyl)amino]-2-chloro-benzoic acid

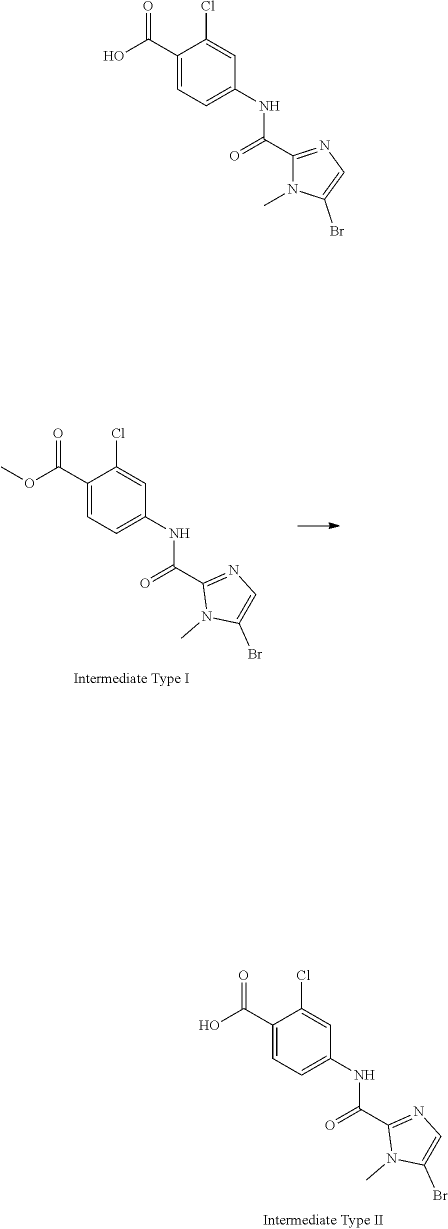 [SMILES: Cn1c(Br)cnc1C(=O)Nc1ccc(C(=O)O)c(Cl)c1 ; COC(=O)c1ccc(NC(=O)c2ncc(Br)n2C)cc1Cl ; Cn1c(Br)cnc1C(=O)Nc1ccc(C(=O)O)c(Cl)c1]

To a solution of methyl 4-[(5-bromo-1-methyl-imidazole-2-carbonyl)amino]-2-chloro-benzoate (7.6 g, 20.4 mmol) in MeOH (2 mL) and THF (48 mL) was added a solution of lithium hydroxide monohydrate (2.57 g, 61.2 mmol) in water (24 mL). The mixture was stirred at room temperature overnight. Then the mixture was concentrated and the water layer was acidified by 6N HCl aqueous solution. The solids precipitated from the concentrated solution. The solids were collected, washed with water and dried to afford 4-[(5-bromo-1-methyl-imidazole-2-carbonyl)amino]-2-chloro-benzoic acid as a white solid (6 g, 82% yield). MS (ESI, m/z): 358.0 [M+H]⁺.

The following Type II Intermediate was prepared in analogy to intermediate 313.

| | | | ESI MS | |
|---|---|---|---|---|
| Int. | Name | Structure | [M + H]⁺ | Starting Material |
| 314 | 4-[(5-bromo-1- methyl-imidazole-2- carbonyl)amino]-2- methyl-benzoic acid | 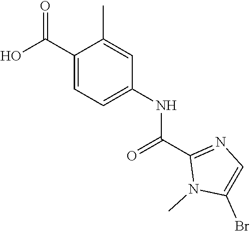 [SMILES: Cc1cc(NC(=O)c2ncc(Br)n2C)ccc1C(=O)O] | 338.0 | tert-butyl 4-[(5- bromo-1-methyl- imidazole-2- carbonyl)amino]-2- methyl-benzoate |

### Intermediate Type III: 315

### 2-(4-(Difluoromethoxy)-2,3-difluorophenyl)-4,4,5,5-tetramethyl-1,3,2-dioxaborolane

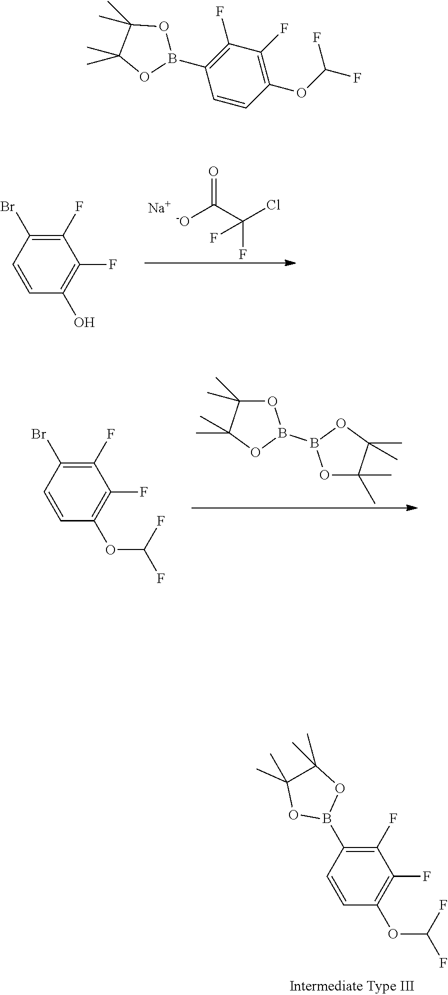

### Step 1:1-bromo-4-(difluoromethoxy)-2,3-difluorobenzene

A solution of 4-bromo-2,3-difluorophenol (25 g, 120 mmol), sodium 2-chloro-2,2-difluoroacetate (36.5 g, 239 mmol) and K₂CO₃ (19.8 g, 144 mmol) in DMF (250 mL) and water (57 mL) was stirred for 3.0 h at 100° C. under N₂. The reaction mixture was poured into 1.5 L H₂O and extracted with EtOAc (3×250 mL). The organic layers were combined, washed with sat NaCl (1×200 mL), The organic layers were dried over Na₂SO₄ and concentrated in vacuo. The crude material was purified by flash chromatography (silica gel, 120 g, 0% to 20% DCM in PE) to afford 1-bromo-4-(difluoromethoxy)-2,3-difluorobenzene (25.2 g, 97.3 mmol, 81.3% yield).

### Step 2: 2-(4-(difluoromethoxy)-2,3-difluorophenyl)-4,4,5,5-tetramethyl-1,3,2-dioxaborolane

To a 250 mL RBF were added 4,4,4′,4′,5,5,5′,5′-octamethyl-2,2′-bi(1,3,2-dioxaborolane) (24.5 g, 96.5 mmol), 1-bromo-4-(difluoromethoxy)-2,3-difluorobenzene (25 g, 96.5 mmol), PdCl₂(DPPF)—CH₂Cl₂ adduct (3.53 g, 4.83 mmol) and potassium acetate (18.9 g, 193 mmol) in dioxane (150 mL). The mixture was stirred at at 80° C. for 15 h under N₂. The crude reaction mixture was concentrated in vacuo. The reaction mixture was poured into 50 mL H₂O and extracted with EtOAc (3×50 mL). The organic layers were combined, washed with sat. NaCl (1×50 mL). The organic layers were dried over Na₂SO₄ and concentrated in vacuo. The crude material was purified by flash chromatography (silica ge1,330 g, 0% to 20% DCM in PE) to afford 2-(4-(difluoromethoxy)-2,3-difluorophenyl)-4,4,5,5-tetramethyl-1,3,2-dioxaborolane (25 g, 81.7 mmol, 84.6% yield).

The following Type III intermediates were prepared in analogy to intermediate 315.

| Int. | Name | Structure | Starting Material |
|---|---|---|---|
| 316 | 2-[4-(difluoromethoxy)- 2-fluoro-phenyl]- 4,4,5,5-tetramethyl- 1,3,2-dioxaborolane | 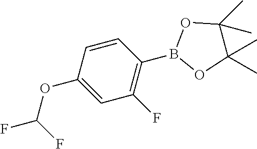 [SMILES: CC1(C)OB(c2ccc(OC(F)F)cc2F)OC1(C)C] | 4-bromo-3-fluoro- phenol and sodium 2- chloro-2,2- difluoroacetate |
| 317 | 2-[4-(difluoromethoxy)- 3-fluoro-phenyl]- 4,4,5,5-tetramethyl- 1,3,2-dioxaborolane | 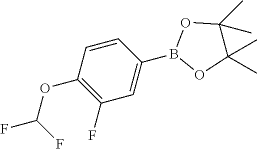 [SMILES: CC1(C)OB(c2ccc(OC(F)F)c(F)c2)OC1(C)C] | 4-bromo-2-fluoro- phenol and sodium 2- chloro-2,2- difluoroacetate |
| 318 | 2-[2-chloro-4- (difluoromethoxy)-3- fluoro-phenyl]-4,4,5,5- tetramethyl-1,3,2- dioxaborolane | 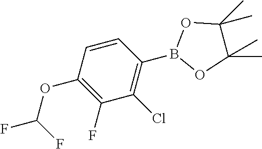 [SMILES: CC1(C)OB(c2ccc(OC(F)F)c(F)c2Cl)OC1(C)C] | 4-bromo-3-chloro-2- fluoro-phenol and sodium 2-chloro-2,2- difluoroacetate |

### Intermediate Type III: 319

### 2-[2,3-difluoro-4-(fluoromethoxy)phenyl]-4,4,5,5-tetramethyl-1,3,2-dioxaborolane

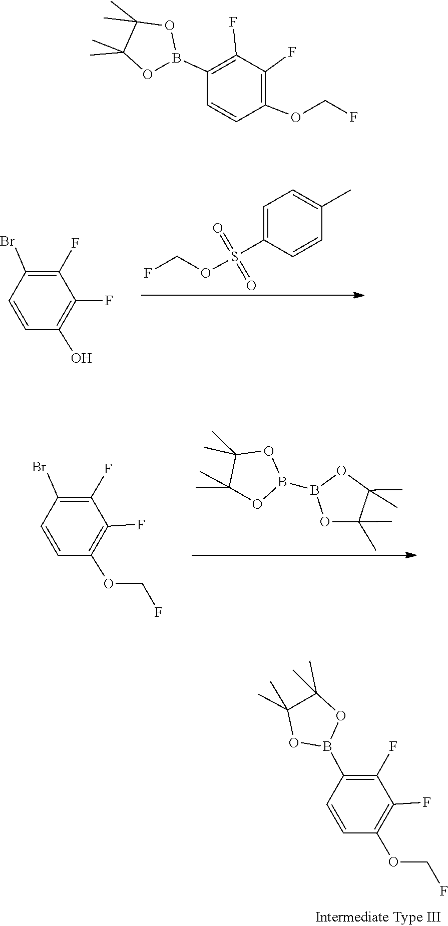

### Step 1: 1-bromo-2,3-difluoro-4-(fluoromethoxy)benzene

To a 5 mL microwave vial were added 4-bromo-2,3-difluorophenol (150 mg, 718 μmol, Eq: 1), fluoromethyl 4-methylbenzenesulfonate (176 mg, 861 μmol) and Cs₂CO₃ (351 mg, 1.08 mmol) in DMF (3 mL). The vial was capped and heated in the microwave at 90° C. overnight. The reaction mixture was poured into 50 mL H₂O and extracted with EtOAc (3×25 mL). The organic layers were combined, washed with sat NaCl (1×50 mL), then dried over Na₂SO₄ and concentrated in vacuo to afford 1-bromo-2,3-difluoro-4-(fluoromethoxy)benzene (153 mg, 635 μmol, 88.4% yield).

### Step 2:

### 2-[2,3-difluoro-4-(fluoromethoxy)phenyl]-4,4,5,5-tetramethyl-1,3,2-dioxaborolane

To a 5 mL microwave vial were added bis(pinacolato)diboron (161 mg, 635 μmol), 1-bromo-2,3-difluoro-4-(fluoromethoxy)benzene (153 mg, 635 μmol), PdCl₂(DPPF)—CH₂Cl₂ adduct (46.5 mg, 63.5 μmol) and potassium acetate (125 mg, 1.27 mmol) in Dioxane (3 mL). The vial was placed under nitrogen, capped and heated by microwave at 80° C. overnight. The crude reaction mixture was concentrated in vacuo. The reaction mixture was poured into 25 mL H₂O and extracted with EtOAc (3×25 mL). The organic layers were combined, washed with sat NaCl (1×25 mL), then dried over Na₂SO₄ and concentrated in vacuo to afford 2-[2,3-difluoro-4-(fluoromethoxy)phenyl]-4,4,5,5-tetramethyl-1,3,2-dioxaborolane (180 mg, 625 μmol, 98.4% yield).

The following Type III Intermediates were prepared in analogy to intermediate 319.

| Int. | Name | Structure | Starting Material |
|---|---|---|---|
| 320 | 2-[3-chloro-2-fluoro-4- (fluoromethoxy)phenyl]- 4,4,5,5-tetramethyl- 1,3,2-dioxaborolane | 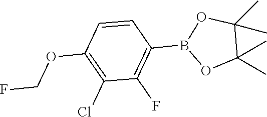 [SMILES: CC1(C)OB(c2ccc(OCF)c(Cl)c2F)OC1(C)C] | 4-bromo-2-chloro-3- fluorophenol and fluoromethyl 4- methylbenzenesulfonate |
| 321 | 2-[2-chloro-3-fluoro-4- (fluoromethoxy)phenyl]- 4,4,5,5-tetramethyl- 1,3,2-dioxaborolane | 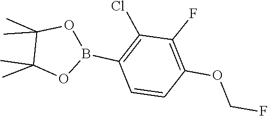 [SMILES: CC1(C)OB(c2ccc(OCF)c(F)c2Cl)OC1(C)C] | 4-bromo-3-chloro-2- fluoro-phenol and fluoromethyl 4- methylbenzenesulfonate |

### Intermediate Type III: 322

### 4,4,5,5-Tetramethyl-2-(2,3,5-trifluoro-4-methoxy-phenyl)-1,3,2-dioxaborolane

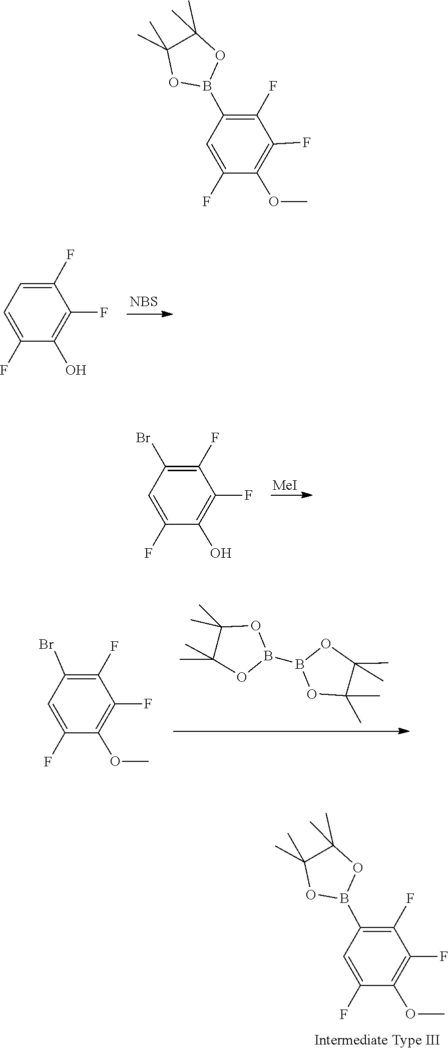

### Step 1: 4-bromo-2,3,6-trifluorophenol

In a 100 mL round-bottomed flask, 2,3,6-trifluorophenol (215 mg, 1.45 mmol) was combined with CHCl₃ (10 mL) to give a colorless solution. NBS (284 mg, 1.6 mmol) was added at 0° C. The reaction was warmed to room temperature with stirring for 2 h. The crude reaction mixture was concentrated in vacuo to afford 4-bromo-2,3,6-trifluorophenol (330 mg, 1.45 mmol, 100% yield), which was directly used to the next step. (ESI, m/z): 227.0 [M−H]⁻.

### Step 2: 1-bromo-2,3,5-trifluoro-4-methoxybenzene

In a 100 mL round-bottomed flask, 4-bromo-2,3,6-trifluorophenol (330 mg, 1.45 mmol), MeI (413 mg, 182 μl, 2.91 mmol) and K₂CO₃ (301 mg, 2.18 mmol) were combined with acetonitrile (10 mL) to give a light yellow solution. The reaction mixture was heated to 50° C. and stirred for 2 h. The reaction mixture was filtered through glass fiber paper. The crude material was purified by flash chromatography to afford 1-bromo-2,3,5-trifluoro-4-methoxybenzene (220 mg, 913 μmol, 62.8% yield).

### Step 3: 4,4,5,5-tetramethyl-2-(2,3,5-trifluoro-4-methoxy-phenyl)-1,3,2-dioxaborolane

To a 100 mL microwave vial were added 1-bromo-2,3,5-trifluoro-4-methoxybenzene (220 mg, 913 μmol), 4,4,4′,4′,5,5,5′,5′-octamethyl-2,2′-bi(1,3,2-dioxaborolane) (255 mg, 1 mmol), Pd₂(dba)₃ (83.6 mg, 91.3 μmol), X-PHOS (87 mg, 183 μmol) and potassium acetate (269 mg, 171 μl, 2.74 mmol) in dioxane (10 mL). The vial was placed under N₂, capped and heated by microwave at 100° C. for 2 h. The reaction mixture was directly used in the next step without further purification.

### Intermediate Type III: 323

### 2,3-Difluoro-N,N-dimethyl-4-(4,4,5,5-tetramethyl-1,3,2-dioxaborolan-2-yl)aniline

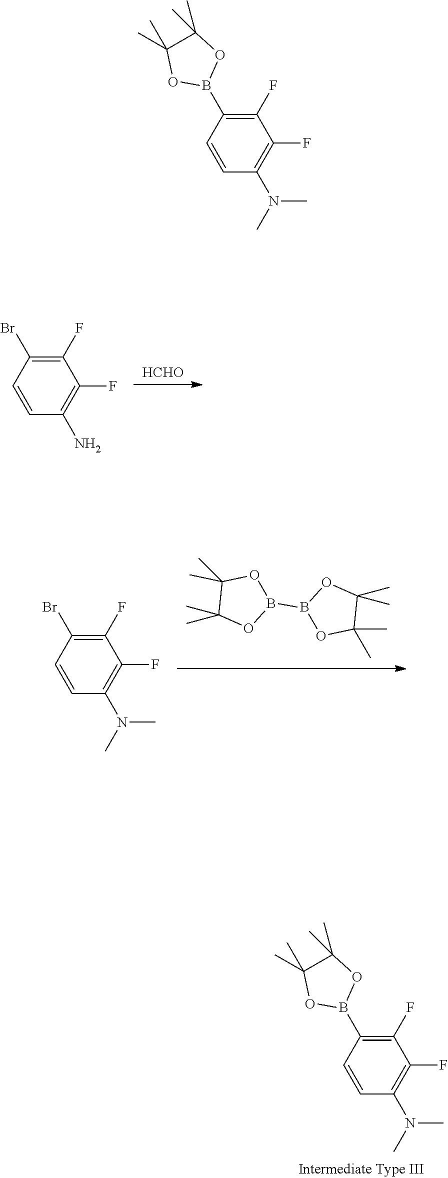

### Step 1: 4-bromo-2,3-difluoro-N,N-dimethylaniline

In a 100 mL round-bottomed flask, 4-bromo-2,3-difluoroaniline (300 mg, 1.44 mmol), formaldehyde (433 mg, 397 μl, 14.4 mmol) and formic acid (6.64 g, 5.53 mL, 144 mmol) were combined to give a light red solution. The reaction mixture was heated to 50° C. and stirred for 3 h. The crude reaction mixture was concentrated in vacuo. The reaction mixture was poured into 50 mL sat NaHCO₃ and extracted with EtOAc (3×25 mL). The organic layers were combined, washed with sat NaCl (1×25 mL), then dried over Na₂SO₄ and concentrated in vacuo. The crude material was purified by flash chromatography to afford 4-bromo-2,3-difluoro-N,N-dimethylaniline (270 mg, 1.14 mmol, 79.3% yield). (ESI, m/z): 238.0 [M+H]⁺.

### Step 2: 2,3-difluoro-N,N-dimethyl-4-(4,4,5,5-tetramethyl-1,3,2-dioxaborolan-2-yl)aniline

In a 100 mL round-bottomed flask, 4,4,4′,4′,5,5,5′,5′-octamethyl-2,2′-bi(1,3,2-dioxaborolane) (70.5 mg, 277 μmol), 4-bromo-2,3-difluoro-N,N-dimethylaniline (65.5 mg, 277 μmol), PdCl₂(DPPF)—CH₂Cl₂ adduct (20.3 mg, 27.7 μmol) and potassium acetate (81.7 mg, 832 μmol) were combined with dioxane (12 mL) to give a light brown solution under N₂. The reaction mixture was heated to 100° C. and stirred for 5 h. The reaction mixture was directly used in the next step.

### Intermediate Type III: 324

### 2,3-Difluoro-N,N-dimethyl-4-(4,4,5,5-tetramethyl-1,3,2-dioxaborolan-2-yl)benzamide

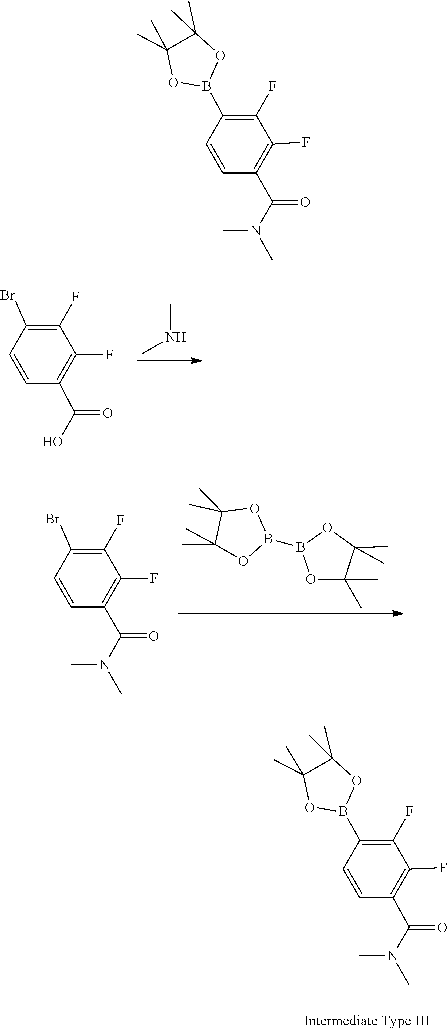

### Step 1: 4-bromo-2,3-difluoro-N,N-dimethylbenzamide

In a 100 mL round-bottomed flask, 4-bromo-2,3-difluorobenzoic acid (300 mg, 1.27 mmol), dimethylamine (in THF) (1.9 mL, 3.8 mmol) and DIEA (327 mg, 442 μl, 2.53 mmol) were combined with DMF (5 mL) to give a colorless solution. HATU (578 mg, 1.52 mmol) was added. The reaction was stirred at room temperature for 1 h. The reaction mixture was poured into 100 mL H₂O and extracted with EtOAc (3×25 mL). The organic layers were combined, washed with sat NaCl (1×25 mL), then dried over Na₂SO₄ and concentrated in vacuo to afford 4-bromo-2,3-difluoro-N,N-dimethylbenzamide (334 mg, 1.26 mmol, 99.9% yield). (ESI, m/z): 264.0 [M+H]⁺.

### Step 2: 2,3-difluoro-N,N-dimethyl-4-(4,4,5,5-tetramethyl-1,3,2-dioxaborolan-2-yl)benzamide

To a 25 mL microwave vial were added bis(pinacolato)diboron (70.7 mg, 278 μmol), 4-bromo-2,3-difluoro-N,N-dimethylbenzamide (70 mg, 265 μmol), Pd₂(dba)₃ (24.3 mg, 26.5 μmol), X-PHOS (25.3 mg, 53 μmol) and potassium acetate (78 mg, 795 μmol) in dioxane (6 mL). The vial was capped and heated in the microwave at 100° C. for 3 h under N₂. The reaction mixture was directly used to the next step.

### Intermediate Type III: 325

### 2-[2-Chloro-4-(4,4,5,5-tetramethyl-1,3,2-dioxaborolan-2-yl)phenoxy]acetonitrile

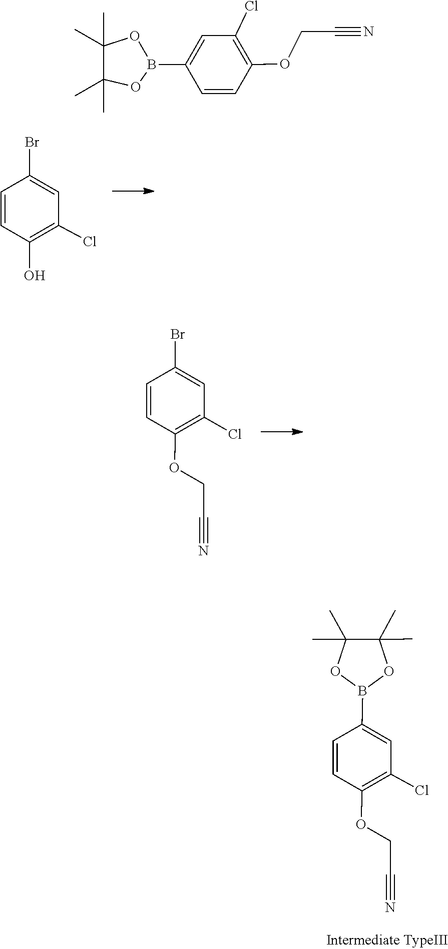

### Step 1: 2-(4-bromo-2-chloro-phenoxy)acetonitrile

The mixture of 4-bromo-2-chlorophenol (3 g, 14.5 mmol), 2-bromoacetonitrile (2.08 g, 17.4 mmol) and K₂CO₃ (3 g, 21.7 mmol) in acetone (30 mL) was stirred at 60° C. for 3 h and then filtered. The solid was washed with EtOAc. The organic phase was washed with water, dried and concentrated to give the title compound (3.5 g, 98.2% yield) as a yellow oil.

### Step 2: 2-[2-chloro-4-(4,4,5,5-tetramethyl-1,3,2-dioxaborolan-2-yl)phenoxy]acetonitrile

A mixture of 2-(4-bromo-2-chlorophenoxy)acetonitrile (3.5 g, 14.2 mmol), 4,4,5,5-tetramethyl-2-(4,4,5,5-tetramethyl-1,3,2-dioxaborolan-2-yl)-1,3,2-dioxaborolane (3.61 g, 14.2 mmol), potassium acetate (2.79 g, 28.4 mmol) and 1,1′-bis(diphenylphosphino)ferrocene-palladium(II)dichloride dichloromethane complex (1.74 g, 2.13 mmol) in 1,4-dioxane (30 mL) was stirred at 80° C. overnight. Then the mixture was concentrated. Water (10 mL) was added. The water layer was extracted with DCM. The organic layer was concentrated and the residue was purified by flash column chromatography to give the title compound (2.8 g, 67.2% yield). MS (ESI, m/z): 293.7 [M+H]⁺.

The following Type III Intermediates were prepared in analogy to intermediate 325.

| Int. | Name | Structure | Starting Material |
|---|---|---|---|
| 326 | 2-[3-chloro-2- fluoro-4-(4,4,5,5- tetramethyl-1,3,2- dioxaborolan-2- yl)phenoxy] acetonitrile | 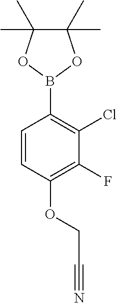 [SMILES: CC1(C)OB(c2ccc(OCC#N)c(F)c2Cl)OC1(C)C] | 4-bromo-3-chloro-2- fluoro-phenol and 2-bromoacetonitrile |
| 327 | 2-[2,3-difluoro-4- (4,4,5,5- tetramethyl-1,3,2- dioxaborolan-2- yl)phenoxy] acetonitrile | 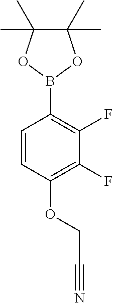 [SMILES: CC1(C)OB(c2ccc(OCC#N)c(F)c2F)OC1(C)C] | 4-bromo-2,3-difluoro- phenol and 2- bromoacetonitrile |
| 328 | 2-(2-chloro-3- fluoro-4- methoxy-phenyl)- 4,4,5,5- tetramethyl-1,3,2- dioxaborolane | 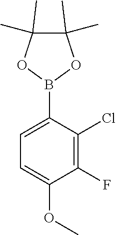 [SMILES: COc1ccc(B2OC(C)(C)C(C)(C)O2)c(Cl)c1F] | 4-bromo-3-chloro-2- fluoro-phenol and iodomethane |
| 329 | 2-[3-fluoro-4- (4,4,5,5- tetramethyl-1,3,2- dioxaborolan-2- yl)phenyl] acetonitrile | 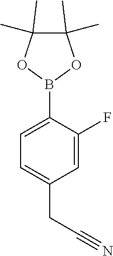 [SMILES: CC1(C)OB(c2ccc(CC#N)cc2F)OC1(C)C] | 2-(4-bromo-3- fluorophenyl)acetonitrile |
| 330 | 2-(2-fluoro-3,4- dimethoxy- phenyl)-4,4,5,5- tetramethyl-1,3,2- dioxaborolane | 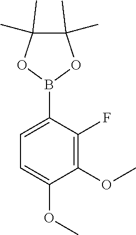 | 4-bromo-3-fluoro- benzene-1,2-diol and iodomethane |
| 331 | 2-[3-fluoro-4- (fluoromethoxy)- 2-methyl-phenyl]- 4,4,5,5- tetramethyl-1,3,2- dioxaborolane | 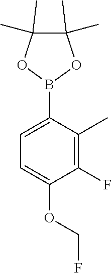 [SMILES: Cc1c(B2OC(C)(C)C(C)(C)O2)ccc(OCF)c1F] | 4-bromo-3-fluoro- benzene-1,2-diol and fluoromethyl 4- methylbenzenesulfonate |
| 332 | 2-fluoro-3- methoxy-6- (4,4,5,5- tetramethyl-1,3,2- dioxaborolan-2- yl)benzonitrile | 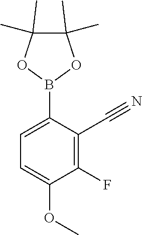 [SMILES: COc1ccc(B2OC(C)(C)C(C)(C)O2)c(C#N)c1F] | Intermediate 333 |
| 334 | 2-[2- (difluoromethyl)- 3-fluoro-4- methoxy-phenyl]- 4,4,5,5- tetramethyl-1,3,2- dioxaborolane | 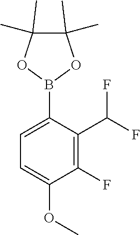 [SMILES: COc1ccc(B2OC(C)(C)C(C)(C)O2)c(C(F)F)c1F] | Intermediate 335 |
| 336 | 2-[2,3-difluoro-4- (2- methoxyethoxy) phenyl]-4,4,5,5- tetramethyl-1,3,2- dioxaborolane | 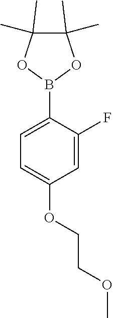 | 1-bromo-2,3-difluoro-4- (2- methoxyethoxy)benzene and 1-bromo-2- methoxyethane |
| 337 | 2-(3,4-difluoro-5- methoxy-phenyl)- 4,4,5,5- tetramethyl-1,3,2- dioxaborolane | 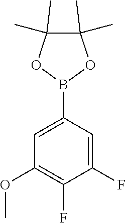 [SMILES: COc1cc(B2OC(C)(C)C(C)(C)O2)cc(F)c1F] | 5-bromo-1,2-difluoro-3- methoxybenzene |
| 338 | 2-[2,3-difluoro-4- (4,4,5,5- tetramethyl-1,3,2- dioxaborolan-2- yl)phenoxy] pyridine | 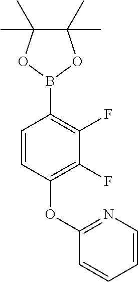 [SMILES: CC1(C)OB(c2ccc(Oc3ccccn3)c(F)c2F)OC1(C)C] | 4-bromo-2,3- difluorophenol and 2- fluoropyridine |
| 339 | 4-[2,3-difluoro-4- (4,4,5,5- tetramethyl-1,3,2- dioxaborolan-2- yl)phenoxy] pyridine | 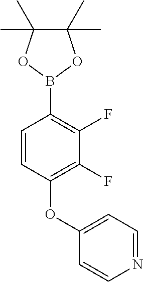 [SMILES: CC1(C)OB(c2ccc(Oc3ccncc3)c(F)c2F)OC1(C)C] | 4-bromo-2,3- difluorophenol and 4- fluoropyridine hydrochloride |
| 340 | 2-[2,3-difluoro-4- (4,4,5,5- tetramethyl-1,3,2- dioxaborolan-2- yl)phenoxy] pyrimidine | 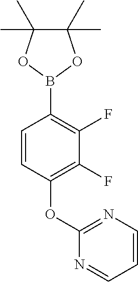 [SMILES: CC1(C)OB(c2ccc(Oc3ncccn3)c(F)c2F)OC1(C)C] | 4-bromo-2,3- difluorophenol and 2- chloropyrimidine |

### Intermediate Type IV: 341

### 2-Chloro-4-[[5-(2,3-difluoro-4-methoxy-phenyl)-1-methyl-imidazole-2-carbonyl]amino]benzoic acid

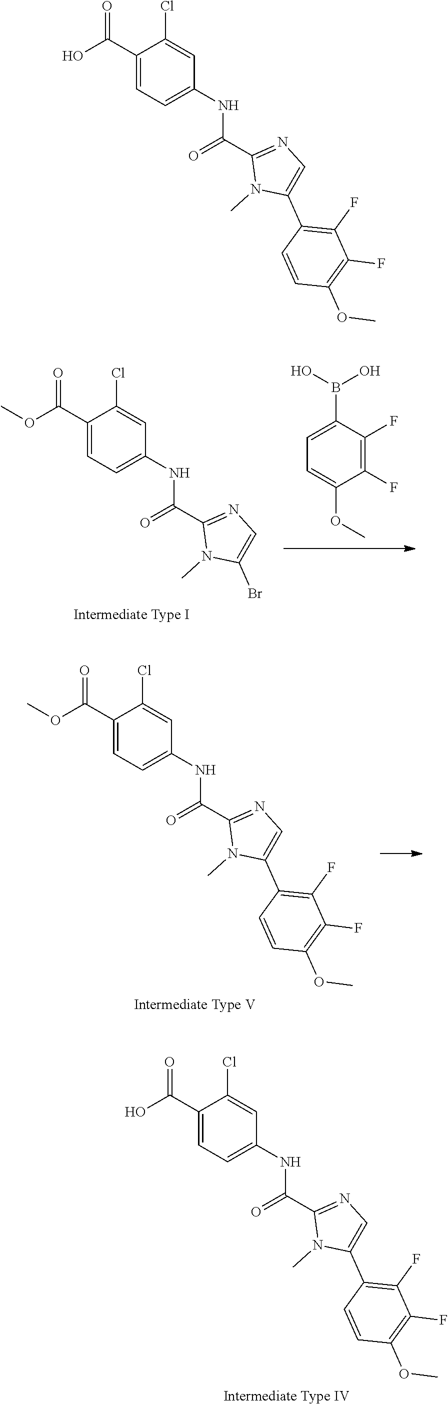 [SMILES: COc1ccc(-c2cnc(C(=O)Nc3ccc(C(=O)O)c(Cl)c3)n2C)c(F)c1F ; COC(=O)c1ccc(NC(=O)c2ncc(Br)n2C)cc1Cl ; COc1ccc(B(O)O)c(F)c1F ; COC(=O)c1ccc(NC(=O)c2ncc(-c3ccc(OC)c(F)c3F)n2C)cc1Cl ; COc1ccc(-c2cnc(C(=O)Nc3ccc(C(=O)O)c(Cl)c3)n2C)c(F)c1F]

### Step 1: methyl 2-chloro-4-[[5-(2,3-difluoro-4-methoxy-phenyl)-1-methyl-imidazole-2-carbonyl]amino]benzoate (Intermediate Type V)

A mixture of methyl 4-[(5-bromo-1-methyl-imidazole-2-carbonyl)amino]-2-chloro-benzoate (intermediate 308, 1 g, 2.68 mmol), (2,3-difluoro-4-methoxyphenyl)boronic acid (504 mg, 2.68 mmol), Na₂CO₃ (853 mg, 8.05 mmol) and 1,1′-bis(di-tert-butylphosphino)ferrocene palladium dichloride (350 mg, 537 μmol) in 1,4-dioxane (15 mL) and water (1.5 mL) was irradiated under microwave at 100° C. for 60 min. This procedure was performed 8 times in total. The individual reaction mixtures were combined and concentrated in vacuo. Water (40 mL) was added. The water phase was extracted with DCM. The combined organic phases were washed with water, dried over anhydrous Na₂SO₄ and concentrated and the solid was dried to give the crude title compound (8.3 g) as a brown solid. MS (ESI, m/z): 435.9 [M+H]⁺.

### Step 2: 2-chloro-4-[[5-(2,3-difluoro-4-methoxy-phenyl)-1-methyl-imidazole-2-carbonyl]amino]benzoic acid

To a solution of methyl 2-chloro-4-[[5-(2,3-difluoro-4-methoxy-phenyl)-1-methyl-imidazole-2-carbonyl]amino]benzoate (8.3 g, 19 mmol) in MeOH (2 mL), THF (48 mL) and water (24 mL) was added a solution of lithium hydroxide monohydrate (3.2 g, 76.2 mmol) in water (24 mL). The mixture was stirred at room temperature overnight. Then the mixture was concentrated and acidified by 6N HCl under stirring until pH 3-4. Some solids precipitated from the concentrated solution. The solids were filtered and dried to give the title compound (7 g, 87% yield) as a brown solid. MS (ESI, m/z): 422.3 [M+H]⁺.

The following Type IV Intermediate was prepared in analogy to intermediate 341.

| | | | ESI MS | |
|---|---|---|---|---|
| Int. | Name | Structure | [M + H]⁺ | Starting Material |
| 342 | 2-chloro-4-[[5-[2,3- difluoro-4- (fluoromethoxy) phenyl]-1-methyl- imidazole-2- carbonyl]amino] benzoic acid | 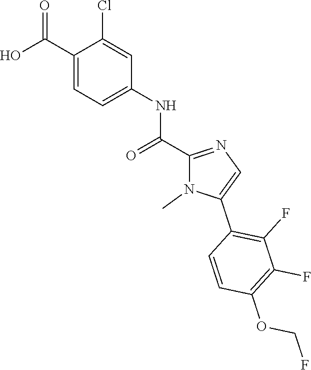 [SMILES: Cn1c(-c2ccc(OCF)c(F)c2F)cnc1C(=O)Nc1ccc(C(=O)O)c(Cl)c1] | 440.1 | tert-butyl 4-(5- bromo-1-methyl- 1H-imidazole-2- carboxamido)-2- chlorobenzoate and Intermediate 319 |
| 343 | 2-chloro-4-[[5-[4- (difluoromethoxy)- 2,3-difluoro-phenyl]- 1-methyl-imidazole- 2-carbonyl]amino] benzoic acid | 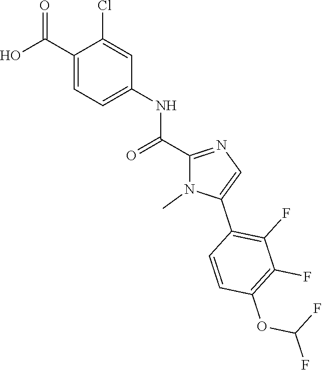 [SMILES: Cn1c(-c2ccc(OC(F)F)c(F)c2F)cnc1C(=O)Nc1ccc(C(=O)O)c(Cl)c1] | 458.1 | tert-butyl 4-(5- bromo-1-methyl- 1H-imidazole-2- carboxamido)-2- chlorobenzoate and Intermediate 315 |
| 344 | 2-chloro-4-[[5-(2- chloro-3-fluoro-4- methoxy-phenyl)-1- methyl-imidazole-2- carbonyl]amino] benzoic acid | 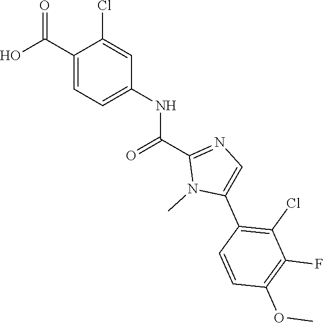 [SMILES: COc1ccc(-c2cnc(C(=O)Nc3ccc(C(=O)O)c(Cl)c3)n2C)c(Cl)c1F] | 438.0 | Intermediate 308 and Intermediate 328 |
| 345 | 4-[[5-(2-chloro-3- fluoro-4-methoxy- phenyl)-1-methyl- imidazole-2- carbonyl]amino]-2- methyl-benzoic acid | 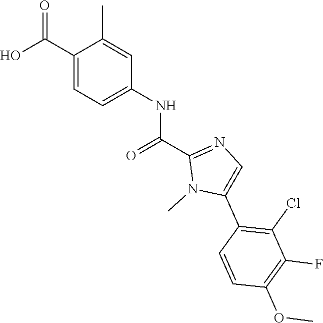 [SMILES: COc1ccc(-c2cnc(C(=O)Nc3ccc(C(=O)O)c(C)c3)n2C)c(Cl)c1F] | 418.1 | Intermediate 309 and Intermediate 328 |
| 346 | 2-chloro-4-[[5-[2- chloro-3-fluoro-4- (fluoromethoxy) phenyl]-1-methyl- imidazole-2- carbonyl]amino] benzoic acid | 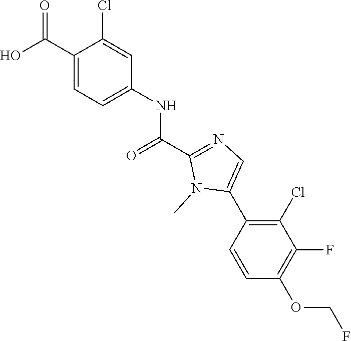 [SMILES: Cn1c(-c2ccc(OCF)c(F)c2Cl)cnc1C(=O)Nc1ccc(C(=O)O)c(Cl)c1] | 456.0 | Intermediate 308 and intermediate 448 |
| 347 | 2-chloro-4-[[5-[4- (difluoromethoxy)-2- fluoro-phenyl]-1- methyl-imidazole-2- carbonyl]amino] benzoic acid | 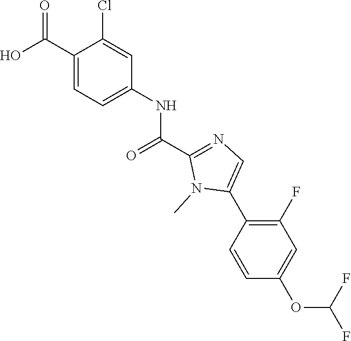 [SMILES: Cn1c(-c2ccc(OC(F)F)cc2F)cnc1C(=O)Nc1ccc(C(=O)O)c(Cl)c1] | 440.1 | Intermediate 308 and intermediate 449 |
| 348 | 2-chloro-4-[[5-[4- (difluoromethoxy)-3- fluoro-phenyl]-1- methyl-imidazole-2- carbonyl]amino] benzoic acid | 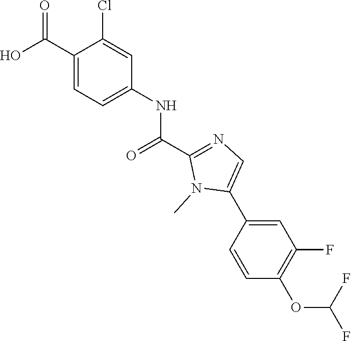 [SMILES: Cn1c(-c2ccc(OC(F)F)c(F)c2)cnc1C(=O)Nc1ccc(C(=O)O)c(Cl)c1] | 440.0 | Intermediate 308 and intermediate 317 |
| 349 | 2-chloro-4-(5-(2,3- difluoro-4- methoxyphenyl)-1- methyl-1H- imidazole-2- carboxamido)benzoic acid | 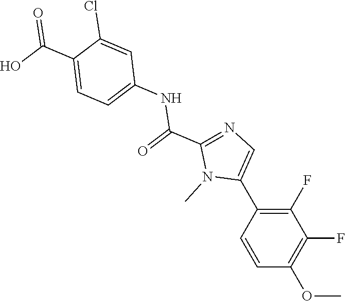 [SMILES: COc1ccc(-c2cnc(C(=O)Nc3ccc(C(=O)O)c(Cl)c3)n2C)c(F)c1F] | 422.1 | (2,3-difluoro-4- methoxyphenyl) boronic acid and intermediate 311 |
| 350 | 4-(5-(4- (cyanomethoxy)-2,3- difluorophenyl)-1- methyl-1H- imidazole-2- carboxamido)-2- methylbenzoic acid | 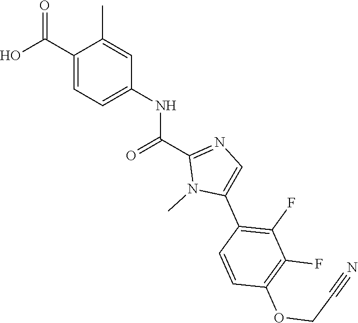 [SMILES: Cc1cc(NC(=O)c2ncc(-c3ccc(OCC#N)c(F)c3F)n2C)ccc1C(=O)O] | 427.1 | Intermediate 312 and intermediate 351 |
| 352 | 2-chloro-4-(5-(2,3- difluoro-4- (fluoromethoxy) phenyl)-1-methyl-1H- imidazole-2- carboxamido)benzoic acid | 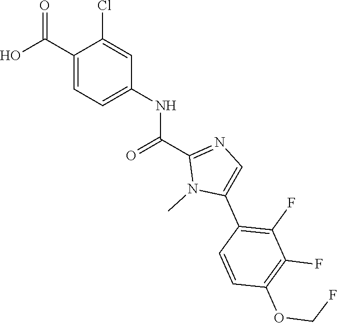 [SMILES: Cn1c(-c2ccc(OCF)c(F)c2F)cnc1C(=O)Nc1ccc(C(=O)O)c(Cl)c1] | 440.1 | 4-bromo-2,3- difluorophenol and intermediate 311 |
| 353 | 2-chloro-4-(5-(2- fluoro-4- (fluoromethoxy) phenyl)-1-methyl-1H- imidazole-2- carboxamido)benzoic acid | 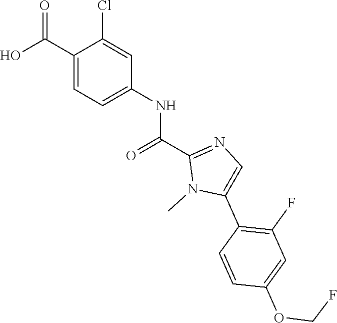 [SMILES: Cn1c(-c2ccc(OCF)cc2F)cnc1C(=O)Nc1ccc(C(=O)O)c(Cl)c1] | 422.1 | 4-bromo-3- fluorophenol and intermediate 311 |
| 354 | 2-chloro-4-(5-(4- (difluoromethoxy)- 2,3-difluorophenyl)- 1-methyl-1H- imidazole-2- carboxamido)benzoic acid | 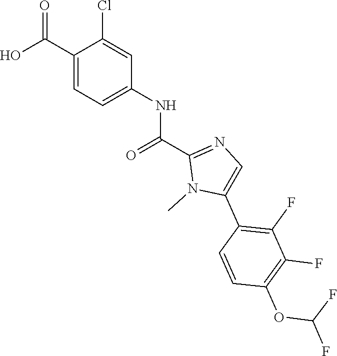 [SMILES: Cn1c(-c2ccc(OC(F)F)c(F)c2F)cnc1C(=O)Nc1ccc(C(=O)O)c(Cl)c1] | 458.1 | 4-bromo-2,3- difluorophenol and intermediate 311 |
| 355- 000- 001 | 4-(5-(4- (difluoromethoxy) phenyl)-1-methyl-1H- imidazole-2- carboxamido)-2- ethylbenzoic acid | 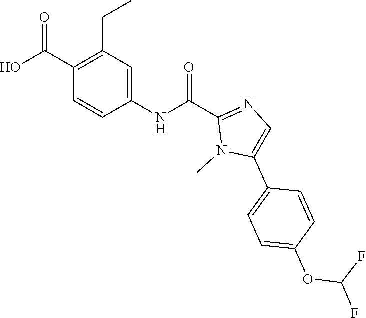 [SMILES: CCc1cc(NC(=O)c2ncc(-c3ccc(OC(F)F)cc3)n2C)ccc1C(=O)O] | 416.2 | 2-(4- (difluoromethoxy) phenyl)-4,4,5- trimethyl-1,3,2- dioxaborolane |
| 357- 000- 001 | 2-ethyl-4-(5-(3- fluoro-4- isopropoxyphenyl)-1- methyl-1H- imidazole-2- carboxamido)benzoic acid | 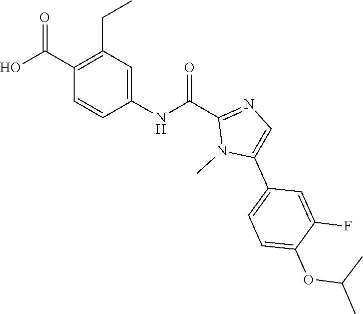 [SMILES: CCc1cc(NC(=O)c2ncc(-c3ccc(OC(C)C)c(F)c3)n2C)ccc1C(=O)O] | 426.3 | 4-(5-bromo-1- methyl-1H- imidazole-2- carboxamido)-2- ethylbenzoate |
| 359- 000- 001 | 4-(5-(2-chloro-4- methoxyphenyl)-1- methyl-1H- imidazole-2- carboxamido)-2- ethylbenzoic acid | 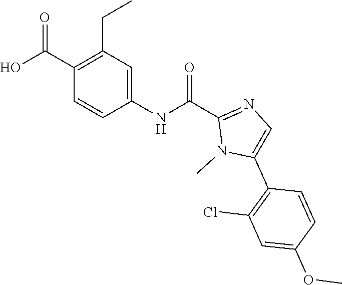 [SMILES: CCc1cc(NC(=O)c2ncc(-c3ccc(OC)cc3Cl)n2C)ccc1C(=O)O] | 414.2 | 4-(5-bromo-1- methyl-1H- imidazole-2- carboxamido)-2- ethylbenzoate |
| 361 | 4-(5-(2,3-difluoro-4- methoxyphenyl)-1- methyl-1H- imidazole-2- carboxamido)-2- ethylbenzoic acid | 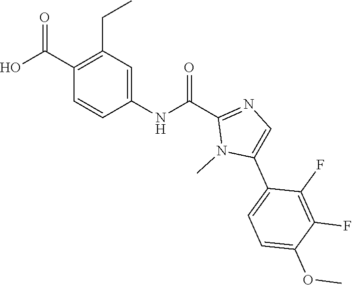 [SMILES: CCc1cc(NC(=O)c2ncc(-c3ccc(OC)c(F)c3F)n2C)ccc1C(=O)O] | 414.3 | (2,3-difluoro-4- methoxyphenyl) boronic acid |

### Intermediate Type VI: 362

### 1-[2-Chloro-4-[[5-(2,3-difluoro-4-methoxy-phenyl)-1-methyl-imidazole-2-carbonyl]amino]benzoyl]piperidine-4-carboxylic acid

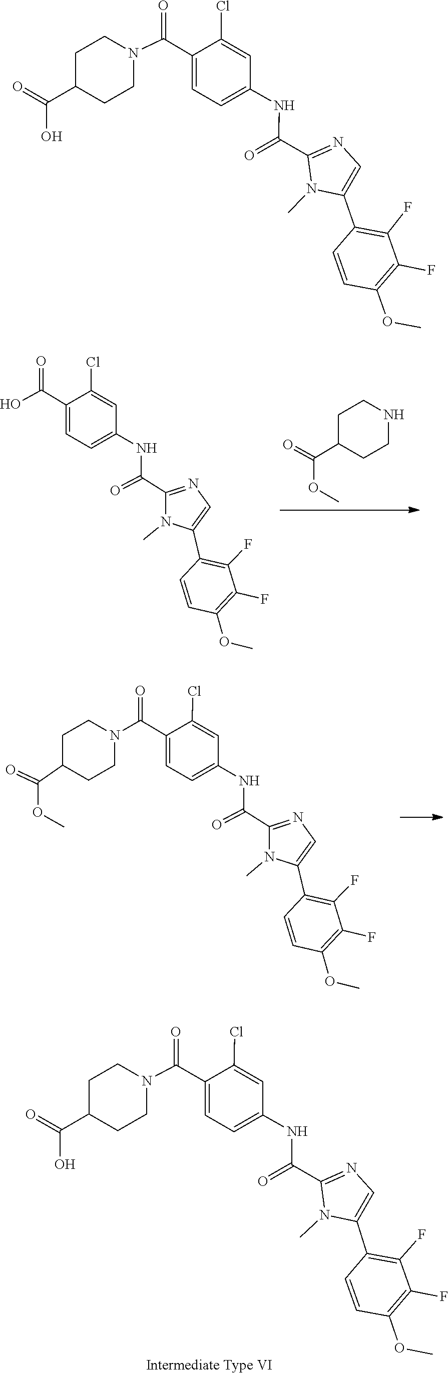

### Step 1: methyl 1-[2-chloro-4-[[5-(2,3-difluoro-4-methoxy-phenyl)-1-methyl-imidazole-2-carbonyl]amino]benzoyl]piperidine-4-carboxylate

The mixture of 2-chloro-4-[[5-(2,3-difluoro-4-methoxy-phenyl)-1-methyl-imidazole-2-carbonyl]amino]benzoic acid (intermediate 341, 3 g, 7.11 mmol), methyl piperidine-4-carboxylate (1.22 g, 8.54 mmol), DIPEA (2.76 g, 3.73 mL, 21.3 mmol) and 2,4,6-tripropyl-1,3,5,2,4,6-trioxatriphosphinane 2,4,6-trioxide (3.39 g, 10.7 mmol) in DMF (10 mL) was stirred at 25° C. overnight. Then the mixture was poured into water. The water phase was extracted with DCM. The combined organic phases were dried over anhydrous Na₂SO₄ and concentrated to give the title compound (3 g, 77.1% yield) as a black oil. MS (ESI, m/z): 547.47 [M+H]⁺.

### Step 2: 1-[2-chloro-4-[[5-(2,3-difluoro-4-methoxy-phenyl)-1-methyl-imidazole-2-carbonyl]amino]benzoyl]piperidine-4-carboxylic acid

A mixture of methyl 1-(2-chloro-4-(5-(2,3-difluoro-4-methoxyphenyl)-1-methyl-1H-imidazole-2-carboxamido)benzoyl)piperidine-4-carboxylate (3.5 g, 6.4 mmol) and lithium hydroxide monohydrate (1.07 g, 25.6 mmol) in THF (12 mL), water (6 mL) and MeOH (0.5 mL) was stirred at room temperature overnight. Then the mixture was acidified by 6N HCl under stirring until pH 2-3. The mixture was concentrated and the water phase was extracted with DCM. The combined organic phases were washed with water, dried and concentrated to give the title compound (2.8 g, 82% yield) as a black solid. MS (ESI, m/z): 533.60 [M+H]⁺.

The following Type VI Intermediates were prepared in analogy to Intermediate 362.

| | | | ESI MS | Starting |
|---|---|---|---|---|
| Int. | Name | Structure | [M + H]⁺ | Material |
| 363 | 1-[2-chloro-4-[[5- [2,3-difluoro-4- (fluoromethoxy) phenyl]-1-methyl- imidazole-2- carbonyl]amino] benzoyl]piperidine-4- carboxylic acid | 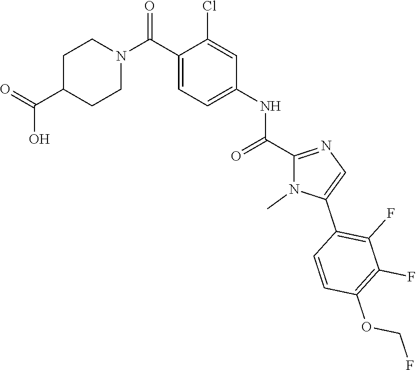 [SMILES: Cn1c(-c2ccc(OCF)c(F)c2F)cnc1C(=O)Nc1ccc(C(=O)N2CCC(C(=O)O)CC2)c(Cl)c1] | 551.2 | Intermediate 342 and methyl piperidine-4- carboxylate |
| 364 | 1-[2-chloro-4-[[5- [4- (difluoromethoxy)- 2,3-difluoro- phenyl]-1-methyl- imidazole-2- carbonyl]amino] benzoyl]piperidine- 4-carboxylic acid | 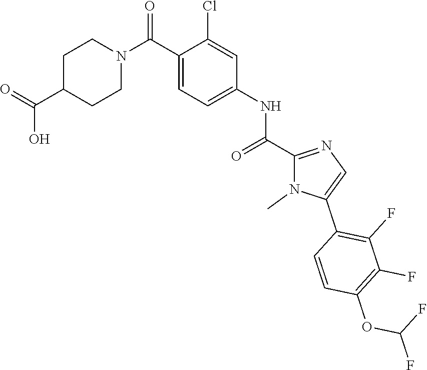 [SMILES: Cn1c(-c2ccc(OC(F)F)c(F)c2F)cnc1C(=O)Nc1ccc(C(=O)N2CCC(C(=O)O)CC2)c(Cl)c1] | 569.1 | Intermediate 343 and methyl piperidine-4- carboxylate |
| 365 | 2-[2-chloro-4-[[5- (2,3-difluoro-4- methoxy-phenyl)-1- methyl-imidazole-2- carbonyl]amino] benzoyl]-2- azaspiro[3.3]heptane- 6-carboxylic acid | 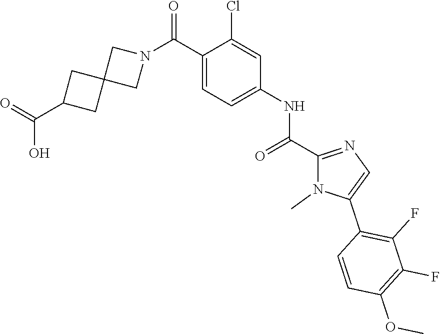 [SMILES: COc1ccc(-c2cnc(C(=O)Nc3ccc(C(=O)N4CC5(CC(C(=O)O)C5)C4)c(Cl)c3)n2C)c(F)c1F] | 545.2 | Intermediate 341 and methyl 2- azaspiro[3.3] heptane-6- carboxylate |
| 366 | 1-[2-chloro-4-[[5- (2-chloro-3-fluoro- 4-methoxy-phenyl)- 1-methyl-imidazole- 2-carbonyl]amino] benzoyl]piperidine-4- carboxylic acid | 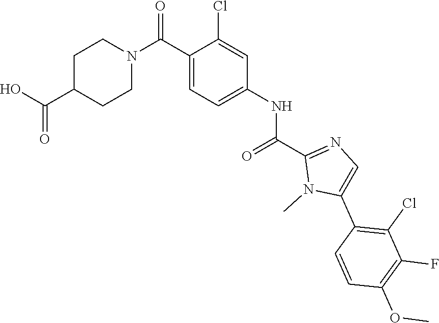 [SMILES: COc1ccc(-c2cnc(C(=O)Nc3ccc(C(=O)N4CCC(C(=O)O)CC4)c(Cl)c3)n2C)c(Cl)c1F] | 549.0 | Intermediate 344 and methyl piperidine-4- carboxylate |
| 367 | 1-[2-chloro-4-[[5- (2,3-difluoro-4- methoxy-phenyl)-1- methyl-imidazole-2- carbonyl]amino] benzoyl]azetidine-3- carboxylic acid | 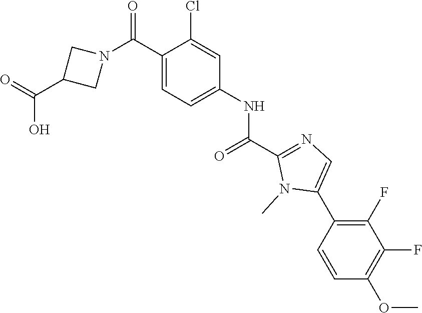 [SMILES: COc1ccc(-c2cnc(C(=O)Nc3ccc(C(=O)N4CC(C(=O)O)C4)c(Cl)c3)n2C)c(F)c1F] | 456.9 | Intermediate 341 and methyl azetidine-3- carboxylate |
| 368 | 1-[2-chloro-4-[[5- (2,3-difluoro-4- methoxy-phenyl)-1- methyl-imidazole-2- carbonyl]amino] benzoyl]pyrrolidine-3- carboxylic acid | 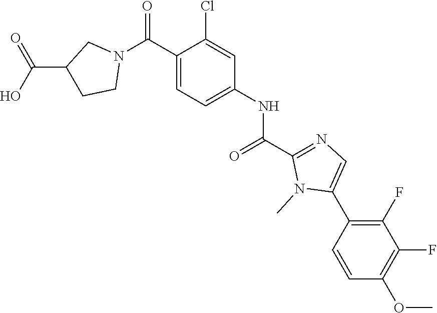 [SMILES: COc1ccc(-c2cnc(C(=O)Nc3ccc(C(=O)N4CCC(C(=O)O)C4)c(Cl)c3)n2C)c(F)c1F] | 519.2 | Intermediate 341 andtert- butyl pyrrolidine-3- carboxylate |
| 369 | 1-[2-chloro-4-[[5- [2-chloro-3-fluoro- 4- (fluoromethoxy) phenyl]-1-methyl- imidazole-2- carbonyl]amino] benzoyl]piperidine-4- carboxylic acid | 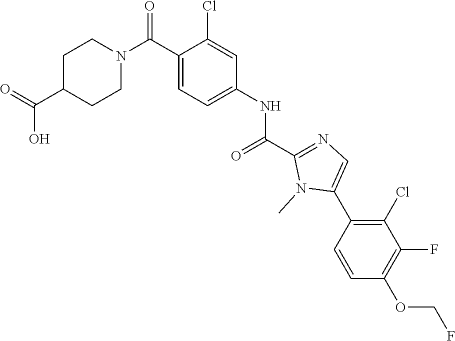 [SMILES: Cn1c(-c2ccc(OCF)c(F)c2Cl)cnc1C(=O)Nc1ccc(C(=O)N2CCC(C(=O)O)CC2)c(Cl)c1] | 566.8 | Intermediate 346 and methyl piperidine-4- carboxylate |
| 370 | 1-[2-chloro-4-[[5- [4-(cyanomethoxy)- 2,3-difluoro- phenyl]-1-methyl- imidazole-2- carbonyl]amino] benzoyl]piperidine-4- carboxylic acid | 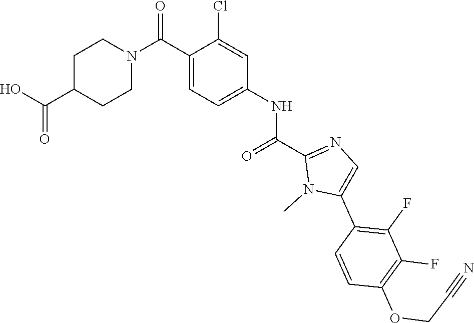 [SMILES: Cn1c(-c2ccc(OCC#N)c(F)c2F)cnc1C(=O)Nc1ccc(C(=O)N2CCC(C(=O)O)CC2)c(Cl)c1] | 558.1 | 371 and methyl piperidine-4- carboxylate |
| 372 | 1-(4-(5-(4- (cyanomethoxy)- 2,3-difluorophenyl)- 1-methyl-1H- imidazole-2- carboxamido)-2- methylbenzoyl) piperidine- 4-carboxylic acid | 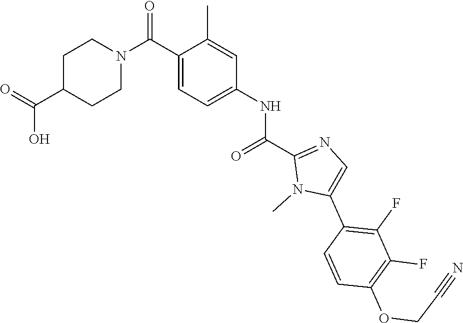 [SMILES: Cc1cc(NC(=O)c2ncc(-c3ccc(OCC#N)c(F)c3F)n2C)ccc1C(=O)N1CCC(C(=O)O)CC1] | 538.2 | Intermediate 350 and tert- butyl piperidine-4- carboxylate |
| 3 | 1-(4-(5-(2,3- difluoro-4- methoxyphenyl)-1- methyl-1H- imidazole-2- carboxamido)-2- ethylbenzoyl) piperidine-4- carboxylic acid | 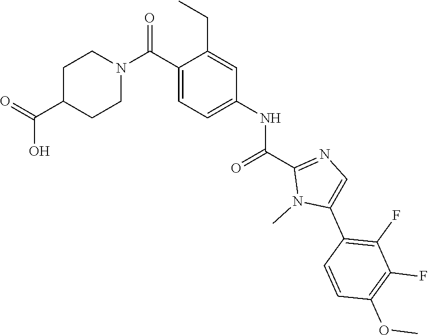 [SMILES: CCc1cc(NC(=O)c2ncc(-c3ccc(OC)c(F)c3F)n2C)ccc1C(=O)N1CCC(C(=O)O)CC1] | 525.4 | Intermediate 361/4 and ethyl piperidine-4- carboxylate |

### Intermediate Type VI: 373

### N-[4-(4-Aminopiperidine-1-carbonyl)-3-chloro-phenyl]-5-(2,3-difluoro-4-methoxy-phenyl)-1-methyl-imidazole-2-carboxamide

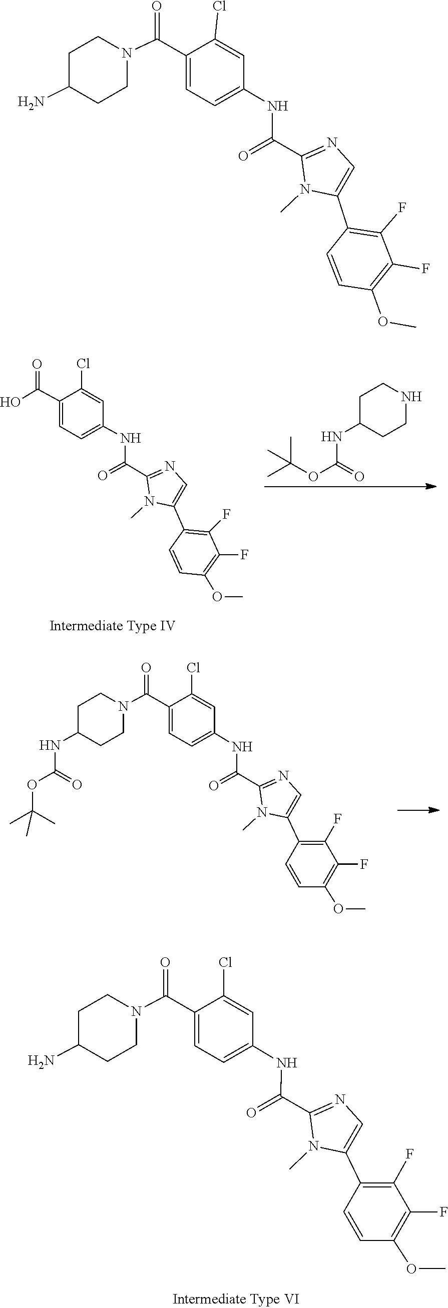 [SMILES: COc1ccc(-c2cnc(C(=O)Nc3ccc(C(=O)N4CCC(N)CC4)c(Cl)c3)n2C)c(F)c1F ; COc1ccc(-c2cnc(C(=O)Nc3ccc(C(=O)O)c(Cl)c3)n2C)c(F)c1F ; CC(C)(C)OC(=O)NC1CCNCC1 ; COc1ccc(-c2cnc(C(=O)Nc3ccc(C(=O)N4CCC(NC(=O)OC(C)(C)C)CC4)c(Cl)c3)n2C)c(F)c1F ; COc1ccc(-c2cnc(C(=O)Nc3ccc(C(=O)N4CCC(N)CC4)c(Cl)c3)n2C)c(F)c1F]

### Step 1: tert-butyl N-[1-[2-chloro-4-[[5-(2,3-difluoro-4-methoxy-phenyl)-1-methyl-imidazole-2-carbonyl]amino]benzoyl]-4-piperidyl]carbamate

A mixture of tert-butyl piperidin-4-ylcarbamate (684 mg, 3.41 mmol), 2-chloro-4-[[5-(2,3-difluoro-4-methoxy-phenyl)-1-methyl-imidazole-2-carbonyl]amino]benzoic acid (Intermediate 341, 1.2 g, 2.85 mmol), HATU (1.3 g, 3.41 mmol) and DIPEA (1.1 g, 1.49 mL, 8.54 mmol) in DMF (10 mL) was stirred at room temperature for 2 h. Then the mixture was poured into water. The solid was collected and dried to give the title compound (1.5 g, 87.3% yield) as a brown solid. MS (ESI, m/z): 604.3 [M+H]⁺.

### Step 2: N-[4-(4-aminopiperidine-1-carbonyl)-3-chloro-phenyl]-5-(2,3-difluoro-4-methoxy-phenyl)-1-methyl-imidazole-2-carboxamide

At room temperature, a solution of tert-butyl N-[1-[2-chloro-4-[[5-(2,3-difluoro-4-methoxy-phenyl)-1-methyl-imidazole-2-carbonyl]amino]benzoyl]-4-piperidyl]carbamate (1.5 g, 2.48 mmol) in DCM (10 mL) and TFA (10 mL) was stirred for 1 h. Under ice cooling, the solution was basified with NH₃.H₂O until pH 8-9. The water layer was extracted with DCM. The organic layer was dried over anhydrous Na₂SO₄ and concentrated to give the crude title compound (900 mg, 66% yield) as a black solid. MS (ESI, m/z): 504.2 [M+H]⁺.

The following Type VI Intermediates were prepared in analogy to intermediate 373.

| | | | ESI MS | Starting |
|---|---|---|---|---|
| Int. | Name | Structure | [M+H]⁺ | Material |
| 374 | N-[3-chloro-4- (piperazine-1- carbonyl)phenyl]-5- (2,3-difluoro-4- methoxy-phenyl)-1- methyl-imidazole-2- carboxamide | 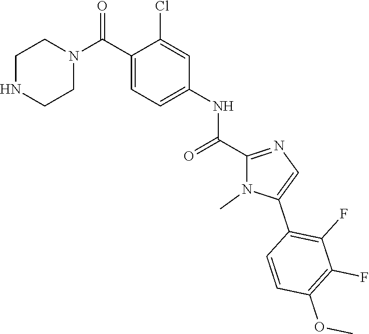 [SMILES: COc1ccc(-c2cnc(C(=O)Nc3ccc(C(=O)N4CCNCC4)c(Cl)c3)n2C)c(F)c1F] | 490.2 | Intermediate 341 and tert- butyl piperazine-1- carboxylate |
| 375 | N-[4-(3- aminopyrrolidine-1- carbonyl)-3-chloro- phenyl]-5-(2,3- difluoro-4-methoxy- phenyl)-1-methyl- imidazole-2- carboxamide | 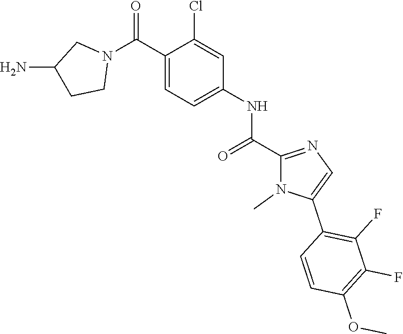 [SMILES: COc1ccc(-c2cnc(C(=O)Nc3ccc(C(=O)N4CCC(N)C4)c(Cl)c3)n2C)c(F)c1F] | 490.1 | Intermediate 341 and tert- butyl pyrrolidin-3- ylcarbamate |
| 376 | N-[3-chloro-4-(2,6- diazaspiro[3.3]heptane- 2-carbonyl)phenyl]-5- (2,3-difluoro-4- methoxy-phenyl)-1- methyl-imidazole-2- carboxamide | 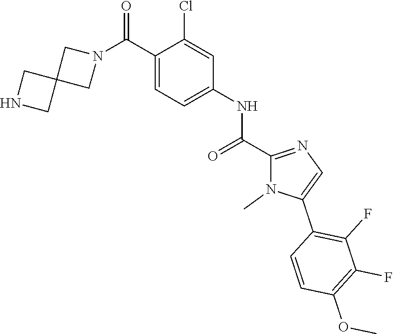 [SMILES: COc1ccc(-c2cnc(C(=O)Nc3ccc(C(=O)N4CC5(CNC5)C4)c(Cl)c3)n2C)c(F)c1F] | 502.23 | Intermediate 341 and tert- butyl 2,6- diazaspiro[3.3] heptane-2- carboxylate oxalate |
| 377 | 5-(2-chloro-3-fluoro-4- methoxy-phenyl)-N-[3- chloro-4-(piperazine-1- carbonyl)phenyl]-1- methyl-imidazole-2- carboxamide; 2,2,2- trifluoroacetic acid | 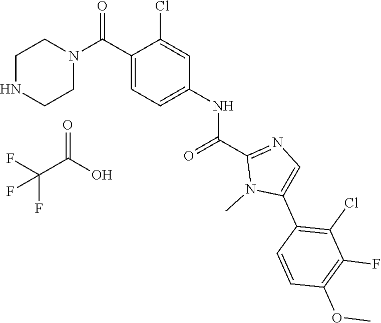 | 506.2 | Intermediate 344 and tert- butyl piperazine-1- carboxylate |
| 378 | 5-(2-chloro-3-fluoro-4- methoxy-phenyl)-1- methyl-N-[3-methyl-4- (piperazine-1- carbonyl)phenyl] imidazole-2- carboxamide; 2,2,2- trifluoroacetic acid | 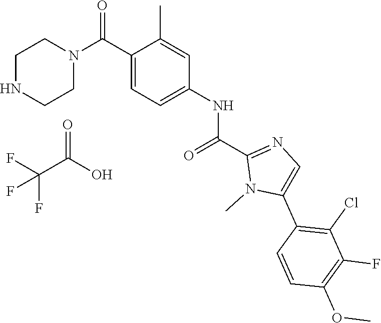 | 486.2 | Intermediate 345 and tert- butyl piperazine-1- carboxylate |
| 379 | N-[4-(3- aminoazetidine-1- carbonyl)-3-chloro- phenyl]-5-(2,3- difluoro-4-methoxy- phenyl)-1-methyl- imidazole-2- carboxamide | 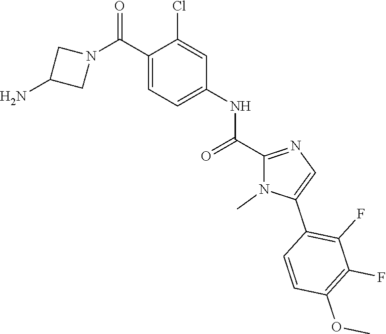 [SMILES: COc1ccc(-c2cnc(C(=O)Nc3ccc(C(=O)N4CC(N)C4)c(Cl)c3)n2C)c(F)c1F] | 476.3 | Intermediate 341 and tert- butyl N- (azetidin-3- yl)carbamate |
| 380 | N-[4-[(3aR,6aS)- 2,3,3a,4,6,6a- hexahydro-1H- pyrrolo[3,4-c]pyrrole- 5-carbonyl]-3-chloro- phenyl]-5-(2,3- difluoro-4-methoxy- phenyl)-1-methyl- imidazole-2- carboxamide | 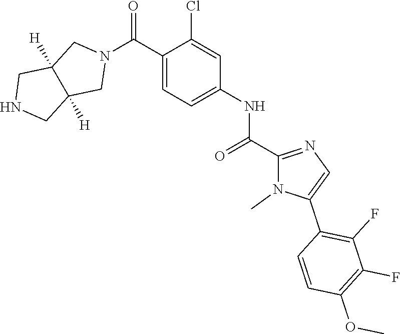 [SMILES: COc1ccc(-c2cnc(C(=O)Nc3ccc(C(=O)N4C[C@H]5CNC[C@H]5C4)c(Cl)c3)n2C)c(F)c1F] | 516.3 | Intermediate 341 and tert- butyl rac- (3aS,6aR)- 2,3,3a,4,6,6a- hexahydro-1H- pyrrolo[3,4- c]pyrrole-5- carboxylate |
| 381 | N-[3-chloro-4- (piperazine-1- carbonyl)phenyl]-5-[4- (difluoromethoxy)-2,3- difluoro-phenyl]-1- methyl-imidazole-2- carboxamide | 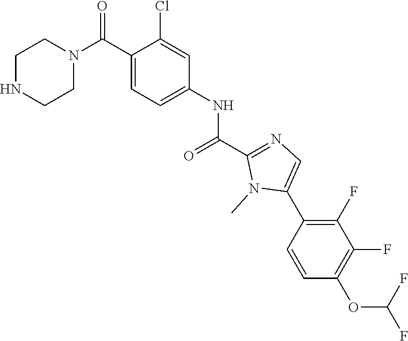 [SMILES: Cn1c(-c2ccc(OC(F)F)c(F)c2F)cnc1C(=O)Nc1ccc(C(=O)N2CCNCC2)c(Cl)c1] | 526.2 | Intermediate 343 and tert- butyl piperazine-1- carboxylate |
| 382 | N-[3-chloro-4- (piperazine-1- carbonyl)phenyl]-5-[4- (difluoromethoxy)-2- fluoro-phenyl]-1- methyl-imidazole-2- carboxamide | 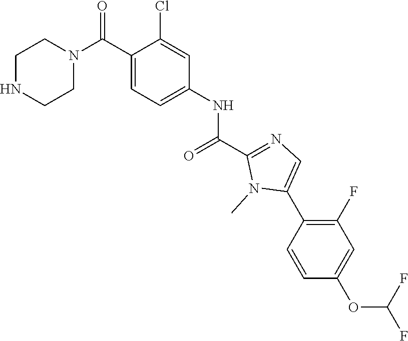 [SMILES: Cn1c(-c2ccc(OC(F)F)cc2F)cnc1C(=O)Nc1ccc(C(=O)N2CCNCC2)c(Cl)c1] | 508.3 | Intermediate 347 and tert- butyl piperazine-1- carboxylate |
| 383 | N-[3-chloro-4- (piperazine-1- carbonyl)phenyl]-5-[4- (difluoromethoxy)-3- fluoro-phenyl]-1- methyl-imidazole-2- carboxamide | 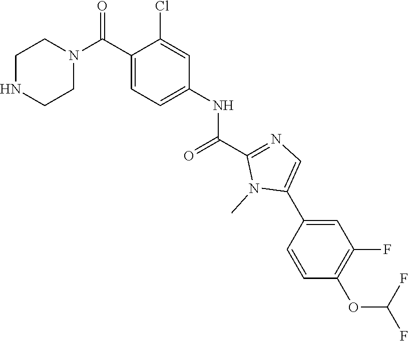 [SMILES: Cn1c(-c2ccc(OC(F)F)c(F)c2)cnc1C(=O)Nc1ccc(C(=O)N2CCNCC2)c(Cl)c1] | 508.2 | Intermediate 348 and tert- butyl piperazine-1- carboxylate |
| 384 | N-(3-chloro-4- (piperazine-1- carbonyl)phenyl)-5-(4- (cyanomethoxy)-2,3- difluorophenyl)-1- methyl-1H-imidazole- 2-carboxamide | 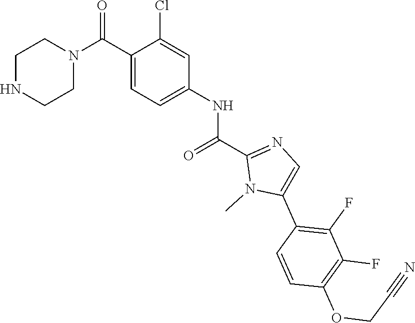 [SMILES: Cn1c(-c2ccc(OCC#N)c(F)c2F)cnc1C(=O)Nc1ccc(C(=O)N2CCNCC2)c(Cl)c1] | 515.2 | 371 and 1- Boc-piperazine |
| 385 | 5-(4-(cyanomethoxy)- 2,3-difluorophenyl)-1- methyl-N-(3-methyl-4- (piperazine-1- carbonyl)phenyl)-1H- imidazole-2- carboxamide | 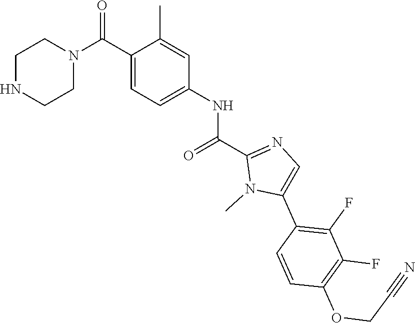 [SMILES: Cc1cc(NC(=O)c2ncc(-c3ccc(OCC#N)c(F)c3F)n2C)ccc1C(=O)N1CCNCC1] | 495.2 | Intermediate 350 and tert- butyl piperazine-1- carboxylate |
| 386 | N-(3-chloro-4- (piperazine-1- carbonyl)phenyl)-5- (2,3-difluoro-4- methoxyphenyl)-1- methyl-1H-imidazole- 2-carboxamide | 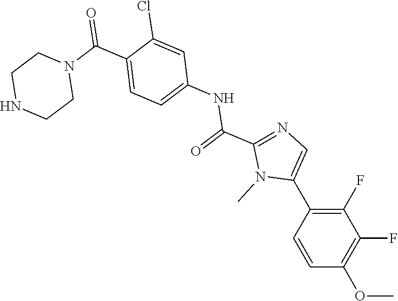 [SMILES: COc1ccc(-c2cnc(C(=O)Nc3ccc(C(=O)N4CCNCC4)c(Cl)c3)n2C)c(F)c1F] | 490.1 | Intermediate 349 and tert- butyl piperazine-1- carboxylate |
| 387 | N-(4-(3- aminoazetidine-1- carbonyl)-3- chlorophenyl)-5-(2,3- difluoro-4- methoxy phenyl)-1- methyl-1H-imidazole- 2-carboxamide | 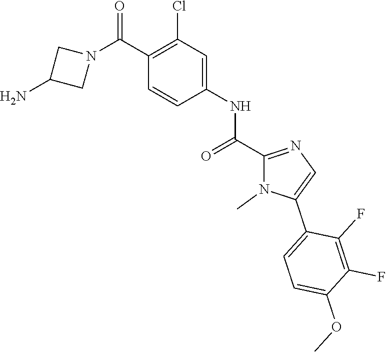 [SMILES: COc1ccc(-c2cnc(C(=O)Nc3ccc(C(=O)N4CC(N)C4)c(Cl)c3)n2C)c(F)c1F] | 476.1 | Intermediate 349 and tert- butyl azetidin- 3-ylcarbarnate |
| 388 | N-(3-chloro-4- (piperazine-1- carbonyl)phenyl)-5- (2,3-difluoro-4- (fluoromethoxy) phenyl)-1-methyl- 1H-imidazole-2- carboxamide | 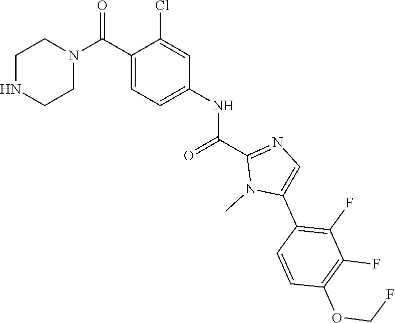 [SMILES: Cn1c(-c2ccc(OCF)c(F)c2F)cnc1C(=O)Nc1ccc(C(=O)N2CCNCC2)c(Cl)c1] | 508.1 | Intermediate 352 and tert- butyl piperazine-1- carboxylate |
| 389 | N-(3-chloro-4- (piperazine-1- carbonyl)phenyl)-5-(2- fluoro-4- (fluoromethoxy) phenyl)-1-methyl- 1H-imidazole-2- carboxamide | 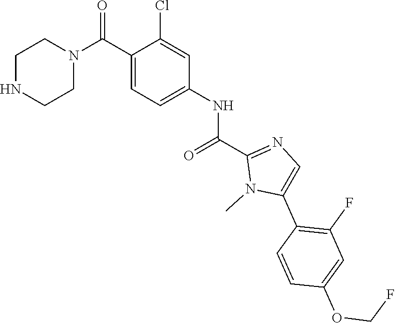 [SMILES: Cn1c(-c2ccc(OCF)cc2F)cnc1C(=O)Nc1ccc(C(=O)N2CCNCC2)c(Cl)c1] | 490.1 | Intermediate 353 and tert- butyl piperazine-1- carboxylate |
| 390 | N-(3-chloro-4- (piperazine-1- carbonyl)phenyl)-5-(4- (difluoromethoxy)-2,3- difluorophenyl)-1- methyl-1H-imidazole- 2-carboxamide | 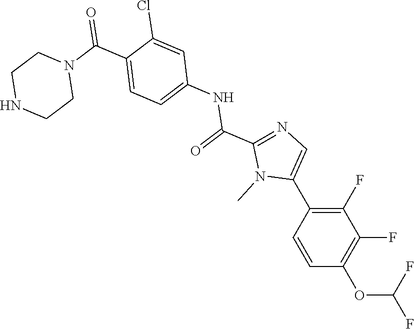 [SMILES: Cn1c(-c2ccc(OC(F)F)c(F)c2F)cnc1C(=O)Nc1ccc(C(=O)N2CCNCC2)c(Cl)c1] | 526.1 | Intermediate 354 and tert- butyl piperazine-1- carboxylate |
| 391 | N-[4-[4- (aminomethyl) piperidine-1- carbonyl]-3- chloro-phenyl]-5-(2,3- difluoro-4-methoxy- phenyl)-1-methyl- imidazole-2- carboxamide | 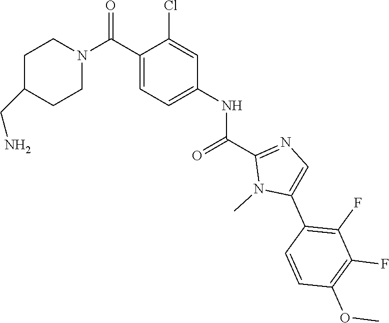 [SMILES: COc1ccc(-c2cnc(C(=O)Nc3ccc(C(=O)N4CCC(CN)CC4)c(Cl)c3)n2C)c(F)c1F] | 518.2 | Intermediate 349 and tert- butyl N-(4- piperidylmethyl) carbamate |

### Intermediate Type VI:392

### N-(3-Chloro-4-(piperazine-1-carbonyl)phenyl)-5-(2,3-difluoro-4-(fluoromethoxy)phenyl)-1-methyl-1H-imidazole-2-carboxamide

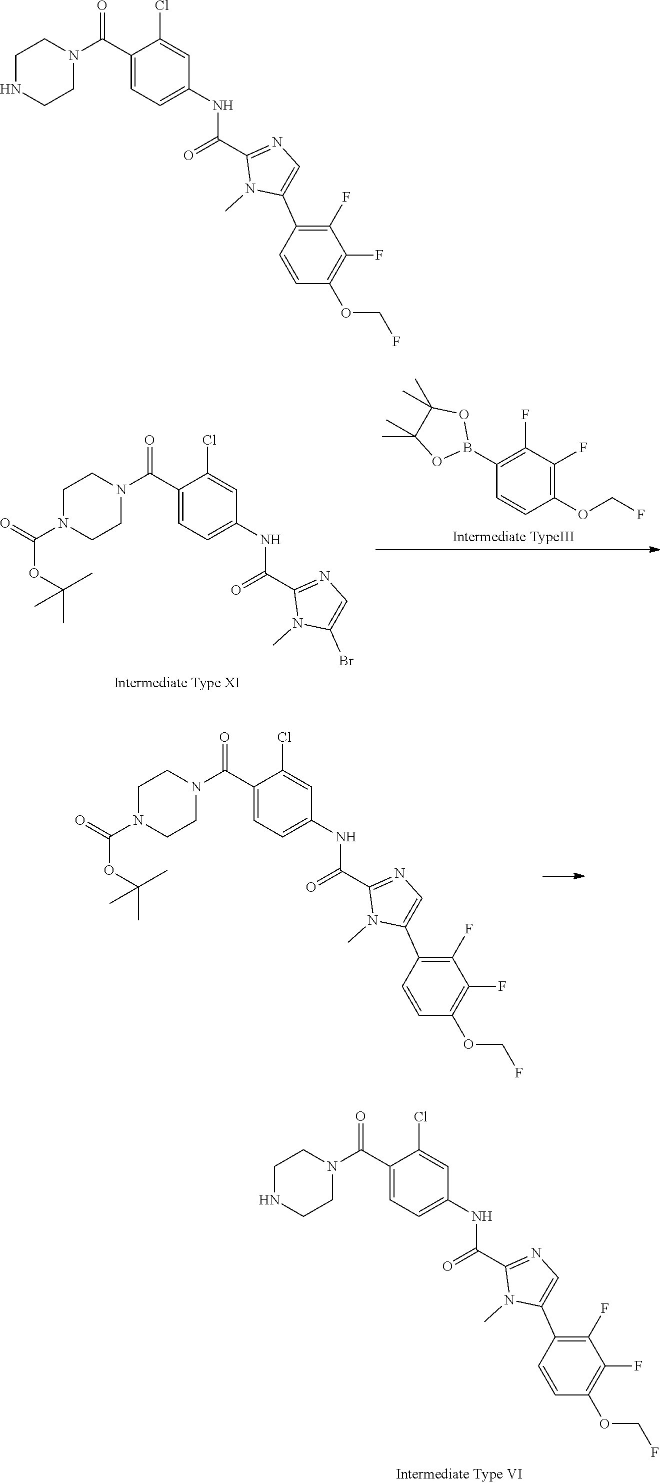

### Step 1

### tert-butyl 4-(2-chloro-4-(5-(2,3-difluoro-4-(fluoromethoxy)phenyl)-1-methyl-1H-imidazole-2-carboxamido)benzoyl)piperazine-1-carboxylate

To a 25 mL microwave vial were added tert-butyl 4-(4-(5-bromo-1-methyl-1H-imidazole-2-carboxamido)-2-chlorobenzoyl)piperazine-1-carboxylate (1 g, 1.9 mmol), 2-(2,3-difluoro-4-(fluoromethoxy)phenyl)-4,4,5,5-tetramethyl-1,3,2-dioxaborolane (602 mg, 2.09 mmol), 1,1′-bis(di-tert-butylphosphino)ferrocene palladium dichloride (124 mg, 190 μmol) and Na₂CO₃ (604 mg, 5.69 mmol) in dioxane (18 mL)/water (3 mL). The vial was capped and heated in the microwave at 100° C. for 2 h under N₂. The crude reaction mixture was concentrated in vacuo. The crude material was purified by flash chromatography to afford tert-butyl 4-(2-chloro-4-(5-(2,3-difluoro-4-(fluoromethoxy)phenyl)-1-methyl-1H-imidazole-2-carboxamido)benzoyl)piperazine-1-carboxylate (759 mg, 1.25 mmol, 65.8% yield). MS (ESI, m/z): 608.2 [M+H]⁺.

### Step 2

### Intermediate 392

### N-(3-chloro-4-(piperazine-1-carbonyl)phenyl)-5-(2,3-difluoro-4-(fluoromethoxy)phenyl)-1-methyl-1H-imidazole-2-carboxamide

In a 100 mL round-bottomed flask, tert-butyl 4-(2-chloro-4-(5-(2,3-difluoro-4-(fluoromethoxy) phenyl)-1-methyl-1H-imidazole-2-carboxamido)benzoyl)piperazine-1-carboxylate (759 mg, 1.25 mmol) was combined with THF (8 mL) to give a dark brown solution. HCl (4.16 mL, 49.9 mmol) was added. The reaction was stirred at room temperature for 10 min. The crude reaction mixture was concentrated in vacuo, to afford N-(3-chloro-4-(piperazine-1-carbonyl)phenyl)-5-(2,3-difluoro-4-(fluoromethoxy)phenyl)-1-methyl-1H-imidazole-2-carboxamide (634 mg, 80% yield) which was used directly in the next step. MS (ESI, m/z): 508.2 [M+H]⁺.

The following Type VI Intermediates were prepared in analogy to intermediate 392.

| | | | ESI MS | Starting |
|---|---|---|---|---|
| Int. | Name | Structure | [M + H]⁺ | Material |
| 393 | 5-[2-chloro-4- (difluoromethoxy)-3- fluoro-phenyl]-N-[3- chloro-4-(piperazine-1- carbonyl)phenyl]-1- methyl-imidazole-2- carboxamide | 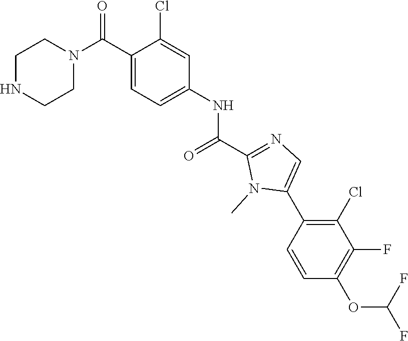 [SMILES: Cn1c(-c2ccc(OC(F)F)c(F)c2Cl)cnc1C(=O)Nc1ccc(C(=O)N2CCNCC2)c(Cl)c1] | 542.0 | Intermediate 394 and Intermediate 318 |
| 395 | N-[3-chloro-4- (piperazine-1- carbonyl)phenyl]-1- methyl-5-(2,3,5- trifluoro-4-methoxy- phenyl)imidazole-2- carboxamide | 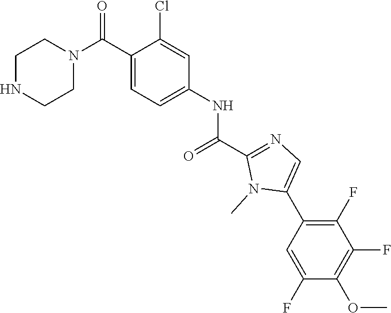 [SMILES: COc1c(F)cc(-c2cnc(C(=O)Nc3ccc(C(=O)N4CCNCC4)c(Cl)c3)n2C)c(F)c1F] | 508.2 | Intermediate 394 and Intermediate 322 |
| 396 | N-[3-chloro-4- (piperazine-1- carbonyl)phenyl]-5-(4- ethoxy-2,3-difluoro- phenyl)-1-methyl- imidazole-2- carboxamide | 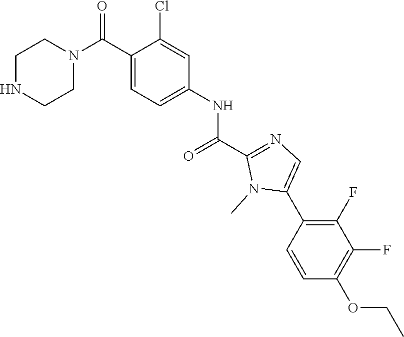 [SMILES: CCOc1ccc(-c2cnc(C(=O)Nc3ccc(C(=O)N4CCNCC4)c(Cl)c3)n2C)c(F)c1F] | 504.1 | Intermediate 313 and (4- ethoxy-2,3- difluoro- phenyl)boronic acid |
| 397 | N-[3-chloro-4- (piperazine-1- carbonyl)phenyl]-1- methyl-5-(2,3,4- trifluorophenyl) imidazole-2- carboxamide | 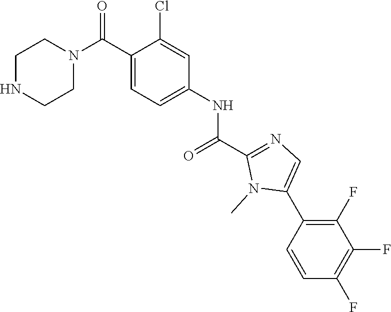 [SMILES: Cn1c(-c2ccc(F)c(F)c2F)cnc1C(=O)Nc1ccc(C(=O)N2CCNCC2)c(Cl)c1] | 478.0 | Intermediate 394 and (2,3,4- trifluorophenyl) boronic acid |
| 398 | N-[3-chloro-4- (piperazine-1- carbonyl)phenyl]-5-(3- cyano-4-methoxy- phenyl)-1-methyl- imidazole-2- carboxamide | 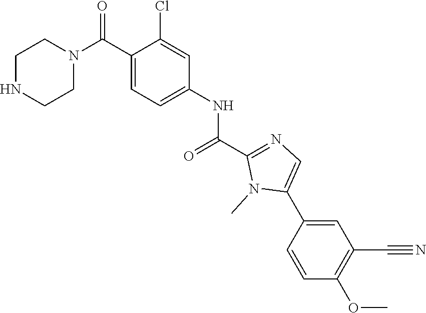 [SMILES: COc1ccc(-c2cnc(C(=O)Nc3ccc(C(=O)N4CCNCC4)c(Cl)c3)n2C)cc1C#N] | 479.4 | Intermediate 394 and (3- cyano-4- methoxy- phenyl)boronic acid |
| 399 | N-[3-chloro-4- (piperazine-1- carbonyl)phenyl]-5-(3- cyano-2,4-difluoro- phenyl)-1-methyl- imidazole-2- carboxamide | 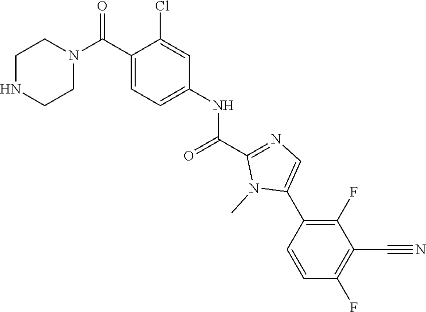 [SMILES: Cn1c(-c2ccc(F)c(C#N)c2F)cnc1C(=O)Nc1ccc(C(=O)N2CCNCC2)c(Cl)c1] | 484.9 | Intermediate 394 and (3- cyano-2,4- difluoro- phenyl)boronic acid |
| 400 | 5-[2-chloro-3-fluoro-4- (fluoromethoxy)phenyl]- N-[3-chloro-4- (piperazine-1- carbonyl)phenyl]-1- methyl-imidazole-2- carboxamide | 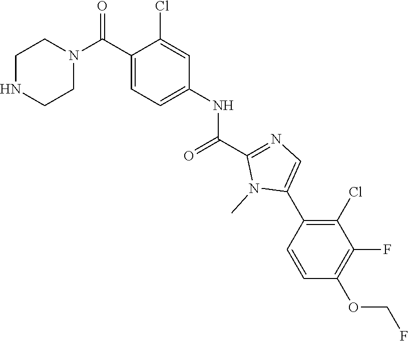 [SMILES: Cn1c(-c2ccc(OCF)c(F)c2Cl)cnc1C(=O)Nc1ccc(C(=O)N2CCNCC2)c(Cl)c1] | 524.0 | Intermediate 394 and Intermediate 321 |
| 5 | 5-[4-(cyanomethoxy)- 2,3-difluoro-phenyl]-1- methyl-N-[3-methyl-4- (piperazine-1- carbonyl)phenyl] imidazole-2- carboxamide | 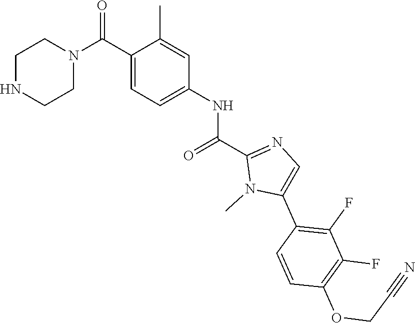 [SMILES: Cc1cc(NC(=O)c2ncc(-c3ccc(OCC#N)c(F)c3F)n2C)ccc1C(=O)N1CCNCC1] | 495.2 | Intermediate 6 and Intermediate 401 |
| 7 | N-[3-chloro-4- (piperazine-1- carbonyl)phenyl]-5-[4- (cyanomethoxy)-2,3- difluoro-phenyl]-1- methyl-imidazole-2- carboxamide | 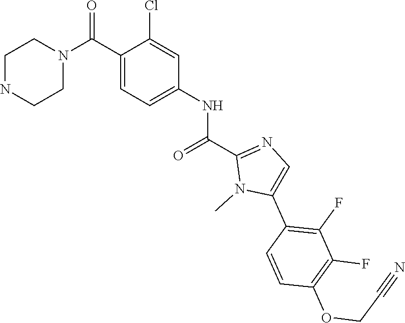 [SMILES: Cn1c(-c2ccc(OCC#N)c(F)c2F)cnc1C(=O)Nc1ccc(C(=O)N2CCNCC2)c(Cl)c1] | 515.2 | Intermediate 8 and Intermediate 401 |
| 9 | N-[3-chloro-4- (piperazine-1- carbonyl)phenyl]-5-(2,3- difluoro-4-methoxy- phenyl)-1-methyl- imidazole-2- carboxamide | 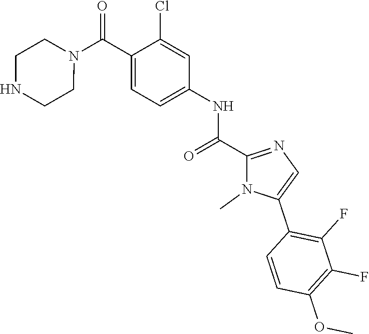 [SMILES: COc1ccc(-c2cnc(C(=O)Nc3ccc(C(=O)N4CCNCC4)c(Cl)c3)n2C)c(F)c1F] | 490.1 | Intermediate 8 and (2,3- difluoro-4- methoxy- phenyl)boronic acid |
| 10 | 5-(2,3-difluoro-4- methoxy-phenyl)-1- methyl-N-[3-methyl-4- (piperazine-1- carbonyl)phenyl] imidazole-2- carboxamide | 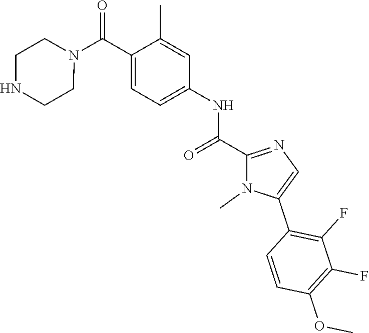 [SMILES: COc1ccc(-c2cnc(C(=O)Nc3ccc(C(=O)N4CCNCC4)c(C)c3)n2C)c(F)c1F] | 470.1 | Intermediate 6 and 2,3- difluoro-4- methoxyphenyl- boronic acid |

### Intermediate Type VII: 402

### tert-Butyl N-[2-(4-piperidyloxy)ethyl]carbamate

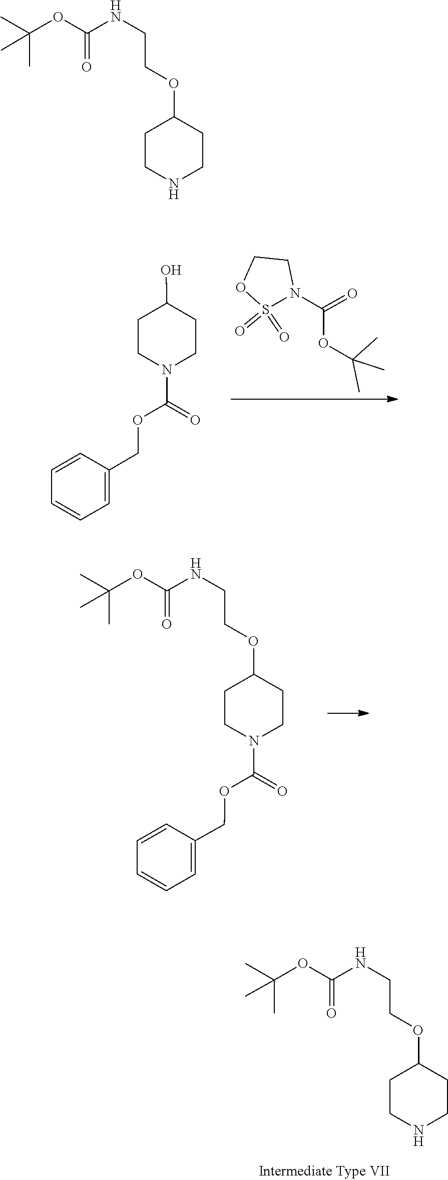

### Step 1: benzyl 4-[2-(tert-butoxycarbonylamino)ethoxy]piperidine-1-carboxylate

To a solution of benzyl 4-hydroxypiperidine-1-carboxylate (500 mg, 2.13 mmol) in DMF (10 mL) was added sodium hydride (170 mg, 4.25 mmol) in portions at 0° C. The reaction mixture was stirred for 30 minutes, then tert-butyl 1,2,3-oxathiazolidine-3-carboxylate 2,2-dioxide (474 mg, 2.13 mmol) was added. The reaction was slowly warmed to room temperature and stirred for overnight. The reaction mixture was washed with brine and extracted in DCM. The organic layer was dried over anhydrous Na₂SO₄ and concentrated in vacuum to give benzyl 4-(2-((tert-butoxycarbonyl)amino)ethoxy)piperidine-1-carboxylate (760 mg, 94.5% yield). MS (ESI, m/z): 379.2 [M+H]⁺.

### Step 2: tert-butyl N-[2-(4-piperidyloxy)ethyl]carbamate

To a solution of benzyl 4-(2-((tert-butoxycarbonyl)amino)ethoxy)piperidine-1-carboxylate (760 mg, 2.01 mmol) in EtOH (20 mL) was added Pd(OH)₂ (300 mg), the reaction was stirred for 6 hours under atmosphere of hydrogen at room temperature. The mixture was filtered and the filtrate was concentrated in vacuum to give tert-butyl (2-(piperidin-4-yloxy)ethyl)carbamate (320 mg, 65.2% yield). MS (ESI, m/z): 245.1 [M+H]⁺.

### Intermediate Type VII: 403

### tert-Butyl 3-(4-piperidyloxymethyl)azetidine-1-carboxylate

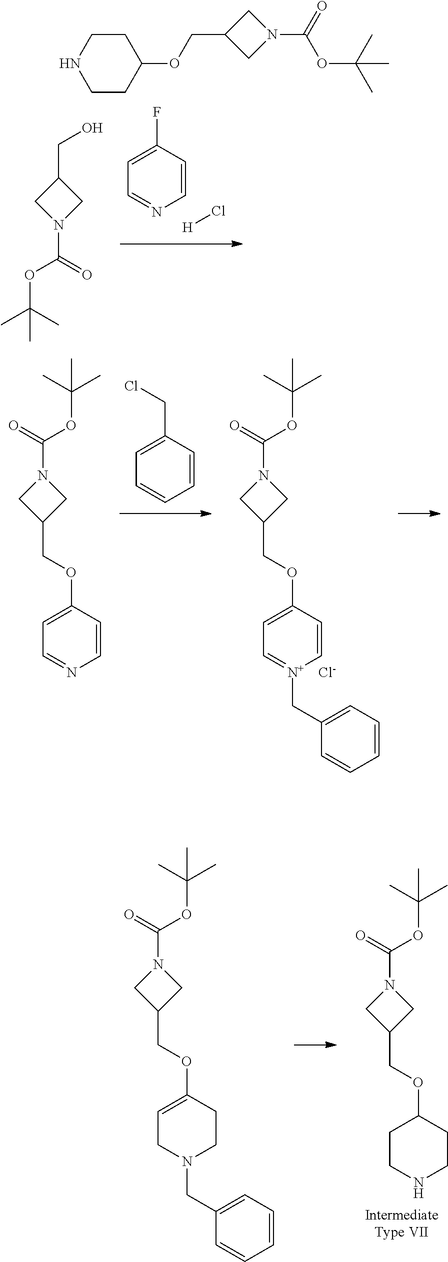

### Step 1: tert-butyl 3-(4-pyridyloxymethyl)azetidine-1-carboxylate

To a solution of tert-butyl 3-(hydroxymethyl)azetidine-1-carboxylate (500 mg, 2.67 mmol) in DMF (10 mL) was added sodium hydride (320 mg, 8.01 mmol) in portions at 0° C., the reaction mixture was stirred for 30 minutes, then 4-fluoropyridine hydrochloride (357 mg, 2.67 mmol) was added. The reaction mixture was slowly warmed to 60° C. and stirred for one hour. The reaction was quenched with water and extracted in EtOAc. The organic layer was dried over anhydrous Na₂SO₄ and concentrated in vacuum to give tert-butyl 3-((pyridin-4-yloxy) methyl)azetidine-1-carboxylate (600 mg, 85% yield). MS (ESI, m/z): 265.1 [M+H]⁺.

### Step 2: tert-butyl 3-[(1-benzylpyridin-1-ium-4-yl)oxymethyl]azetidine-1-carboxylate; chloride

To a solution of tert-butyl 3-((pyridin-4-yloxy)methyl)azetidine-1-carboxylate (600 mg, 2.27 mmol) in MeCN (10 mL) was added (chloromethyl)benzene (287 mg, 2.27 mmol). The reaction was stirred for 15 hours at 70° C. The reaction mixture was cooled to room temperature and concentrated in vacuum to give 1-benzyl-4-((1-(tert-butoxycarbonyl) azetidin-3-yl)methoxy)pyridin-1-ium chloride (882 mg, 99.4% yield). MS (ESI, m/z): 355.2 [M]⁺.

### Step 3: tert-butyl 3-[(1-benzyl-3,6-dihydro-2H-pyridin-4-yl)oxymethyl]azetidine-1-carboxylate

To a solution of 1-benzyl-4-((1-(tert-butoxycarbonyl)azetidin-3-yl)methoxy)pyridin-1-ium chloride (800 mg, 2.05 mmol) in MeOH (15 mL) cooled with an ice bath was added sodium borohydride (387 mg, 10.2 mmol) in portions. The reaction was slowly warmed to room temperature and stirred overnight. The reaction was quenched with ammonium chloride and washed with brine. The mixture was extracted in EtOAc and the organic layer was dried over anhydrous Na₂SO₄ and concentrated in vacuum to give tert-butyl 3-(((1-benzyl-1,2,3,6-tetrahydropyridin-4-yl)oxy)methyl)azetidine-1-carboxylate (700 mg, 95.4% yield). MS (ESI, m/z): 359.2 [M+H]⁺.

### Step 4: tert-butyl 3-(4-piperidyloxymethyl)azetidine-1-carboxylate

To a solution of tert-butyl 3-(((l-benzyl-1,2,3,6-tetrahydropyridin-4-yl)oxy)methyl)azetidine-1-carboxylate (700 mg, 1.95 mmol) in EtOH (20 mL) was added Pd(OH)₂ (350 mg), the reaction was stirred for two hours at room temperature under an hydrogen atmosphere. The reaction mixture was filtered and the filtrate was concentrated in vacuum to give tert-butyl 3-((piperidin-4-yloxy)methyl)azetidine-1-carboxylate (430 mg, 81.4% yield). MS (ESI, m/z): 271.2 [M+H]⁺.

### Intermediate Type VII: 404

### tert-Butyl (2-(azetidin-3-yloxy)ethyl)carbamate

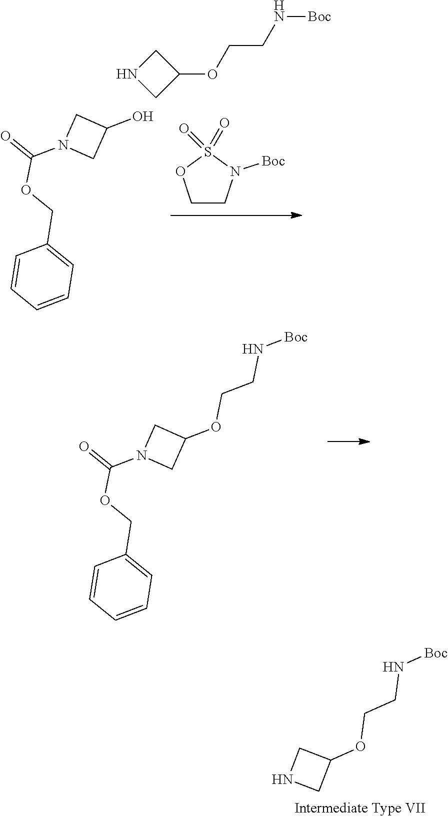

### Step 1: benzyl 3-(2-((tert-butoxycarbonyl)amino)ethoxy)azetidine-1-carboxylate

In a 100 mL round-bottomed flask, NaH (255 mg, 6.37 mmol) was combined with DMF (15 mL) to give a colorless solution. Benzyl 3-hydroxyazetidine-1-carboxylate (1.1 g, 5.31 mmol) was added at 0° C. The reaction was stirred at 0° C. for 30 min, then the reaction was stirred at room temperature for 30 min. Then tert-butyl 1,2,3-oxathiazolidine-3-carboxylate 2,2-dioxide (1.42 g, 6.37 mmol) was added at 0° C. The reaction was stirred at room temperature overnight. The reaction mixture was poured into 50 mL H₂O and extracted with EtOAc (3×50 mL). The organic layers were combined, washed with sat NaCl (1×50 mL). The organic layers were dried over Na₂SO₄ and concentrated in vacuo to afford benzyl 3-(2-((tert-butoxycarbonyl) amino)ethoxy)azetidine-1-carboxylate (1.86 g, 100% yield).

### Step 2: tert-butyl (2-(azetidin-3-yloxy)ethyl)carbamate

In a 100 mL round-bottomed flask, benzyl 3-(2-((tert-butoxycarbonyl)amino)ethoxy)azetidine-1-carboxylate (350 mg, 999 μmol) was combined with MeOH (30 mL) to give a colorless solution. Pd—C (21.3 mg, 200 μmol) was added. The reaction was purged with hydrogen three times and was then stirred at room temperature 1 h. The reaction mixture was filtered through celite. The filtrate was concentrated in vacuo to afford tert-butyl (2-(azetidin-3-yloxy)ethyl) carbamate (122 mg, 56.5% yield). (ESI, m/z): 217.3 [M+H]+.

### Intermediate Type VII: 405

### tert-Butyl (R)-(1-(piperazine-1-carbonyl)pyrrolidin-3-yl)carbamate

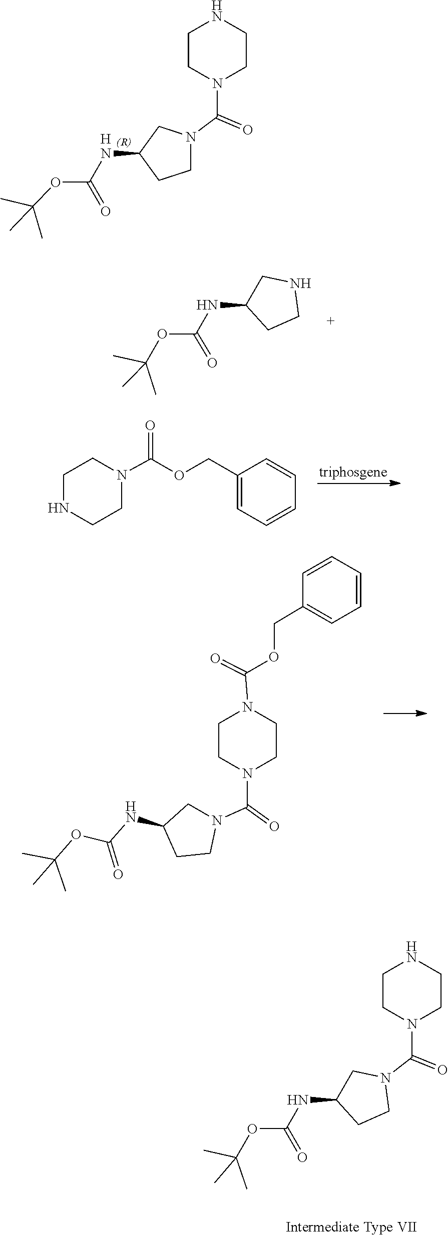

### Step 1: benzyl (R)-4-(3-((tert-butoxycarbonyl)amino)pyrrolidine-1-carbonyl)piperazine-1-carboxylate

A mixture of (R)-3-(Boc-amino)pyrrolidine (0.5 g, 2.68 mmol), 1-Cbz-piperazine (0.59 g, 2.68 mmol), triphosgene (0.48 g, 0.810 mmol) and triethylamine (0.93 mL, 6.71 mmol) in DMF (15 mL) was stirred at 25° C. for 16 h. The mixture was added to water (50 mL) and extracted with ethyl acetate (20 mL×3). The combined organic layers were dried over anhydrous Na₂SO₄ and concentrated under reduced pressure. The residue was then purified by flash column chromatography to afford the title compound (178 mg) as a white solid. MS (ESI, m/z): 433.2 [M+H]⁺.

### Step 2: tert-butyl (R)-(1-(piperazine-1-carbonyl)pyrrolidin-3-yl)carbamate

A solution of benzyl 4-[3-(tert-butoxycarbonylamino)pyrrolidine-1-carbonyl]piperazine-1-carboxylate (148.0 mg, 0.340 mmol) and Pd/C (20.0 mg, 15% w/w) in methanol (10 mL) was stirred at 25° C. for 6 h under hydrogen atmosphere. After filtration through Celite, the filtrate was concentrated under reduced pressure to afford the title compound (100 mg) as a white solid, which was used in next step without any purification. MS (ESI, m/z): 299.2 [M+H]⁺.

### Intermediate Type VII: 406

### tert-butyl 4-(4-piperidylsulfonyl)piperazine-1-carboxylate

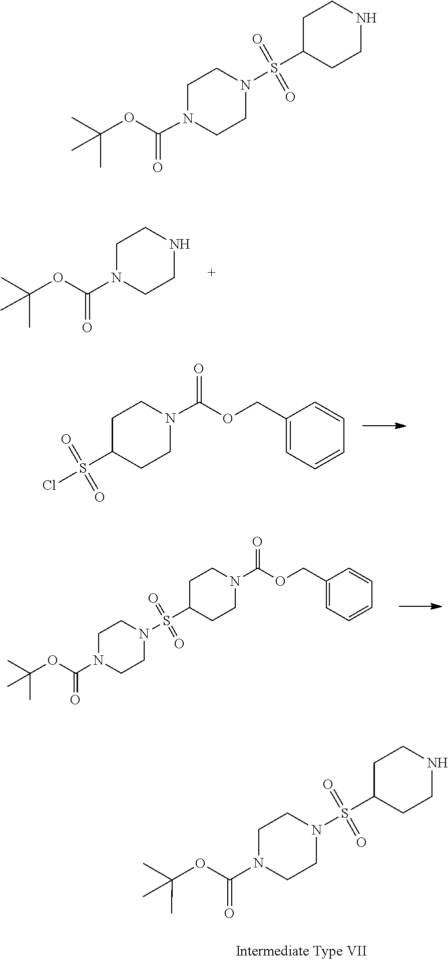 [SMILES: CC(C)(C)OC(=O)N1CCN(S(=O)(=O)C2CCNCC2)CC1 ; CC(C)(C)OC(=O)N1CCNCC1 ; O=C(OCc1ccccc1)N1CCC(S(=O)(=O)Cl)CC1 ; CC(C)(C)OC(=O)N1CCN(S(=O)(=O)C2CCN(C(=O)OCc3ccccc3)CC2)CC1 ; CC(C)(C)OC(=O)N1CCN(S(=O)(=O)C2CCNCC2)CC1]

### Step 1: tert-butyl 4-[(1-benzyloxycarbonyl-4-piperidyl)sulfonyl]piperazine-1-carboxylate

A mixture of N,N-diisopropylethylamine (0.69 mL, 3.93 mmol), 1-Boc-piperazine (439.56 mg, 2.36 mmol), benzyl 4-chlorosulfonylpiperidine-1-carboxylate (0.5 g, 1.57 mmol) in DCM (10 mL) was stirred at 25° C. under nitrogen for 3 h. To the mixture was added water (5 mL) and it was extracted with DCM (10 mL×3). The combined organic layers were concentrated under reduced pressure. The residue was then purified by flash column to afford the title compound (570 mg) as white solid. MS (ESI, m/z): 490.2 [M+H]⁺.

### Step 2: tert-butyl 4-(4-piperidylsulfonyl)piperazine-1-carboxylate

A solution of Pd/C (100.0 mg, 18% w/w) and tert-butyl 4-[(1-benzyloxycarbonyl-4-piperidyl)sulfonyl]piperazine-1-carboxylate (570 mg, 1.22 mmol) in methanol (20 mL) was stirred under hydrogen atmosphere at 25° C. for 12 h. After filtration, the filtrate was concentrated under reduced pressure to give the title compound (455 mg) as colorless oil. MS (ESI, m/z): 334.2 [M+H]⁺.

The following Type VII Intermediates were prepared in analogy to 406.

| | | | ESI MS | |
|---|---|---|---|---|
| Int. | Name | Structure | [M + H]⁺ | Starting Material |
| 407 | tert-butyl 4- (piperidine-4- carbonyl) piperazine-1- carboxylate | 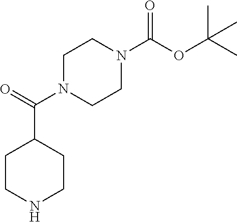 [SMILES: CC(C)(C)OC(=O)N1CCN(C(=O)C2CCNCC2)CC1] | 298.2 | 1-((benzyloxy) carbonyl) piperidine-4- carboxylic acid and tert-butyl piperazine-1- carboxylate |
| 408 | tert-butyl 4- (piperazine-1- carbonyl) piperidine-1- carboxylate | 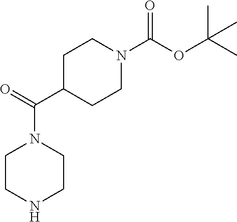 [SMILES: CC(C)(C)OC(=O)N1CCC(C(=O)N2CCNCC2)CC1] | 298.2 | tert-butyl piperazine-1- carboxylate and 1- (tert- butoxycarbonyl) piperidine-4- carboxylic acid |

### Intermediate Type VII: 409

### Methyl 3-(3-oxo-3-piperazin-1-yl-propoxy)propanoate (409) and 3-methoxy-1-(piperazin-1-yl)propan-1-one (409a)

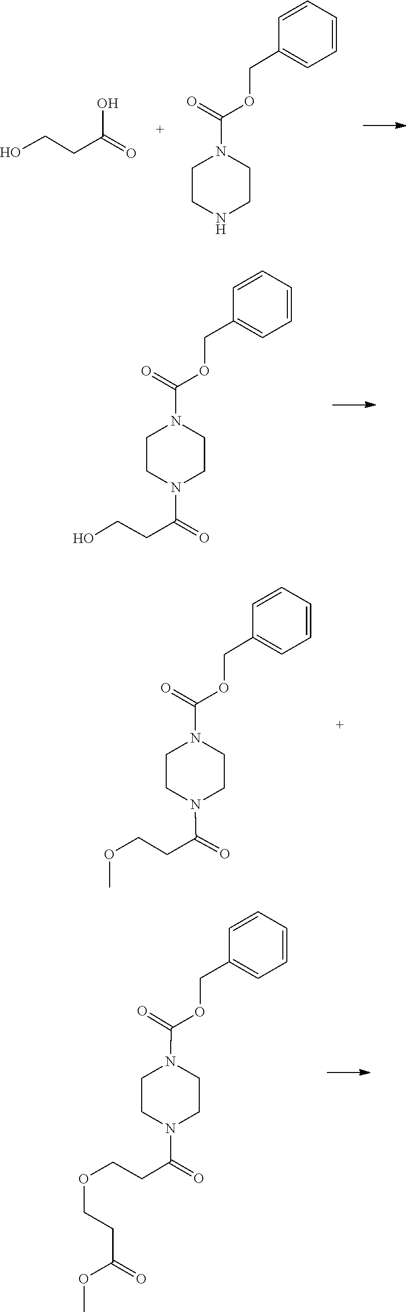 [SMILES: O=C(O)CCO ; O=C(OCc1ccccc1)N1CCNCC1 ; O=C(CCO)N1CCN(C(=O)OCc2ccccc2)CC1 ; COCCC(=O)N1CCN(C(=O)OCc2ccccc2)CC1 ; COC(=O)CCOCCC(=O)N1CCN(C(=O)OCc2ccccc2)CC1]

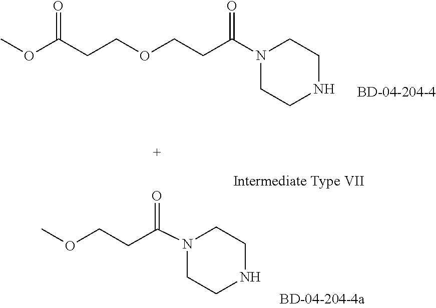 [SMILES: COC(=O)CCOCCC(=O)N1CCNCC1 ; COCCC(=O)N1CCNCC1]

### Step 1: benzyl 4-(3-hydroxypropanoyl)piperazine-1-carboxylate

A mixture of 1-Cbz-piperazine (5.0 g, 22.7 mmol), 3-hydroxypropionic acid (3.07 g, 34.05 mmol), 1-propylphosphonic anhydride solution, 50 wt % in ethyl acetate (28.89 g, 45.4 mmol) and N,N-diisopropylethylamine (9.88 mL, 56.75 mmol) in DCM (113.5 mL) was stirred at 25° C. for 16 h. The mixture was added to water (20 mL) and extracted with ethyl acetate (20 mL×3). The combined organic layers were concentrated under reduced pressure. The residue was purified by column to afford the title compound (2.91 g) as brown oil. MS (ESI, m/z): 293.1 [M+H]⁺.

### Step 2: benzyl 4-[3-(3-methoxy-3-oxo-propoxy)propanoyl]piperazine-1-carboxylate and benzyl 4-(3-methoxypropanoyl)piperazine-1-carboxylate

To a mixture of benzyl 4-(3-hydroxypropanoyl)piperazine-1-carboxylate (2.5 g, 8.55 mmol) and sodium methoxide (1386.01 mg, 25.66 mmol) in THF (42.76 mL), was added methyl acrylate (0.93 mL, 10.26 mmol). The mixture was stirred at 25° C. for 12 h. The mixture was quenched with water (10 mL) and extracted with ethyl acetate (10 mL×3). The combined organic layers were concentrated under reduced pressure. The crude was then purified by flash column chromatography to give a mixture of the title compounds (732 mg) as a yellow oil.

### Step 3: methyl 3-(3-oxo-3-piperazin-1-yl-propoxy)propanoate and 3-methoxy-1-(piperazin-1-yl)propan-1-one

A mixture of benzyl 4-[3-(3-methoxy-3-oxo-propoxy)propanoyl]piperazine-1-carboxylate and benzyl 4-(3-methoxypropanoyl)piperazine-1-carboxylate (732 mg) and Pd/C (73 mg, 10% w/w) in methanol (20 mL) was stirred under hydrogen atmosphere for 48 h. The mixture was filtered by Celite and the filtrate was concentrated under reduced pressure to afford a mixture of the title compounds (476 mg) as brown gum.

### Intermediate Type VII: 410

### 3-Cyclobutyl-5-(piperidin-4-ylmethyl)-1,2,4-oxadiazole

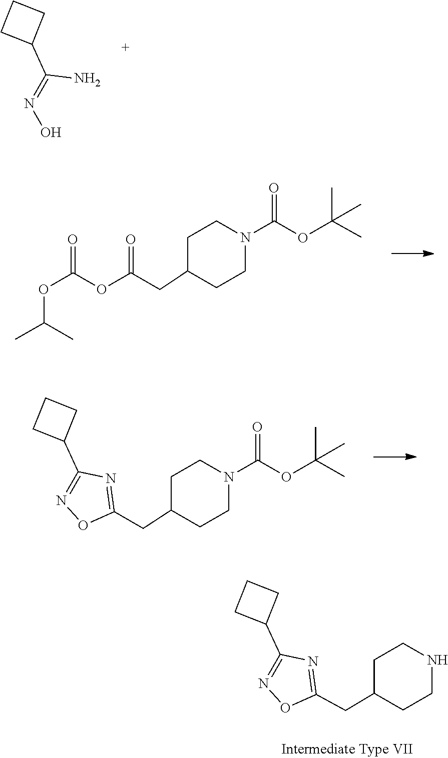 [SMILES: N/C(=N\O)C1CCC1 ; CC(C)OC(=O)OC(=O)CC1CCN(C(=O)OC(C)(C)C)CC1 ; CC(C)(C)OC(=O)N1CCC(Cc2nc(C3CCC3)no2)CC1 ; C1CC(c2noc(CC3CCNCC3)n2)C1]

### Step 1: tert-butyl 4-((3-cyclobutyl-1,2,4-oxadiazol-5-yl)methyl)piperidine-1-carboxylate

To a solution of (Z)-N′-hydroxycyclobutanecarboximidamide (4.68 g, 41 mmol, Eq: 0.99) in DMF (70 mL) and pyridine (6.85 g, 7 mL, 86.5 mmol, Eq: 2.09) at 50° C., a solution of 2-(1-(tert-butoxycarbonyl)piperidin-4-yl)acetic (isopropyl carbonic) anhydride (13.64 g, 41.4 mmol) in DMF (7 mL) was added dropwise over a period of 30 min. The mixture was stirred for 1 h at the same temperature. Then the light yellow clear solution was heated up to 100° C. and stirred overnight. The mixture was evaporated and absorbed with Isolute® HM-N column, dried and purified by flash chromatography to afford tert-butyl 4-((3-cyclobutyl-1,2,4-oxadiazol-5-yl) methyl)piperidine-1-carboxylate (8.866 g, 27.5 mmol, 66.6% yield) as a colorless oil. MS (ESI, m/z): 266.2 [M−tBu+H]⁺.

### Step 2: 3-cyclobutyl-5-(piperidin-4-ylmethyl)-1,2,4-oxadiazole

To a solution of tert-butyl 4-((3-cyclobutyl-1,2,4-oxadiazol-5-yl)methyl)piperidine-1-carboxylate (27.55 g, 85.7 mmol, Eq: 1) in dichloromethane (240 mL), 4M HCl in dioxane (85 mL, 340 mmol) was added dropwise over a period of 1.5 h and the mixture was stirred for 2.5 h at room temperature and was then evaporated. As the product started to crystallize, the evaporation was stopped, 300 mL diethylether was added and the mixture was stirred for 30 min at room temperature. The white crystals were filtered off, washed twice with 100 mL of diethylether and dried under reduced pressure to afford 3-cyclobutyl-5-(piperidin-4-ylmethyl)-1,2,4-oxadiazole (21.618 g, 83.9 mmol, 97.8% yield) as white crystals. MS (ESI, m/z): 222.2 [M+H]⁺.

### Intermediate Type VIII: 411

### tert-Butyl (2-amino-2-oxoethyl)(2-(piperazin-1-yl)ethyl)carbamate

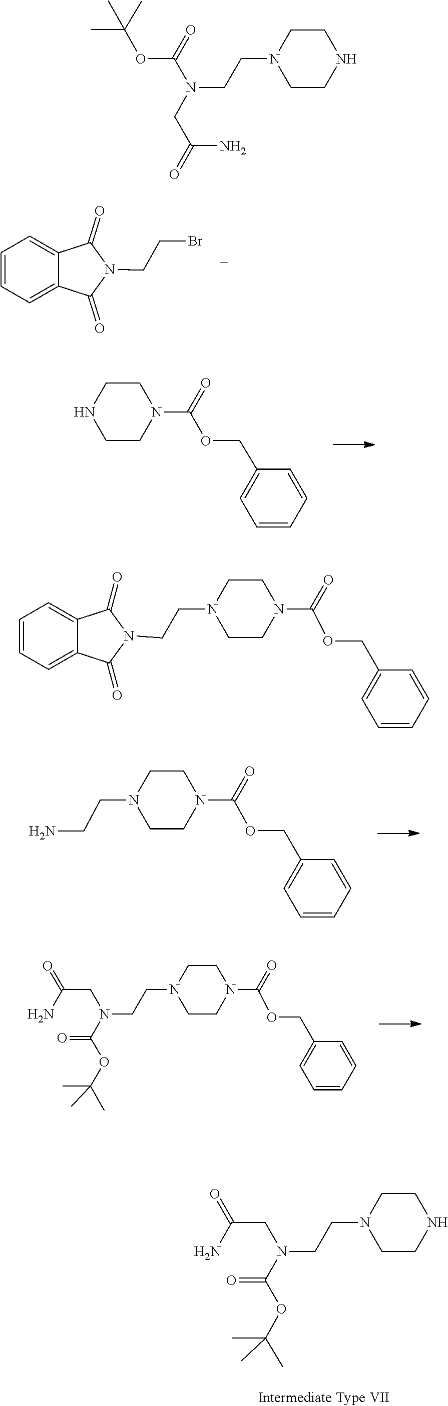

### Step 1: benzyl 4-(2-(1,3-dioxoisoindolin-2-yl)ethyl)piperazine-1-carboxylate

A mixture of N-(2-bromoethyl)phthalimide (5.0 g, 19.68 mmol), 1-Cbz-piperazine (5.2 g, 23.61 mmol) and triethylamine (4.11 mL, 29.52 mmol) in THF (30 mL) was stirred at 70° C. for 14 h. To the mixture was added H₂O (100 mL) and it was extracted with ethyl acetate (50 mL×3). The combined organic layers were dried over anhydrous sodium sulfate and concentrated under reduced pressure. The residue was purified by column chromatography to afford the title compound (4.29 g) as brown oil. MS (ESI, m/z): 394.2 [M+H]⁺.

### Step 2: benzyl 4-(2-aminoethyl)piperazine-1-carboxylate

A mixture of benzyl 4-[2-(1,3-dioxoisoindolin-2-yl)ethyl]piperazine-1-carboxylate (4.26 g, 10.83 mmol) and hydrazine hydrate (1.28 g, 21.7 mmol) in ethanol (50 mL) was stirred at 80° C. for 2 h. The mixture was filtered and the filtrate was concentrated under reduced pressure to afford the title compound (2.78 g) as a white solid, which was used in next step without further purification. MS (ESI, m/z): 264.2 [M+H]⁺.

### Step 3: benzyl 4-(2-((2-amino-2-oxoethyl)(tert-butoxycarbonyl)amino)ethyl)piperazine-1-carboxylate trifluoroacetate

To a mixture of benzyl 4-(2-aminoethyl)piperazine-1-carboxylate (1.5 g, 5.7 mmol) and N,N-diisopropylethylamine (4.96 mL, 28.48 mmol) in tetrahydrofuran (20 mL) was added 2-bromoacetamide (0.79 g, 5.7 mmol). The mixture was stirred at 25° C. for 14 h. To the mixture was added di-t-butyldicarbonate (2.49 g, 11.39 mmol). The mixture was stirred at 25° C. for 14 h. To the mixture was added water (30 mL) and it was extracted with ethyl acetate (20 mL×3). The combined organic layers were washed with saturated aqueous NaHCO₃ (20 mL) solution, dried over anhydrous sodium sulfate and concentrated under reduced pressure. The residue was purified by prep-HPLC (Chromatography column: Kromasil-C18 100×21.2 mm Sum; 5%-95% ACN in H₂O with 0.1% TFA as eluent) to afford the title compound (237 mg) as a white solid. MS (ESI, m/z): 421.2 [M+H]⁺.

### Step 4: tert-butyl (2-amino-2-oxoethyl)(2-(piperazin-1-yl)ethyl)carbamate

A mixture of benzyl 4-[2-[(2-amino-2-oxo-ethyl)-tert-butoxycarbonyl-amino]ethyl]piperazine-1-carboxylate trifluoroacetate (237.0 mg, 0.560 mmol) and Pd/C (47.4 mg, 20% w/w) in methanol (10 mL) was stirred at 25° C. for 14 h. The mixture was filtered by Celite and the filtrate was concentrated under reduced pressure to afford the title compound (122 mg) as a brown gum. MS (ESI, m/z): 287.2 [M+H]⁺.

The following Type VIII Intermediate was prepared in analogy to 411.

| | | | ESI MS | |
|---|---|---|---|---|
| Int. | Name | Structure | [M + H]⁺ | Starting Material |
| 412 | 2-(4-(piperazine- 1- carbonyl)piperidin- 1-yl)acetamide | 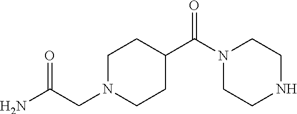 [SMILES: NC(=O)CN1CCC(C(=O)N2CCNCC2)CC1] | 255.2 | Intermediate 407 and 2- bromoacetamide |

### Intermediate Type IX: 413

### N-[3-Chloro-4-[4-(piperidine-4-carbonyl)piperazine-1-carbonyl]phenyl]-5-(2,3-difluoro-4-methoxy-phenyl)-1-methyl-imidazole-2-carboxamide

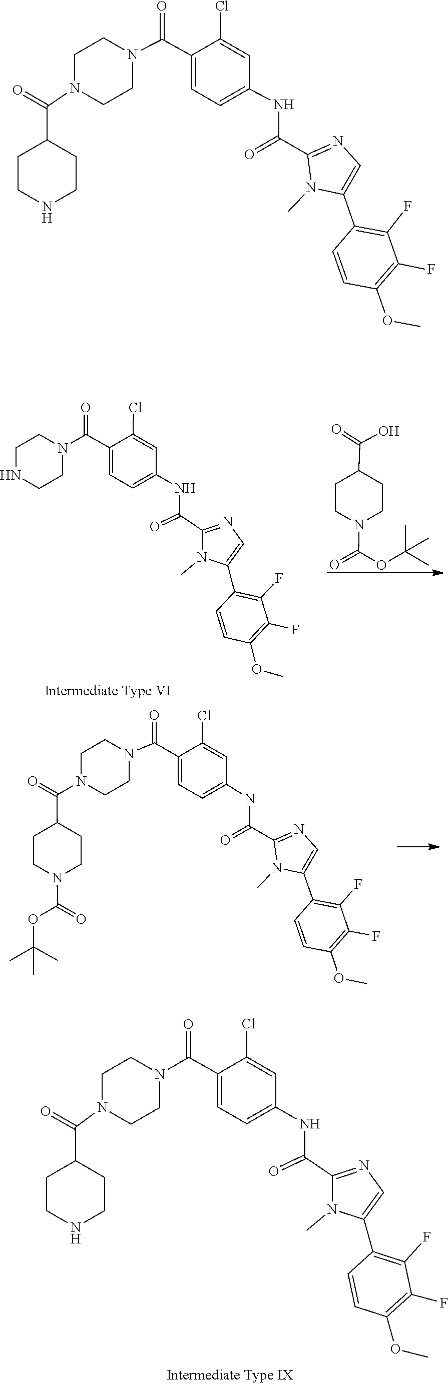

The title compound was obtained in analogy to Example 11, step 1-2 from N-[3-chloro-4-(piperazine-1-carbonyl)phenyl]-5-(2,3-difluoro-4-methoxy-phenyl)-1-methyl-imidazole-2-carboxamide (intermediate 374) and 1-(tert-butoxycarbonyl)piperidine-4-carboxylic acid. MS (ESI, m/z): 601.3 [M+H]⁺.

### Intermediate Type X: 415

### 2-(3-tert-Butoxycarbonylazetidin-1-yl)acetic acid

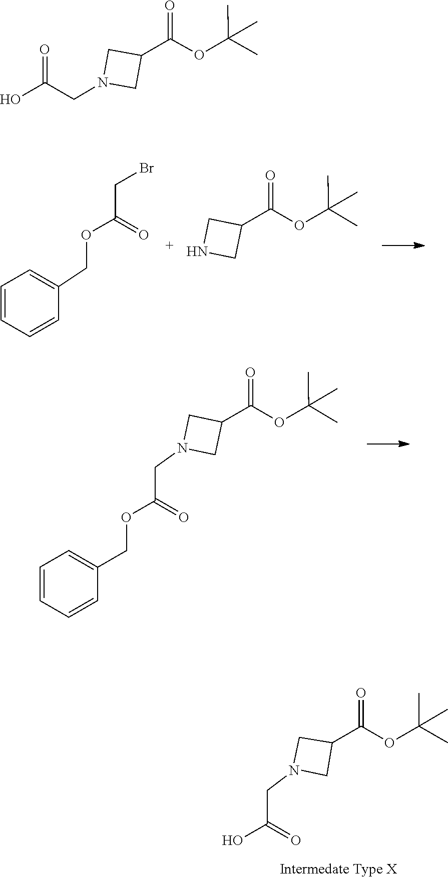 [SMILES: CC(C)(C)OC(=O)C1CN(CC(=O)O)C1 ; O=C(CBr)OCc1ccccc1 ; CC(C)(C)OC(=O)C1CNC1 ; CC(C)(C)OC(=O)C1CN(CC(=O)OCc2ccccc2)C1 ; CC(C)(C)OC(=O)C1CN(CC(=O)O)C1]

### Step 1: tert-butyl 1-(2-benzyloxy-2-oxo-ethyl)azetidine-3-carboxylate

To a solution of tert-butyl azetidine-3-carboxylate hydrochloride (910 mg, 4.7 mmol) and N,N-diisopropylethylamine (2.05 mL, 11.75 mmol) in DCM (15 mL) was added benzyl 2-bromoacetate (1.4 g, 6.11 mmol). The mixture was stirred at 25° C. for 5 h. After removal of solvent in vacuo, the crude was purified by column chromatography to give the title compound (980 mg) as colorless oil. MS (ESI, m/z): 306.2 [M+H]⁺.

### Step 2: 2-(3-tert-butoxycarbonylazetidin-1-yl)acetic acid

To a solution of tert-butyl 1-(2-benzyloxy-2-oxo-ethyl)azetidine-3-carboxylate (900 mg, 2.95 mmol) in methanol (5 mL) was added Pd/C (50.0 mg, 6% w/w) under nitrogen atmosphere. The mixture was stirred at 25° C. for 18 h under hydrogen atmosphere. The mixture was filtered by celite and the filtrate was concentrated in vacuo to give the title compound (660 mg) as a white solid. MS (ESI, m/z): 216.2 [M+H]⁺.

The following Type X Intermediate was prepared in analogy to 415.

| | | | ESI MS | |
|---|---|---|---|---|
| Int. | Name | Structure | [M + H]⁺ | Starting Material |
| 416 | (R)-2-(3-(tert- butoxycarbonyl) pyrrolidin-1-yl) acetic acid | 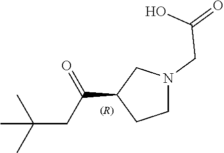 | 230.1 | tert-butyl (R)- pyrrolidine-3- carboxylate |

### Intermediate Type XI: 394

### tert-Butyl 4-(4-(5-bromo-1-methyl-1H-imidazole-2-carboxamido)-2-chlorobenzoyl)piperazine-1-carboxylate

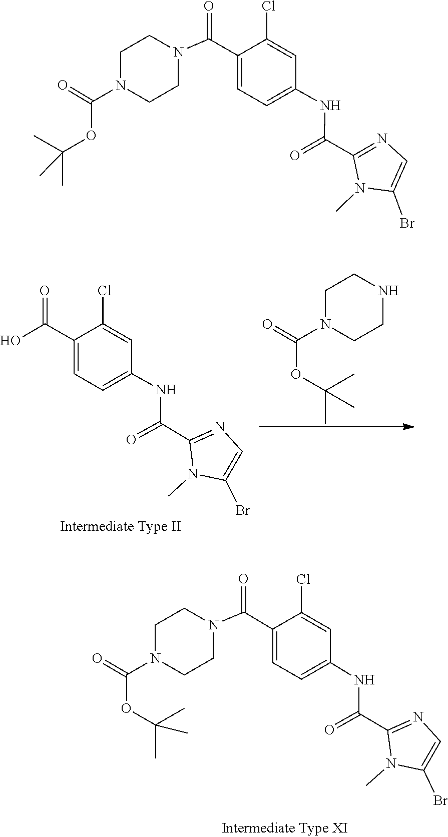 [SMILES: Cn1c(Br)cnc1C(=O)Nc1ccc(C(=O)N2CCN(C(=O)OC(C)(C)C)CC2)c(Cl)c1 ; Cn1c(Br)cnc1C(=O)Nc1ccc(C(=O)O)c(Cl)c1 ; CC(C)(C)OC(=O)N1CCNCC1 ; Cn1c(Br)cnc1C(=O)Nc1ccc(C(=O)N2CCN(C(=O)OC(C)(C)C)CC2)c(Cl)c1]

At room temperature, a mixture of 4-[(5-bromo-1-methyl-imidazole-2-carbonyl)amino]-2-chloro-benzoic acid (intermediate 313, 5.2 g, 14.5 mmol), tert-butyl piperazine-1-carboxylate (3.24 g, 17.4 mmol), HATU (6.62 g, 17.4 mmol) and DIPEA (5.62 g, 7.6 mL, 43.5 mmol) in DMF (5 mL) was stirred for 2 h. Then the mixture was poured into water. The water layer was extracted with DCM. The organic layer was washed with water, dried and concentrated to give the title compound (6.5 g, 85.1% yield) as a solid. MS (ESI, m/z): 526.1 [M+H]⁺.

The following Type XI Intermediates were prepared in analogy to intermediate 394.

| | | | ESI MS | |
|---|---|---|---|---|
| Int. | Name | Structure | [M + H]⁺ | Starting Material |
| 6 | tert-butyl 4-[4-[(5- bromo-1-methyl- imidazole-2- carbonyl)amino]-2- methyl- benzoyl]piperazine- 1-carboxylate | 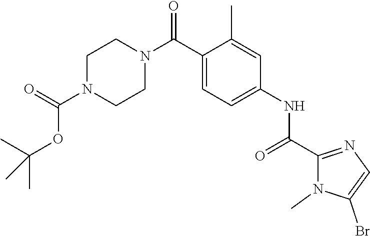 [SMILES: Cc1cc(NC(=O)c2ncc(Br)n2C)ccc1C(=O)N1CCN(C(=O)OC(C)(C)C)CC1] | 506.1 | 310 and tert-butyl piperazine-1- carboxylate |
| 417 | tert-butyl N-[3-[[4- [(5-bromo-1-methyl- imidazole-2- carbonyl)amino]-2- chloro- benzoyl]amino] cyclobutyl] carbamate | 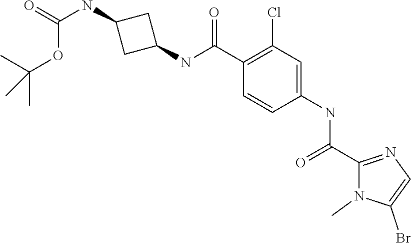 [SMILES: Cn1c(Br)cnc1C(=O)Nc1ccc(C(=O)N[C@H]2C[C@@H](NC(=O)OC(C)(C)C)C2)c(Cl)c1] | 550.1 | tert-butyl N-[3-[[4- [(5-bromo-1- methyl-imidazole- 2-carbonyl)amino]- 2-chloro- benzoyl]amino] cyclobutyl] carbamate |

### Intermediate Type XI: 418

### 5-Bromo-N-[3-chloro-4-(piperazine-1-carbonyl)phenyl]-1-methyl-imidazole-2-carboxamide

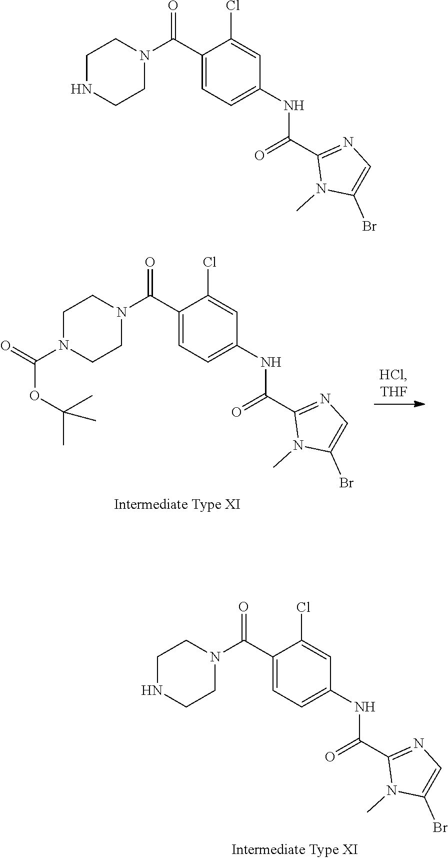 [SMILES: Cn1c(Br)cnc1C(=O)Nc1ccc(C(=O)N2CCNCC2)c(Cl)c1 ; Cn1c(Br)cnc1C(=O)Nc1ccc(C(=O)N2CCN(C(=O)OC(C)(C)C)CC2)c(Cl)c1 ; Cn1c(Br)cnc1C(=O)Nc1ccc(C(=O)N2CCNCC2)c(Cl)c1]

At room temperature, 12N HCl (10 mL) was added to a suspension of tert-butyl 4-[4-[(5-bromo-1-methyl-imidazole-2-carbonyl)amino]-2-chloro-benzoyl]piperazine-1-carboxylate (intermediate 394, 6.5 g, 12.3 mmol) in THF (50 mL). After stirring for 1 h, the solution was basified by NH₃.H₂O. The water phase was extracted with DCM. The organic layer was washed with water, dried and concentrated to give the title compound (5 g, 95% yield) as a yellow solid. MS (ESI, m/z): 426.2 [M+H]⁺.

The following Type XI Intermediate was prepared in analogy to intermediate 418.

| | | | ESI MS | |
|---|---|---|---|---|
| Int. | Name | Structure | [M + H]⁺ | Starting Material |
| 419 | 5-bromo-1-methyl- N-[3-methyl-4- (piperazine-1- carbonyl)phenyl] imidazole-2- carboxamide | 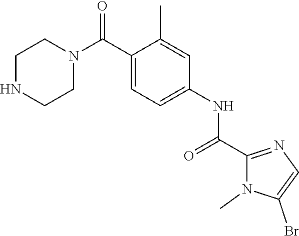 [SMILES: Cc1cc(NC(=O)c2ncc(Br)n2C)ccc1C(=O)N1CCNCC1] | 406.2 | Intermediate 314 and tert-butyl piperazine-1- carboxylate |

### Intermediate Type XI: 420

### 1-[4-[(5-Bromo-1-methyl-imidazole-2-carbonyl)amino]-2-chloro-benzoyl]piperidine-4-carboxylic acid

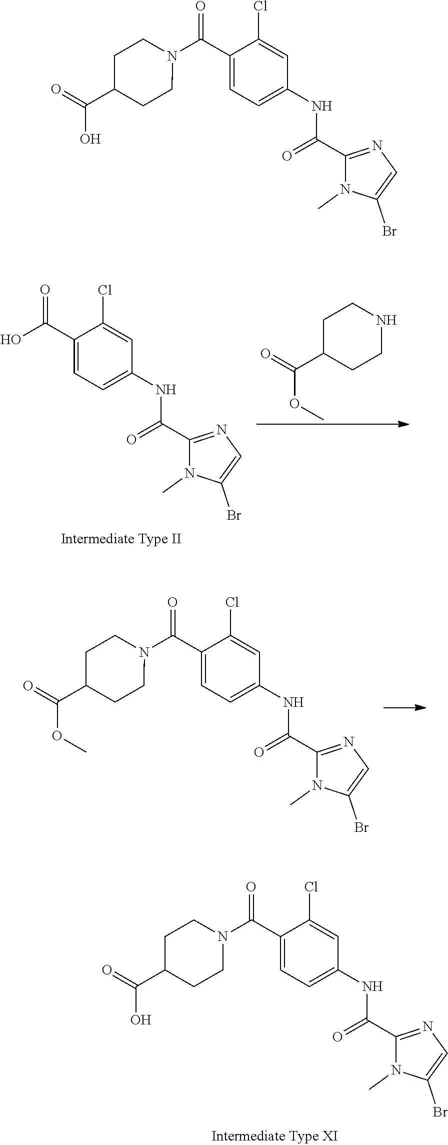

### Step 1: methyl 1-[4-[(5-bromo-1-methyl-imidazole-2-carbonyl)amino]-2-chloro-benzoyl]piperidine-4-carboxylate

The mixture of 4-(5-bromo-1-methyl-1H-imidazole-2-carboxamido)-2-chlorobenzoic acid (intermediate 313 2.8 g, 7.81 mmol), methyl piperidine-4-carboxylate (1.34 g, 9.37 mmol) and HATU (3.86 g, 10.2 mmol), DIPEA (5.05 g, 6.82 mL, 39 mmol) in DMF (15 mL) was stirred at 25° C. overnight. Then the mixture was poured into water. The water phase was extracted with DCM. The organic phase was dried and concentrated to give the crude product (1.9 g, 50.3% yield) as a yellow oil. MS (ESI, m/z): 483.1 [M+H]⁺.

### Step 2: 1-[4-[(5-bromo-1-methyl-imidazole-2-carbonyl)amino]-2-chloro-benzoyl]piperidine-4-carboxylic acid

A solution of methyl 1-(4-(5-bromo-1-methyl-1H-imidazole-2-carboxamido)-2-chlorobenzoyl) piperidine-4-carboxylate (1.9 g, 3.93 mmol) and lithium hydroxide monohydrate (824 mg, 19.6 mmol) in THF (24 mL) and water (12 mL), MeOH (1 mL) was stirred for 3 h. Then the solution was concentrated and the water layer was acidified by CH₃COOH. The water layer was extracted with DCM. The organic layer was washed with water, dried and concentrated to give the title compound (1.6 g, 86.7% yield) as a yellow oil. MS (ESI, m/z): 469.2 [M+H]⁺.

### Intermediate Type XII: 421

### tert-Butyl 3-[[[1-[4-[(5-bromo-1-methyl-imidazole-2-carbonyl)amino]-2-chloro-benzoyl]piperidine-4-carbonyl]amino]methyl]azetidine-1-carboxylate

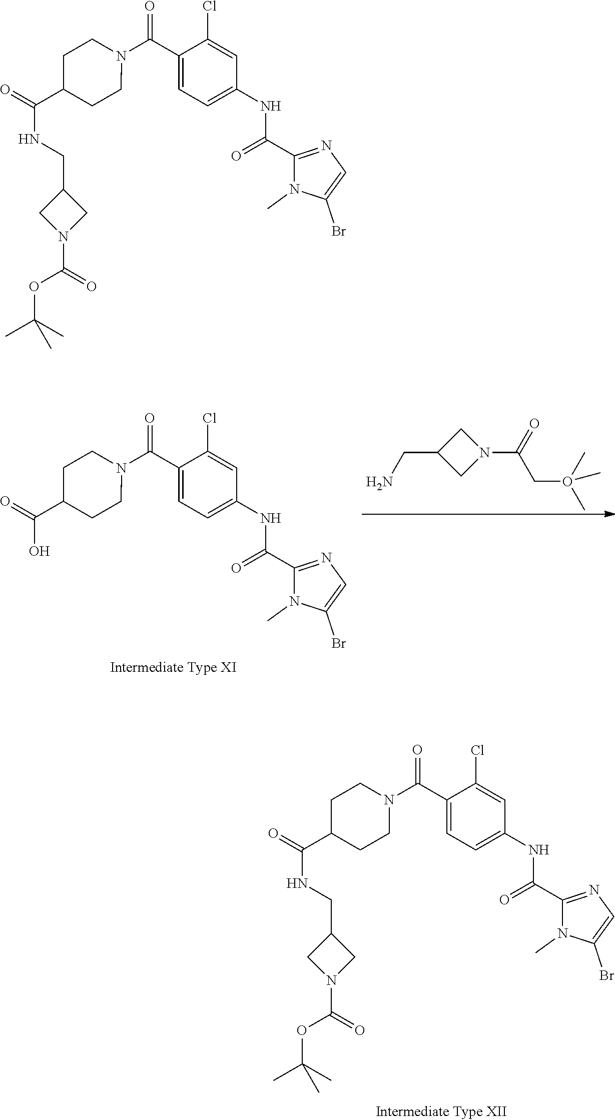

At room temperature, a mixture of 1-(4-(5-bromo-1-methyl-1H-imidazole-2-carboxamido)-2-chlorobenzoyl)piperidine-4-carboxylic acid (intermediate 420, 1.6 g, 3.41 mmol), tert-butyl 3-(aminomethyl)azetidine-1-carboxylate (1.9 g, 10.2 mmol), DIPEA (1.32 g, 1.78 mL, 10.2 mmol) and HATU (1.94 g, 5.11 mmol) in DMF (15 mL) was stirred for 1 h. Then the mixture was poured into water and the water phase was extracted with DCM. The organic phase was washed with water, dried and concentrated. The residue was purified by flash column to give the title compound (1.8 g, 82.8% yield) as a yellow oil. MS (ESI, m/z): 637.2 [M+H]⁺.

The following Type XII Intermediates were prepared in analogy to intermediate 421.

| | | | ESI MS | |
|---|---|---|---|---|
| Int. | Name | Structure | [M + H]⁺ | Starting Material |
| 422 | tert-butyl N-[2-[3-[4- [4-[(5-bromo-1- methyl-imidazole-2- carbonyl)amino]-2- chloro- benzoyl]piperazin-1- yl]-3-oxo- propoxy]ethyl] carbamate | 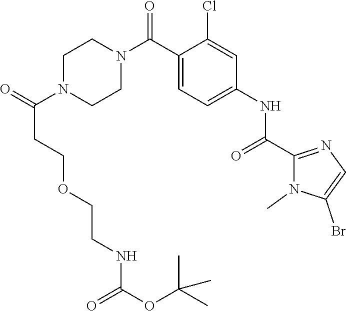 [SMILES: Cn1c(Br)cnc1C(=O)Nc1ccc(C(=O)N2CCN(C(=O)CCOCCNC(=O)OC(C)(C)C)CC2)c(Cl)c1] | 641.4 | Intermediate 418 and 3-(2-((tert- butoxycarbonyl) amino)ethoxy) propanoic acid |
| 423 | tert-butyl (2S,4R)-2- [4-[4-[(5-bromo-1- methyl-imidazole-2- carbonyl)amino]-2- chloro- benzoyl]piperazine- 1-carbonyl]-4- hydroxy-pyrrolidine- 1-carboxylate | 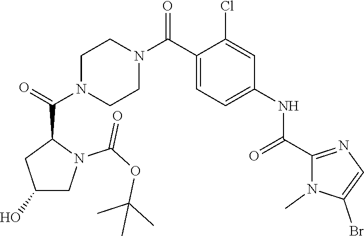 [SMILES: Cn1c(Br)cnc1C(=O)Nc1ccc(C(=O)N2CCN(C(=O)[C@@H]3C[C@@H](O)CN3C(=O)OC(C)(C)C)CC2)c(Cl)c1] | 638.20 | Intermediate 418 and (2S,4R)-1- (tert- butoxycarbonyl)-4- hydroxypyrrolidine- 2-carboxylic acid |
| 424 | tert-butyl (2S,4R)-2- [4-[4-[(5-bromo-1- methyl-imidazole-2- carbonyl)amino]-2- methyl- benzoyl]piperazine- 1-carbonyl]-4- hydroxy-pyrrolidine- 1-carboxylate | 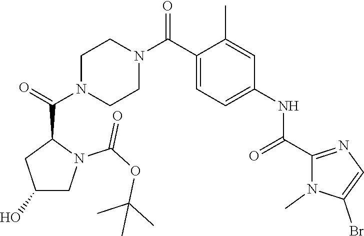 [SMILES: Cc1cc(NC(=O)c2ncc(Br)n2C)ccc1C(=O)N1CCN(C(=O)[C@@H]2C[C@@H](O)CN2C(=O)OC(C)(C)C)CC1] | 618.30 | Intermediate 419 and (2S,4R)-1- (tert- butoxycarbonyl)-4- hydroxypyrrolidine- 2-carboxylic acid |
| 425 | tert-butyl 3-[[1-[4- [(5-bromo-1-methyl- imidazole-2- carbonyl)amino]-2- chloro- benzoyl]piperidine- 4-carbonyl]amino] azetidine-1- carboxylate | 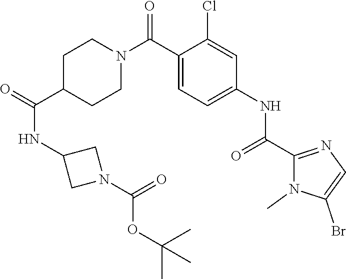 [SMILES: Cn1c(Br)cnc1C(=O)Nc1ccc(C(=O)N2CCC(C(=O)NC3CN(C(=O)OC(C)(C)C)C3)CC2)c(Cl)c1] | 623.2 | Intermediate 420 and tert-butyl 3- aminoazetidine-1- carboxylate |
| 426 | tert-butyl 4-[4-[4- [(5-bromo-1-methyl- imidazole-2- carbonyl)amino]-2- chloro- benzoyl]piperazine- 1-carbonyl] piperidine- 1-carboxylate | 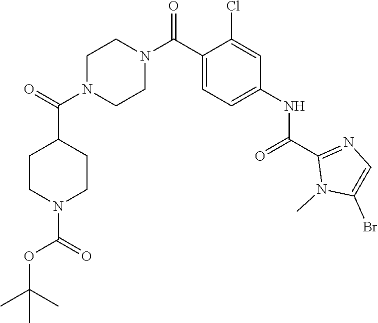 [SMILES: Cn1c(Br)cnc1C(=O)Nc1ccc(C(=O)N2CCN(C(=O)C3CCN(C(=O)OC(C)(C)C)CC3)CC2)c(Cl)c1] | 637.1 | Intermediate 418 and 1-(tert- butoxycarbonyl) piperidine-4- carboxylic acid |
| 427 | tert-butyl 4-[4-[4- [(5-bromo-1-methyl- imidazole-2- carbonyl)amino]-2- chloro- benzoyl]piperazine- 1-carbonyl]-4- hydroxy-piperidine- 1-carboxylate | 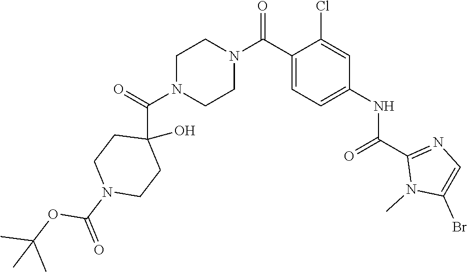 [SMILES: Cn1c(Br)cnc1C(=O)Nc1ccc(C(=O)N2CCN(C(=O)C3(O)CCN(C(=O)OC(C)(C)C)CC3)CC2)c(Cl)c1] | 653.4 | Intermediate 418 and 1-(tert- butoxycarbonyl)-4- hydroxypiperidine- 4-carboxylic acid |
| 428 | tert-butyl (3S)-3-[4- [4-[(5-bromo-1- methyl-imidazole-2- carbonyl)amino]-2- chloro- benzoyl]piperazine- 1-carbonyl] pyrrolidine- 1-carboxylate | 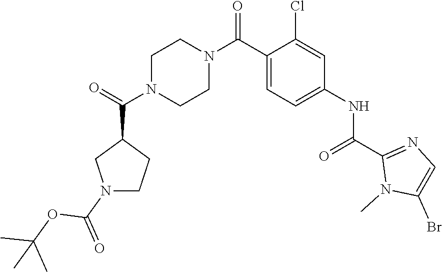 [SMILES: Cn1c(Br)cnc1C(=O)Nc1ccc(C(=O)N2CCN(C(=O)[C@H]3CCN(C(=O)OC(C)(C)C)C3)CC2)c(Cl)c1] | 623.1 | Intermediate 418 and (S)-1-(tert- butoxycarbonyl) pyrrolidine-3- carboxylic acid |
| 429 | tert-butyl (3R)-3-[4- [4-[(5-bromo-1- methyl-imidazole-2- carbonyl)amino]-2- chloro- benzoyl]piperazine- 1-carbonyl] pyrrolidine- 1-carboxylate | 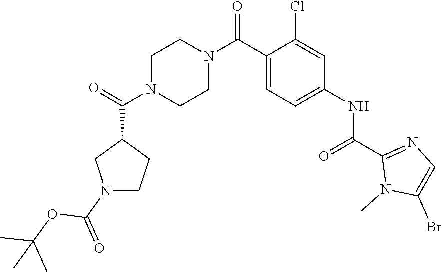 [SMILES: Cn1c(Br)cnc1C(=O)Nc1ccc(C(=O)N2CCN(C(=O)[C@@H]3CCN(C(=O)OC(C)(C)C)C3)CC2)c(Cl)c1] | 623.1 | Intermediate 418 and (R)-1-(tert- butoxycarbonyl) pyrrolidine-3- carboxylic acid |
| 430 | tert-butyl (3S)-3-[2- [4-[4-[(5-bromo-1- methyl-imidazole-2- carbonyl)amino]-2- chloro- benzoyl]piperazin-1- yl]-2-oxo- ethyl]pyrrolidine-1- carboxylate | 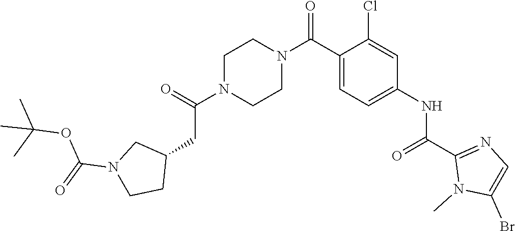 [SMILES: Cn1c(Br)cnc1C(=O)Nc1ccc(C(=O)N2CCN(C(=O)C[C@@H]3CCN(C(=O)OC(C)(C)C)C3)CC2)c(Cl)c1] | 637.2 | Intermediate 418 and (S)-2-(1-(tert- butoxycarbonyl) pyrrolidin-2-yl) acetic acid |

### Intermediate Type XII: 431

### 5-Bromo-N-[3-chloro-4-[4-[2-(dimethylamino)ethyl]piperazine-1-carbonyl]phenyl]-1-methyl-imidazole-2-carboxamide

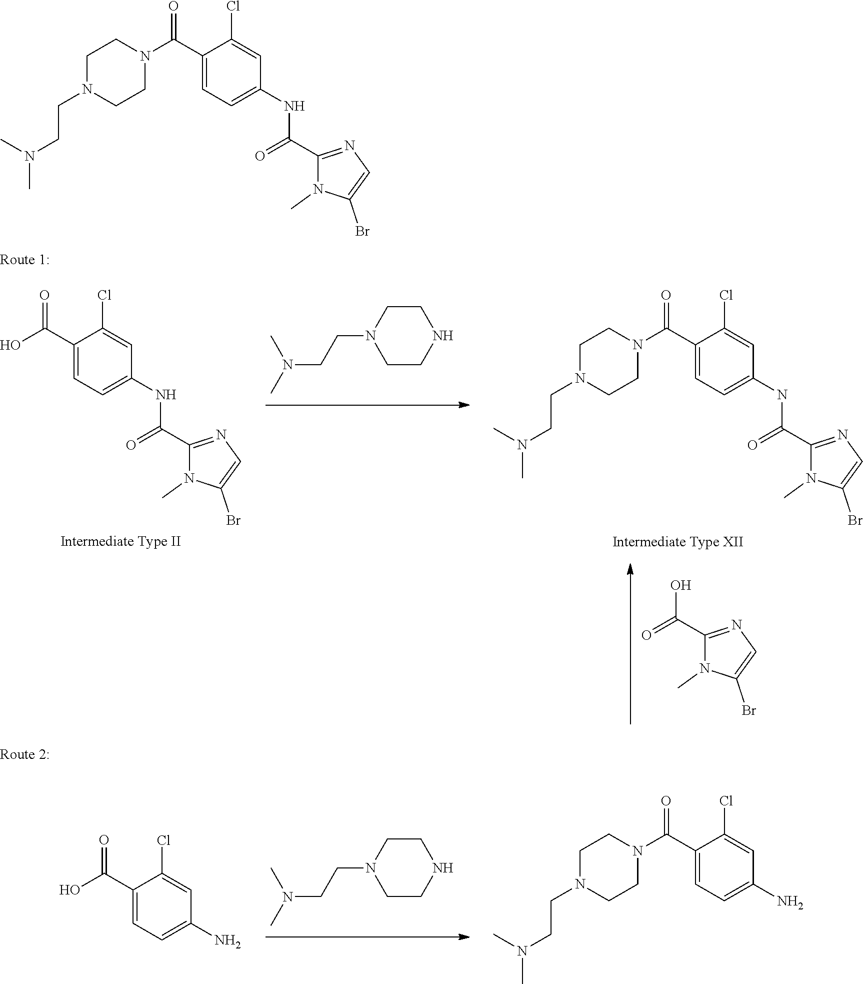

### Route 1:

At room temperature, 2,4,6-tripropyl-1,3,5,2,4,6-trioxatriphosphinane 2,4,6-trioxide (7.28 g, 22.9 mmol) was added to a mixture of 4-(5-bromo-1-methyl-1H-imidazole-2-carboxamido)-2-chlorobenzoic acid (Intermediate 313, 4.1 g, 11.4 mmol), N,N-dimethyl-2-(piperazin-1-yl)ethan-1-amine (2.7 g, 17.2 mmol) and DIPEA (4.43 g, 5.99 mL, 34.3 mmol) in DMF (10 mL). After stirring for 20 min, the reaction was completed. The mixture was poured into water. The water phase was extracted with DCM. The organic phase was washed with water, dried and concentrated. The residue was dried by freeze dryer to give the title compound (5.2 g, 91.4% yield) as a white solid. MS (ESI, m/z): 496.8 [M+H]⁺.

### Route 2:

### Step 1: (4-amino-2-chloro-phenyl)-[4-[2-(dimethylamino)ethyl]piperazin-1-yl]methanone (Intermediate 433)

To a solution of 1-(2-dimethylaminoethyl)piperazine (0.35 g, 2.24 mmol), 4-amino-2-chlorobenzoic acid (0.35 g, 2.04 mmol) and N,N-diisopropylethylamine (0.71 mL, 4.08 mmol) in DMF (10 mL) was added 0-(7-azabenzotriazol-1-yl)-N,N,N′,N′-tetramethyluronium hexafluorophosphate (0.93 g, 2.45 mmol). The mixture was stirred for 3 h at 30° C. The mixture was diluted with water (60 mL) and extracted with EtOAc (75 mL×2). The organic layer was washed with brine (50 mL×2), dried over sodium sulfate, filtered and concentrated in vacuum to afford the title compound (600 mg, 1.93 mmol, 94.63% yield) as a light brown oil. MS (ESI, m/z): 311.1 [M+H]⁺.

### Step 2: 5-bromo-N-[3-chloro-4-[4-[2-(dimethylamino)ethyl]piperazine-1-carbonyl]phenyl]-1-methyl-imidazole-2-carboxamide (Intermediate 432)

To a solution of (4-amino-2-chloro-phenyl)[4-[2-(dimethylamino)ethyl]piperazin-1-yl]methanone (295.64 mg, 0.950 mmol), 5-bromo-1-methyl-imidazole-2-carboxylic acid (150 mg, 0.730 mmol) and N,N-diisopropylethylamine (0.23 mL, 1.33 mmol) in DMF (3 mL) was added 0-(7-azabenzotriazol-1-yl)-N,N,N′,N′-tetramethyluronium hexafluorophosphate (303.49 mg, 0.800 mmol). The mixture was stirred for 3 h at 30° C. The mixture was diluted with water (60 mL) and extracted with EtOAc (75 mL×2). The organic layer was washed with brine (50 mL×2), dried over sodium sulfate, filtered and concentrated in vacuum to afford the title compound (70 mg, 0.140 mmol, 19.22% yield) as a light brown solid. MS (ESI, m/z): 499.0 [M+H]⁺.

### Intermediate Type XIII: 434

### (2S,4R)-1-tert-butoxycarbonyl-4-hydroxy-4-methyl-pyrrolidine-2-carboxylic acid and (2S,4S)-1-tert-butoxycarbonyl-4-hydroxy-4-methyl-pyrrolidine-2-carboxylic acid

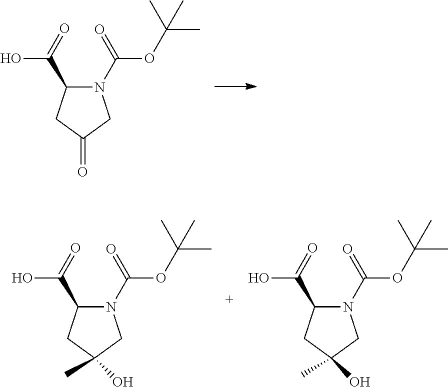 [SMILES: CC(C)(C)OC(=O)N1CC(=O)C[C@H]1C(=O)O ; CC(C)(C)OC(=O)N1C[C@](C)(O)C[C@H]1C(=O)O ; CC(C)(C)OC(=O)N1C[C@@](C)(O)C[C@H]1C(=O)O]

(2S)-1-tert-butoxycarbonyl-4-oxo-pyrrolidine-2-carboxylic acid (2 g, 8.72 mmol) in THF (20 mL) was added dropwise to a solution of 1.5 M methyllithium in diethylether (8.72 mL, 13.09 mmol) at −20° C. under a nitrogen atmosphere. The resulting mixture was stirred at the same temperature for 1 h and then further stirred at 25° C. for 11 h. The reaction mixture was added into 1 N aqueous hydrochloric acid solution (50 mL) under ice cooling, followed by extraction with ethyl acetate (50 mL×3). The organic layer was washed with brine and dried over anhydrous sodium sulfate. The solvent was evaporated under reduced pressure. The crude product was purified by prep-HPLC (FA) to afford 2 final compounds: P1, (2S,4R)-1-tert-butoxycarbonyl-4-hydroxy-4-methyl-pyrrolidine-2-carboxylic acid (50 mg, 0.200 mmol, 2.34% yield) as a dark green solid, MS (ESI, m/z): 190.0 [M+H−56]⁺, and compound P2, (2S,4S)-1-tert-butoxycarbonyl-4-hydroxy-4-methyl-pyrrolidine-2-carboxylic acid (600 mg, 2.45 mmol, 28.04% yield) as an off-white solid. MS (ESI, m/z): 190.0 [M+H−56]⁺.

### Intermediate Type XIII: 436

### (2S,4S)-1-tert-butoxycarbonyl-4-hydroxy-4-ethyl-pyrrolidine-2-carboxylic acid and (2S,4R)-1-tert-butoxycarbonyl-4-ethyl-4-hydroxy-pyrrolidine-2-carboxylic acid

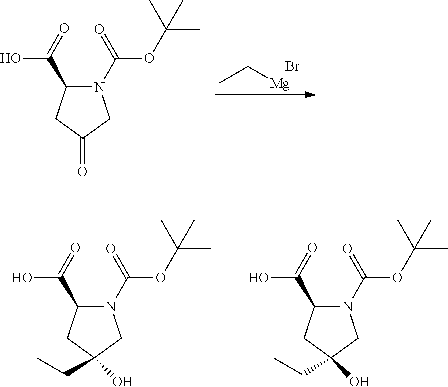

Ethylmagnesium bromide (7.27 mL, 21.81 mmol, 3M in diethyl ether) was added dropwise to a THF (50 mL) solution of (2S)-1-tert-butoxycarbonyl-4-oxo-pyrrolidine-2-carboxylic acid (2 g, 8.72 mmol) at −20° C. under a nitrogen atmosphere. The resulting mixture was stirred at the same temperature for 1 h and then further stirred at 25° C. for 10 h. The reaction mixture was added into 1 N aqueous hydrochloric acid solution (100 mL) under ice cooling, followed by extraction with ethyl acetate. The organic layer was washed with brine and dried over anhydrous sodium sulfate. The solvent was evaporated under reduced pressure. The crude product was purified by prep-HPLC (FA) to afford 2 final compounds: the title compound P1 (2S,4S)-1-tut-butoxycarbonyl-4-ethyl-4-hydroxy-pyrrolidine-2-carboxylic acid (0.8 g, 3.09 mmol, 35.36% yield) as an off-white solid, MS (ESI, m/z): 282.0 [M+Na]+, and the title compound P2, (2S,4R)-1-tert-butoxycarbonyl-4-ethyl-4-hydroxy-pyrrolidine-2-carboxylic acid (0.2 g, 0.770 mmol, 8.84% yield) as an off-white solid. MS (ESI, m/z): 282.0 [M+Na]⁺.

### Intermediate Type XIII: 12

### (3S,4R)-1-tert-Butoxycarbonyl-3-hydroxy-piperidine-4-carboxylic acid

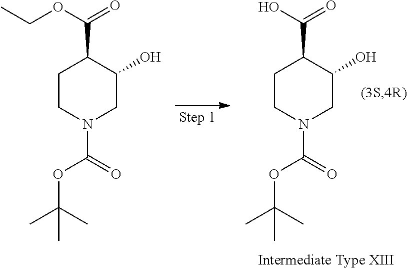 [SMILES: CCOC(=O)[C@@H]1CCN(C(=O)OC(C)(C)C)C[C@H]1O ; CC(C)(C)OC(=O)N1CC[C@@H](C(=O)O)[C@H](O)C1]

### Step 1: (3S,4R)-1-tert-butoxycarbonyl-3-hydroxy-piperidine-4-carboxylic acid (12)

To a solution of 1-(tert-butyl) 4-ethyl (3S,4R)-3-hydroxypiperidine-1,4-dicarboxylate, (2.1 g, 7.68 mmol) in methanol (20 mL)/THF (20 mL)/water (20 mL) was added lithium hydroxide (0.46 g, 19.21 mmol) and the reaction mixture was stirred at 30° C. for 12 h. The crude product was adjusted to pH=7 with a 1 N solution of HCl in water and then the solution was lyophilised to afford the title compound (2.4 g, 9.79 mmol, 76.42% yield) as an off-white solid. It was used without further purification. MS (ESI, m/z): 190.0 [M+H−56]⁺.

### Intermediate 437

### (3S,4S)-1-tert-butoxycarbonyl-3-hydroxy-piperidine-4-carboxylic acid

(3S,4S)-1-tert-butoxycarbonyl-3-hydroxy-piperidine-4-carboxylic acid can be prepared in analogy to intermediate 12 using CAS [2166250-53-7].

### Example Type I: 13

### N-[4-(4-Aminopiperidine-1-carbonyl)-3-chloro-phenyl]-5-(2,3-difluoro-4-methoxy-phenyl)-1-methyl-imidazole-2-carboxamide formate

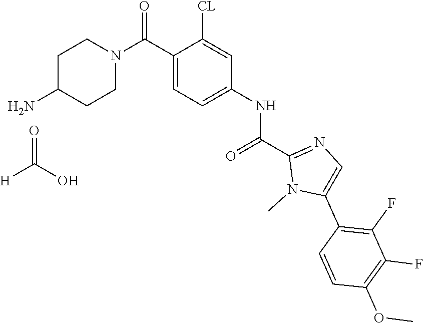

The crude N-[4-(4-aminopiperidine-1-carbonyl)-3-chloro-phenyl]-5-(2,3-difluoro-4-methoxy-phenyl)-1-methyl-imidazole-2-carboxamide (intermediate 373, 200 mg) was purified by Prep-HPLC to give the title compound (50 mg). MS (ESI, m/z): 504.2 [M+H]⁺.

The following Type I Examples were prepared in analogy to example 13.

| | | | ESI MS | |
|---|---|---|---|---|
| Ex. | Name | Structure | [M + H]⁺ | Starting Material |
| 14 | N-[3-chloro-4- (piperazine-1- carbonyl)phenyl]-5- [4-(cyanomethoxy)- 2,3-difluoro-phenyl]- 1-methyl-imidazole- 2-carboxamide | 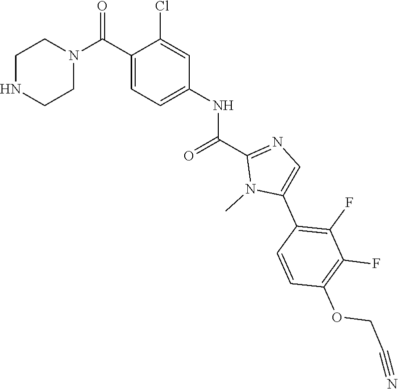 [SMILES: Cn1c(-c2ccc(OCC#N)c(F)c2F)cnc1C(=O)Nc1ccc(C(=O)N2CCNCC2)c(Cl)c1] | 515.2 | Intermediate 438 |
| 16 | N-[4-[4- (aminomethyl) piperidine-1- carbonyl]-3- chloro-phenyl]-5- (2,3-difluoro-4- methoxy-phenyl)-1- methyl-imidazole-2- carboxamide trifluoroacetate | 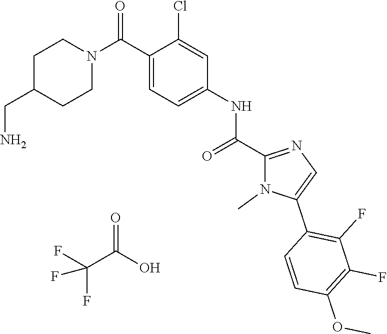 [SMILES: COc1ccc(-c2cnc(C(=O)Nc3ccc(C(=O)N4CCC(CN)CC4)c(Cl)c3)n2C)c(F)c1F ; O=C(O)C(F)(F)F] | 518.2 | Intermediate 391 |

### Example Type I: 17

### N-[4-[4-(2-Aminoethyl)piperidine-1-carbonyl]-3-chloro-phenyl]-5-(2,3-difluoro-4-methoxy-phenyl)-1-methyl-imidazole-2-carboxamide formate

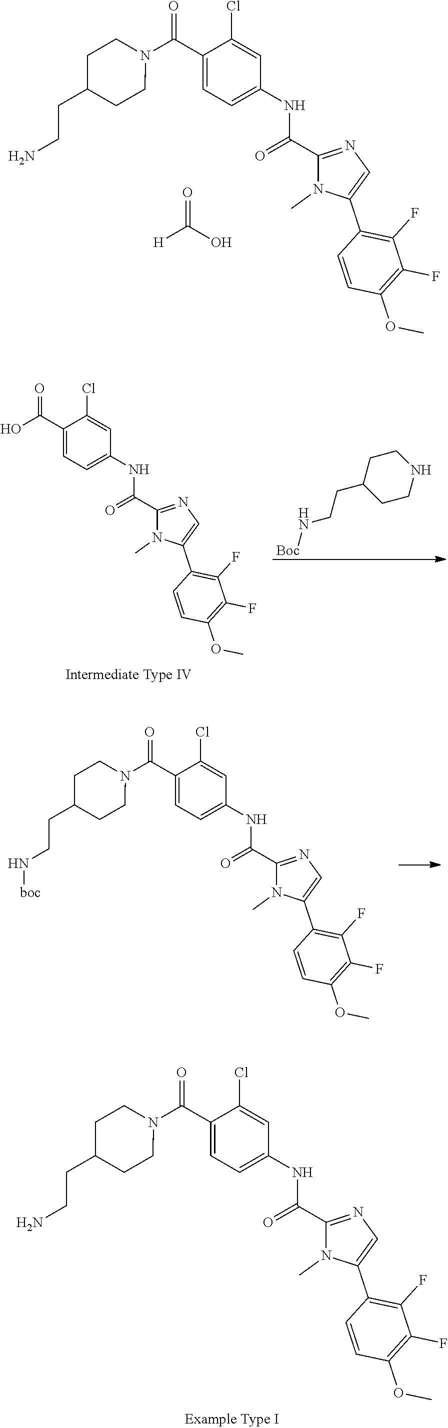

### Step 1

### N-[3-chloro-4-[4-[2-(methylamino)ethyl]piperidine-1-carbonyl]phenyl]-5-(2,3-difluoro-4-methoxy-phenyl)-1-methyl-imidazole-2-carboxamide

In a 100 mL round-bottomed flask, 2-chloro-4-(5-(2,3-difluoro-4-methoxyphenyl)-1-methyl-1H-imidazole-2-carboxamido)benzoic acid (200 mg, 474 μmol), tert-butyl (2-(piperidin-4-yl)ethyl) carbamate (141 mg, 616 μmol) and DIPEA (184 mg, 248 μl, 1.42 mmol) were combined with DMF to give a light brown solution. 2,4,6-tripropyl-1,3,5,2,4,6-trioxatriphosphinane 2,4,6-trioxide (603 mg, 948 μmol) was added. The reaction was stirred at room temperature overnight. The reaction mixture was poured into 50 mL H₂O and extracted with EtOAc (3×25 mL). The organic layers were combined, washed with sat NaCl (3×25 mL). The organic layers were dried over Na₂SO₄ and concentrated in vacuo to afford tert-butyl (2-(1-(2-chloro-4-(5-(2,3-difluoro-4-methoxyphenyl)-1-methyl-1H-imidazole-2-carboxamido)benzoyl)piperidin-4-yl)ethyl)carbamate (295 mg, 98.4% yield). MS (ESI, m/z): 632.1 [M+H]⁺.

### Step 2

### N-[4-[4-(2-aminoethyl)piperidine-1-carbonyl]-3-chloro-phenyl]-5-(2,3-difluoro-4-methoxy-phenyl)-1-methyl-imidazole-2-carboxamide formate

In a 100 mL round-bottomed flask, tert-butyl (2-(1-(2-chloro-4-(5-(2,3-difluoro-4-methoxyphenyl)-1-methyl-1H-imidazole-2-carboxamido)benzoyl)piperidin-4-yl)ethyl)carbamate (295 mg, 467 μmol) was combined with THF (3 mL) to give a light yellow solution. 4 M HCl (3.5 mL, 14 mmol) in dioxane was added. The reaction was stirred at room temperature for 20 min. The crude reaction mixture was concentrated in vacuo. The crude material was purified by preparative HPLC to afford N-(4-(4-(2-aminoethyl)piperidine-1-carbonyl)-3-chlorophenyl)-5-(2,3-difluoro-4-methoxyphenyl)-1-methyl-1H-imidazole-2-carboxamide formate (270 mg, 98.1% yield). MS (ESI, m/z): 532.2 [M+H]⁺.

The following Type II Examples and Type III Examples were prepared in analogy to example 17.

| | | | ESI MS | |
|---|---|---|---|---|
| Ex. | Name | Structure | [M + H]⁺ | Starting Material |
| 18 | N-[4-[3-(2- aminoethoxy)azetidine- 1-carbonyl]-3-chloro- phenyl]-5-(2,3-difluoro- 4-methoxy-phenyl)-1- methyl-imidazole-2- carboxamide formate | 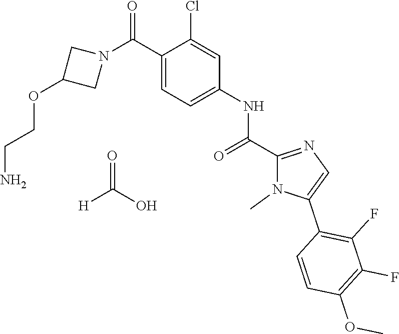 [SMILES: COc1ccc(-c2cnc(C(=O)Nc3ccc(C(=O)N4CC(OCCN)C4)c(Cl)c3)n2C)c(F)c1F ; O=CO] | 520.4 | Intermediate 341 and benzyl 3-(2- ((tert- butoxycarbonyl) amino) ethoxy)azetidine-1- carboxylate |
| 19 | N-[4-[4-(2- aminoethyl)piperazine- 1-carbonyl]-3-chloro- phenyl]-5-[4- (cyanomethoxy)-2,3- difluoro-phenyl]-1- methyl-imidazole-2- carboxamide trifluoroacetate | 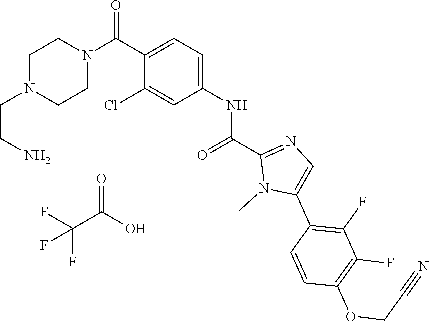 [SMILES: Cn1c(-c2ccc(OCC#N)c(F)c2F)cnc1C(=O)Nc1ccc(C(=O)N2CCN(CCN)CC2)c(Cl)c1 ; O=C(O)C(F)(F)F] | 558.2 | Intermediate 371 and 1-(2-N-Boc- aminoethyl) piperazine |
| 20 | N-(3-chloro-4-(4-(3- (dimethylamino)propyl) piperazine-1- carbonyl)phenyl)-5-(4- (cyanomethoxy)-2,3- difluorophenyl)-1- methyl-1H-imidazole- 2-carboxamide formate | 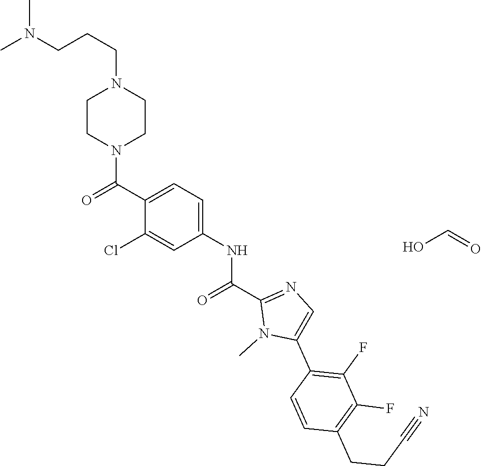 [SMILES: CN(C)CCCN1CCN(C(=O)c2ccc(NC(=O)c3ncc(-c4ccc(CCC#N)c(F)c4F)n3C)cc2Cl)CC1 ; O=CO] | 646.2 | Intermediate 371 and N,N-dimethyl- 3-(piperazin-1- yl)propan-1-amine |
| 21 | N-(3-chloro-4-(4- (piperazin-1- ylsulfonyl)piperidine-1- carbonyl)phenyl)-5- (2,3-difluoro-4- methoxyphenyl)-1- methyl-1H-imidazole- 2-carboxamide formate | 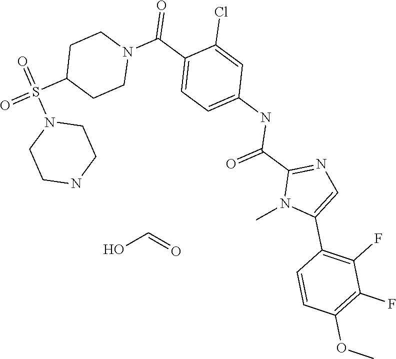 [SMILES: COc1ccc(-c2cnc(C(=O)Nc3ccc(C(=O)N4CCC(S(=O)(=O)N5CCNCC5)CC4)c(Cl)c3)n2C)c(F)c1F ; O=CO] | 683.2 | Intermediate 349 and intermediate 406 |
| 22 | N-[4-[4-(2- aminoethoxy)piperidine- 1-carbonyl]-3-chloro- phenyl]-5-(2,3-difluoro- 4-methoxy-phenyl)-1- methyl-imidazole-2- carboxamide formate | 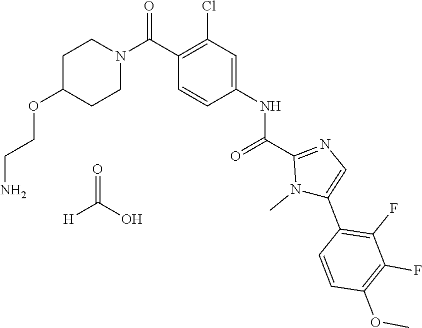 [SMILES: COc1ccc(-c2cnc(C(=O)Nc3ccc(C(=O)N4CCC(OCCN)CC4)c(Cl)c3)n2C)c(F)c1F ; O=CO] | 548.4 | Intermediate 341 and 402 |
| 23 | N-[4-[4-(azetidin-3- ylmethoxy)piperidine- 1-carbonyl]-3-chloro- phenyl]-5-(2,3-difluoro- 4-methoxy-phenyl)-1- methyl-imidazole-2- carboxamide formate | 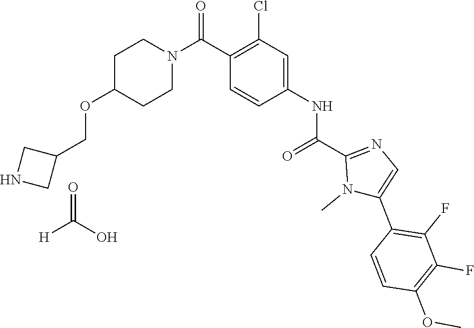 | 574.1 | Intermediate 341 and 403 |
| 24 | N-(4-(4-(1-(2-amino- 2-oxoethyl)piperidine- 4-carbonyl)piperazine- 1-carbonyl)-3- chlorophenyl)-5-(2,3- difluoro-4- (fluoromethoxy)phenyl)- 1-methyl-1H- imidazole-2- carboxamide | 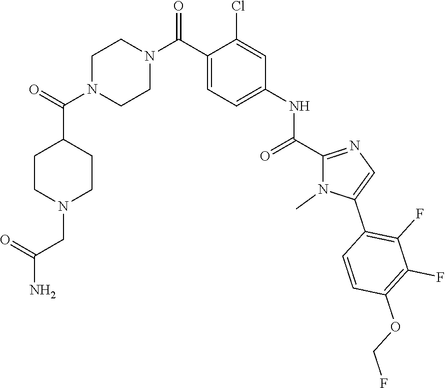 [SMILES: Cn1c(-c2ccc(OCF)c(F)c2F)cnc1C(=O)Nc1ccc(C(=O)N2CCN(C(=O)C3CCN(CC(N)=O)CC3)CC2)c(Cl)c1] | 676.2 | Intermediate 352 and intermediate 412 |
| 25 | N-(3-chloro-4-(4- (piperidine-4- carbonyl)piperazine-l- carbonyl)phenyl)-5-(2- fluoro-4- (fluoromethoxy)phenyl)- 1-methyl-1H- imidazole-2- carboxamide formate | 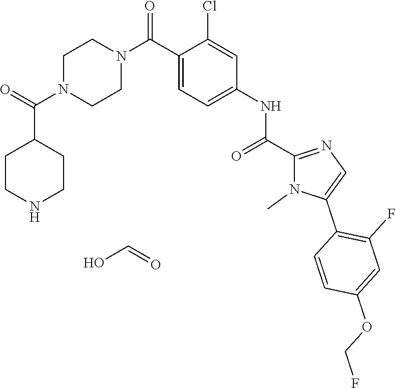 [SMILES: Cn1c(-c2ccc(OCF)cc2F)cnc1C(=O)Nc1ccc(C(=O)N2CCN(C(=O)C3CCNCC3)CC2)c(Cl)c1 ; O=CO] | 647.2 | Intermediate 353 and intermediate 408 |
| 26 | (R)-N-(4-(4-(3- aminopyrrolidine-1- carbonyl)piperazine-1- carbonyl)-3- chlorophenyl)-5-(2- fluoro-4- (fluoromethoxy)phenyl)- 1-methyl-1H- imidazole-2- carboxamide formate | 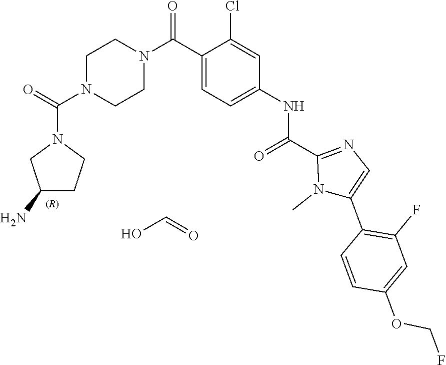 | 648.2 | Intermediate 352 and intermediate 405 |
| 27 | N-[4-[4-[(3-cyclobutyl- 1,2,4-oxadiazol-5- yl)methyl]piperidine-1- carbonyl]-3-ethyl- phenyl]-5-(2,3-difluoro- 4-methoxy-phenyl)-1- methyl-imidazole-2- carboxamide | 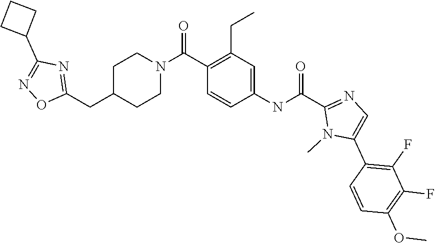 [SMILES: CCc1cc(NC(=O)c2ncc(-c3ccc(OC)c(F)c3F)n2C)ccc1C(=O)N1CCC(Cc2nc(C3CCC3)no2)CC1] | 619.5 | 439-000 and Intermediate 410-P1 |

### Example Type II: 28

### N-[(1S)-2-Amino-1-methyl-ethyl]-1-[2-chloro-4-[[5-(2,3-difluoro-4-methoxy-phenyl)-1-methyl-imidazole-2-carbonyl]amino]benzoyl]piperidine-4-carboxamide trifluoroacetate

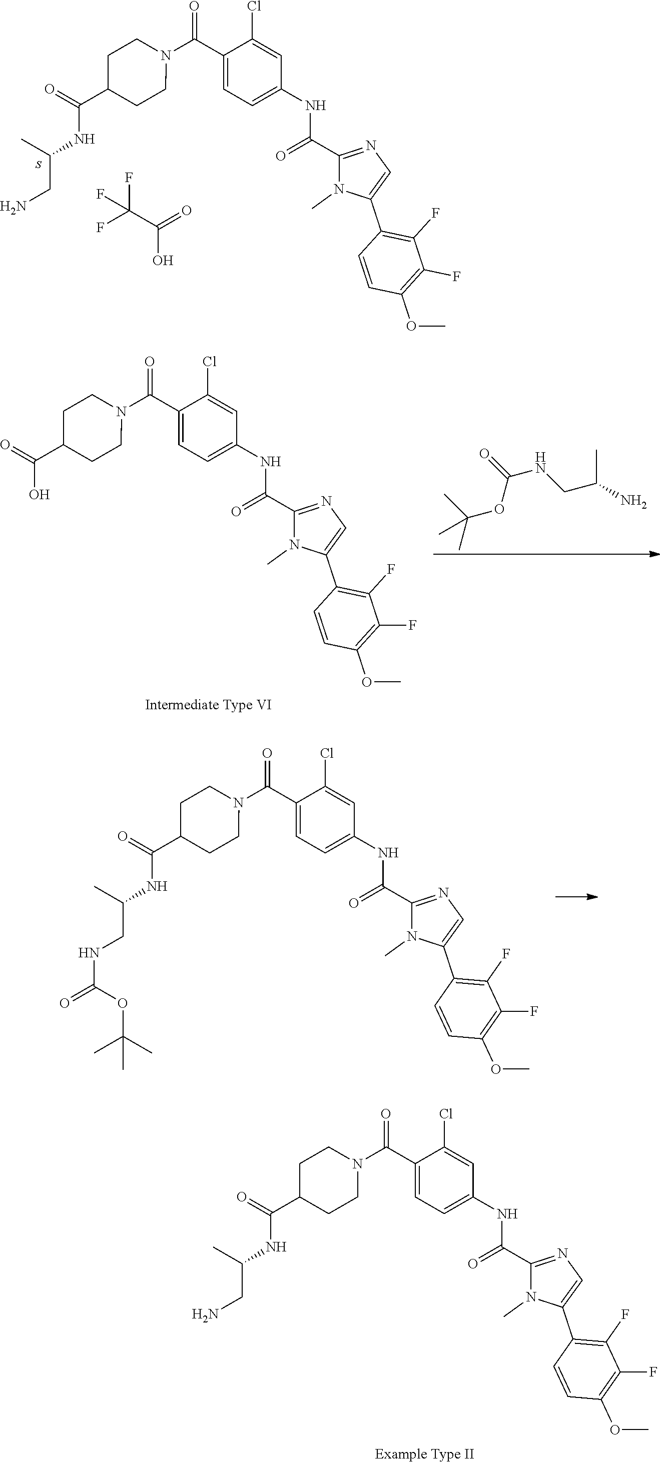

### Step 1: tert-butyl N-[(2S)-2-[[1-[2-chloro-4-[[5-(2,3-difluoro-4-methoxy-phenyl)-1-methyl-imidazole-2-carbonyl]amino]benzoyl]piperidine-4-carbonyl]amino]propyl]carbamate

At room temperature, 2,4,6-tripropyl-1,3,5,2,4,6-trioxatriphosphinane 2,4,6-trioxide (358 mg, 1.13 mmol) was added to a mixture of 1-[2-chloro-4-[[5-(2,3-difluoro-4-methoxy-phenyl)-1-methyl-imidazole-2-carbonyl]amino]benzoyl]piperidine-4-carboxylic acid (intermediate 362, 300 mg, 563 μmol), tert-butyl (S)-(2-aminopropyl)carbamate (98.1 mg, 563 μmol) and DIPEA (218 mg, 295∥l, 1.69 mmol) in DMF (2 mL). After stirring for 1 h, the mixture was poured into water. The water phase was extracted with DCM. The combined organic phases were concentrated and the residue was used into next step reaction without further purification. MS (ESI, m/z): 689.1 [M+H]⁺.

### Step 2: N-[(1S)-2-amino-1-methyl-ethyl]-1-[2-chloro-4-[[5-(2,3-difluoro-4-methoxy-phenyl)-1-methyl-imidazole-2-carbonyl]amino]benzoyl]piperidine-4-carboxamide; 2,2,2-trifluoroacetic acid

At room temperature, a solution of tert-butyl (S)-(2-(1-(2-chloro-4-(5-(2,3-difluoro-4-methoxyphenyl)-1-methyl-1H-imidazole-2-carboxamido)benzoyl)piperidine-4-carboxamido)propyl)carbamate from step 1 in TFA (5 mL) and CH₂Cl₂ (5 mL) was stirred for 2 h. Then the mixture was concentrated. Water (10 mL) was added. The mixture was basified by NH₃.H₂O to pH 8-9. The water phase was extracted with DCM. The organic phase was dried over anhydrous Na₂SO₄ and concentrated. The residue was purified by Prep-HPLC to give the title compound (61 mg). MS (ESI, m/z): 589.4 [M+H]⁺.

The following Type II Examples or Type III Examples were prepared in analogy to example 28, the deprotection step 2 was only applied for intermediates derived from Boc-protected amines.

| | | | MS | |
|---|---|---|---|---|
| | | | ESI | Starting |
| Ex. | Name | Structure | [M + H]⁺ | Material |
| 29 | N-[3-chloro-4-[4-[(3S)-3- (hydroxymethyl)piperazine- 1-carbonyl]piperidine-1- carbonyl]phenyl]-5-(2,3- difluoro-4-methoxy-phenyl)- 1-methyl-imidazole-2- carboxamide | 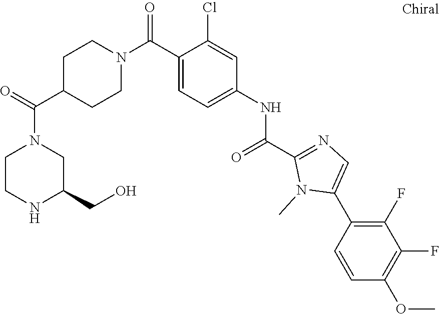 [SMILES: COc1ccc(-c2cnc(C(=O)Nc3ccc(C(=O)N4CCC(C(=O)N5CCN[C@H](CO)C5)CC4)c(Cl)c3)n2C)c(F)c1F] | 631.4 | Intermediate 362 and (S)-1- Boc-2- hydroxymethyl- piperazine |
| 30 | N-[3-chloro-4-[4-[(3R)-3- (hydroxymethyl)piperazine- 1-carbonyl]piperidine-1- carbonyl]phenyl]-5-(2,3- difluoro-4-methoxy-phenyl)- 1-methyl-imidazole-2- carboxamide | 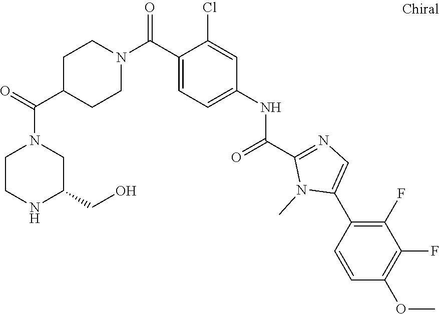 [SMILES: COc1ccc(-c2cnc(C(=O)Nc3ccc(C(=O)N4CCC(C(=O)N5CCN[C@@H](CO)C5)CC4)c(Cl)c3)n2C)c(F)c1F] | 631.4 | Intermediate 362 and (R)-1- Boc-2- hydroxymethyl- piperazine |
| 31 | N-(azetidin-3-ylmethyl)-1-[2- chloro-4-[[5-(2,3-difluoro-4- methoxy-phenyl)-1-methyl- imidazole-2- carbonyl]amino]benzoyl] piperidine-4- carboxamide formate | 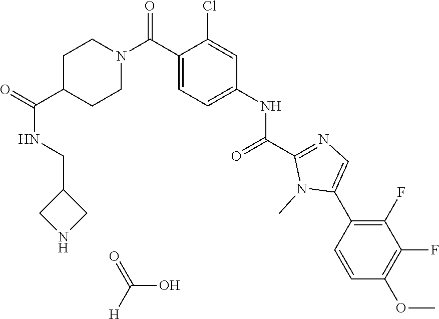 [SMILES: COc1ccc(-c2cnc(C(=O)Nc3ccc(C(=O)N4CCC(C(=O)NCC5CNC5)CC4)c(Cl)c3)n2C)c(F)c1F ; O=CO] | 601.1 | Intermediate 362 and tert- butyl 3- (aminomethyl) azetidine-1- carboxylate |
| 32 | N-[(2S)-2-aminopropyl]-1-[2- chloro-4-[[5-(2,3-difluoro-4- methoxy-phenyl)-1-methyl- imidazole-2- carbonyl]amino]benzoyl] piperidine-4-carboxamide | 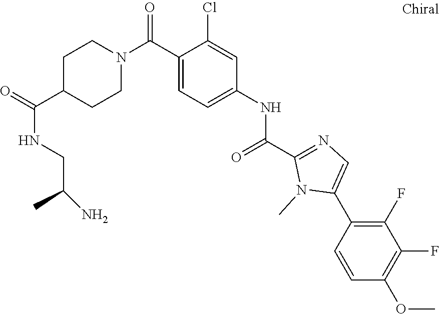 [SMILES: COc1ccc(-c2cnc(C(=O)Nc3ccc(C(=O)N4CCC(C(=O)NC[C@H](C)N)CC4)c(Cl)c3)n2C)c(F)c1F] | 589.5 | Intermediate 362 and tert- butyl (S)-(1- aminopropan- 2-yl)carbamate |
| 33 | N-[4-[4-(3-aminoazetidine-1- carbonyl)piperidine-1- carbonyl]-3-chloro-phenyl]- 5-(2,3-difluoro-4-methoxy- phenyl)-1-methyl-imidazole- 2-carboxamide trifluoroacetate | 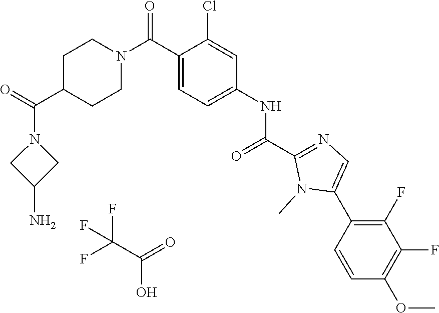 [SMILES: COc1ccc(-c2cnc(C(=O)Nc3ccc(C(=O)N4CCC(C(=O)N5CC(N)C5)CC4)c(Cl)c3)n2C)c(F)c1F ; O=C(O)C(F)(F)F] | 587.2 | Intermediate 362 and 3-N- Boc-amino- azetidine |
| 34 | N-[4-[4-[3- (aminomethyl)azetidine-1- carbonyl]piperidine-1- carbonyl]-3-chloro-phenyl]- 5-(2,3-difluoro-4-methoxy- phenyl)-1-methyl-imidazole- 2- carboxamidetrifluoroacetate | 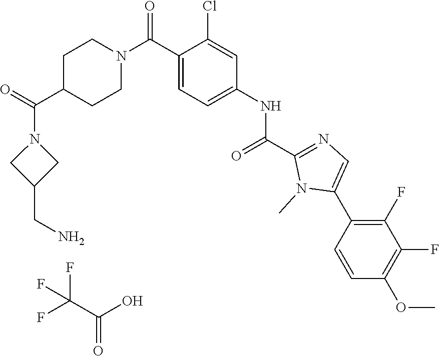 | 601.2 | Intermediate 362 and tert- butyl (azetidin- 3-ylmethyl) carbamate |
| 35 | 1-[2-chloro-4-[[5-(2,3- difluoro-4-methoxy-phenyl)- 1-methyl-imidazole-2- carbonyl]amino]benzoyl]-N- (4- piperidylmethyl)piperidine-4- carboxamide formate | 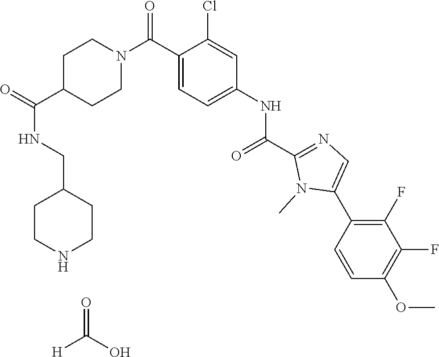 [SMILES: COc1ccc(-c2cnc(C(=O)Nc3ccc(C(=O)N4CCC(C(=O)NCC5CCNCC5)CC4)c(Cl)c3)n2C)c(F)c1F ; O=CO] | 629.2 | Intermediate 362 and tert- butyl 4- (aminomethyl) piperidine-1- carboxylate |
| 36 | 1-[2-chloro-4-[[5-(2,3- difluoro-4-methoxy-phenyl)- 1-methyl-imidazole-2- carbonyl]amino]benzoyl]-N- (4-pyridylmethyl)piperidine- 4-carboxamide | 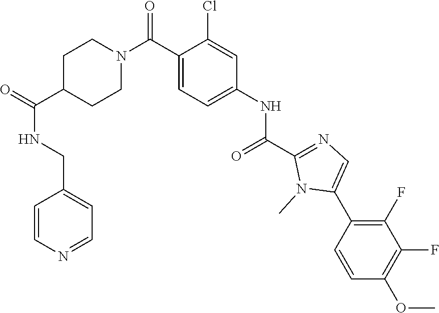 [SMILES: COc1ccc(-c2cnc(C(=O)Nc3ccc(C(=O)N4CCC(C(=O)NCc5ccncc5)CC4)c(Cl)c3)n2C)c(F)c1F] | 623.2 | Intermediate 362 and pyridin-4- ylmethanamine |
| 37 | N-[4-[4-[(3S,4S)-3-amino-4- fluoro-pyrrolidine-1- carbonyl]piperidine-1- carbonyl]-3-chloro-phenyl]- 5-(2,3-difluoro-4-methoxy- phenyl)-1-methyl-imidazole- 2-carboxamide trifluoroacetate | 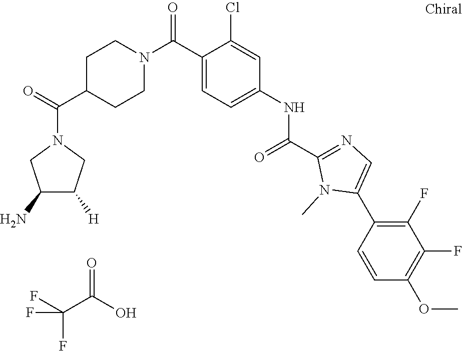 | 619.2 | Intermediate 362 and tert- butyl ((3S,4S)- 4- fluoropyrrolidin- 3-yl)carbamate |
| 38 | 1-[2-chloro-4-[[5-(2,3- difluoro-4-methoxy-phenyl)- 1-methyl-imidazole-2- carbonyl]amino]benzoyl]-N- (4-piperidyl)piperidine-4- carboxamide formate | 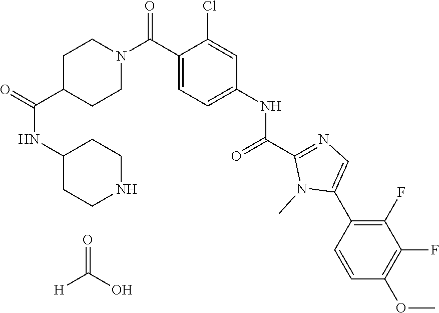 | 615.2 | Intermediate 362 and tert- butyl 4- aminopiperidine- 1-carboxylate |
| 39 | N-[4-[4-(4-aminopiperidine- 1-carbonyl)piperidine-1- carbonyl]-3-chloro-phenyl]- 5-(2,3-difluoro-4-methoxy- phenyl)-1-methyl-imidazole- 2-carboxamide formate | 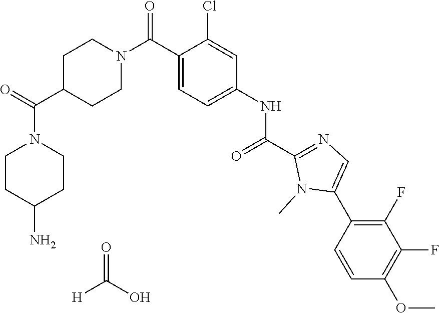 [SMILES: COc1ccc(-c2cnc(C(=O)Nc3ccc(C(=O)N4CCC(C(=O)N5CCC(N)CC5)CC4)c(Cl)c3)n2C)c(F)c1F ; O=CO] | 615.1 | Intermediate 362 and tert- butyl piperidin-4- ylcarbamate |
| 40 | N-[4-[4-[3-(2- aminoethyl)azetidine-1- carbonyl]piperidine-1- carbonyl]-3-chloro-phenyl]- 5-(2,3-difluoro-4-methoxy- phenyl)-1-methyl-imidazole- 2-carboxamide formate | 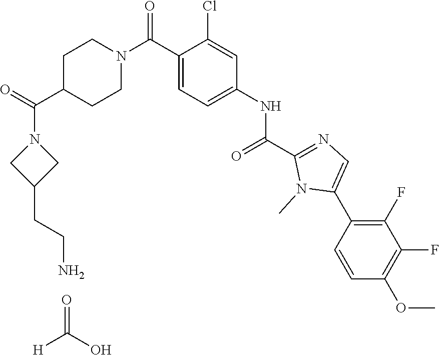 | 615.2 | Intermediate 362 and tert- butyl (2- (azetidin-3- yl)ethyl) carbamate hydrochloride |
| 41 | N-[3-chloro-4-[6-[(3R)-3- (hydroxymethyl)piperazine- 1-carbonyl]-2- azaspiro[3,3]heptane-2- carbonyl]phenyl]-5-(2,3- difluoro-4-methoxy-phenyl)- 1-methyl-imidazole-2- carboxamide | 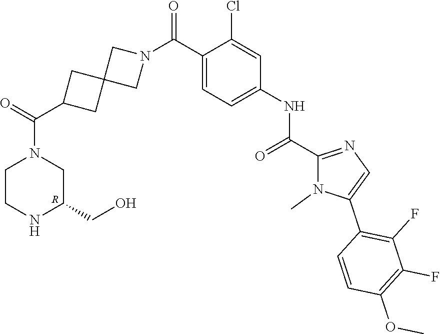 | 643.6 | Intermediate 365 and (R)-1- Boc-2- hydroxymethyl- piperazine |
| 42 | N-[3-chloro-4-(2,6- diazaspiro[3.3]heptane-2- carbonyl)phenyl]-5-(2,3- difluoro-4-methoxy-phenyl)- 1-methyl-imidazole-2- carboxamide trifluoroacetate | 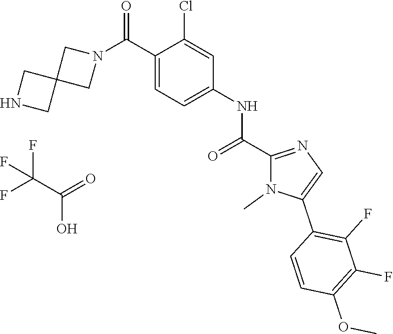 | 502.5 | Intermediate 341 and tert- butyl 2,6- diazaspiro[3.3] heptane-2- carboxylate oxalate |
| 43 | N-[3-chloro-4-[4-[3- (dimethylamino)azetidine-1- carbonyl]piperidine-1- carbonyl]phenyl]-5-[4- (difluoromethoxy)-2,3- difluoro-phenyl]-1-methyl- imidazole-2-carboxamide trifluoioacetate | 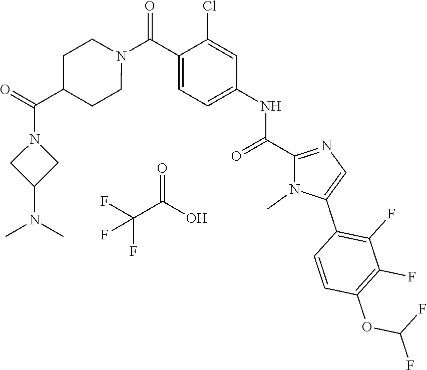 | 651.4 | Intermediate 450 and N,N- dimethylazetidin- 3-amine hydrochloride |
| 44 | 1-[2-chloro-4-[[5-[4- (difluoromethoxy)-2,3- difluoro-phenyl]-1-methyl- imidazole-2- carbonyl]amino]benzoyl]-N- [(3S,4R)-4-fluoropyrrolidin- 3-yl]piperidine-4- carboxamide trifluoroacetate | 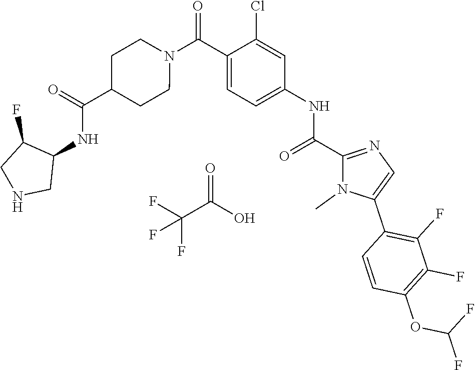 [SMILES: Cn1c(-c2ccc(OC(F)F)c(F)c2F)cnc1C(=O)Nc1ccc(C(=O)N2CCC(C(=O)N[C@H]3CNC[C@H]3F)CC2)c(Cl)c1 ; O=C(O)C(F)(F)F] | 655.3 | Intermediate 450 and tert- butyl (3S,4R)- 3-amino-4- fluoro- pyrrolidine- 1-carboxylate |
| 45 | N-[4-[4-[(3R)-3- aminopyrrolidine-1- carbonyl]piperidine-1- carbonyl]-3-chloro-phenyl]- 5-[4-(difluoromethoxy)-2,3- difluoro-phenyl]-1-methyl- imidazole-2-carboxamide trifluoroacetate | 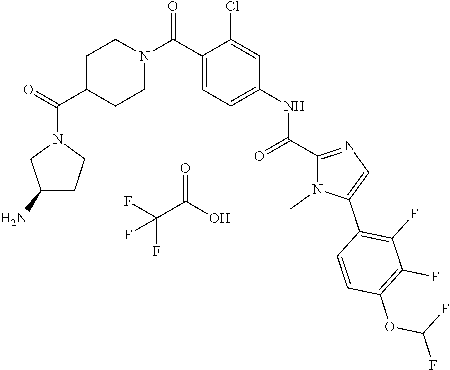 [SMILES: Cn1c(-c2ccc(OC(F)F)c(F)c2F)cnc1C(=O)Nc1ccc(C(=O)N2CCC(C(=O)N3CC[C@@H](N)C3)CC2)c(Cl)c1 ; O=C(O)C(F)(F)F] | 637.3 | Intermediate 450 and tert- butyl (R)- pyrrolidin-3- ylcarbamate |
| 46 | N-[4-[4-[(3S)-3- aminopyrrolidine-1- carbonyl]piperidine-1- carbonyl]-3-chloro-phenyl]- 5-[4-(difluoromethoxy)-2,3- difluoro-phenyl]-1-methyl- imidazole-2-carboxamide trifluoroacetate | 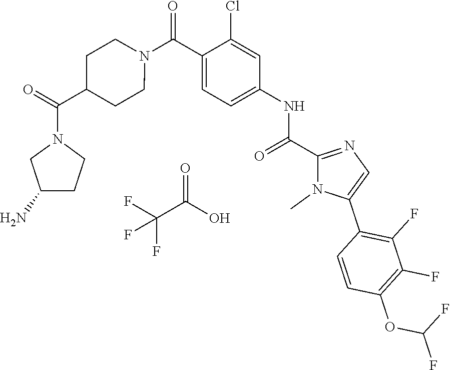 [SMILES: Cn1c(-c2ccc(OC(F)F)c(F)c2F)cnc1C(=O)Nc1ccc(C(=O)N2CCC(C(=O)N3CC[C@H](N)C3)CC2)c(Cl)c1 ; O=C(O)C(F)(F)F] | 637.3 | Intermediate 450 and tert- butyl (S)- pyrrolidin-3- ylcarbamate |
| 47 | 1-[2-chloro-4-[[5-[4- (difluoromethoxy)-2,3- difluoro-phenyl]-1-methyl- imidazole-2- carbonyl]amino]benzoyl]-N- [[(3R)-pyrrolidin-3- yl]methyl]piperidine-4- carboxamide trifluoroacetate | 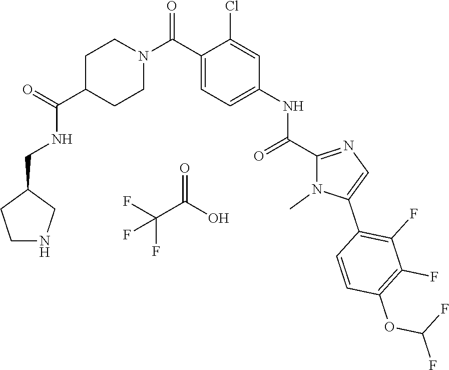 [SMILES: Cn1c(-c2ccc(OC(F)F)c(F)c2F)cnc1C(=O)Nc1ccc(C(=O)N2CCC(C(=O)NC[C@@H]3CCNC3)CC2)c(Cl)c1 ; O=C(O)C(F)(F)F] | 651.4 | Intermediate 450 and tert- butyl (R)-3- (aminomethyl) pyrrolidine-1- carboxylate |
| 48 | 1-[2-chloro-4-[[5-[4- (difluoromethoxy)-2,3- difluoro-phenyl]-1-methyl- imidazole-2- carbonyl]amino]benzoyl]-N- [[(3S)-pyrrolidin-3- yl]methyl]piperidine-4- carboxamide trifluoroacetate | 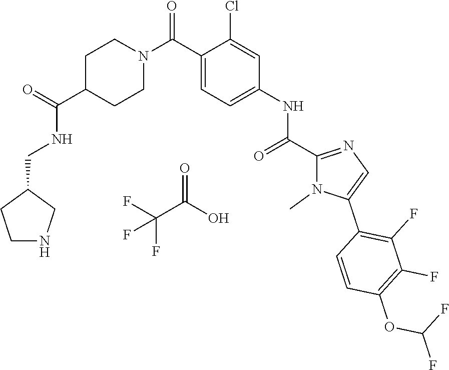 [SMILES: Cn1c(-c2ccc(OC(F)F)c(F)c2F)cnc1C(=O)Nc1ccc(C(=O)N2CCC(C(=O)NC[C@H]3CCNC3)CC2)c(Cl)c1 ; O=C(O)C(F)(F)F] | 651.4 | Intermediate 450 and tert- butyl (S)-3- (aminomethyl) pyrrolidine-1- carboxylate |
| 49 | N-[4-[4-(3- carbamoylpyrrolidine-1- carbonyl)piperidine-1- carbonyl]-3-chloro-phenyl]- 5-[4-(difluoromethoxy)-2,3- difluoro-phenyl]-1-methyl- imidazole-2-carboxamide trifluoroacetate | 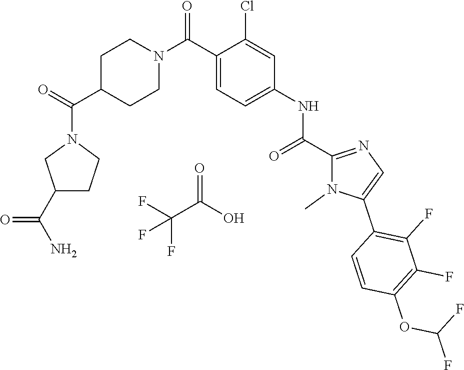 [SMILES: Cn1c(-c2ccc(OC(F)F)c(F)c2F)cnc1C(=O)Nc1ccc(C(=O)N2CCC(C(=O)N3CCC(C(N)=O)C3)CC2)c(Cl)c1 ; O=C(O)C(F)(F)F] | 665.3 | Intermediate 450 and pyrrolidine-3- carboxamide |
| 50 | 1-[2-chloro-4-[[5-[4- (difluoromethoxy)-2,3- difluoro-phenyl]-1-methyl- imidazole-2- carbonyl]amino]benzoyl]-N- [(3S,4S)-4- hydroxypyrrolidin-3- yl]piperidine-4-carboxamide trifluoroacetate | 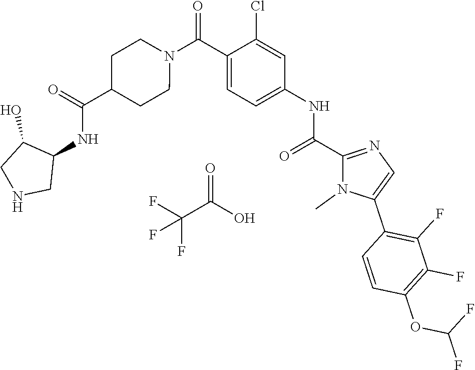 [SMILES: Cn1c(-c2ccc(OC(F)F)c(F)c2F)cnc1C(=O)Nc1ccc(C(=O)N2CCC(C(=O)N[C@H]3CNC[C@@H]3O)CC2)c(Cl)c1 ; O=C(O)C(F)(F)F] | 653.5 | Intermediate 450 and tert- butyl (3R,4R)- 3-amino-4- hydroxy- pyrrolidine- 1-carboxylate |
| 51 | 1-[2-chloro-4-[[5-[2,3- difluoro-4- (fluoromethoxy)phenyl]-1- methyl-imidazole-2- carbonyl]amino]benzoyl]-N- [(3S,4R)-4-fluoropyrrolidin- 3-yl]piperidine-4- carboxamide trifluoroacetate | 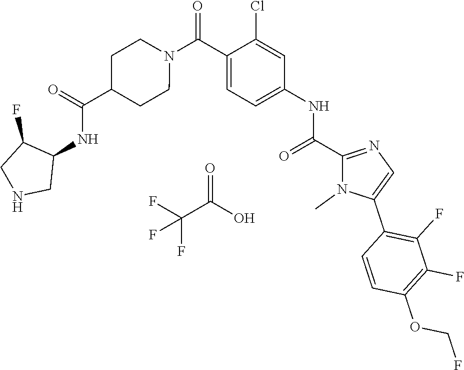 [SMILES: Cn1c(-c2ccc(OCF)c(F)c2F)cnc1C(=O)Nc1ccc(C(=O)N2CCC(C(=O)N[C@H]3CNC[C@H]3F)CC2)c(Cl)c1 ; O=C(O)C(F)(F)F] | 637.2 | Intermediate 451 and tert- butyl (3S,4R)- 3-amino-4- fluoro- pyrrolidine- 1-carboxylate |
| 52 | N-[3-chloro-4-[4-[[(1-methyl- 4- piperidyl)amino]carbamoyl] piperidine-1-carbonyl]phenyl]- 5-[2,3-difluoro-4- (fluoromethoxy)phenyl]-1- methyl-imidazole-2- carboxamide trifluoroacetate | 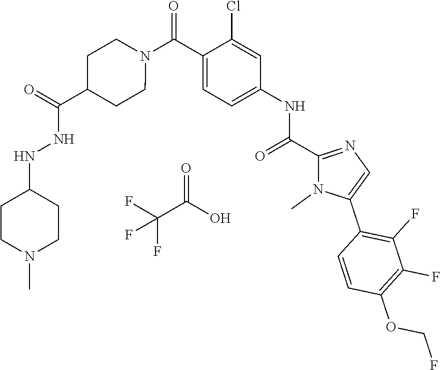 [SMILES: CN1CCC(NNC(=O)C2CCN(C(=O)c3ccc(NC(=O)c4ncc(-c5ccc(OCF)c(F)c5F)n4C)cc3Cl)CC2)CC1 ; O=C(O)C(F)(F)F] | 662.4 | Intermediate 451 and 4- hydrazineyl-1- methyl- piperidine |
| 53 | 1-[2-chloro-4-[[5-[2-chloro- 3-fluoro-4- (fluoromethoxy)phenyl]-1- methyl-imidazole-2- carbonyl]amino]benzoyl]-N- [(3R,4R)-4- hydroxypyrrolidin-3- yl]piperidine-4-carboxamide formate | 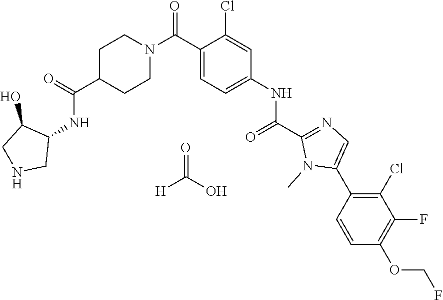 [SMILES: Cn1c(-c2ccc(OCF)c(F)c2Cl)cnc1C(=O)Nc1ccc(C(=O)N2CCC(C(=O)N[C@@H]3CNC[C@H]3O)CC2)c(Cl)c1 ; O=CO] | 653.0 | Intermediate 369 and tert- butyl (3R,4R)- 3-amino-4- hydroxy- pyrrolidine-1- carboxylate |
| 54 | 1-[2-chloro-4-[[5-[2,3- difluoro-4- (fluoromethoxy)phenyl]-1- methyl-imidazole-2- carbonyl]amino]benzoyl]-N- [[(3R)-pyrrolidin-3- yl]methyl]piperidine-4- carboxamide formate | 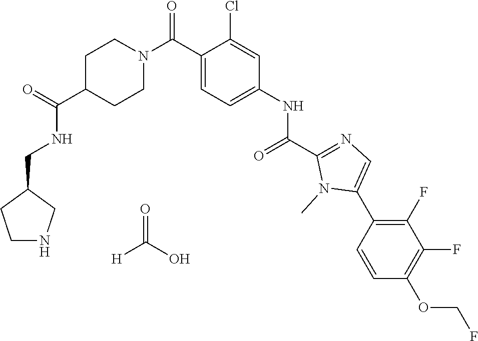 [SMILES: Cn1c(-c2ccc(OCF)c(F)c2F)cnc1C(=O)Nc1ccc(C(=O)N2CCC(C(=O)NC[C@@H]3CCNC3)CC2)c(Cl)c1 ; O=CO] | 632.9 | Intermediate 363 and tert- butyl (3S)-3- (aminomethyl) pyrrolidine-1- carboxylate |
| 55 | 1-[2-chloro-4-[[5-(2,3- difluoro-4-methoxy-phenyl)- 1-methyl-imidazole-2- carbonyl]amino]benzoyl]-N- [[(3R)-pyrrolidin-3- yl]methyl]piperidine-4- carboxamide formate | 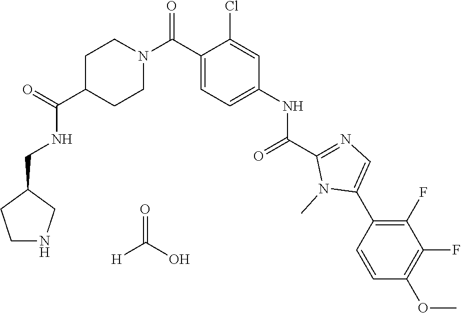 [SMILES: COc1ccc(-c2cnc(C(=O)Nc3ccc(C(=O)N4CCC(C(=O)NC[C@@H]5CCNC5)CC4)c(Cl)c3)n2C)c(F)c1F ; O=CO] | 615.3 | Intermediate 362 and (butyl (3S)-3- (aminomethyl) pyrrolidine-1- carboxylate |
| 56 | 1-[2-chloro-4-[[5-(2,3- difluoro-4-methoxy-phenyl)- 1-methyl-imidazole-2- carbonyl]amino]benzoyl]-N- [[(3S)-pyrrolidin-3- yl]methyl]piperidine-4- carboxamide formate | 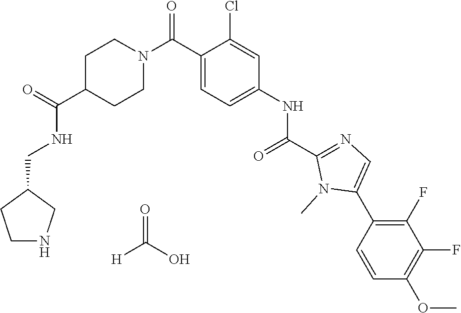 [SMILES: COc1ccc(-c2cnc(C(=O)Nc3ccc(C(=O)N4CCC(C(=O)NC[C@H]5CCNC5)CC4)c(Cl)c3)n2C)c(F)c1F ; O=CO] | 615.4 | Intermediate 362 and tert- butyl (3R)-3- (aminomethyl) pyrrolidine-1- carboxylate |
| 57 | 1-[2-chloro-4-[[5-(2,3- difluoro-4-methoxy-phenyl)- 1-methyl-imidazole-2- carbonyl]amino]benzoyl]-N- [(3-hydroxyazetidin-3- yl)methyl]piperidine-4- carboxamide formate | 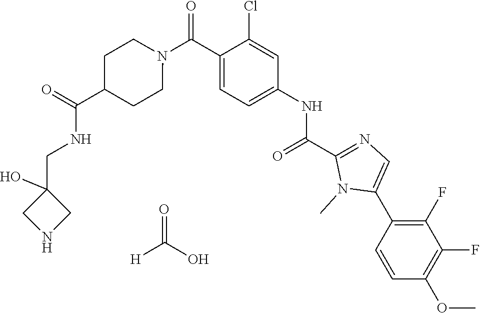 [SMILES: COc1ccc(-c2cnc(C(=O)Nc3ccc(C(=O)N4CCC(C(=O)NCC5(O)CNC5)CC4)c(Cl)c3)n2C)c(F)c1F ; O=CO] | 617.3 | Intermediate 362 and tert- butyl 3- (aminomethyl)- 3-hydroxy- azetidine-1- carboxylate |
| 58 | 1-[2-chloro-4-[[5-(2,3- difluoro-4-methoxy-phenyl)- 1-methyl-imidazole-2- carbonyl]amino]benzoyl]-N- [(3S)-pyrrolidin-3- yl]piperidine-4-carboxamide formate | 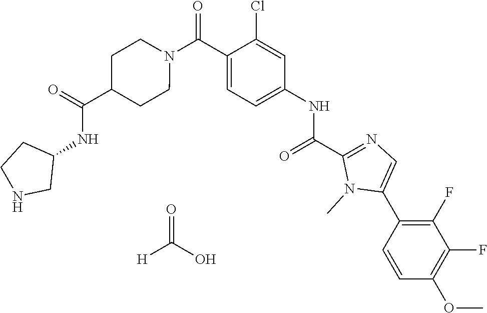 [SMILES: COc1ccc(-c2cnc(C(=O)Nc3ccc(C(=O)N4CCC(C(=O)N[C@H]5CCNC5)CC4)c(Cl)c3)n2C)c(F)c1F ; O=CO] | 601.0 | Intermediate 362 and tert- butyl (3S)-3- amino- pyrrolidine- 1-carboxylate |
| 59 | N-[(1S,5R)-3- azabicyclo[3.1.0]hexan-6-yl]- 1-[2-chloro-4-[[5-(2,3- difluoro-4-methoxy-phenyl)- 1-methyl-imidazole-2- carbonyl]amino]benzoyl] piperidine-4- carboxamide formate | 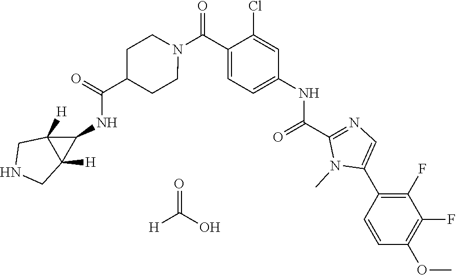 [SMILES: COc1ccc(-c2cnc(C(=O)Nc3ccc(C(=O)N4CCC(C(=O)N[C@H]5[C@@H]6CNC[C@@H]65)CC4)c(Cl)c3)n2C)c(F)c1F ; O=CO] | 613.0 | Intermediate 362 and tert- butyl (1R,5S)- 6-amino-3- azabicyclo [3.1.0] hexane-3- carboxylate |
| 60 | 1-[2-chloro-4-[[5-(2,3- difluoro-4-methoxy-phenyl)- 1-methyl-imidazole-2- carbonyl]amino]benzoyl]-N- [(3R,4R)-4- hydroxypyrrolidin-3- yl]piperidine-4-carboxamide formate | 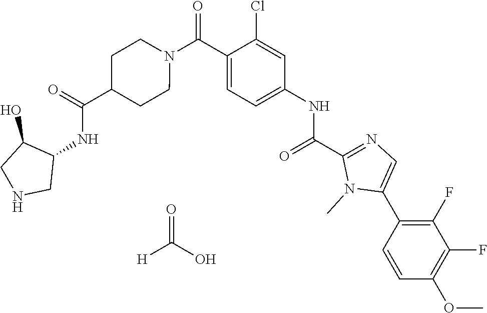 [SMILES: COc1ccc(-c2cnc(C(=O)Nc3ccc(C(=O)N4CCC(C(=O)N[C@@H]5CNC[C@H]5O)CC4)c(Cl)c3)n2C)c(F)c1F ; O=CO] | 617.0 | Intermediate 362 and tert- butyl (3R,4R)- 3-amino-4- hydroxy- pyrrolidine-1- carboxylate |
| 61 | N-[(1S,5R)-3- azabicyclo[3.1.0]hexan-6-yl]- 1-[2-chloro-4-[[5-[4- (difluoromethoxy)-2,3- difluoro-phenyl]-1-methyl- imidazole-2- carbonyl]amino]benzoyl] piperidine-4- carboxamide formate | 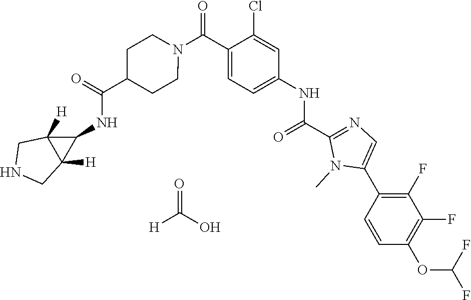 [SMILES: Cn1c(-c2ccc(OC(F)F)c(F)c2F)cnc1C(=O)Nc1ccc(C(=O)N2CCC(C(=O)N[C@H]3[C@@H]4CNC[C@@H]43)CC2)c(Cl)c1 ; O=CO] | 649.3 | Intermediate 364 and tert- butyl (1S,5R)- 6-amino-3- azabicyclo [3.1.0] hexane-3- carboxylate |
| 62 | 1-[2-chloro-4-[[5-[2-chloro- 3-fluoro-4- (fluoromethoxy)phenyl]-1- methyl-imidazole-2- carbonyl]amino]benzoyl]-N- [[(3R)-pyrrolidin-3- yl]methyl]piperidine-4- carboxamide formate | 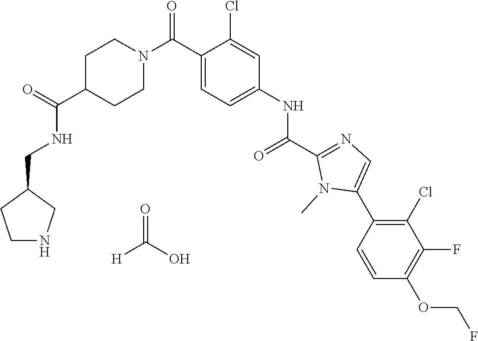 [SMILES: Cn1c(-c2ccc(OCF)c(F)c2Cl)cnc1C(=O)Nc1ccc(C(=O)N2CCC(C(=O)NC[C@@H]3CCNC3)CC2)c(Cl)c1 ; O=CO] | 650.9 | Intermediate 369 and tert- butyl (3S)-3- (aminomethyl) pyrrolidine-1- carboxylate |
| 63 | N-(azetidin-2-ylmethyl)-1-[2- chloro-4-[[5-(2,3-difluoro-4- methoxy-phenyl)-1-methyl- imidazole-2- carbonyl]amino]benzoyl] piperidine-4- carboxamide formate | 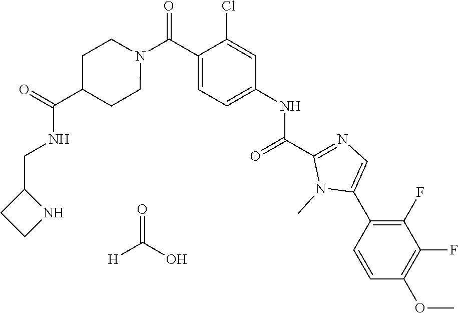 [SMILES: COc1ccc(-c2cnc(C(=O)Nc3ccc(C(=O)N4CCC(C(=O)NCC5CCN5)CC4)c(Cl)c3)n2C)c(F)c1F ; O=CO] | 601.3 | Intermediate 362 and tert- butyl 2- (aminomethyl) azetidine-1- carboxylate |
| 64 | 1-[2-chloro-4-[[5-(2,3- difluoro-4-methoxy-phenyl)- 1-methyl-imidazole-2- carbonyl]amino]benzoyl]-N- [(3R)-pyrrolidin-3- yl]piperidine-4-carboxamide formate | 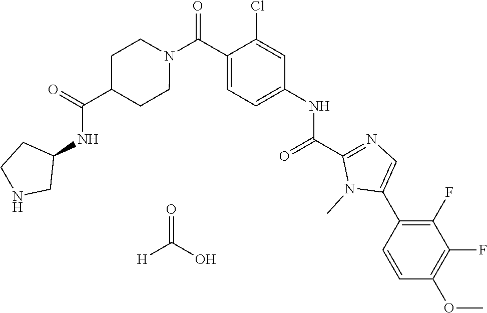 [SMILES: COc1ccc(-c2cnc(C(=O)Nc3ccc(C(=O)N4CCC(C(=O)N[C@@H]5CCNC5)CC4)c(Cl)c3)n2C)c(F)c1F ; O=CO] | 601.0 | Intermediate 362 and tert- butyl (3R)-3- amino- pyrrolidine- 1-carboxylate |
| 65 | 1-[2-chloro-4-[[5-[2-chloro- 3-fluoro-4- (fluoromethoxy)phenyl]-1- methyl-imidazole-2- carbonyl]amino]benzoyl]-N- [(3S)-pyrrolidin-3- yl]piperidine-4-carboxamide formate | 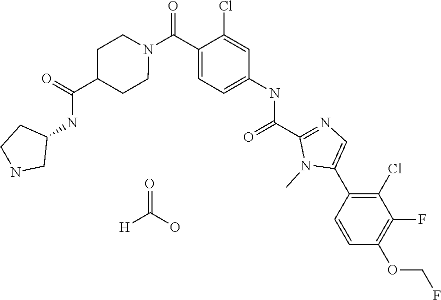 [SMILES: Cn1c(-c2ccc(OCF)c(F)c2Cl)cnc1C(=O)Nc1ccc(C(=O)N2CCC(C(=O)N[C@H]3CCNC3)CC2)c(Cl)c1 ; O=CO] | 636.9 | Intermediate 369 and tert- butyl (3S)-3- amino- pyrrolidine- 1-carboxylate |
| 66 | N-[4-[3-[3- (aminomethyl)azetidine-1- carbonyl]azetidine-1- carbonyl]-3-chloro-phenyl]- 5-(2,3-difluoro-4-methoxy- phenyl)-1-methyl-imidazole- 2-carboxamide formate | 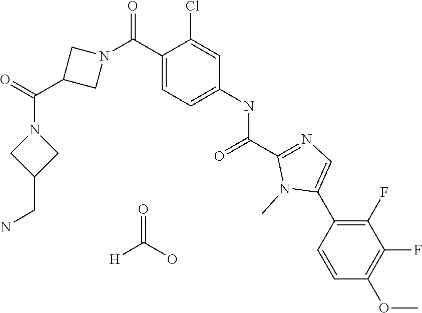 [SMILES: COc1ccc(-c2cnc(C(=O)Nc3ccc(C(=O)N4CC(C(=O)N5CC(CN)C5)C4)c(Cl)c3)n2C)c(F)c1F ; O=CO] | 573.3 | Intermediate 367 and tert- butyl 3- (aminomethyl) azetidine-1- carboxylate |
| 67 | 3-[[1-[2-chloro-4-[[5-(2,3- difluoro-4-methoxy-phenyl)- 1-methyl-imidazole-2- carbonyl]amino]benzoyl] piperidine-4- carbonyl]amino]propanoic acid | 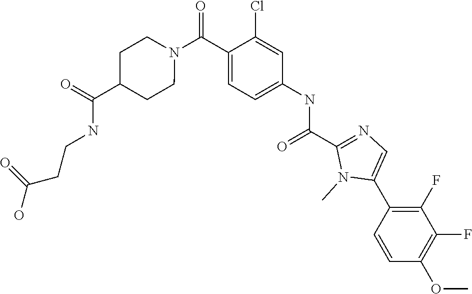 [SMILES: COc1ccc(-c2cnc(C(=O)Nc3ccc(C(=O)N4CCC(C(=O)NCCC(=O)O)CC4)c(Cl)c3)n2C)c(F)c1F] | 604.0 | Intermediate 362 and tert- butyl 3- amino- propanoate |
| 68 | 4-[[1-[2-chloro-4-[[5-(2,3- difluoro-4-methoxy-phenyl)- 1-methyl-imidazole-2- carbonyl]amino]benzoyl] piperidine-4- carbonyl]amino]butanoic acid | 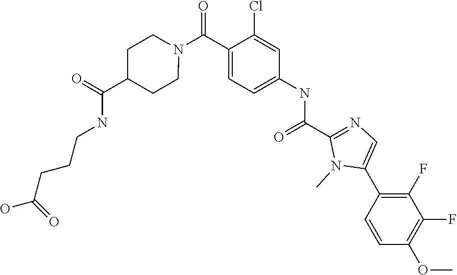 [SMILES: COc1ccc(-c2cnc(C(=O)Nc3ccc(C(=O)N4CCC(C(=O)NCCCC(=O)O)CC4)c(Cl)c3)n2C)c(F)c1F] | 618.0 | Intermediate 362 and tert- butyl 4- amino -butanoate |
| 69 | N-[3-chloro-4-[3-(piperazine- 1-carbonyl)azetidine-1- carbonyl]phenyl]-5-(2,3- difluoro-4-methoxy-phenyl)- 1-methyl-imidazole-2- carboxamide formate | 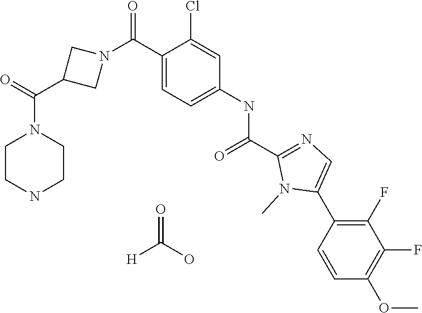 [SMILES: COc1ccc(-c2cnc(C(=O)Nc3ccc(C(=O)N4CC(C(=O)N5CCNCC5)C4)c(Cl)c3)n2C)c(F)c1F ; O=CO] | 573.3 | Intermediate 367 and tert- butyl piperazine-1- carboxylate |
| 70 | N-[4-[3-[(3aR,6aS)- 2,3,3a,4,6,6a-hexahydro-1H- pyrrolo[3,4-c]pyrrole-5- carbonyl]azetidine-1- carbonyl]-3-chloro-phenyl]- 5-(2,3-difluoro-4-methoxy- phenyl)-1-methyl-imidazole- 2-carboxamide formate | 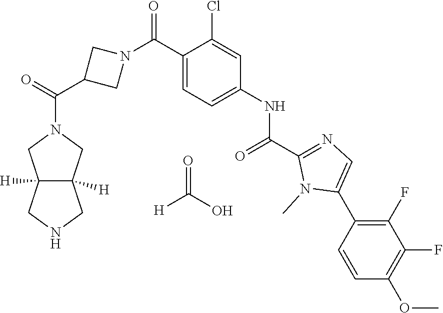 [SMILES: COc1ccc(-c2cnc(C(=O)Nc3ccc(C(=O)N4CC(C(=O)N5C[C@H]6CNC[C@H]6C5)C4)c(Cl)c3)n2C)c(F)c1F ; O=CO] | 599.3 | Intermediate 367 and tert- butyl (3aS,6aR)- 2,3,3a,4,6,6a- hexahydro-1H- pyrrolo[3,4- c]pyrrole-5- carboxylate |
| 71 | N-[4-[3-[(3aR,6aR)- 2,3,3a,4,6,6a-hexahydro-1H- pyrrolo[3,4-b]pyrrole-5- carbonyl]pyrrolidine-1- carbonyl]-3-chloro-phenyl]- 5-(2,3-difluoro-4-methoxy- phenyl)-1-methyl-imidazole- 2-carboxamide formate | 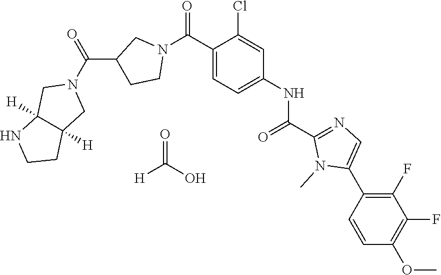 [SMILES: COc1ccc(-c2cnc(C(=O)Nc3ccc(C(=O)N4CCC(C(=O)N5C[C@H]6CCN[C@H]6C5)C4)c(Cl)c3)n2C)c(F)c1F ; O=CO] | 613.3 | Intermediate 368 and tert- butyl (3aR,6aR)- 3,3a,4,5,6,6a- hexahydro-2H- pyrrolo[2,3- c]pyrrole-1- carboxylate |
| 72 | 1-[2-chloro-4-[[5-(2,3- difluoro-4-methoxy-phenyl)- 1-methyl-imidazole-2- carbonyl]amino]benzoyl]-N- [(3S,4S)-4- hydroxypyrrolidin-3- yl]piperidine-4-carboxamide formate | 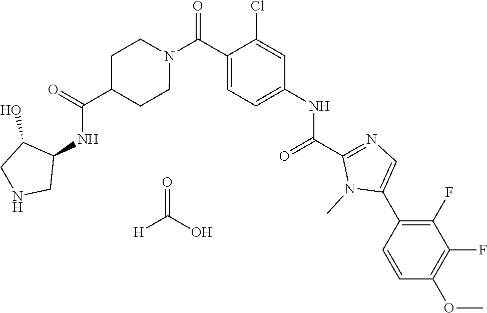 [SMILES: COc1ccc(-c2cnc(C(=O)Nc3ccc(C(=O)N4CCC(C(=O)N[C@H]5CNC[C@@H]5O)CC4)c(Cl)c3)n2C)c(F)c1F ; O=CO] | 616.9 | Intermediate 362 and tert- butyl (3S,4S)- 3-amino-4- hydroxy- pyrrolidine-1- carboxylate |
| 73 | N-(4-(4-(2- (aminomethyl)morpholine-4- carbonyl)piperidine-1- carbonyl)-3-chlorophenyl)-5- (4-(cyanomethoxy)-2,3- difluorophenyl)-1-methyl- 1H-imidazole-2-carboxamide 2,2,2-trifluoroacetate | 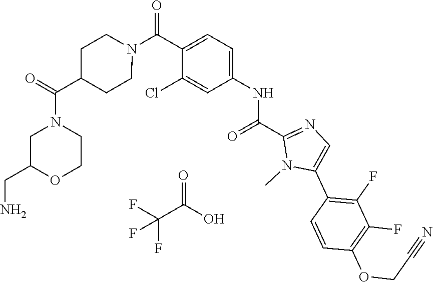 [SMILES: Cn1c(-c2ccc(OCC#N)c(F)c2F)cnc1C(=O)Nc1ccc(C(=O)N2CCC(C(=O)N3CCOC(CN)C3)CC2)c(Cl)c1 ; O=C(O)C(F)(F)F] | 656.2 | Intermediate 370 and tert- butyl N- (morpholin-2- ylmethyl) carbamate |
| 74 | N-(3-chloro-4-(4- (morpholine-4- carbonyl)piperidine-1- carbonyl)phenyl)-5-(4- (cyanomethoxy)-2,3- difluorophenyl)-1-methyl- 1H-imidazole-2-carboxamide | 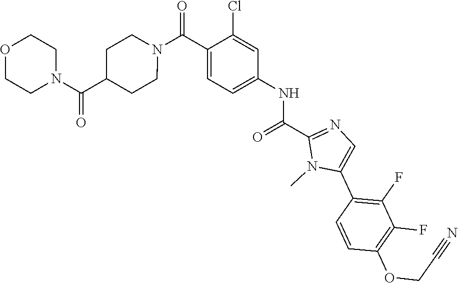 [SMILES: Cn1c(-c2ccc(OCC#N)c(F)c2F)cnc1C(=O)Nc1ccc(C(=O)N2CCC(C(=O)N3CCOCC3)CC2)c(Cl)c1] | 627.2 | Intermediate 370 and morpholine |
| 75 | N-(3-chloro-4-(4-(1,1- dioxidothiomorpholine-4- carbonyl)piperidine-1- carbonyl)phenyl)-5-(4- (cyanomethoxy)-2,3- difluorophenyl)-1-methyl- 1H-imidazole-2-carboxamide | 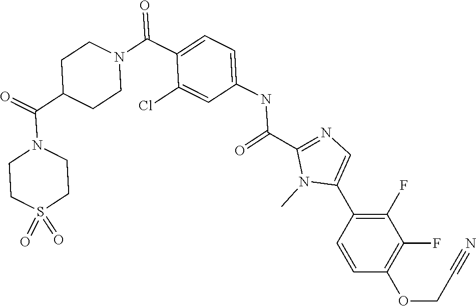 [SMILES: Cn1c(-c2ccc(OCC#N)c(F)c2F)cnc1C(=O)Nc1ccc(C(=O)N2CCC(C(=O)N3CCS(=O)(=O)CC3)CC2)c(Cl)c1] | 675.1 | Intermediate 370 and thiomorpholine 1,1-dioxide |
| 76 | N-(3-chloro-4-(4- (thiomorpholine-4- carbonyl)piperidine-1- carbonyl)phenyl)-5-(4- (cyanomethoxy)-2,3- difluorophenyl)-1-methyl- 1H-imidazole-2-carboxamide | 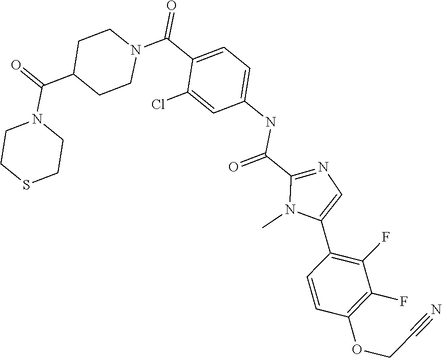 [SMILES: Cn1c(-c2ccc(OCC#N)c(F)c2F)cnc1C(=O)Nc1ccc(C(=O)N2CCC(C(=O)N3CCSCC3)CC2)c(Cl)c1] | 643.2 | Intermediate 370 and thiomorpholine |
| 77 | 5-(4-(cyanomethoxy)-2,3- difluorophenyl)-N-(4-(4-(1,1- dioxidothiomorpholine-4- carbonyl)piperidine-1- carbonyl)-3-methylphenyl)-1- methyl-1H-imidazole-2- carboxamide | 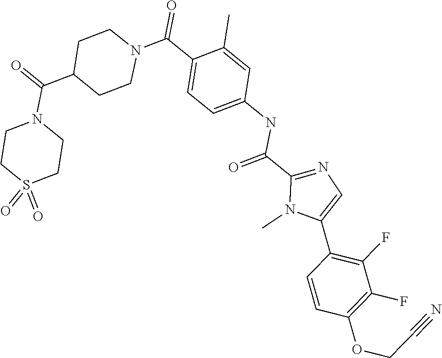 [SMILES: Cc1cc(NC(=O)c2ncc(-c3ccc(OCC#N)c(F)c3F)n2C)ccc1C(=O)N1CCC(C(=O)N2CCS(=O)(=O)CC2)CC1] | 655.2 | Intermediate 372 and thiomorpholine 1,1-dioxide |
| 78 | 5-(4-(cyanomethoxy)-2,3- difluorophenyl)-1-methyl-N- (3-methyl-4-(4-(morpholine- 4-carbonyl)piperidine-1- carbonyl)phenyl)-1H- imidazole-2-carboxamide | 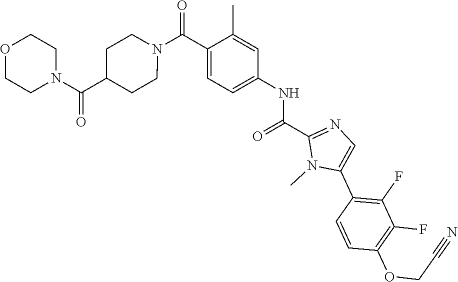 [SMILES: Cc1cc(NC(=O)c2ncc(-c3ccc(OCC#N)c(F)c3F)n2C)ccc1C(=O)N1CCC(C(=O)N2CCOCC2)CC1] | 607.2 | Intermediate 372 and morpholine |
| 79 | N-(4-(4-(3-aminoazetidine-1- carbonyl)piperidine-l- carbonyl)-3-methylphenyl)-5- (4-(cyanomethoxy)-2,3- difluorophenyl)-1-methyl- 1H-imidazole-2-carboxamide 2,2,2-trifluoroacetate | 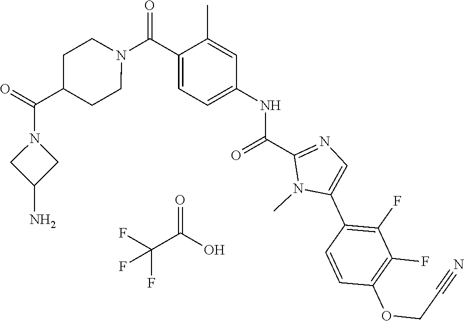 [SMILES: Cc1cc(NC(=O)c2ncc(-c3ccc(OCC#N)c(F)c3F)n2C)ccc1C(=O)N1CCC(C(=O)N2CC(N)C2)CC1 ; O=C(O)C(F)(F)F] | 706.2 | Intermediate 372 and tert- butyl azetidin- 3-ylcarbamate |
| 80 | (R)-N-(4-(4-(3- aminopyrrolidine-1- carbonyl)piperidine-1- carbonyl)-3-chlorophenyl)-5- (4-(cyanomethoxy)-2,3- difluorophenyl)-1-methyl- 1H-imidazole-2-carboxamide formate | 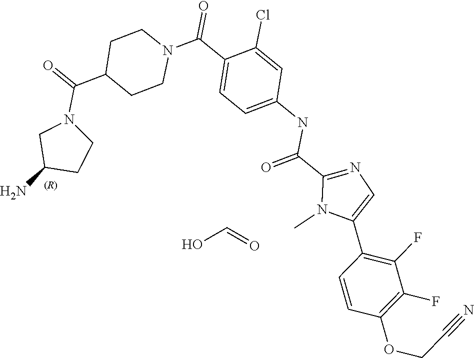 | 672.2 | Intermediate 370 and tert- butyl (R)- pyrrolidin-3- ylcarbamate |
| 81 | (R)-N-(3-chloro-4-(4-(3- (hydroxymethyl)piperazine- 1-carbonyl)piperidine-1- carbonyl)phenyl)-5-(4- (cyanomethoxy)-2,3- difluorophenyl)-1-methyl- 1H-imidazole-2-carboxamide 2,2,2-trifluoroacetate | 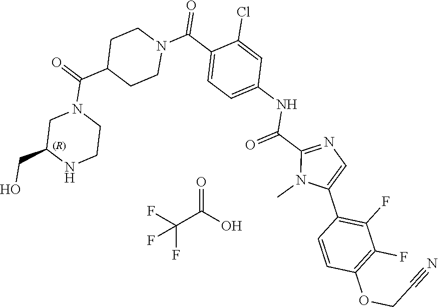 | 770.2 | Intermediate 370 and tert- butyl (R)-2- (hydroxymethyl) piperazine-1- carboxylate |
| 82 | 1-(2-chloro-4-(5-(4- (cyanomethoxy)-2,3- difluorophenyl)-1-methyl- 1H-imidazole-2- carboxamido)benzoyl)-N-(2- (methylamino)ethyl) piperidine-4- carboxamide formate | 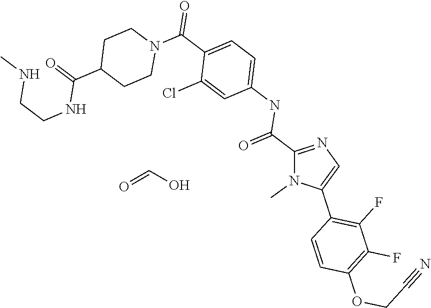 [SMILES: CNCCNC(=O)C1CCN(C(=O)c2ccc(NC(=O)c3ncc(-c4ccc(OCC#N)c(F)c4F)n3C)cc2Cl)CC1 ; O=CO] | 660.2 | Intermediate 370 and tert- butyl (2- aminoethyl) (methyl) carbamate |
| 83 | (R)-N-(4-(4-(2- (aminomethyl)morpholine-4- carbonyl)piperidine-1- carbonyl)-3-methylphenyl)-5- (4-(cyanomethoxy)-2,3- difluorophenyl)-1-methyl- 1H-imidazole-2-carboxamide 2,2,2-trifluoroacetate | 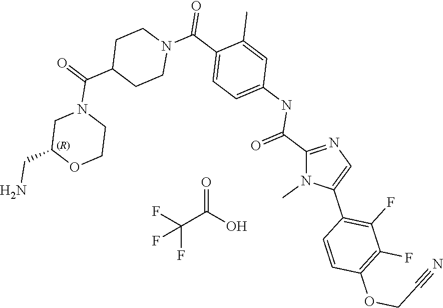 | 750.2 | Intermediate 372 and tert- butyl (S)- (morpholin-2- ylmethyl) carbamate |
| 84 | N-(3-amino-2- hydroxypropyl)-1-(2-chloro- 4-(5-(4-(cyanomethoxy)-2,3- difluorophenyl)-1-methyl- 1H-imidazole-2- carboxamido)benzoyl) piperidine-4- carboxamide 2,2,2- trifluoroacetate | 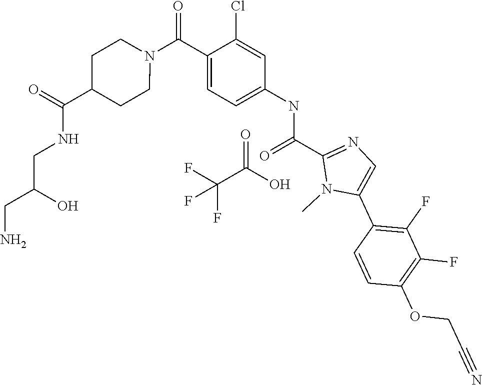 | 744.2 | Intermediate 370 and tert- butyl (3- amino-2- hydroxypropyl) carbamate |
| 85 | N-(4-(4-(2,6- diazaspiro[3.3]heptane-2- carbonyl)piperidine-1- carbonyl)-3-chlorophenyl)-5- (4-(cyanomethoxy)-2,3- difluorophenyl)-1-methyl- 1H-imidazole-2-carboxamide 2,2,2-trifluoroacetate | 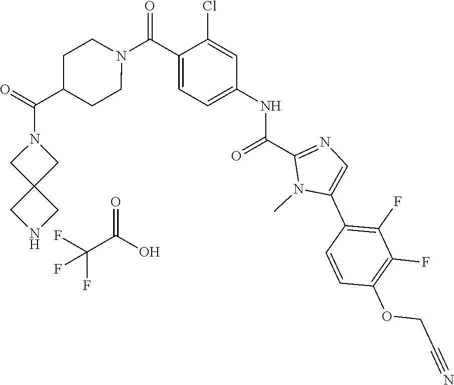 | 752.2 | Intermediate 370 and tert- butyl 2,6- diazaspiro[3.3] heptane-2- carboxylate |
| 86 | (S)-N-(3-ehloro-4-(4-(3- (hydroxymethyl)piperazine- 1-carbonyl)piperidine-1- carbonyl)phenyl)-5-(4- (cyanomethoxy)-2,3- difluorophenyl)-1-methyl- 1H-imidazole-2-carboxamide 2,2,2-trifluoroacetate | 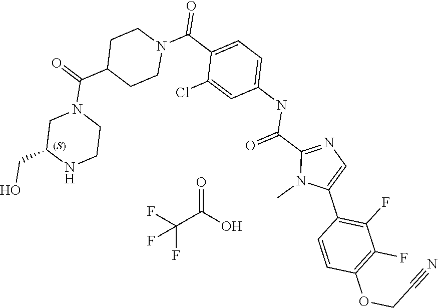 | 770.2 | Intermediate 370 and tert- butyl (S)-2- (hydroxymethyl) piperazine-1- carboxylate |
| 87 | (S)-N-(4-(4-(2- (aminomethyl)morpholine-4- carbonyl)piperidine-1- carbonyl)-3-methylphenyl)-5- (4-(cyanomethoxy)-2,3- difluorophenyl)-1-methyl- 1H-imidazole-2-carboxamide 2,2,2-trifluoroacetate | 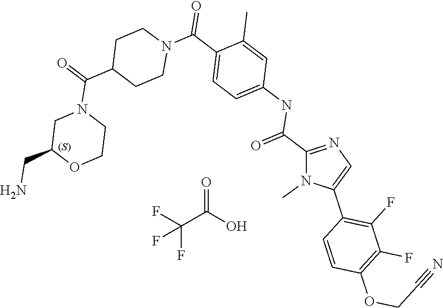 | 750.2 | Intermediate 372 and tert- butyl (R)- (morpholin-2- ylmethyl) carbamate |
| 88 | N-(2-aminoethyl)-1-(2- chloro-4-(5-(2,3-difluoro-4- (fluoromethoxy)phenyl)-1- methyl-1H-imidazole-2- carboxamido)benzoyl) piperidine-4-carboxamide 2,2,2-trifluoroacetate | 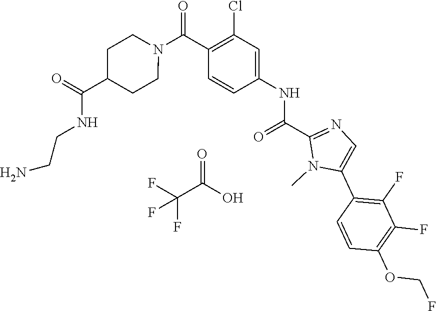 [SMILES: Cn1c(-c2ccc(OCF)c(F)c2F)cnc1C(=O)Nc1ccc(C(=O)N2CCC(C(=O)NCCN)CC2)c(Cl)c1 ; O=C(O)C(F)(F)F] | 707.2 | Intermediate 441 and tert- butyl (2- aminoethyl) carbamate |
| 89 | 1-(2-chloro-4-(5-(2,3- difluoro-4- (fluoromethoxy)phenyl)-1- methyl-1H-imidazole-2- carboxamido)benzoyl)-N- (piperidin-4-yl)piperidine-4- carboxamide 2,2,2- trifluoroacetate | 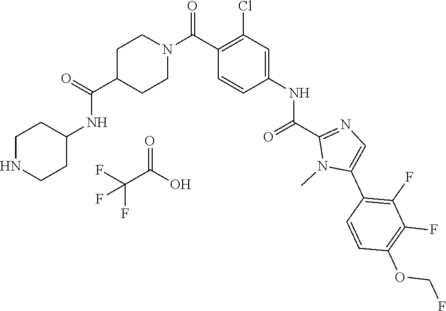 | 747.2 | Intermediate 441 and tert- butyl 4- amino- piperidine- 1-carboxylate |
| 90 | (R)-1-(2-chloro-4-(5-(2,3- difluoro-4- (fluoromethoxy)phenyl)-1- methyl-1H-imidazole-2- carboxamido)benzoyl)-N- (pyrrolidin-3-yl)piperidine-4- carboxamide 2,2,2- trifluoroacetate | 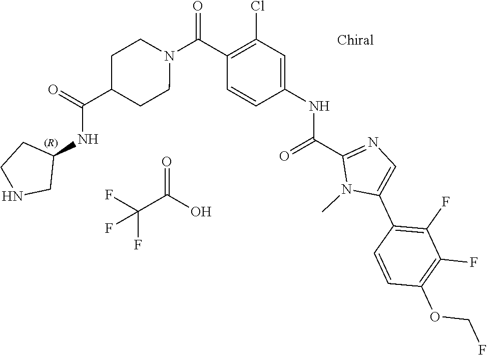 | 733.2 | Intermediate 441 and tert- butyl (R)-3- amino- pyrrolidine- 1-carboxylate |
| 91 | (R)-1-(2-chloro-4-(5-(4- (difluoromethoxy)-2,3- difluorophenyl)-1-methyl- 1H-imidazole-2- carboxamido)benzoyl)-N- (pyrrolidin-3-yl)piperidine-4- carboxamide formate | 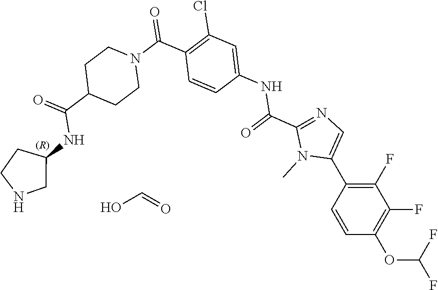 | 683.2 | Intermediate 442 and tert- butyl (R)-3- amino- pyrrolidine- 1-carboxylate |
| 92 | N-(2-aminoethyl)-1-(2- chloro-4-(5-(4- (difluoromethoxy)-2,3- difluorophenyl)-1-methyl- 1H-imidazole-2- carboxamido)benzoyl) piperidine-4- carboxamide formate | 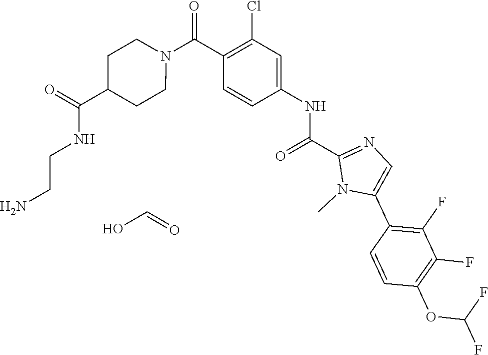 [SMILES: Cn1c(-c2ccc(OC(F)F)c(F)c2F)cnc1C(=O)Nc1ccc(C(=O)N2CCC(C(=O)NCCN)CC2)c(Cl)c1 ; O=CO] | 657.2 | Intermediate 442 and tert- butyl (2- aminoethyl) carbamate |
| 93 | 1-(2-chloro-4-(5-(4- (difluoromethoxy)-2,3- difluorophenyl)-1-methyl- 1H-imidazole-2- carboxamido)benzoyl)-N- ((3S,4S)-4- hydroxypyrrolidin-3- yl)piperidine-4-carboxamide formate | 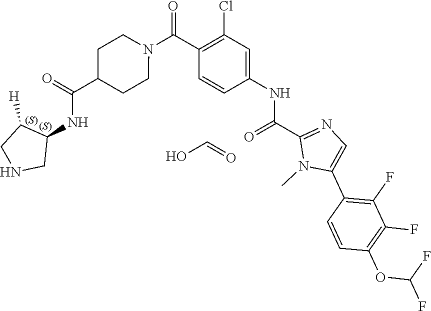 | 699.2 | Intermediate 442 and tert- butyl (3S,4S)- 3-amino-4- hydroxy- pyrrolidine- 1-carboxylate |
| 94 | 1-(2-chloro-4-(5-(4- (difluoromethoxy)-2,3- difluorophenyl)-1-methyl- 1H-imidazole-2- carboxamido)benzoyl)-N- (piperidin-4-yl)piperidine-4- carboxamide formate | 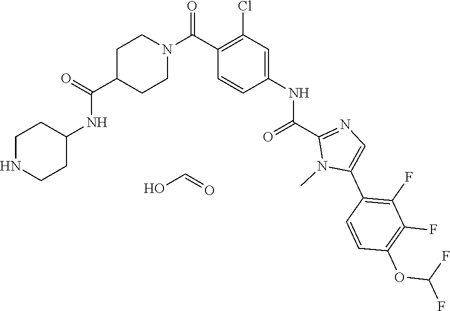 [SMILES: Cn1c(-c2ccc(OC(F)F)c(F)c2F)cnc1C(=O)Nc1ccc(C(=O)N2CCC(C(=O)NC3CCNCC3)CC2)c(Cl)c1 ; O=CO] | 697.2 | Intermediate 442 and tert- butyl 4- amino- piperidine- 1-carboxylate |
| 95 | (S)-1-(2-chloro-4-(5-(4- (difluoromethoxy)-2,3- difluorophenyl)-1-methyl- 1H-imidazole-2- carboxamido)benzoyl)-N- (pyrrolidin-3-yl)piperidine-4- carboxamide formate | 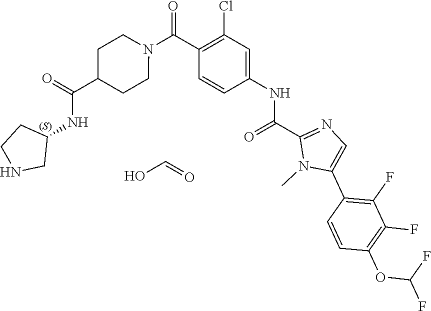 | 683.2 | Intermediate 442 and tert- butyl (S)-3- amino- pyrrolidine- 1-carboxylate |
| 96 | 1-(2-chloro-4-(5-(4- (difluoromethoxy)-2,3- difluorophenyl)-1-methyl- 1H-imidazole-2- carboxamido)benzoyl)-N- ((3R,4R)-4- hydroxypyrrolidin-3- yl)piperidine-4-carboxamide formate | 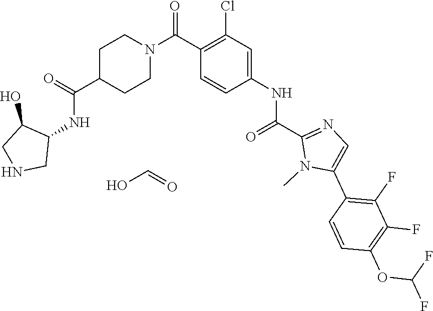 [SMILES: Cn1c(-c2ccc(OC(F)F)c(F)c2F)cnc1C(=O)Nc1ccc(C(=O)N2CCC(C(=O)N[C@@H]3CNC[C@H]3O)CC2)c(Cl)c1 ; O=CO] | 699.2 | Intermediate 442 and tert- butyl (3R,4R)- 3-amino-4- hydroxy- pyrrolidine- 1-carboxylate |
| 97 | 5-(2,3-difluoro-4-methoxy- phenyl)-N-[4-[4-[4-[3- (dimethylamino)propyl] piperazine- 1-carbonyl]piperidine- 1-carbonyl]-3-ethyl-phenyl]- 1-methyl-imidazole-2- carboxamide | 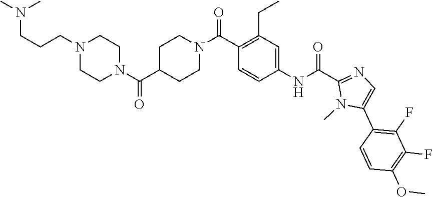 [SMILES: CCc1cc(NC(=O)c2ncc(-c3ccc(OC)c(F)c3F)n2C)ccc1C(=O)N1CCC(C(=O)N2CCN(CCCN(C)C)CC2)CC1] | 680.6 | Intermediate 3 and N,N- dimethyl-3- (piperazin-1- yl)propan-1- amine |
| 99 | 1-[4-[[5-(2,3-difluoro-4- methoxy-phenyl)-1-methyl- imidazole-2- carbonyl]amino]-2-ethyl- benzoyl]-N-[2-[2- (dimethylamino)ethoxy]ethyl] piperidine-4-carboxamid | 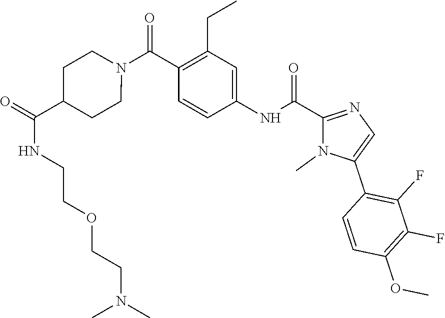 [SMILES: CCc1cc(NC(=O)c2ncc(-c3ccc(OC)c(F)c3F)n2C)ccc1C(=O)N1CCC(C(=O)NCCOCCN(C)C)CC1] | 641.3 | Intermediate 3 and 2-(2- aminoethoxy)- N,N- dimethylethan- 1-amine |
| 100 | N-(2-aminoethyl)-1-[4-[[5- (2,3-difluoro-4-methoxy- phenyl)-1-methyl-imidazole- 2-carbonyl]amino]-2-ethyl- benzoyl]piperidine-4- carboxamide | 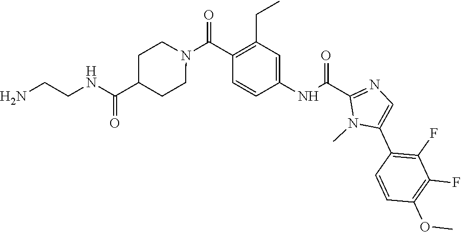 [SMILES: CCc1cc(NC(=O)c2ncc(-c3ccc(OC)c(F)c3F)n2C)ccc1C(=O)N1CCC(C(=O)NCCN)CC1] | 569.3 | Intermediate 3/ and tert-butyl (2-aminoethyl) carbamate |

### Example Type II: 11

### N-[3-Chloro-4-[4-[(2-pyrrolidin-3-ylacetyl)amino]piperidine-1-carbonyl]phenyl]-5-(2,3-difluoro-4-methoxy-phenyl)-1-methyl-imidazole-2-carboxamide formate

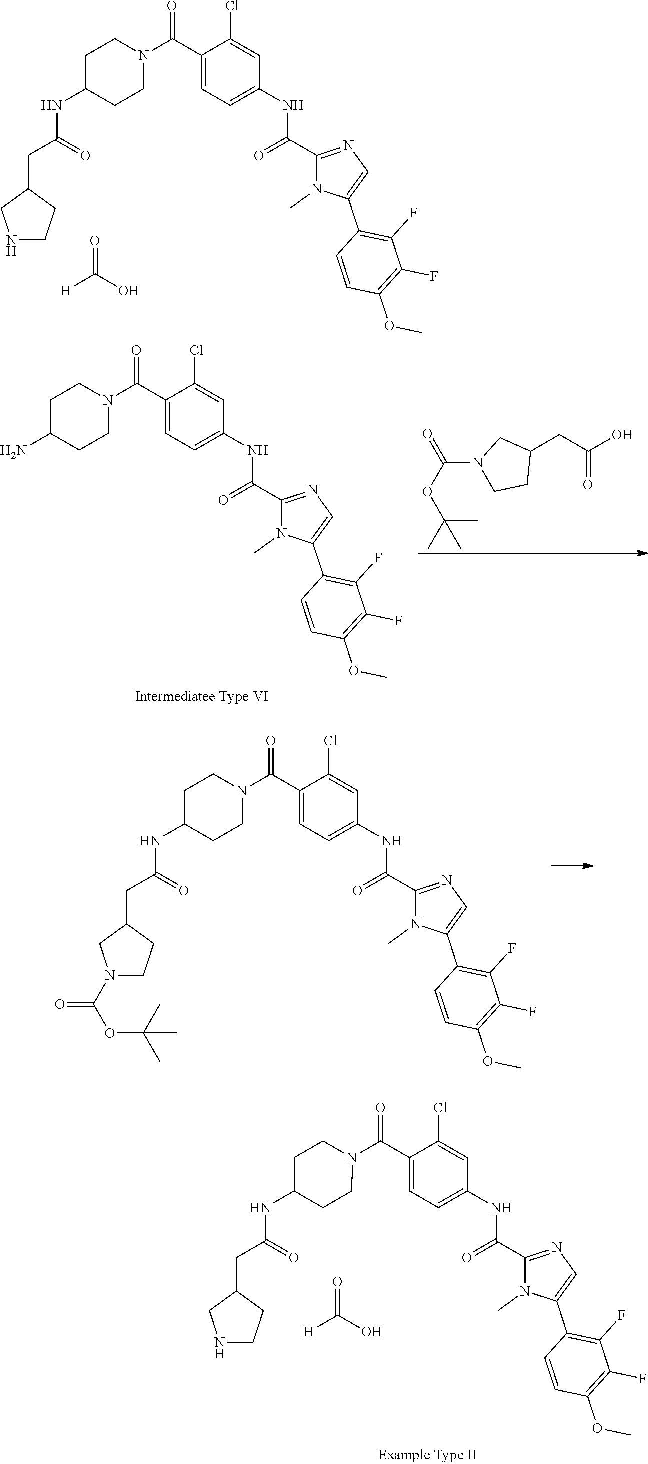

### Step 1: tert-butyl 3-[2-[[1-[2-chloro-4-[[5-(2,3-difluoro-4-methoxy-phenyl)-1-methyl-imidazole-2-carbonyl]amino]benzoyl]-4-piperidyl]amino]-2-oxo-ethyl]pyrrolidine-1-carboxylate

A mixture of N-[4-(4-aminopiperidine-1-carbonyl)-3-chloro-phenyl]-5-(2,3-difluoro-4-methoxy-phenyl)-1-methyl-imidazole-2-carboxamide (intermediate 373, 200 mg, 397 μmol), 2-(1-(tert-butoxycarbonyl)pyrrolidin-3-yl)acetic acid (182 mg, 794 μmol), HATU (226 mg, 595 μmol) and DIPEA (154 mg, 208 μl, 1.19 mmol) in DMF (5 mL) was stirred overnight. Then the mixture was poured into water. The water layer was extracted with DCM. The organic layer was washed with water, dried over anhydrous Na₂SO₄ and concentrated to give the crude product (200 mg), which was used into next step reaction directly. MS (ESI, m/z): 715.1 [M+H]⁺.

### Step 2: N-[3-chloro-4-[4-[(2-pyrrolidin-3-ylacetyl)amino]piperidine-1-carbonyl]phenyl]-5-(2,3-difluoro-4-methoxy-phenyl)-1-methyl-imidazole-2-carboxamide; formic acid

At room temperature, a mixture of 3-[2-[[1-[2-chloro-4-[[5-(2,3-difluoro-4-methoxy-phenyl)-1-methyl-imidazole-2-carbonyl]amino]benzoyl]-4-piperidyl]amino]-2-oxo-ethyl]pyrrolidine-1-carboxylate (200 mg, 280 μmol) in DCM (2 mL) and TFA (3 mL) was stirred for 1 h. Under ice cooling Et3N was added under stirring until pH 8-9. Then water (10 mL) was added. The water layer was extracted with DCM. The organic layer was concentrated to give the crude product which was purified by Prep-HPLC to give the title compound (62 mg). MS (ESI, m/z): 615.1 [M+H]⁺.

The following Type II Examples or Type III Examples were prepared in analogy to example 11.

| | | | MS | |
|---|---|---|---|---|
| | | | ESI | Starting |
| Ex. | Name | Structure | [M + H]⁺ | Material |
| 101 | N-[3-chloro-4-[4-[3- (dimethylamino)propanoyl amino]piperidine-1- carbonyl]phenyl]-5-(2,3- difluoro-4-methoxy- phenyl)-1-methyl- imidazole-2-carboxamide formate | 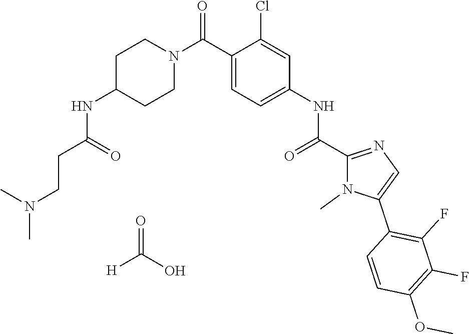 [SMILES: COc1ccc(-c2cnc(C(=O)Nc3ccc(C(=O)N4CCC(NC(=O)CCN(C)C)CC4)c(Cl)c3)n2C)c(F)c1F ; O=CO] | 603.3 | Intermediate 373 and 3- (dimethylamino) propanoic acid |
| 102 | N-[3-chloro-4-[4- [[(2S,4R)-4- hydroxypyrrolidine-2- carbonyl]amino]piperidine- 1-carbonyl]phenyl]-5- (2,3-difluoro-4-methoxy- phenyl)-1-methyl- imidazole-2-carboxamide | 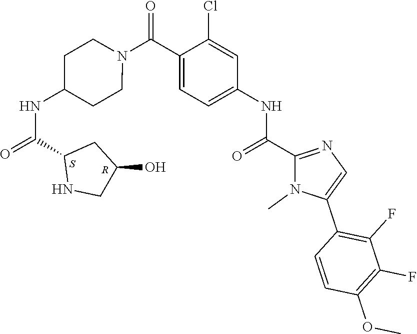 | 617.2 | Intermediate 373 and (2S,4R)-1- (tert- butoxycarbonyl)- 4- hydroxy- pyrrolidine- 2- carboxylic acid |
| 103 | N-[4-[4-(3- aminopropanoylamino) piperidine-1-carbonyl]-3- chloro-phenyl]-5-(2,3- difluoro-4-methoxy- phenyl)-1-methyl- imidazole-2-carboxamide formate | 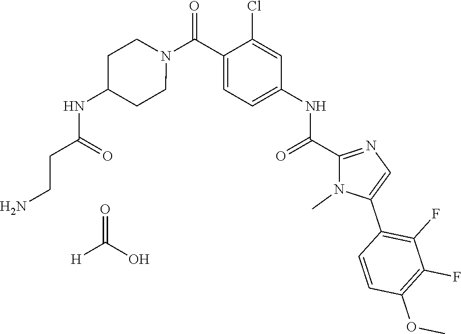 [SMILES: COc1ccc(-c2cnc(C(=O)Nc3ccc(C(=O)N4CCC(NC(=O)CCN)CC4)c(Cl)c3)n2C)c(F)c1F ; O=CO] | 575.0 | Intermediate 373 and 3- ((tert- butoxycarbonyl) amino) propanoic acid |
| 104 | N-[3-chloro-4-[4-[2- (dimethylamino)acetyl] piperazine-1- carbonyl]phenyl]-5-(2,3- difluoro-4-methoxy- phenyl)-1-methyl- imidazole-2-carboxamide trifluoroacetate | 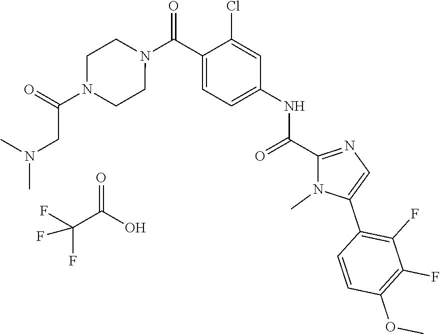 | 575.3 | Intermediate 374 and 2- (dimethylamino) acetic acid |
| 105 | N-[3-chloro-4-[4-[2- (methylamino)acetyl] piperazine-1-carbonyl] phenyl]-5-(2,3-difluoro-4- methoxy-phenyl)-1- methyl-imidazole-2- carboxamide trifluoroacetate | 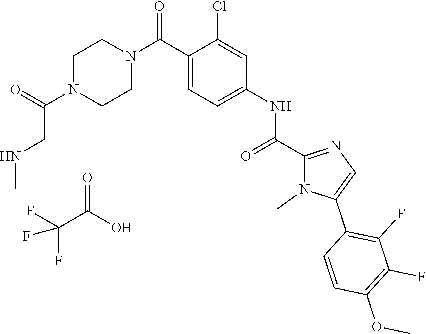 | 561.3 | Intermediate 374 and 2- (tert- butoxycarbonyl- amino) acetic acid |
| 106 | N-[3-chloro-4-[4- [(2S,4R)-4- hydroxypyrrolidine-2- carbonyl]piperazine-1- carbonyl]phenyl]-5-(2,3- difluoro-4-methoxy- phenyl)-1-methyl- imidazole-2-carboxamide trifluoroacetate | 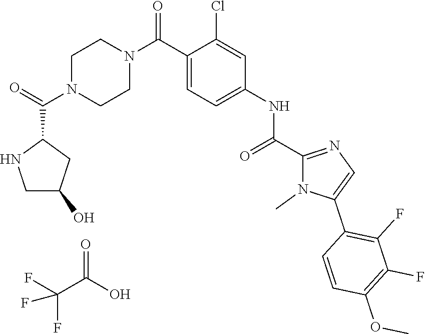 | 603.2 | Intermediate 374 and (2S,4R)-1-tert- butoxycarbonyl- 4-hydroxy- pyrrolidine-2- carboxylic acid |
| 107 | N-[3-chloro-4-[4-[(2S,4S)- 4-hydroxypyrrolidine-2- carbonyl]piperazine-1- carbonyl]phenyl]-5-(2,3- difluoro-4-methoxy- phenyl)-1-methyl- imidazole-2-carboxamide trifluoroacetate | 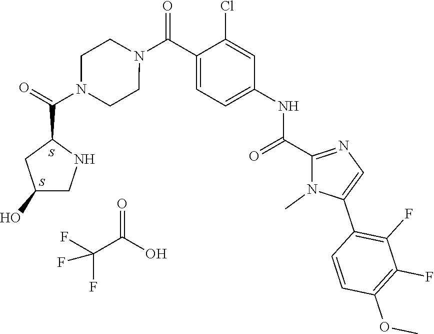 | 603.3 | Intermediate 374 and (2S,4S)-1-tert- butoxycarbonyl- 4-hydroxy- pyrrolidine-2- carboxylic acid |
| 108 | N-[4-[4-[(1S,3R)-3- aminocyclopentane- carbonyl] piperazine-1-carbonyl]- 3-chloro-phenyl]-5-(2,3- difluoro-4-methoxy- phenyl)-1-methyl- imidazole-2-carboxamide; formate | 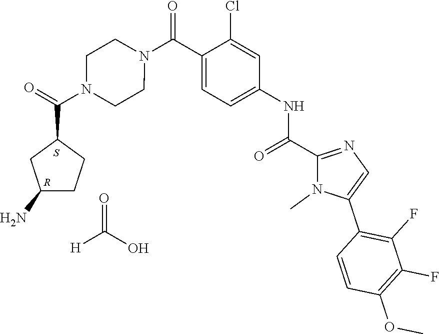 | 601.2 | Intermediate 374 and (1S,3R)-3- aminocyclo- pentane- carboxylic acid |
| 109 | N-[3-chloro-4-[3-[[(3R)- pyrrolidine-3- carbonyl]amino]pyrrolidine- 1-carbonyl]phenyl]-5- (2,3-difluoro-4-methoxy- phenyl)-1-methyl- imidazole-2-carboxamide formate | 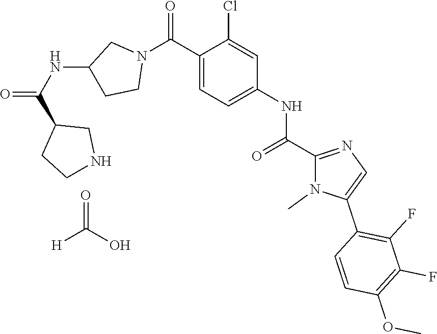 | 587.5 | Intermediate 375 and (R)-1- (tert- butoxycarbonyl) pyrrolidine- 3-carboxylic acid |
| 110 | N-[1-[2-chloro-4-[[5-(2,3- difluoro-4-methoxy- phenyl)-1-methyl- imidazole-2- carbonyl]amino]benzoyl] pyrrolidin-3-yl]piperidine- 4-carboxamide formate | 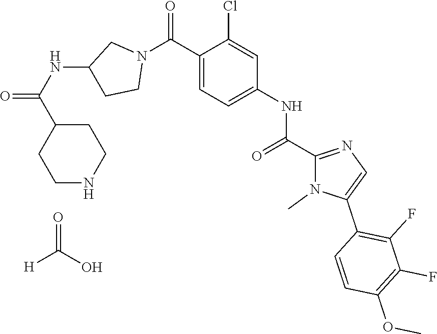 | 601.0 | Intermediate 375 and 1- (tert- butoxycarbonyl) piperidine-4- carboxylic acid |
| 111 | N-[4-[3-[3-(2- aminoethoxy) propanoylamino] pyrrolidine-1- carbonyl]-3-chloro- phenyl]-5-(2,3-difluoro-4- methoxy-phenyl)-1- methyl-imidazole-2- carboxamide formate | 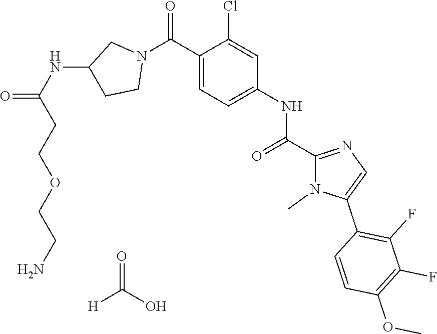 [SMILES: COc1ccc(-c2cnc(C(=O)Nc3ccc(C(=O)N4CCC(NC(=O)CCOCCN)C4)c(Cl)c3)n2C)c(F)c1F ; O=CO] | 605.2 | Intermediate 375 and 3-(2- ((tert- butoxycarbonyl) amino)ethoxy) propanoic acid |
| 112 | N-[3-chloro-4-[2- [(2S,4R)-4- hydroxypyrrolidine-2- carbonyl]-2,6- diazaspiro[3.3]heptane-6- carbonyl]phenyl]-5-(2,3- difluoro-4-methoxy- phenyl)-1-methyl- imidazole-2-carboxamide | 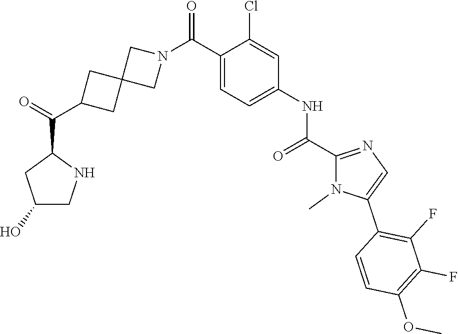 [SMILES: COc1ccc(-c2cnc(C(=O)Nc3ccc(C(=O)N4CC5(CC(C(=O)[C@@H]6C[C@@H](O)CN6)C5)C4)c(Cl)c3)n2C)c(F)c1F] | 615.4 | Intermediate 376 and (2S,4R)-1- (tert- butoxycarbonyl)- 4- hydroxy- pyrrolidine- 2- carboxylic acid |
| 113 | 5-(2-chloro-3-fluoro-4- methoxy-phenyl)-N-[3- chloro-4-[4-(4- hydroxypiperidine-4- carbonyl)piperazine-1- carbonyl]phenyl]-1- methyl-imidazole-2- carboxamide formate | 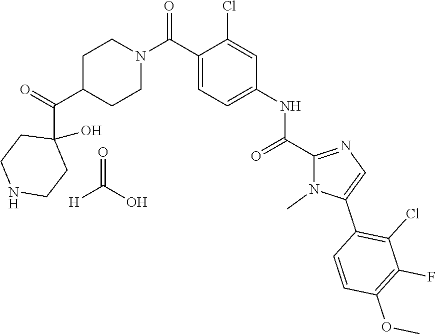 | 632.9 | Intermediate 377 and 1- (tert- butoxycarbonyl)- 4- hydroxy- piperidine- 4- carboxylic acid |
| 114 | 5-(2-chloro-3-fluoro-4- methoxy-phenyl)-N-[3- chloro-4-[4-[(2S,4S)-4- hydroxypyrrolidine-2- carbonyl]piperazine-1- carbonyl]phenyl]-1- methyl-imidazole-2- carboxamide formate | 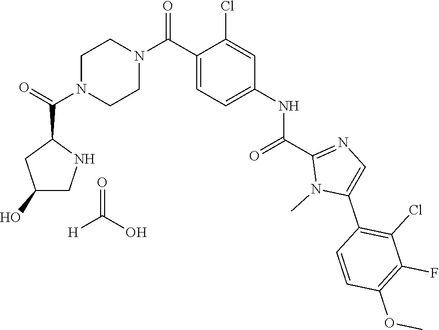 | 618.9 | Intermediate 377 and (2S,4S)-1-tert- butoxycarbonyl- 4-hydroxy- pyrrolidine-2- carboxylic acid |
| 115 | 5-(2-chloro-3-fluoro-4- methoxy-phenyl)-N-[3- chloro-4-[4-[(2R,4R)-4- hydroxypyrrolidine-2- carbonyl]piperazine-1- carbonyl]phenyl]-1- methyl-imidazole-2- carboxamide formate | 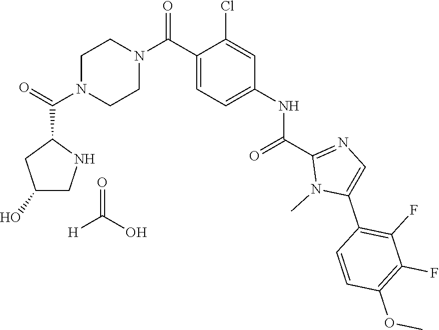 | 618.9 | Intermediate 377 and (2R,4R)-1-tert- butoxycarbonyl- 4-hydroxy- pyrrolidine-2- carboxylic acid |
| 116 | 5-(2-chloro-3-fluoro-4- methoxy-phenyl)-N-[3- chloro-4-[4-[(3R)-3- (hydroxymethyl)piperazine- 1-carbonyl]piperidine-1- carbonyl]phenyl]-1- methyl-imidazole-2- carboxamide | 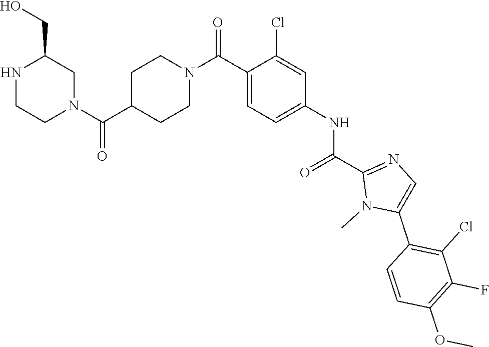 [SMILES: COc1ccc(-c2cnc(C(=O)Nc3ccc(C(=O)N4CCC(C(=O)N5CCN[C@@H](CO)C5)CC4)c(Cl)c3)n2C)c(Cl)c1F] | 647.1 | Intermediate 366 and tert- butyl (2R)-2- (hydroxymethyl) piperazine-1- carboxylate |
| 117 | 5-(2-chloro-3-fluoro-4- methoxy-phenyl)-1- methyl-N-[3-methyl-4-[4- (piperidine-4- carbonyl)piperazine-1- carbonyl]phenyl]imidazole- 2-carboxamide formate | 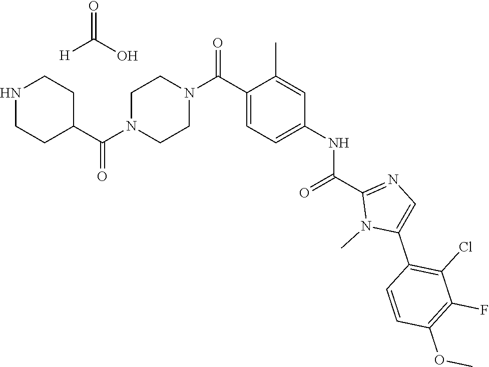 | 597.1 | Intermediate 378 and 1-tert- butoxycarbonyl- piperidine-4- carboxylic acid |
| 118 | 5-(2-chloro-3-fluoro-4- methoxy-phenyl)-N-[4-[4- (4-hydroxypiperidine-4- carbonyl)piperazine-1- carbonyl]-3-methyl- phenyl]-1-methyl- imidazole-2-carboxamide formate | 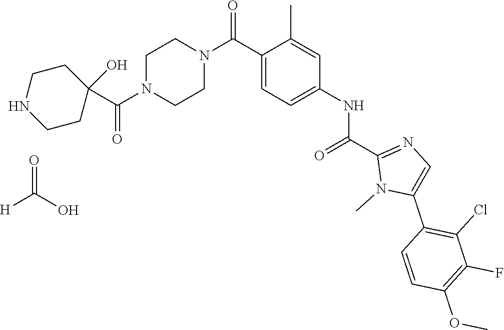 | 613.2 | Intermediate 378 and 1- (tert- butoxycarbonyl)- 4- hydroxy- piperidine- 4- carboxylic acid |
| 119 | N-[4-[4-(2- azaspiro[3.3]heptane-6- carbonyl)piperazine-1- carbonyl]-3-methyl- phenyl]-5-(2-chloro-3- fluoro-4-methoxy-phenyl)- 1-methyl-imidazole-2- carboxamide; formic acid | 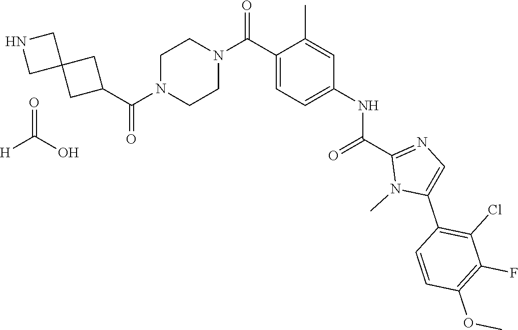 | 609.2 | Intermediate 378 and 2-tert- butoxycarbonyl- 2- azaspiro[3.3] heptane-6- carboxylic acid |
| 120 | N-[3-chloro-4-[4-[1-[2- (dimethylamino)acetyl] piperidine-4- carbonyl]piperazine-1- carbonyl]phenyl]-5-(2,3- difluoro-4-methoxy- phenyl)-1-methyl- imidazole-2-carboxamide | 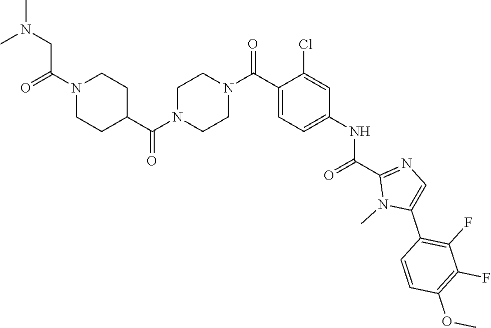 [SMILES: COc1ccc(-c2cnc(C(=O)Nc3ccc(C(=O)N4CCN(C(=O)C5CCN(C(=O)CN(C)C)CC5)CC4)c(Cl)c3)n2C)c(F)c1F] | 686.1 | Intermediate 443 and 2- (dimethylamino) acetic acid |
| 121 | N-[4-[4-(4- aminobutanoyl)piperazine- 1-carbonyl]-3-chloro- phenyl]-5-(2,3-difluoro-4- methoxy-phenyl)-1- methyl-imidazole-2- carboxamide; formic acid | 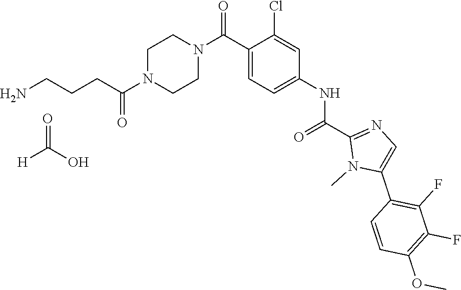 | 575.2 | Intermediate 374 and 4- (tert- butoxycarbonyl- amino) butanoic acid |
| 122 | N-[4-[4-(5-amino-5-oxo- pentanoyl)piperazine-1- carbonyl]-3-chloro- phenyl]-5-(2,3-difluoro-4- methoxy-phenyl)-1- methyl-imidazole-2- carboxamide | 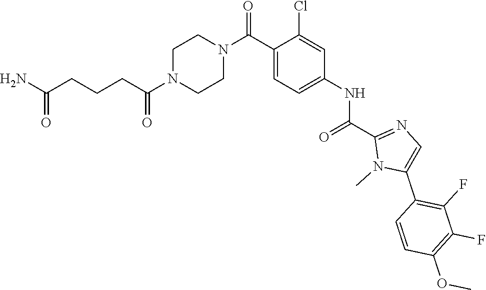 [SMILES: COc1ccc(-c2cnc(C(=O)Nc3ccc(C(=O)N4CCN(C(=O)CCCC(N)=O)CC4)c(Cl)c3)n2C)c(F)c1F] | 603.2 | Intermediate 374 and 5- amino-5-oxo- pentanoic acid |
| 123 | N-[4-[4-(3- aminopropanoyl)piperazine- 1-carbonyl]-3-chloro- phenyl]-5-(2,3-difluoro-4- methoxy-phenyl)-1- methyl-imidazole-2- carboxamide formate | 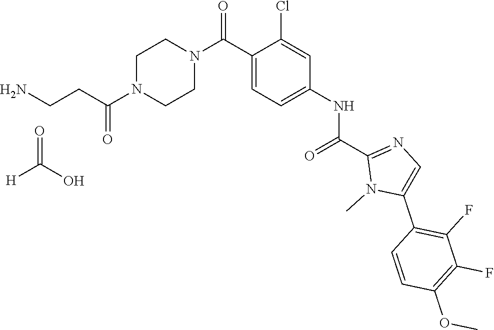 | 561.3 | Intermediate 374 and 3- (tert- butoxycarbonyl- amino) propanoic acid |
| 124 | N-[4-[4-(5- aminopentanoyl)piperazine- 1-carbonyl]-3-chloro- phenyl]-5-(2,3-difluoro-4- methoxy-phenyl)-1- methyl-imidazole-2- carboxamide formate | 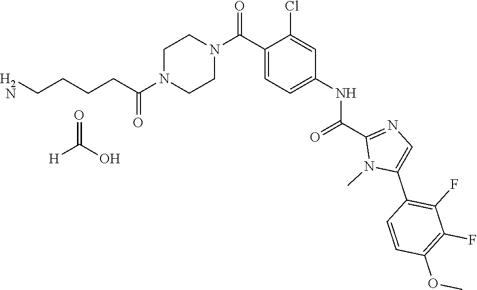 | 589.3 | Intermediate 374 and 5- (tert- butoxycarbonyl- amino) pentanoic acid |
| 125 | N-[3-chloro-4-[4-(6- oxopiperidine-3- carbonyl)piperazine-1- carbonyl]phenyl]-5-(2,3- difluoro-4-methoxy- phenyl)-1-methyl- imidazole-2-carboxamide | 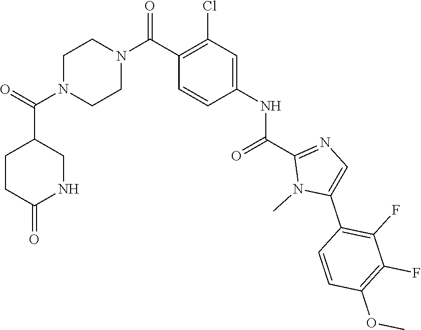 [SMILES: COc1ccc(-c2cnc(C(=O)Nc3ccc(C(=O)N4CCN(C(=O)C5CCC(=O)NC5)CC4)c(Cl)c3)n2C)c(F)c1F] | 614.9 | Intermediate 374 and 6- oxopiperidine- 3-carboxylic acid |
| 126 | N-[3-chloro-4-[4-(5- oxopyrrolidine-2- carbonyl)piperazine-1- carbonyl]phenyl]-5-(2,3- difluoro-4-methoxy- phenyl)-1-methyl- imidazole-2-carboxamide | 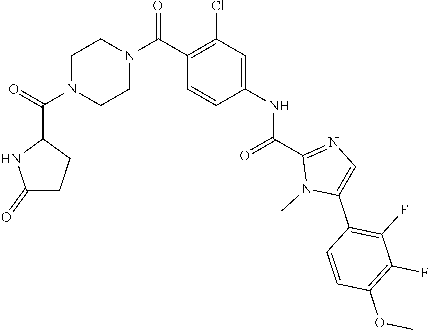 [SMILES: COc1ccc(-c2cnc(C(=O)Nc3ccc(C(=O)N4CCN(C(=O)C5CCC(=O)N5)CC4)c(Cl)c3)n2C)c(F)c1F] | 601.0 | Intermediate 374 and 5- oxopyrrolidine- 2-carboxylic acid |
| 127 | N-[3-chloro-4-[4-[2-(5- oxopyrrolidin-2- yl)acetyl]piperazine-1- carbonyl]phenyl]-5-(2,3- difluoro-4-methoxy- phenyl)-1-methyl- imidazole-2-carboxamide | 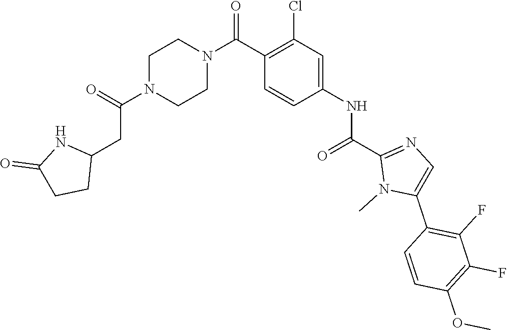 [SMILES: COc1ccc(-c2cnc(C(=O)Nc3ccc(C(=O)N4CCN(C(=O)CC5CCC(=O)N5)CC4)c(Cl)c3)n2C)c(F)c1F] | 614.9 | Intermediate 374 and 2-(5- oxopyrrolidin- 2-yl)acetic acid |
| 128 | N-[3-chloro-4-[4-[2-(2,5- dioxoimidazolidin-4- yl)acetyl]piperazine-1- carbonyl]phenyl]-5-(2,3- difluoro-4-methoxy- phenyl)-1-methyl- imidazole-2-carboxamide | 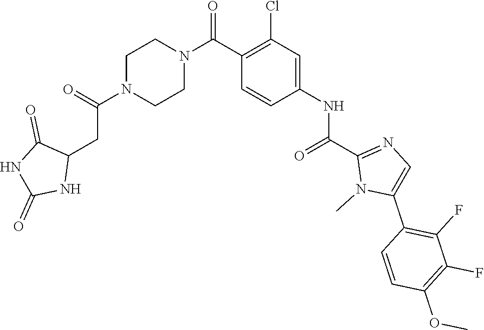 [SMILES: COc1ccc(-c2cnc(C(=O)Nc3ccc(C(=O)N4CCN(C(=O)CC5NC(=O)NC5=O)CC4)c(Cl)c3)n2C)c(F)c1F] | 614.9 | Intermediate 374 and 2-(2,5- dioxoimidazolidin- 4-yl)acetic acid |
| 129 | N-[3-chloro-4-[4-[2-(5- oxopyrrolidin-3- yl)acetyl]piperazine-1- carbonyl]phenyl]-5-(2,3- difluoro-4-methoxy- phenyl)-1-methyl- imidazole-2-carboxamide | 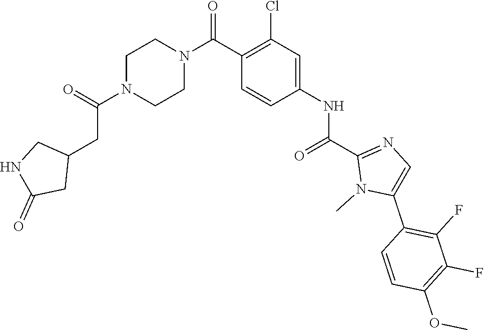 [SMILES: COc1ccc(-c2cnc(C(=O)Nc3ccc(C(=O)N4CCN(C(=O)CC5CNC(=O)C5)CC4)c(Cl)c3)n2C)c(F)c1F] | 614.9 | Intermediate 374 and 2-(5- oxopyrrolidin- 3-yl)acetic acid |
| 130 | N-[4-[4-[5-[(3aS,4S,6aR)- 2-oxo-1,3,3a,4,6,6a- hexahydrothieno[3,4- d]imidazol-4- yl]pentanoyl]piperazine-1- carbonyl]-3-chloro- phenyl]-5-(2,3-difluoro-4- methoxy-phenyl)-1- methyl-imidazole-2- carboxamide | 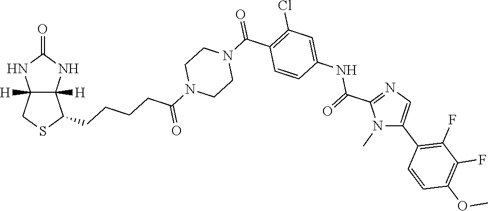 [SMILES: COc1ccc(-c2cnc(C(=O)Nc3ccc(C(=O)N4CCN(C(=O)CCCC[C@@H]5SC[C@@H]6NC(=O)N[C@@H]65)CC4)c(Cl)c3)n2C)c(F)c1F] | 716.2 | Intermediate 374 and 5- [(3aS,4S,6aR)- 2-oxo- 1,3,3a,4,6,6a- hexahydrothieno [3,4- d]imidazol-4- yl]pentanoic acid |
| 131 | N-[4-[4-[3-[2-(2- aminoethoxy)ethoxy] propanoyl]piperazine-1- carbonyl]-3-chloro- phenyl]-5-(2,3-difluoro-4- methoxy-phenyl)-1- methyl-imidazole-2- carboxamide formate | 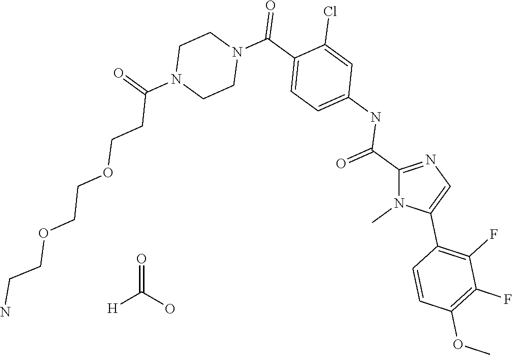 [SMILES: COc1ccc(-c2cnc(C(=O)Nc3ccc(C(=O)N4CCN(C(=O)CCOCCOCCN)CC4)c(Cl)c3)n2C)c(F)c1F ; O=CO] | 649.3 | Intermediate 374 and 3-[2- [2-(tert- butoxycarbonyl- amino)ethoxy] ethoxy] propanoic acid |
| 132 | N-[4-[4-(3- aminobicyclo[1.1.1] pentane-1- carbonyl)piperazine-1- carbonyl]-3-chloro- phenyl]-5-(2,3-difluoro-4- methoxy-phenyl)-1- methyl-imidazole-2- carboxamide formate | 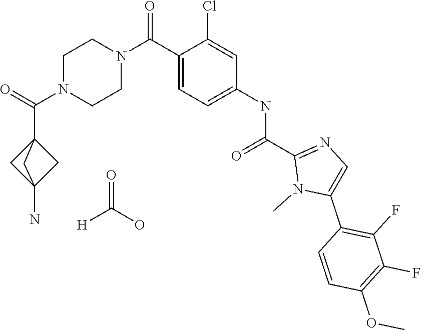 [SMILES: COc1ccc(-c2cnc(C(=O)Nc3ccc(C(=O)N4CCN(C(=O)C56CC(N)(C5)C6)CC4)c(Cl)c3)n2C)c(F)c1F ; O=CO] | 599.2 | Intermediate 374 and 3- (tert- butoxycarbonyl- amino)bicyclo [1.1.1]pentane- 1-carboxylic acid |
| 133 | N-[3-chloro-4-[4-(2- oxoimidazolidine-4- carbonyl)piperazine-1- carbonyl]phenyl]-5-(2,3- difluoro-4-methoxy- phenyl)-1-methyl- imidazole-2-carboxamide | 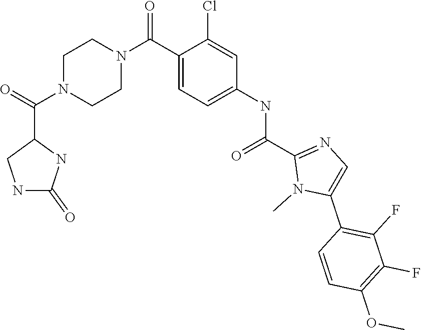 [SMILES: COc1ccc(-c2cnc(C(=O)Nc3ccc(C(=O)N4CCN(C(=O)C5CNC(=O)N5)CC4)c(Cl)c3)n2C)c(F)c1F] | 599.2 | Intermediate 374 and 2- oxoimidazolidine- 4-carboxylic acid |
| 134 | N-[3-chloro-4-[4-(3- cyanopropanoyl)piperazine- 1-carbonyl]phenyl]-5- (2,3-difluoro-4-methoxy- phenyl)-1-methyl- imidazole-2-carboxamide | 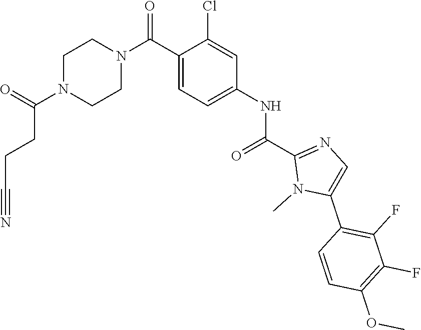 [SMILES: COc1ccc(-c2cnc(C(=O)Nc3ccc(C(=O)N4CCN(C(=O)CCC#N)CC4)c(Cl)c3)n2C)c(F)c1F] | 571.1 | Intermediate 374 and 3- cyanopropanoic acid |
| 135 | N-[3-chloro-4-[4-(4- guanidinobutanoyl) piperazine- 1-carbonyl]phenyl]-5- (2,3-difluoro-4-methoxy- phenyl)-1-methyl- imidazole-2-carboxamide formate | 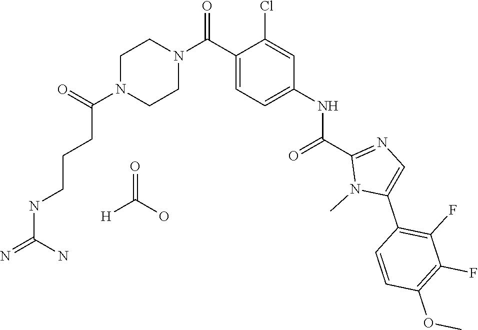 [SMILES: COc1ccc(-c2cnc(C(=O)Nc3ccc(C(=O)N4CCN(C(=O)CCCNC(=N)N)CC4)c(Cl)c3)n2C)c(F)c1F ; O=CO] | 617.1 | Intermediate 444 and tert- butyl (NZ)-N- [(tert- butoxycarbonyl- amino)- pyrazol-1-yl- methylene] carbamate |
| 136 | N-[4-[4-[(1R,5S)-3- azabicyclo[3.1.0]hexane- 6-carbonyl]piperazine-1- carbonyl]-3-chloro- phenyl]-5-[2,3-difluoro-4- (fluoromethoxy)phenyl]-1- methyl-imidazole-2- carboxamide formate | 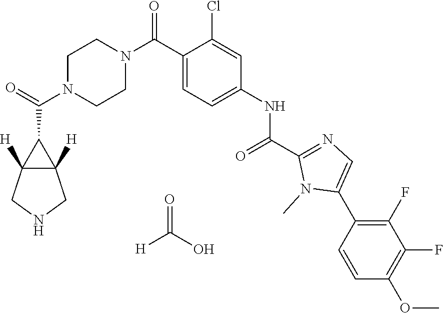 [SMILES: COc1ccc(-c2cnc(C(=O)Nc3ccc(C(=O)N4CCN(C(=O)[C@@H]5[C@@H]6CNC[C@@H]65)CC4)c(Cl)c3)n2C)c(F)c1F ; O=CO] | 617.1 | Intermediate 392 and (1S,5R)-3-tert- butoxycarbonyl- 3- azabicyclo[3.1.0] hexane-6- carboxylic acid |
| 137 | N-[4-[4-(3- aminocyclobutanecarbonyl) piperazine-1-carbonyl]-3- chloro-phenyl]-5-(2,3- difluoro-4-methoxy- phenyl)-1-methyl- imidazole-2-carboxamide formate | 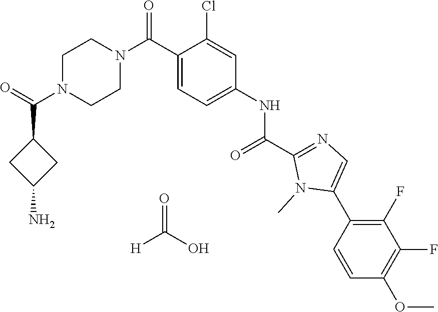 [SMILES: COc1ccc(-c2cnc(C(=O)Nc3ccc(C(=O)N4CCN(C(=O)[C@H]5C[C@H](N)C5)CC4)c(Cl)c3)n2C)c(F)c1F ; O=CO] | 587.4 | Intermediate 374 and 3- (tert- butoxycarbonyl- amino) cyclobutane- carboxylic acid |
| 138 | N-[3-chloro-4-[4-[(3R)- pyrrolidine-3- carbonyl]piperazine-1- carbonyl]phenyl]-5-(2,3- difluoro-4-methoxy- phenyl)-1-methyl- imidazole-2-carboxamide formate | 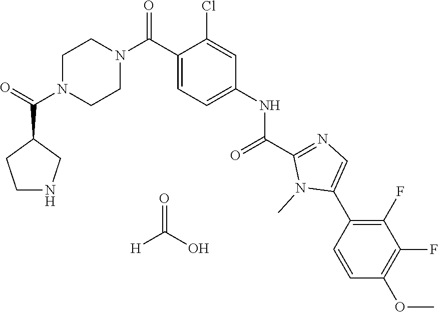 [SMILES: COc1ccc(-c2cnc(C(=O)Nc3ccc(C(=O)N4CCN(C(=O)[C@@H]5CCNC5)CC4)c(Cl)c3)n2C)c(F)c1F ; O=CO] | 587.2 | Intermediate 374 and (3R)- 1-tert- butoxycarbonyl pyrrolidine-3- carboxylic acid |
| 139 | N-[3-chloro-4-[4-[(3S)- pyrrolidine-3- carbonyl]piperazine-1- carbonyl]phenyl]-5-(2,3- difluoro-4-methoxy- phenyl)-1-methyl- imidazole-2-carboxamide formate | 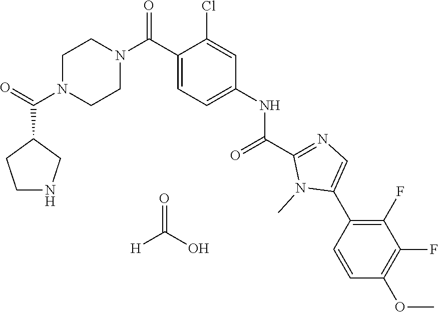 [SMILES: COc1ccc(-c2cnc(C(=O)Nc3ccc(C(=O)N4CCN(C(=O)[C@H]5CCNC5)CC4)c(Cl)c3)n2C)c(F)c1F ; O=CO] | 587.2 | Intermediate 374 and (3S)- 1-tert- butoxycarbonyl- pyrrolidine-3- carboxylic acid |
| 140 | N-[4-[4-(3- aminocyclobutanecarbonyl) piperazine-1-carbonyl]-3- chloro-phenyl]-5-(2,3- difluoro-4-methoxy- phenyl)-1-methyl- imidazole-2-carboxamide formate | 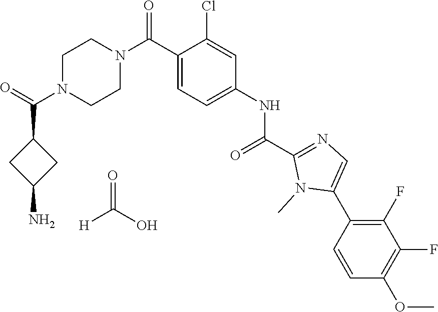 [SMILES: COc1ccc(-c2cnc(C(=O)Nc3ccc(C(=O)N4CCN(C(=O)[C@H]5C[C@@H](N)C5)CC4)c(Cl)c3)n2C)c(F)c1F ; O=CO] | 587.0 | Intermediate 374 and 3- (tert- butoxycarbonyl- amino) cyclobutane- carboxylic acid |
| 141 | N-[3-chloro-4-[4-(3- hydroxypyrrolidine-3- carbonyl)piperazine-1- carbonyl]phenyl]-5-(2,3- difluoro-4-methoxy- phenyl)-1-methyl- imidazole-2-carboxamide formate | 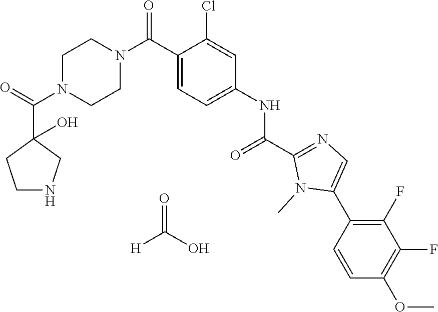 [SMILES: COc1ccc(-c2cnc(C(=O)Nc3ccc(C(=O)N4CCN(C(=O)C5(O)CCNC5)CC4)c(Cl)c3)n2C)c(F)c1F ; O=CO] | 603.3 | Intermediate 374 and 1-tert- butoxycarbonyl- 3-hydroxy- pyrrolidine-3- carboxylic acid |
| 142 | N-[3-chloro-4-[4-[2-(4- piperidyl)acetyl]piperazine- 1-carbonyl]phenyl]-5- (2,3-difluoro-4-methoxy- phenyl)-1-methyl- imidazole-2- carboxamide; formic acid | 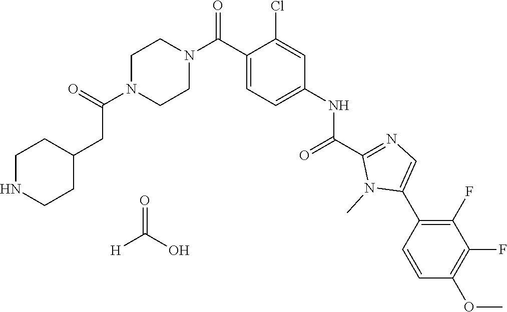 [SMILES: COc1ccc(-c2cnc(C(=O)Nc3ccc(C(=O)N4CCN(C(=O)CC5CCNCC5)CC4)c(Cl)c3)n2C)c(F)c1F ; O=CO] | 615.3 | Intermediate 374 and 2-(1- tert- butoxycarbonyl- 4-piperidyl) acetic acid |
| 143 | N-[3-chloro-4-[4- (piperidine-4- carbonyl)piperazine-1- carbonyl]phenyl]-5-[2,3- difluoro-4- (fluoromethoxy)phenyl]-1- methyl-imidazole-2- carboxamide formate | 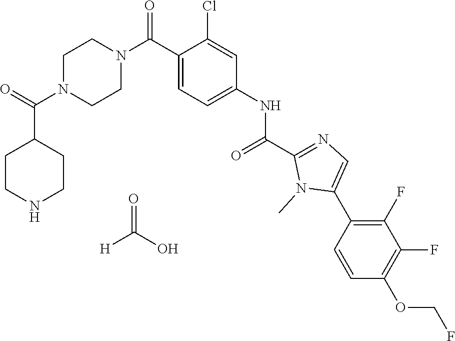 [SMILES: Cn1c(-c2ccc(OCF)c(F)c2F)cnc1C(=O)Nc1ccc(C(=O)N2CCN(C(=O)C3CCNCC3)CC2)c(Cl)c1 ; O=CO] | 619.5 | Intermediate 392 and 1-tert- butoxycarbonyl- piperidine-4- carboxylic acid |
| 144 | N-[3-chloro-4-[4- (piperidine-4- carbonyl)piperazine-1- carbonyl]phenyl]-5-[4- (difluoromethoxy)-3- fluoro-phenyl]-1-methyl- imidazole-2-carboxamide formate | 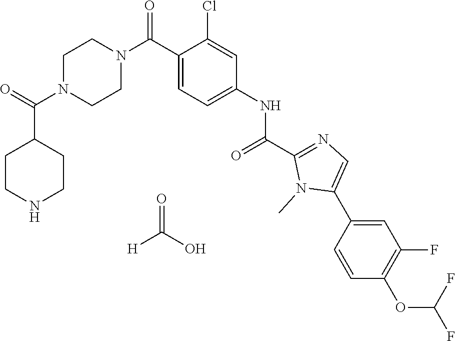 [SMILES: Cn1c(-c2ccc(OC(F)F)c(F)c2)cnc1C(=O)Nc1ccc(C(=O)N2CCN(C(=O)C3CCNCC3)CC2)c(Cl)c1 ; O=CO] | 619.5 | Intermediate 383 and 1-tert- butoxycarbonyl- piperidine-4- carboxylic acid |
| 145 | N-[4-[4-[2-(azetidin-3- yl)acetyl]piperazine-1- carbonyl]-3-chloro- phenyl]-5-(2,3-difluoro-4- methoxy-phenyl)-1- methyl-imidazole-2- carboxamide formate | 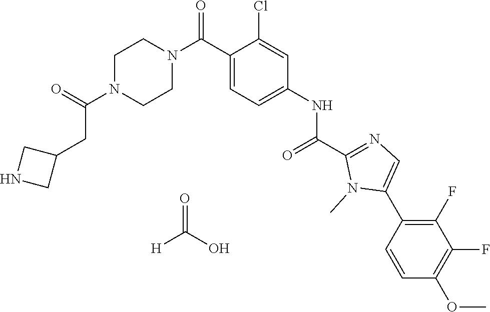 [SMILES: COc1ccc(-c2cnc(C(=O)Nc3ccc(C(=O)N4CCN(C(=O)CC5CNC5)CC4)c(Cl)c3)n2C)c(F)c1F ; O=CO] | 551.2 | Intermediate 374 and 2-(1- tert- butoxycarbonyl- azetidin-3- yl)acetic acid |
| 146 | 5-[2-chloro-3-fluoro-4- (fluoromethoxy)phenyl]- N-[3-chloro-4-[4-[2-[(3S)- pyrrolidin-3- yl]acetyl]piperazine-1- carbonyl]phenyl]-1- methyl-imidazole-2- carboxamide formate | 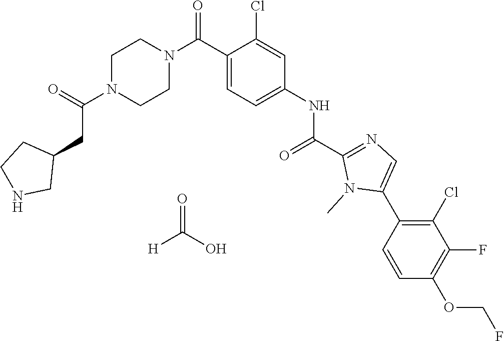 [SMILES: Cn1c(-c2ccc(OCF)c(F)c2Cl)cnc1C(=O)Nc1ccc(C(=O)N2CCN(C(=O)C[C@@H]3CCNC3)CC2)c(Cl)c1 ; O=CO] | 635.1 | Intermediate 400 and 2- [(3S)-1-tert- butoxycarbonyl- pyrrolidin-3- yl]acetic acid |
| 147 | 5-[2-chloro-4- (difluoromethoxy)-3- fluoro-phenyl]-N-[3- chloro-4-[4-[2-[(3S)- pyrrolidin-3- yl]acetyl]piperazine-1- carbonyl]phenyl]-1- methyl-imidazole-2- carboxamide formate | 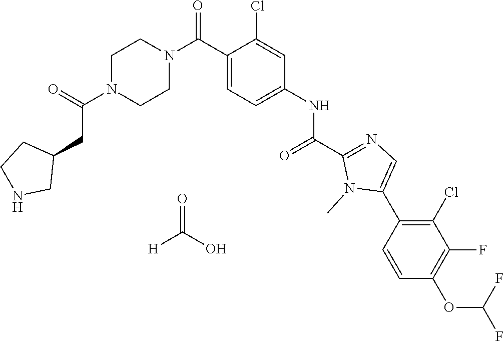 [SMILES: Cn1c(-c2ccc(OC(F)F)c(F)c2Cl)cnc1C(=O)Nc1ccc(C(=O)N2CCN(C(=O)C[C@@H]3CCNC3)CC2)c(Cl)c1 ; O=CO] | 655.1 | Intermediate 393 and 2- [(3S)-1-tert- butoxycarbonyl- pyrrolidin-3- yl]acetic acid |
| 148 | N-[3-chloro-4-[4-(4- hydroxypyrrolidine-3- carbonyl)piperazine-1- carbonyl]phenyl]-5-[2,3- difluoro-4- (fluoromethoxy)phenyl]-1- methyl-imidazole-2- carboxamide formate | 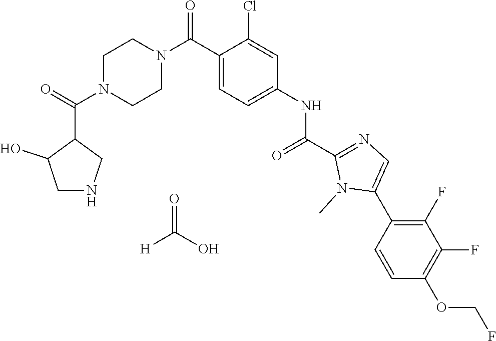 [SMILES: Cn1c(-c2ccc(OCF)c(F)c2F)cnc1C(=O)Nc1ccc(C(=O)N2CCN(C(=O)C3CNCC3O)CC2)c(Cl)c1 ; O=CO] | 621.0 | Intermediate 392 and 1-tert- butoxycarbonyl- 4-hydroxy- pyrrolidine-3- carboxylic acid |
| 149 | N-[4-[4-(2- aminoacetyl)piperazine-1- carbonyl]-3-chloro- phenyl]-5-(2,3-difluoro-4 methoxy-phenyl)-1- methyl-imidazole-2- carboxamide formate | 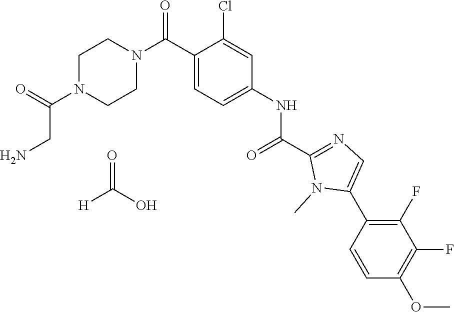 [SMILES: COc1ccc(-c2cnc(C(=O)Nc3ccc(C(=O)N4CCN(C(=O)CN)CC4)c(Cl)c3)n2C)c(F)c1F ; O=CO] | 547.2 | Intermediate 374 and 2- (tert- butoxycarbonyl- amino)acetic acid |
| 150 | N-[4-[4-[(2S)-azetidine-2- carbonyl]piperazine-1- carbonyl]-3-chloro- phenyl]-5-(2,3-difluoro-4- methoxy-phenyl)-1- methyl-imidazole-2- carboxamide formate | 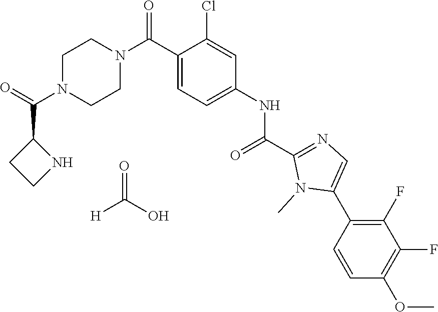 [SMILES: COc1ccc(-c2cnc(C(=O)Nc3ccc(C(=O)N4CCN(C(=O)[C@@H]5CCN5)CC4)c(Cl)c3)n2C)c(F)c1F ; O=CO] | 573.2 | Intermediate 374 and (2S)- 1-tert- butoxycarbonyl- azetidine-2- carboxylic acid |
| 151 | N-[3-chloro-4-[4-(2- pyrrolidin-3- ylacetyl)piperazine-1- carbonyl]phenyl]-5-(2,3- difluoro-4-methoxy- phenyl)-1-methyl- imidazole-2-carboxamide formate | 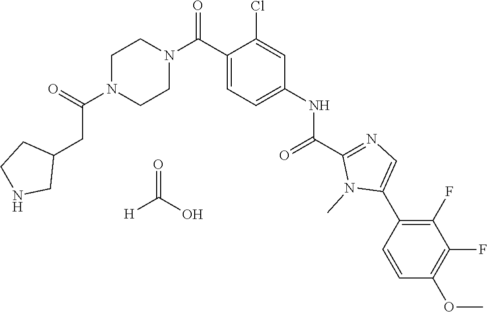 [SMILES: COc1ccc(-c2cnc(C(=O)Nc3ccc(C(=O)N4CCN(C(=O)CC5CCNC5)CC4)c(Cl)c3)n2C)c(F)c1F ; O=CO] | 601.2 | Intermediate 374 and 2-(1- tert- butoxycarbonyl- pyrrolidin-3- yl)acetic acid |
| 152 | N-[3-chloro-4-[4-[2-[(2R)- pyrrolidin-2- yl]acetyl]piperazine-1- carbonyl]phenyl]-5-(2,3- difluoro-4-methoxy- phenyl)-1-methyl- imidazole-2-carboxamide formate | 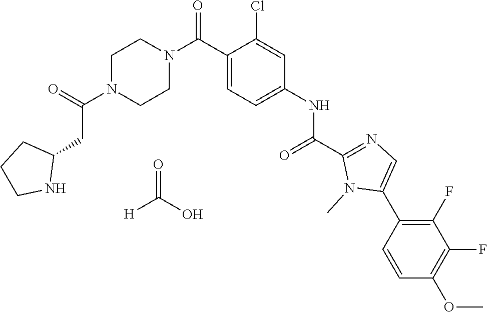 [SMILES: COc1ccc(-c2cnc(C(=O)Nc3ccc(C(=O)N4CCN(C(=O)C[C@H]5CCCN5)CC4)c(Cl)c3)n2C)c(F)c1F ; O=CO] | 601.3 | Intermediate 374 and 2- [(2R)-1-tert- butoxycarbonyl- pyrrolidin-2- yl]acetic acid |
| 153 | N-[3-chloro-4-[4-[2-[(3R)- pyrrolidin-3- yl]acetyl]piperazine-1- carbonyl]phenyl]-5-(2,3- difluoro-4-methoxy- phenyl)-1-methyl- imidazole-2-carboxamide formate | 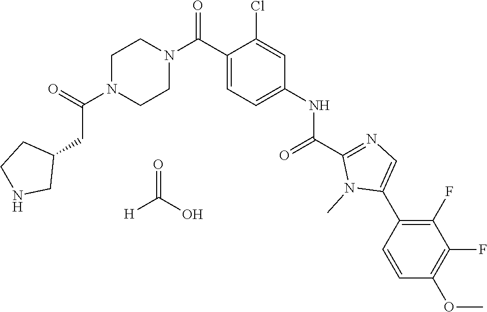 [SMILES: COc1ccc(-c2cnc(C(=O)Nc3ccc(C(=O)N4CCN(C(=O)C[C@H]5CCNC5)CC4)c(Cl)c3)n2C)c(F)c1F ; O=CO] | 601.2 | Intermediate 374 and 2- [(3R)-1-tert- butoxycarbonyl- pyrrolidin-3- yl]acetic acid |
| 154 | N-[3-chloro-4-[4-[2-[(2S)- pyrrolidin-2- yl]acetyl]piperazine-1- carbonyl]phenyl]-5-(2,3- difluoro-4-methoxy- phenyl)-1-methyl- imidazole-2-carboxamide formate | 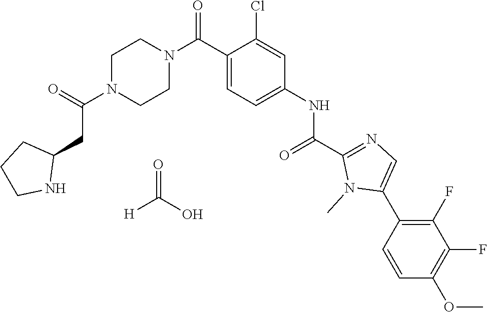 [SMILES: COc1ccc(-c2cnc(C(=O)Nc3ccc(C(=O)N4CCN(C(=O)C[C@@H]5CCCN5)CC4)c(Cl)c3)n2C)c(F)c1F ; O=CO] | 601.3 | Intermediate 374 and 2- [(2S)-1-tert- butoxycarbonyl- pyrrolidin-2- yl]acetic acid |
| 155 | N-[3-chloro-4-[4-[(3R)- pyrrolidine-3- carbonyl]piperazine-1- carbonyl]phenyl]-5-[2,3- difluoro-4- (fluoromethoxy)phenyl]-1- methyl-imidazole-2- carboxamide formate | 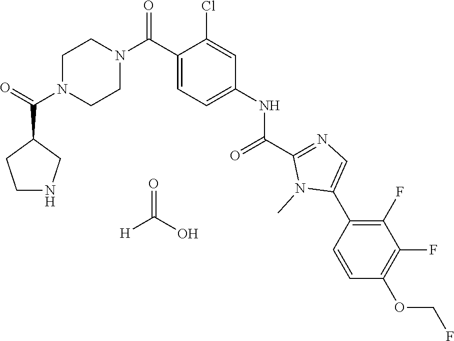 [SMILES: Cn1c(-c2ccc(OCF)c(F)c2F)cnc1C(=O)Nc1ccc(C(=O)N2CCN(C(=O)[C@@H]3CCNC3)CC2)c(Cl)c1 ; O=CO] | 605.3 | Intermediate 392 and (3R)- 1-tert- butoxycarbonyl- pyrrolidine-3- carboxylic acid |
| 156 | N-[3-chloro-4-[4-[2- (dimethylamino)acetyl] piperazine-1- carbonyl]phenyl]-5-[4- (difluoromethoxy)-2,3- difluoro-phenyl]-1-methyl- imidazole-2-carboxamide formate | 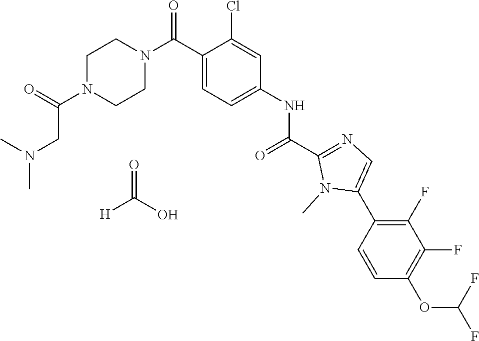 [SMILES: CN(C)CC(=O)N1CCN(C(=O)c2ccc(NC(=O)c3ncc(-c4ccc(OC(F)F)c(F)c4F)n3C)cc2Cl)CC1 ; O=CO] | 612.1 | Intermediate 381 and 2- (dimethylamino) acetic acid |
| 157 | N-[3-chloro-4-[4-[2-[(2S)- pyrrolidin-2- yl]acetyl]piperazine-1- carbonyl]phenyl]-5-[2,3- difluoro-4- (fluoromethoxy)phenyl]-1- methyl-imidazole-2- carboxamide formate | 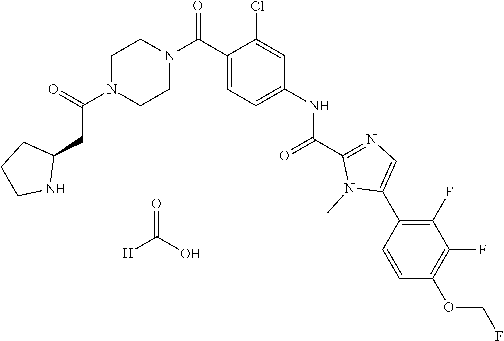 [SMILES: Cn1c(-c2ccc(OCF)c(F)c2F)cnc1C(=O)Nc1ccc(C(=O)N2CCN(C(=O)C[C@@H]3CCCN3)CC2)c(Cl)c1 ; O=CO] | 619.3 | Intermediate 392 and 2- [(2S)-1-tert- butoxycarbonyl- pyrrolidin-2- yl]acetic acid |
| 158 | N-[3-chloro-4-[4-[2-[(3S)- pyrrolidin-3- yl]acetyl]piperazine-1- carbonyl]phenyl]-5-[2,3- difluoro-4- (fluoromethoxy)phenyl]-1- methyl-imidazole-2- carboxamide formate | 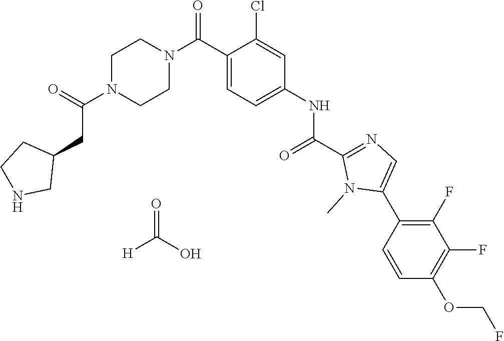 [SMILES: Cn1c(-c2ccc(OCF)c(F)c2F)cnc1C(=O)Nc1ccc(C(=O)N2CCN(C(=O)C[C@@H]3CCNC3)CC2)c(Cl)c1 ; O=CO] | 619.3 | Intermediate 392 and 2- [(3S)-1-tert- butoxycarbonyl- pyrrolidin-3- yl]acetic acid |
| 159 | N-[3-chloro-4-[4-[2-[(2S)- pyrrolidin-2- yl]acetyl]piperazine-1- carbonyl]phenyl]-5-[4- (difluoromethoxy)-2- fluoro-phenyl]-1-methyl- imidazole-2-carboxamide formate | 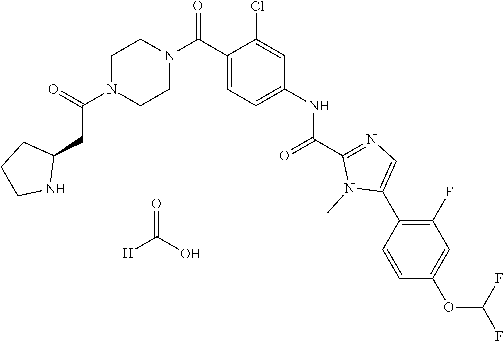 [SMILES: Cn1c(-c2ccc(OC(F)F)cc2F)cnc1C(=O)Nc1ccc(C(=O)N2CCN(C(=O)C[C@@H]3CCCN3)CC2)c(Cl)c1 ; O=CO] | 619.2 | Intermediate 382 and 2- [(2S)-1-tert- butoxycarbonyl- pyrrolidin-2- yl]acetic acid |
| 160 | N-[3-chloro-4-[4-[2-(3- hydroxyazetidin-3- yl)acetyl]piperazine-1- carbonyl]phenyl]-5-[2,3- difluoro-4- (fluoromethoxy)phenyl]-1- methyl-imidazole-2- carboxamide formate | 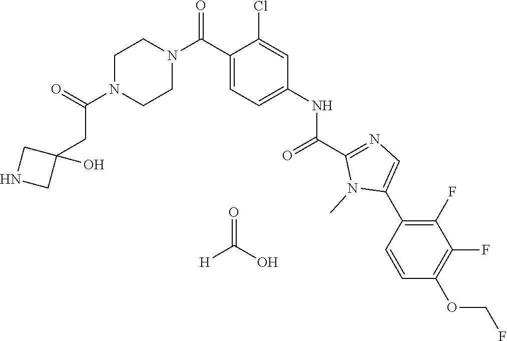 [SMILES: Cn1c(-c2ccc(OCF)c(F)c2F)cnc1C(=O)Nc1ccc(C(=O)N2CCN(C(=O)CC3(O)CNC3)CC2)c(Cl)c1 ; O=CO] | 621.1 | Intermediate 392 and 2-(1- tert- butoxycarbonyl- 3-hydroxy- azetidin-3- yl)acetic acid |
| 161 | N-[3-chloro-4-[4-[2-(4- hydroxy-4- piperidyl)acetyl]piperazine- 1-carbonyl]phenyl]-5- (2,3-difluoro-4-methoxy- phenyl)-1-methyl- imidazole-2-carboxamide formate | 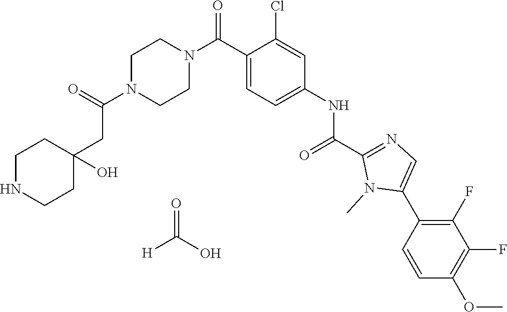 [SMILES: COc1ccc(-c2cnc(C(=O)Nc3ccc(C(=O)N4CCN(C(=O)CC5(O)CCNCC5)CC4)c(Cl)c3)n2C)c(F)c1F ; O=CO] | 631.3 | Intermediate 374 and 2-(1- tert- butoxycarbonyl- 4-hydroxy-4- piperidyl) acetic acid |
| 162 | N-[3-chloro-4-[4-(4- hydroxypiperidine-4- carbonyl)piperazine-1- carbonyl]phenyl]-5-[2,3- difluoro-4- (fluoromethoxy)phenyl]-1- methyl-imidazole-2- carboxamide formate | 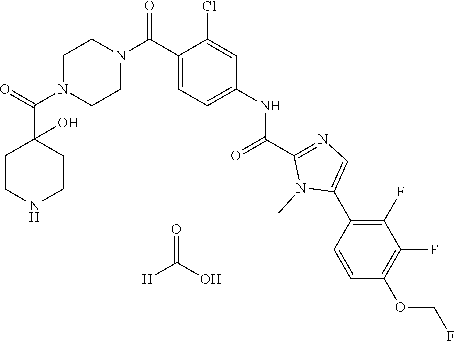 [SMILES: Cn1c(-c2ccc(OCF)c(F)c2F)cnc1C(=O)Nc1ccc(C(=O)N2CCN(C(=O)C3(O)CCNCC3)CC2)c(Cl)c1 ; O=CO] | 635.3 | Intermediate 392 and 1-tert- butoxycarbonyl- 4-hydroxy- piperidine-4- carboxylic acid |
| 163 | N-[3-chloro-4-[4- [(2S,4R)-4- hydroxypyrrolidine-2- carbonyl]piperazine-1- carbonyl]phenyl]-5-[4- (difluoromethoxy)-2,3- difluoro-phenyl]-1-methyl- imidazole-2-carboxamide formate | 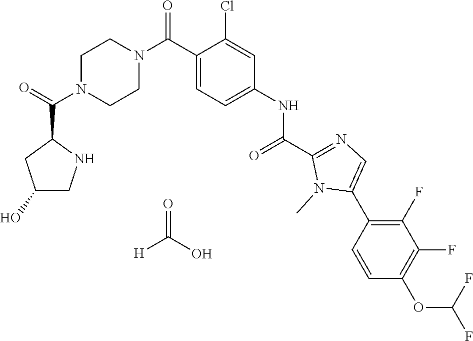 [SMILES: Cn1c(-c2ccc(OC(F)F)c(F)c2F)cnc1C(=O)Nc1ccc(C(=O)N2CCN(C(=O)[C@@H]3C[C@@H](O)CN3)CC2)c(Cl)c1 ; O=CO] | 639.7 | Intermediate 381 and (2S,4R)-1-tert- butoxycarbonyl- 4-hydroxy- pyrrolidine-2- carboxylic acid |
| 164 | N-[3-chloro-4-[4-[2-(4- hydroxy-4- piperidyl)acetyl]piperazine- 1-carbonyl]phenyl]-5- [2,3-difluoro-4- (fluoromethoxy)phenyl]-1- methyl-imidazole-2- carboxamide formate | 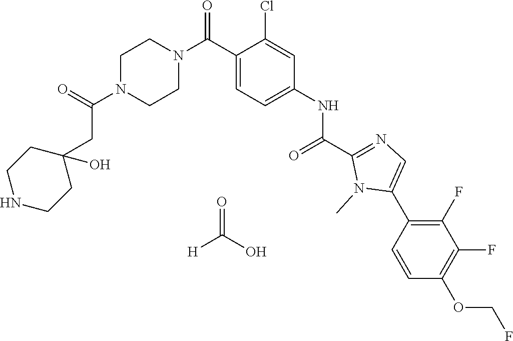 [SMILES: Cn1c(-c2ccc(OCF)c(F)c2F)cnc1C(=O)Nc1ccc(C(=O)N2CCN(C(=O)CC3(O)CCNCC3)CC2)c(Cl)c1 ; O=CO] | 649.3 | Intermediate 392 and 2-(1- tert- butoxycarbonyl- 4-hydroxy-4- piperidyl) acetic acid |
| 165 | N-[4-[4-(azetidine-3- carbonyl)piperazine-1- carbonyl]-3-chloro- phenyl]-5-(2,3-difluoro-4- methoxy-phenyl)-1- methyl-imidazole-2- carboxamide formate | 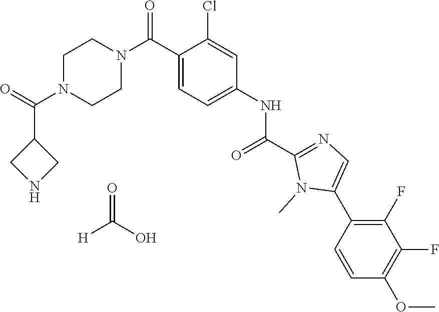 [SMILES: COc1ccc(-c2cnc(C(=O)Nc3ccc(C(=O)N4CCN(C(=O)C5CNC5)CC4)c(Cl)c3)n2C)c(F)c1F ; O=CO] | 573.3 | Intermediate 374 and 1-tert- butoxycarbonyl- azetidine-3- carboxylic acid |
| 166 | N-[3-chloro-4-[4-(4- hydroxypiperidine-4- carbonyl)piperazine-1- carbonyl]phenyl]-5-(2,3- difluoro-4-methoxy- phenyl)-1-methyl- imidazole-2-carboxamide formate | 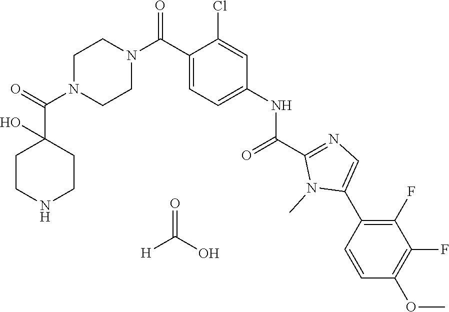 [SMILES: COc1ccc(-c2cnc(C(=O)Nc3ccc(C(=O)N4CCN(C(=O)C5(O)CCNCC5)CC4)c(Cl)c3)n2C)c(F)c1F ; O=CO] | 617.1 | Intermediate 374 and 1-tert- butoxycarbonyl- 4-hydroxy- piperidine-4- carboxylic acid |
| 167 | 5-(2-chloro-3-fluoro-4- methoxy-phenyl)-N-[3- chloro-4-[4-[2-[(3S)- pyrrolidin-3- yl]acetyl]piperazine-1- carbonyl]phenyl]-1- methyl-imidazole-2- carboxamide formate | 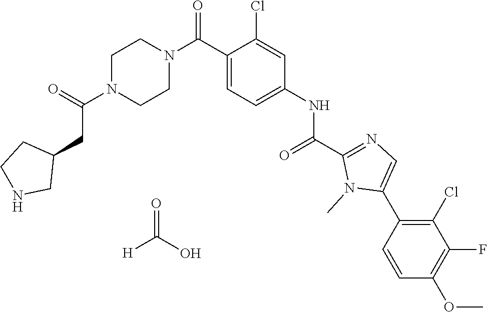 [SMILES: COc1ccc(-c2cnc(C(=O)Nc3ccc(C(=O)N4CCN(C(=O)C[C@@H]5CCNC5)CC4)c(Cl)c3)n2C)c(Cl)c1F ; O=CO] | 617.2 | Intermediate 377 and 2- [(3S)-1-tert- butoxycarbonyl- pyrrolidin-3- yl]acetic acid |
| 168 | N-[3-chloro-4-[4- (piperidine-4- carbonyl)piperazine-1- carbonyl]phenyl]-1- methyl-5-(2,3,5-trifluoro- 4-methoxy- phenyl)imidazole-2- carboxamide formate | 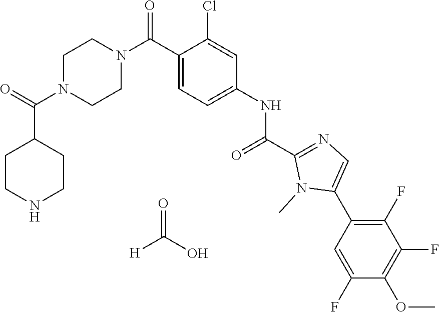 [SMILES: COc1c(F)cc(-c2cnc(C(=O)Nc3ccc(C(=O)N4CCN(C(=O)C5CCNCC5)CC4)c(Cl)c3)n2C)c(F)c1F ; O=CO] | 619.3 | Intermediate 395 and 1-tert- butoxycarbonyl- piperidine-4- carboxylic acid |
| 169 | 5-[2-chloro-4- (difluoromethoxy)-3- fluoro-phenyl]-N-[3- chloro-4-[4-(4- hydroxypiperidine-4- carbonyl)piperazine-1- carbonyl]phenyl]-1- methyl-imidazole-2- carboxamide formate | 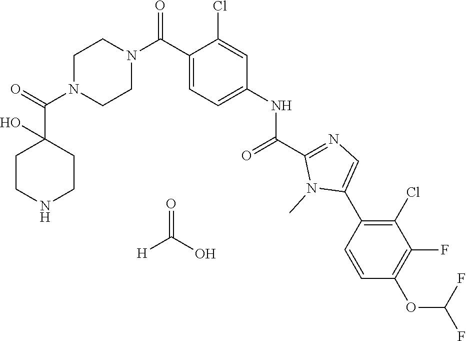 [SMILES: Cn1c(-c2ccc(OC(F)F)c(F)c2Cl)cnc1C(=O)Nc1ccc(C(=O)N2CCN(C(=O)C3(O)CCNCC3)CC2)c(Cl)c1 ; O=CO] | 671.2 | Intermediate 393 and 1-tert- butoxycarbonyl- 4-hydroxy- piperidine-4- carboxylic acid |
| 170 | N-[3-chloro-4-[4-(4- hydroxypiperidine-4- carbonyl)piperazine-1- carbonyl]phenyl]-5-(4- ethoxy-2,3-difluoro- phenyl)-1-methyl- imidazole-2-carboxamide formate | 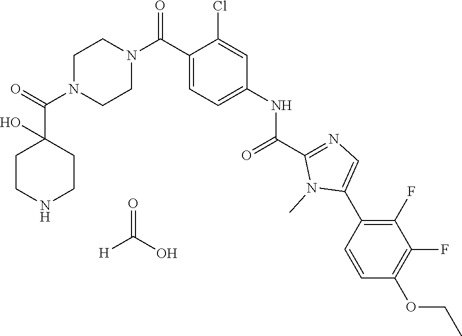 [SMILES: CCOc1ccc(-c2cnc(C(=O)Nc3ccc(C(=O)N4CCN(C(=O)C5(O)CCNCC5)CC4)c(Cl)c3)n2C)c(F)c1F ; O=CO] | 631.0 | Intermediate 396 and 1-tert- butoxycarbonyl- 4-hydroxy- piperidine-4- carboxylic acid |
| 171 | N-[3-chloro-4-[4-(5- hydroxypiperidine-3- carbonyl)piperazine-1- carbonyl]phenyl]-5-[2,3- difluoro-4- (fluoromethoxy)phenyl]- methyl-imidazole-2- carboxamide formate | 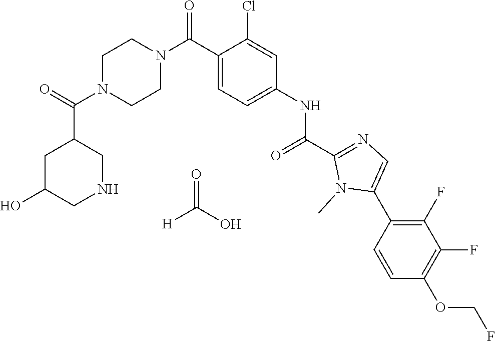 [SMILES: Cn1c(-c2ccc(OCF)c(F)c2F)cnc1C(=O)Nc1ccc(C(=O)N2CCN(C(=O)C3CNCC(O)C3)CC2)c(Cl)c1 ; O=CO] | 635.1 | Intermediate 392 and 1-tert- butoxycarbonyl- hydroxy- piperidine-3- carboxylic acid |
| 172 | 5-[2-chloro-4- (difluoromethoxy)-3- fluoro-phenyl]-N-[3- chloro-4-[4-[2-(4-hydroxy- 4- piperidyl)acetyl]piperazine- 1-carbonyl]phenyl]-1- methyl-imidazole-2- carboxamide formate | 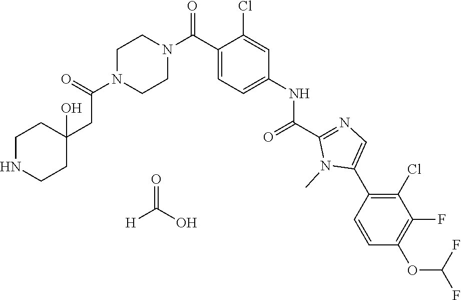 [SMILES: Cn1c(-c2ccc(OC(F)F)c(F)c2Cl)cnc1C(=O)Nc1ccc(C(=O)N2CCN(C(=O)CC3(O)CCNCC3)CC2)c(Cl)c1 ; O=CO] | 685.2 | Intermediate 393 and 2-(1- tert- butoxycarbonyl- 4-hydroxy-4- piperidyl) acetic acid |
| 173 | 5-[2-chloro-3-fluoro-4- (fluoromethoxy)phenyl]- N-[3-chloro-4-[4-[2-(4- hydroxy-4- piperidyl)acetyl]piperazine- 1-carbonyl]phenyl]-1- methyl-imidazole-2- carboxamide formate | 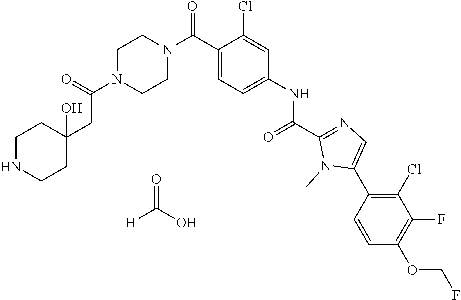 [SMILES: Cn1c(-c2ccc(OCF)c(F)c2Cl)cnc1C(=O)Nc1ccc(C(=O)N2CCN(C(=O)CC3(O)CCNCC3)CC2)c(Cl)c1 ; O=CO] | 665.1 | Intermediate 400 and 2-(1- tert- butoxycarbonyl- 4-hydroxy-4- piperidyl) acetic acid |
| 174 | N-[4-[4-[1-(azetidine-3- carbonyl)piperidine-4- carbonyl]piperazine-1- carbonyl]-3-chloro- phenyl]-5-[4- (difluoromethoxy)-2,3- difluoro-phenyl]-1-methyl- imidazole-2-carboxamide formate | 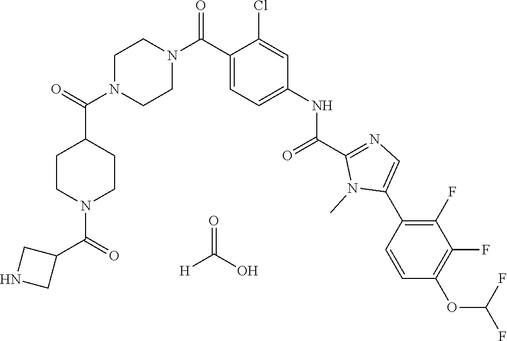 [SMILES: Cn1c(-c2ccc(OC(F)F)c(F)c2F)cnc1C(=O)Nc1ccc(C(=O)N2CCN(C(=O)C3CCN(C(=O)C4CNC4)CC3)CC2)c(Cl)c1 ; O=CO] | 720.2 | Intermediate 381 and 1-tert- butoxycarbonyl- piperidine-4- carboxylic acid |
| 175 | N-[3-chloro-4-[4-[2- (dimethylamino)acetyl] piperazine-1- carbonyl]phenyl]-5-[4- (difluoromethoxy)-2- fluoro-phenyl]-1-methyl- imidazole-2-carboxamide formate | 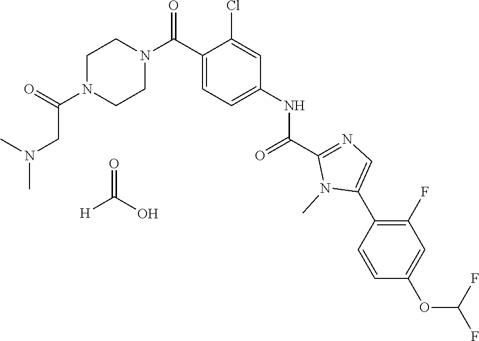 [SMILES: CN(C)CC(=O)N1CCN(C(=O)c2ccc(NC(=O)c3ncc(-c4ccc(OC(F)F)cc4F)n3C)cc2Cl)CC1 ; O=CO] | 593.3 | Intermediate 382 and 2- (dimethylamino) acetic acid |
| 176 | N-[3-chloro-4-[4-[2- (dimethylamino)acetyl] piperazine-1- carbonyl]phenyl]-5-[4- (difluoromethoxy)-3- fluoro-phenyl]-1-methyl- imidazole-2-carboxamide formate | 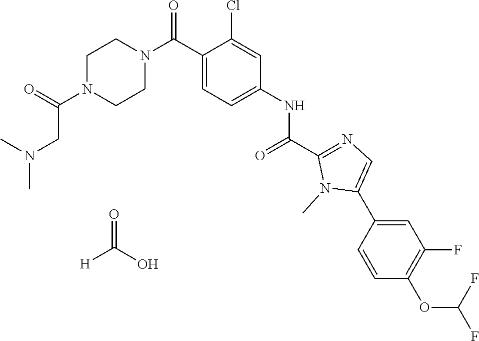 [SMILES: CN(C)CC(=O)N1CCN(C(=O)c2ccc(NC(=O)c3ncc(-c4ccc(OC(F)F)c(F)c4)n3C)cc2Cl)CC1 ; O=CO] | 593.3 | Intermediate 383 and 2- (dimethylamino) acetic acid |
| 177 | N-[3-chloro-4-[4-[2-(3- hydroxyazetidin-3- yl)acetyl]piperazine-1- carbonyl]phenyl]-5-(2,3- difluoro-4-methoxy- phenyl)-1-methyl- imidazole-2-carboxamide formate | 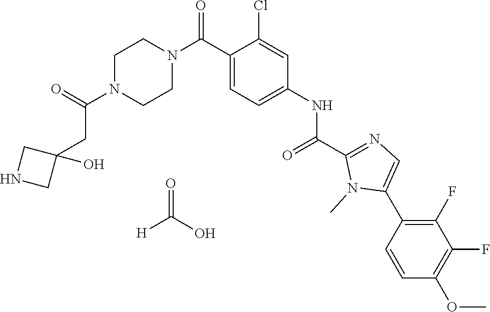 [SMILES: COc1ccc(-c2cnc(C(=O)Nc3ccc(C(=O)N4CCN(C(=O)CC5(O)CNC5)CC4)c(Cl)c3)n2C)c(F)c1F ; O=CO] | 603.3 | Intermediate 377 and 2-(1- tert- butoxycarbonyl- 3-hydroxy- azetidin-3- yl)acetic acid |
| 178 | N-[4-[4-[2-(azetidin-3- yl)acetyl]piperazine-1- carbonyl]-3-chloro- phenyl]-5-[4- (difluoromethoxy)-2- fluoro-phenyl]-1-methyl- imidazole-2-carboxamide formate | 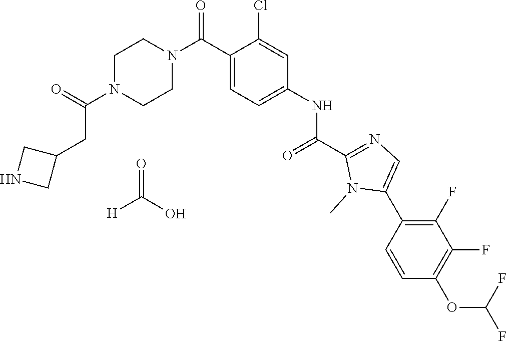 [SMILES: Cn1c(-c2ccc(OC(F)F)c(F)c2F)cnc1C(=O)Nc1ccc(C(=O)N2CCN(C(=O)CC3CNC3)CC2)c(Cl)c1 ; O=CO] | 605.3 | Intermediate 382 and 2-(1- tert- butoxycarbonyl- azetidin-3- yl)acetic acid |
| 179 | N-[3-chloro-4-[4-[1-(4- hydroxypiperidine-4- carbonyl)piperidine-4- carbonyl]piperazine-1- carbonyl]phenyl]-5-[4- (difluoromethoxy)-2,3- difluoro-phenyl]-1-methyl- imidazole-2-carboxamide formate | 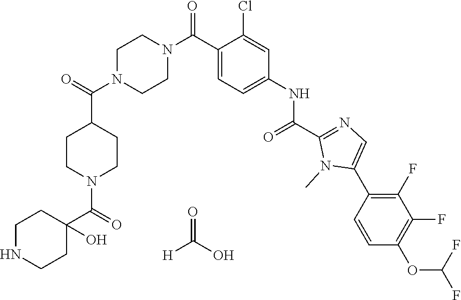 [SMILES: Cn1c(-c2ccc(OC(F)F)c(F)c2F)cnc1C(=O)Nc1ccc(C(=O)N2CCN(C(=O)C3CCN(C(=O)C4(O)CCNCC4)CC3)CC2)c(Cl)c1 ; O=CO] | 764.2 | Intermediate 381 and 1-tert- butoxycarbonyl- piperidine-4- carboxylic acid |
| 180 | N-[3-chloro-4-[4- (piperidine-4- carbonyl)piperazine-1- carbonyl]phenyl]-5-[4- (difluoromethoxy)-2- fluoro-phenyl]-1-methyl- imidazole-2-carboxamide formate | 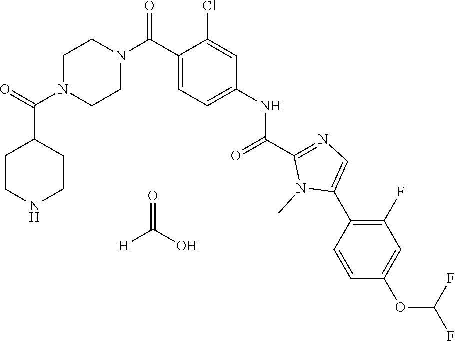 [SMILES: Cn1c(-c2ccc(OC(F)F)cc2F)cnc1C(=O)Nc1ccc(C(=O)N2CCN(C(=O)C3CCNCC3)CC2)c(Cl)c1 ; O=CO] | 619.3 | Intermediate 382 and 1-tert- butoxycarbonyl- piperidine-4- carboxylic acid |
| 181 | N-[3-chloro-4-[4-[2-[(2S)- pyrrolidin-2- yl]acetyl]piperazine-1- carbonyl]phenyl]-5-[4- (difluoromethoxy)-3- fluoro-phenyl]-1-methyl- imidazole-2-carboxamide formate | 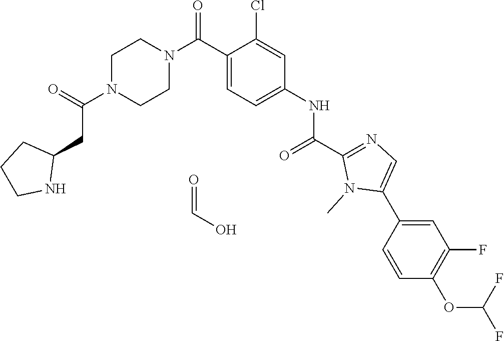 [SMILES: Cn1c(-c2ccc(OC(F)F)c(F)c2)cnc1C(=O)Nc1ccc(C(=O)N2CCN(C(=O)C[C@@H]3CCCN3)CC2)c(Cl)c1 ; O=CO] | 619.2 | Intermediate 383 and 2- [(2S)-1-tert- butoxycarbonyl- pyrrolidin-2- yl]acetic acid |
| 182 | 5-(2-chloro-3-fluoro-4- methoxy-phenyl)-N-[3- chloro-4-[4-[2-(4-hydroxy- 4- piperidyl)acetyl]piperazine- 1-carbonyl]phenyl]-1- methyl-imidazole-2- carboxamide formate | 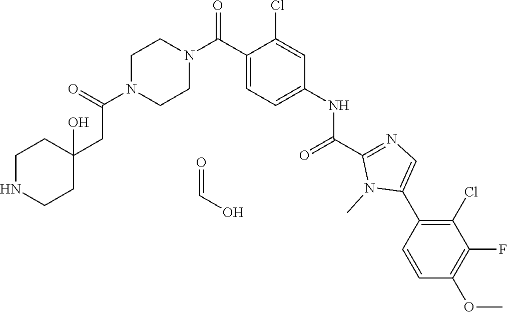 [SMILES: COc1ccc(-c2cnc(C(=O)Nc3ccc(C(=O)N4CCN(C(=O)CC5(O)CCNCC5)CC4)c(Cl)c3)n2C)c(Cl)c1F ; O=CO] | 649.1 | Intermediate 377 and 2-(1- tert- butoxycarbonyl- 4-hydroxy-4- piperidyl) acetic acid |
| 183 | N-[3-chloro-4-[4-[2-(4- hydroxy-4- piperidyl)acetyl]piperazine- 1-carbonyl]phenyl]-5-(4- ethoxy-2,3-difluoro- phenyl)-1-methyl- imidazole-2-carboxamide formate | 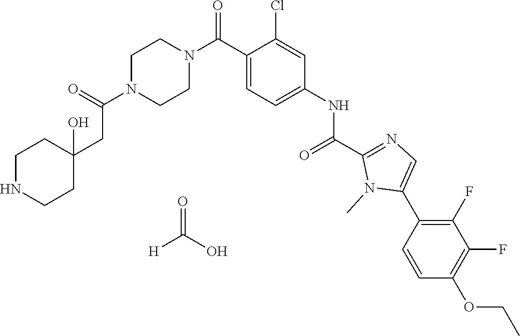 [SMILES: CCOc1ccc(-c2cnc(C(=O)Nc3ccc(C(=O)N4CCN(C(=O)CC5(O)CCNCC5)CC4)c(Cl)c3)n2C)c(F)c1F ; O=CO] | 645.1 | Intermediate 396 and 2-(1- tert- butoxycarbonyl- 4-hydroxy-4- piperidyl) acetic acid |
| 184 | N-[4-[4-[(1R,5S)-3- azabicyclo[3.1.0]hexane- 6-carbonyl]piperazine-1- carbonyl]-3-chloro- phenyl]-5-(2,3-difluoro-4- methoxy-phenyl)-1- methyl-imidazole-2- carboxamide trifluoroacetate | 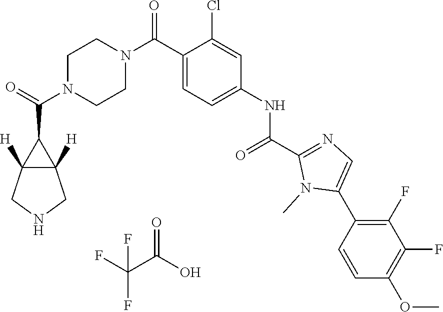 [SMILES: COc1ccc(-c2cnc(C(=O)Nc3ccc(C(=O)N4CCN(C(=O)[C@H]5[C@@H]6CNC[C@@H]65)CC4)c(Cl)c3)n2C)c(F)c1F ; O=C(O)C(F)(F)F] | 599.2 | Intermediate 377 and (1R,5S)-3-tert- butoxycarbonyl- 3-azabicyclo [3.1.0] hexane-6- carboxylic acid |
| 185 | N-[4-[4-[3- (aminomethyl)cyclobutane carbonyl]piperazine-1- carbonyl]-3-chloro- phenyl]-5-(2,3-difluoro-4- methoxy-phenyl)-1- methyl-imidazole-2- carboxamide formate | 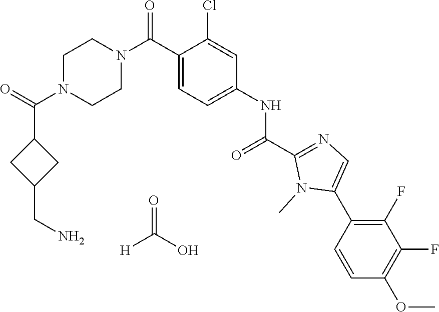 [SMILES: COc1ccc(-c2cnc(C(=O)Nc3ccc(C(=O)N4CCN(C(=O)C5CC(CN)C5)CC4)c(Cl)c3)n2C)c(F)c1F ; O=CO] | 601.3 | Intermediate 377 and 3- [(tert- butoxycarbonyl- amino)methyl] cyclobutane- carboxylic acid |
| 186 | N-[4-[(3aR,6aS)-5- (piperidine-4-carbonyl)- 1,3,3a,4,6,6a- hexahydropyrrolo[3,4- c]pyrrole-2-carbonyl]-3- chloro-phenyl]-5-(2,3- difluoro-4-methoxy- phenyl)-1-methyl- idazole-2-carboxamide formate | 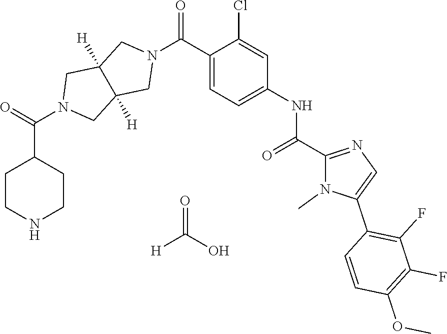 [SMILES: COc1ccc(-c2cnc(C(=O)Nc3ccc(C(=O)N4C[C@@H]5CN(C(=O)C6CCNCC6)C[C@@H]5C4)c(Cl)c3)n2C)c(F)c1F ; O=CO] | 627.3 | Intermediate 380 and 1-tert- butoxycarbonyl- piperidine-4- carboxylic acid |
| 187 | N-[1-[2-chloro-4-[[5-(2,3- difluoro-4-methoxy- phenyl)-1-methyl- imidazole-2- carbonyl]amino]benzoyl] azetidin-3-yl]piperidine-4- carboxamide formate | 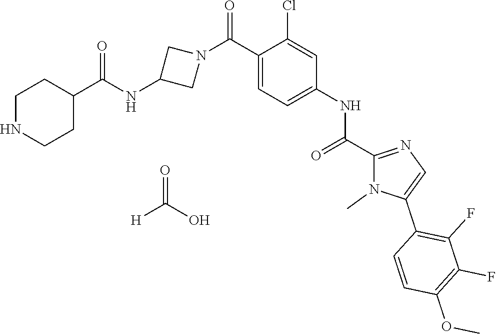 [SMILES: COc1ccc(-c2cnc(C(=O)Nc3ccc(C(=O)N4CC(NC(=O)C5CCNCC5)C4)c(Cl)c3)n2C)c(F)c1F ; O=CO] | 587.3 | Intermediate 379 and 1-tert- butoxycarbonyl- piperidine-4- carboxylic acid |
| 188 | N-[4-[(3aR,6aS)-5-[(3R)- pyrrolidine-3-carbonyl]- 1,3,3a,4,6,6a- hexahydropyrrolo[3,4- c]pyrrole-2-carbonyl]-3- chloro-phenyl]-5-(2,3- difluoro-4-methoxy- phenyl)-1-methyl- imidazole-2-carboxamide formate | 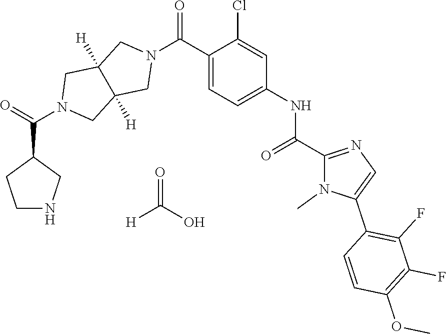 [SMILES: COc1ccc(-c2cnc(C(=O)Nc3ccc(C(=O)N4C[C@@H]5CN(C(=O)[C@@H]6CCNC6)C[C@@H]5C4)c(Cl)c3)n2C)c(F)c1F ; O=CO] | 613.3 | Intermediate 380 and (3R)- 1-tert- butoxycarbonyl- pyrrolidine-3- carboxylic acid |
| 189 | N-[3-chloro-4-[3-[[2- (dimethylamino)acetyl] amino]azetidine-1- carbonyl]phenyl]-5-(2,3- difluoro-4-methoxy- phenyl)-1-methyl- imidazole-2-carboxamide formate | 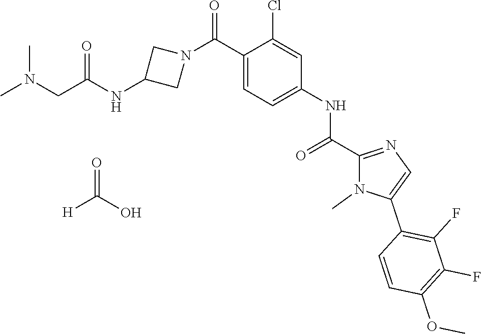 [SMILES: COc1ccc(-c2cnc(C(=O)Nc3ccc(C(=O)N4CC(NC(=O)CN(C)C)C4)c(Cl)c3)n2C)c(F)c1F ; O=CO] | 561.2 | Intermediate 379 and 2- (dimethylamino) acetic acid |
| 190 | N-[4-[(3aS,6aR)-2-[2- (dimethylamino)acetyl]- 1,3,3a,4,6,6a- hexahydropyrrolo[3,4- c]pyrrole-5-carbonyl]-3- chloro-phenyl]-5-(2,3- difluoro-4-methoxy- phenyl)-1-methyl- imidazole-2-carboxamide formate | 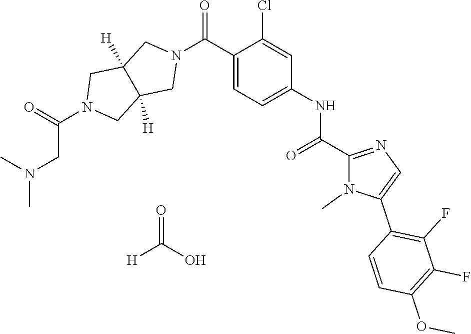 [SMILES: COc1ccc(-c2cnc(C(=O)Nc3ccc(C(=O)N4C[C@H]5CN(C(=O)CN(C)C)C[C@H]5C4)c(Cl)c3)n2C)c(F)c1F ; O=CO] | 601.3 | Intermediate 380 and 2- (dimethylamino) acetic acid |
| 191 | N-[3-chloro-4-[4- (piperidine-4- carbonyl)piperazine-1- carbonyl]phenyl]-1- methyl-5-(2,3,4- trifluorophenyl)imidazole- 2-carboxamide formate | 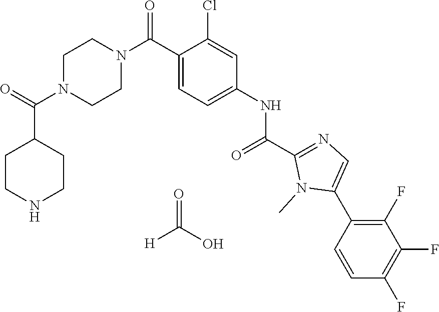 [SMILES: Cn1c(-c2ccc(F)c(F)c2F)cnc1C(=O)Nc1ccc(C(=O)N2CCN(C(=O)C3CCNCC3)CC2)c(Cl)c1 ; O=CO] | 589.3 | Intermediate 397 and 1-tert- butoxycarbonyl- piperidine-4- carboxylic acid |
| 192 | N-[3-chloro-4-[4- (piperidine-4- carbonyl)piperazine-1- carbonyl]phenyl]-5-(3- cyano-4-methoxy-phenyl)- 1-methyl-imidazole-2- carboxamide formate | 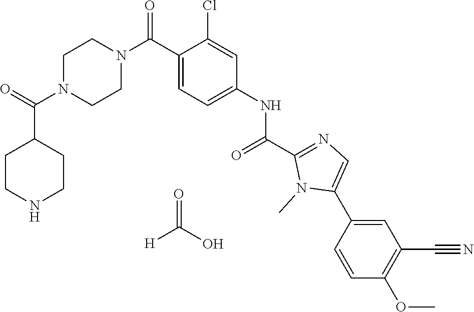 [SMILES: COc1ccc(-c2cnc(C(=O)Nc3ccc(C(=O)N4CCN(C(=O)C5CCNCC5)CC4)c(Cl)c3)n2C)cc1C#N ; O=CO] | 590.0 | Intermediate 398 and 1-tert- butoxycarbonyl- piperidine-4- carboxylic acid |
| 193 | N-[3-chloro-4-[4- (piperidine-4- carbonyl)piperazine-1- carbonyl]phenyl]-5-(3- cyano-2,4-difluoro- phenyl)-1-methyl- imidazole-2-carboxamide formate | 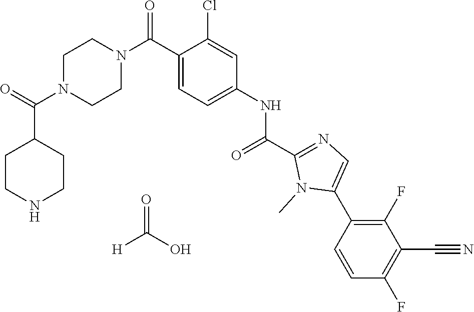 [SMILES: Cn1c(-c2ccc(F)c(C#N)c2F)cnc1C(=O)Nc1ccc(C(=O)N2CCN(C(=O)C3CCNCC3)CC2)c(Cl)c1 ; O=CO] | 596.3 | Intermediate 399 and 1-tert- butoxycarbonyl- piperidine-4- carboxylic acid |
| 194 | N-[3-chloro-4-[4-[2-[(3S)- pyrrolidin-3- yl]acetyl]piperazine-1- carbonyl]phenyl]-5-(2,3- difluoro-4-methoxy- phenyl)-1-methyl- imidazole-2-carboxamide formate | 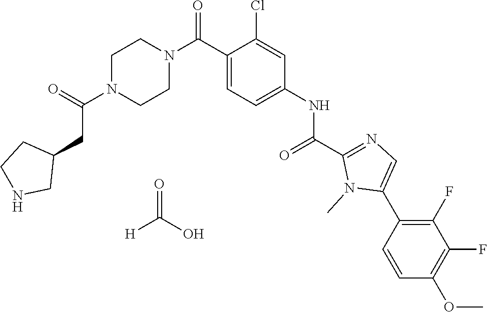 [SMILES: COc1ccc(-c2cnc(C(=O)Nc3ccc(C(=O)N4CCN(C(=O)C[C@@H]5CCNC5)CC4)c(Cl)c3)n2C)c(F)c1F ; O=CO] | 601.3 | Intermediate 377 and 2- [(3S)-1-tert- butoxycarbonyl- pyrrolidin-3- yl]acetic acid |
| 195 | N-[3-chloro-4-[4-[[2- (dimethylamino)acetyl] amino]piperidine-1- carbonyl]phenyl]-5-(2,3- difluoro-4-methoxy- phenyl)-1-methyl- imidazole-2-carboxamide | 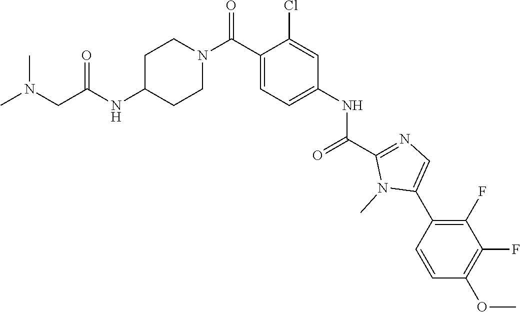 | 589.5 | Intermediate 373 and 2- (dimethylamino) acetic acid |
| 196 | N-(3-chloro-4-(4- (piperidine-4- carbonyl)piperazine-1- carbonyl)phenyl)-5-(4- (cyanomethoxy)-2,3- difluorophenyl)-1-methyl 1H-imidazole-2- carboxamide formate | 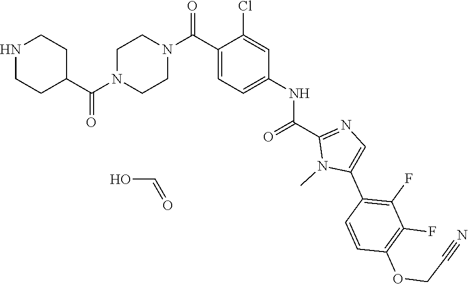 [SMILES: Cn1c(-c2ccc(OCC#N)c(F)c2F)cnc1C(=O)Nc1ccc(C(=O)N2CCN(C(=O)C3CCNCC3)CC2)c(Cl)c1 ; O=CO] | 626.2 | Intermediate 384 and N- Boc- isonipecotic acid |
| 197 | (R)-N-(3-chloro-4-(4- prolylpiperazine-1- carbonyl)phenyl)-5-(4- (cyanomethoxy)-2,3- difluorophenyl)-1-methyl 1H-imidazole-2- carboxamide 2,2,2- trifluoroacetate | 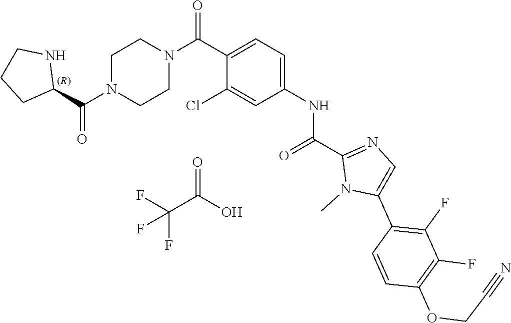 | 726.2 | Intermediate 384 and (tert- butoxycarbonyl)- D-proline |
| 198 | (S)-N-(3-chloro-4-(4- prolylpiperazine-1- carbonyl)phenyl)-5-(4- (cyanomethoxy)-2,3- difluorophenyl)-1-methyl- 1H-imidazole-2- carboxamide 2,2,2- trifluoroacetate | 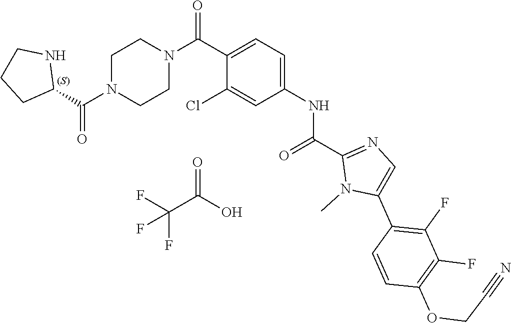 | 726.2 | Intermediate 384 and (tert- butoxycarbonyl)- L-proline |
| 199 | (R)-N-(3-chloro-4-(4- (pyrrolidine-3- carbonyl)piperazine-1- carbonyl)phenyl)-5-(4- (cyanomethoxy)-2,3- difluorophenyl)-1-methyl- 1H-imidazole-2- carboxamide 2,2,2- trifluoroacetate | 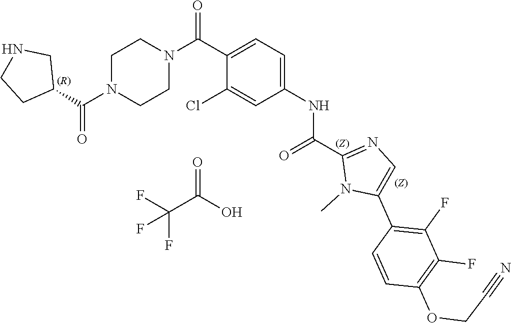 | 726.2 | Intermediate 384 and (R)-1- (tert- butoxycarbonyl) pyrrolidine- 3-carboxylic acid |
| 200 | (S)-N-(3-chloro-4-(4- (pyrrolidine-3- carbonyl)piperazine-1- carbonyl)phenyl)-5-(4- (cyanomethoxy)-2,3- difluorophenyl)-1-methyl 1H-imidazole-2- carboxamide 2,2,2- trifluoroacetate | 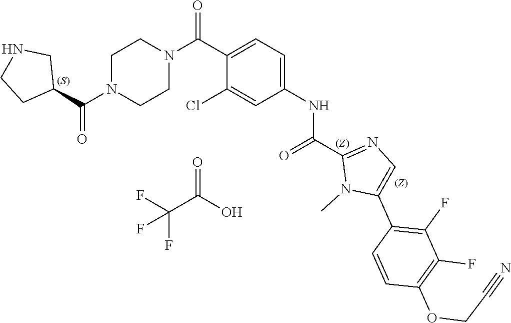 | 726.2 | Intermediate 384 and (S)-1- (tert- butoxycarbonyl) pyrrolidine- 3-carboxylic acid |
| 201 | N-(3-chloro-4-(4-((2S,4S)- 4-hydroxypyrrolidine-2- carbonyl)piperazine-1- carbonyl)phenyl)-5-(4- (cyanomethoxy)-2,3- difluorophenyl)-1-methyl- 1H-imidazole-2- carboxamide formate | 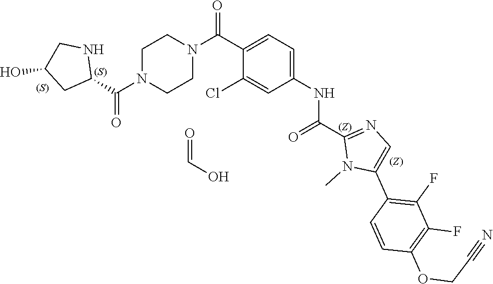 | 674.2 | Intermediate 384 and (2S,4S)-1-(tert- butoxycarbonyl)- 4- hydroxy- pyrrolidine- 2- carboxylic acid |
| 202 | N-(3-chloro-4-(4- ((2S,4R)-4- hydroxypyrrolidine-2- carbonyl)piperazine-1- carbonyl)phenyl)-5-(4- (cyanomethoxy)-2,3- difluorophenyl)-1-methyl- 1H-imidazole-2- carboxamide 2,2,2- trifluoroacetate | 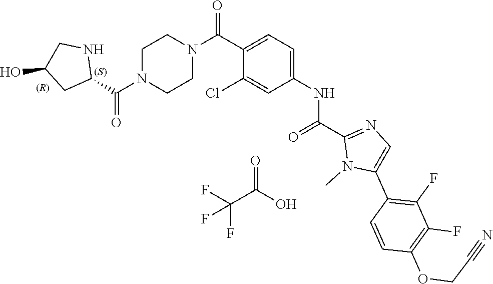 | 742.2 | Intermediate 384 and (2S,4R)-1- (tert- butoxycarbonyl)- 4- hydroxy- pyrrolidine- 2- carboxylic acid |
| 203 | (R)-5-(4-(cyanomethoxy)- 2,3-difluorophenyl)-1- methyl-N-(3-methyl-4-(4- (pyrrolidine-3- carbonyl)piperazine-1- carbonyl)phenyl)-1H- imidazole-2-carboxamide 2,2,2-trifluoroacetate | 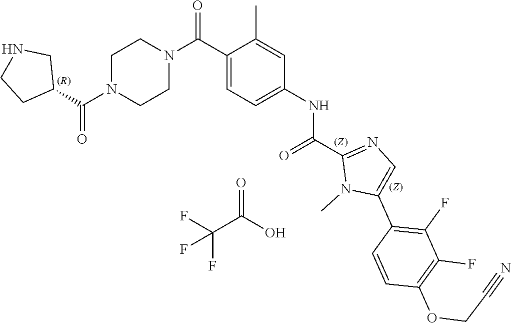 | 706.2 | Intermediate 385 and (R)-1- (tert- butoxycarbonyl) pyrrolidine- 3-carboxylic acid |
| 204 | N-(3-chloro-4-(4- (dimethylglycyl)piperazine- 1-carbonyl)phenyl)-5-(4- (cyanomethoxy)-2,3- difluorophenyl)-1-methyl- 1H-imidazole-2- carboxamide formate | 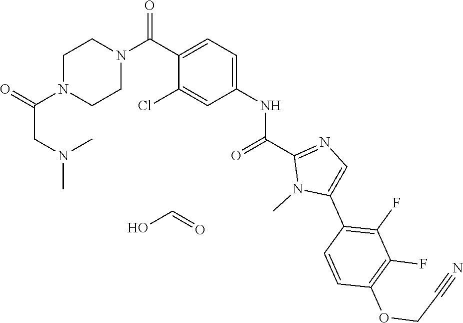 [SMILES: CN(C)CC(=O)N1CCN(C(=O)c2ccc(NC(=O)c3ncc(-c4ccc(OCC#N)c(F)c4F)n3C)cc2Cl)CC1 ; O=CO] | 646.2 | Intermediate 384 and dimethylglycine |
| 205 | N-(3-chloro-4-(4- (methylglycyl)piperazine- 1-carbonyl)phenyl)-5-(4- (cyanomethoxy)-2,3- difluorophenyl)-1-methyl- 1H-imidazole-2- carboxamide formate | 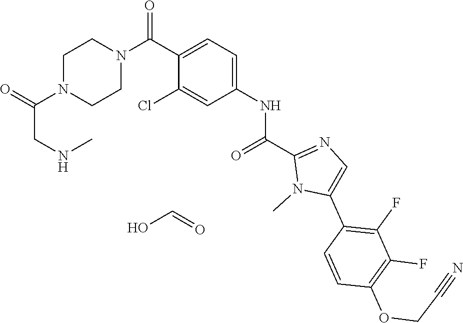 [SMILES: CNCC(=O)N1CCN(C(=O)c2ccc(NC(=O)c3ncc(-c4ccc(OCC#N)c(F)c4F)n3C)cc2Cl)CC1 ; O=CO] | 632.2 | Intermediate 384 and N- (tert- butoxycarbonyl)- N-methylglycine |
| 206 | 5-(4-(cyanomethoxy)-2,3- difluorophenyl)-1-methyl- N-(3-methyl-4-(4- (methylglycyl)piperazine- 1-carbonyl)phenyl)-1H- imidazole-2-carboxamide 2,2,2-trifluoroacetate | 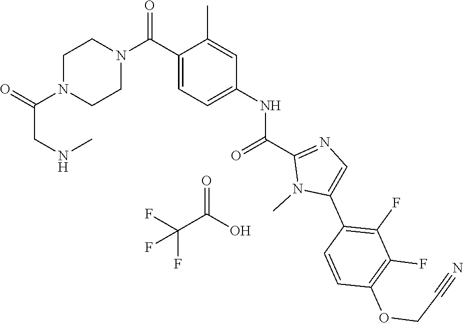 | 680.2 | Intermediate 385 and N- (tert- butoxycarbonyl)- N-methylglycine |
| 207 | N-(3-chloro-4-(4-(4- hydroxypiperidine-4- carbonyl)piperazine-1- carbonyl)phenyl)-5-(4- (cyanomethoxy)-2,3- difluorophenyl)-1-methyl- 1H-imidazole-2- carboxamide 2,2,2- trifluoroacetate | 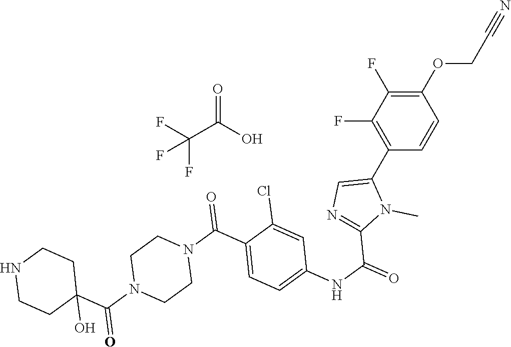 | 756.2 | Intermediate 384 and 1- (tert- butoxycarbonyl)- 4- hydroxy- piperidine- 4- carboxylic acid |
| 208 | (S)-N-(3-chloro-4-(4- (piperidine-3- carbonyl)piperazine-1- carbonyl)phenyl)-5-(4- (cyanomethoxy)-2,3- difluorophenyl)-1-methyl 1H-imidazole-2- carboxamide 2,2,2- trifluoroacetate | 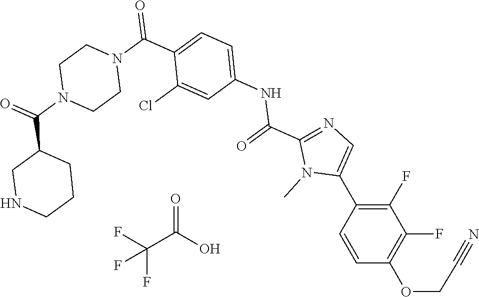 [SMILES: Cn1c(-c2ccc(OCC#N)c(F)c2F)cnc1C(=O)Nc1ccc(C(=O)N2CCN(C(=O)[C@H]3CCCNC3)CC2)c(Cl)c1 ; O=C(O)C(F)(F)F] | 740.2 | Intermediate 384 and (S)-1- (tert- butoxycarbonyl) piperidine-3- carboxylic acid |
| 209 | N-(4-(4-((2S,4S)-4- aminopyrrolidine-2- carbonyl)piperazine-1- carbonyl)-3- chlorophenyl)-5-(4- (cyanomethoxy)-2,3- difluorophenyl)-1-methyl 1H-imidazole-2- carboxamide | 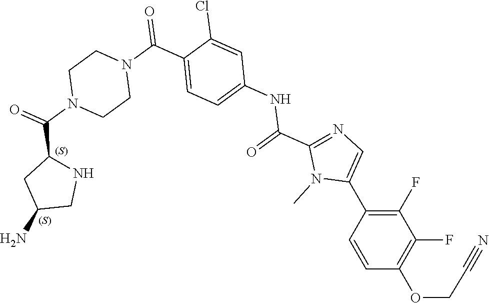 | 627.2 | Intermediate 384 and (2S,4S)-1-(tert- butoxycarbonyl)- 4-((tert- butoxycarbonyl) amino) pyrrolidine-2- carboxylic acid |
| 210 | N-(3-chloro-4-(4- ((2R,3S)-3- hydroxypyrrolidine-2- carbonyl)piperazine-1- carbonyl)phenyl)-5-(4- (cyanomethoxy)-2,3- difluorophenyl)-1-methyl- 1H-imidazole-2- carboxamide formate | 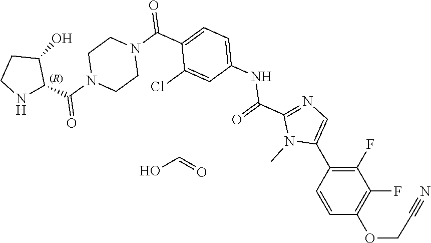 | 674.2 | Intermediate 384 and (2S,3S)-1-(tert- butoxycarbonyl)- 3- hydroxy- pyrrolidine- carboxylic acid |
| 211 | (S)-N-(3-chloro-4-(4- (piperidine-2- carbonyl)piperazine-1- carbonyl)phenyl)-5-(4- (cyanomethoxy)-2,3- difluorophenyl)-1-methyl 1H-imidazole-2- carboxamide formate | 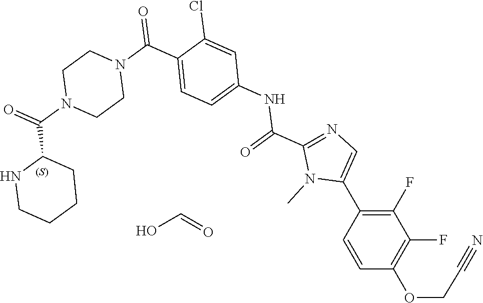 | 672.2 | Intermediate 384 and (S)-1- (tert- butoxycarbonyl) piperidine-2- carboxylic acid |
| 212 | 1-(2-(4-(2-chloro-4-(5-(4- (cyanomethoxy)-2,3- difluorophenyl)-1-methyl- 1H-imidazole-2- carboxamido)benzoyl) piperazin-1-yl)-2- oxoethyl)azetidine-3- carboxylic acid 2,2,2- trifluoroacetate | 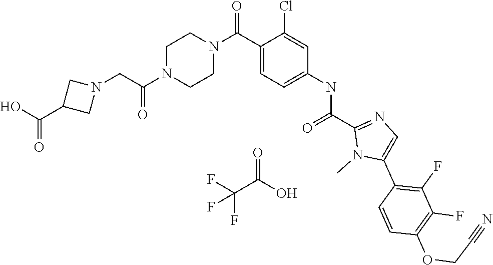 [SMILES: Cn1c(-c2ccc(OCC#N)c(F)c2F)cnc1C(=O)Nc1ccc(C(=O)N2CCN(C(=O)CN3CC(C(=O)O)C3)CC2)c(Cl)c1 ; O=C(O)C(F)(F)F] | 770.2 | Intermediate 384 and Intermediate 415 |
| 213 | (R)-N-(3-chloro-4-(4-(2- (pyrrolidine-2- carboxamido)ethyl) piperazine-1- carbonyl)phenyl)-5- (4-(cyanomethoxy)-2,3- difluorophenyl)-1-methyl- 1H-imidazole-2- carboxamide 2,2,2- trifluoroacetate | 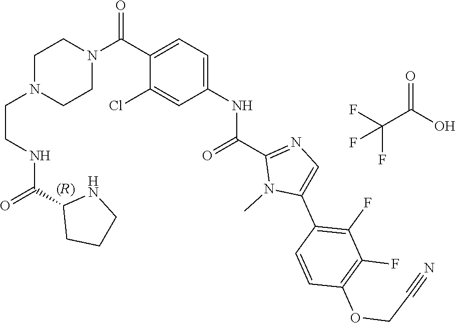 | 769.2 | 19 and (tert- butoxycarbonyl)- L-proline |
| 214 | N-(3-chloro-4-(4-(5- hydroxypiperidine-3- carbonyl)piperazine-1- carbonyl)phenyl)-5-(4- (cyanomethoxy)-2,3- difluorophenyl)-1-methyl- 1H-imidazole-2- carboxamide 2,2,2- trifluoroacetate | 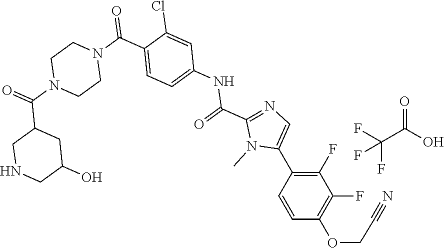 | 756.2 | Intermediate 384 and 1- (tert- butoxycarbonyl)- 5- hydroxy- piperidine-3- carboxylic acid |
| 215 | (R)-1-(2-(4-(2-chloro-4-(5- (4-(cyanomethoxy)-2,3- difluorophenyl)-1-methyl- 1H-imidazole-2- carboxamido)benzoyl) piperazin-1-yl)-2- oxoethyl)pyrrolidine-3- carboxylic acid 2,2,2- trifluoroacetic acid | 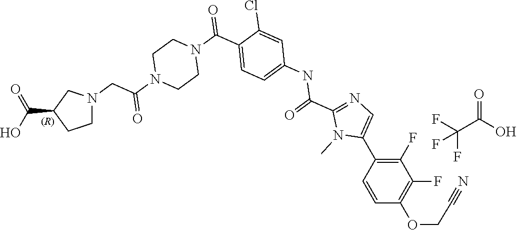 | 784.2 | Intermediate 384 and Intermediate 416 |
| 216 | N-(3-chloro-4-(4- (piperidin-4- ylsulfonyl)piperazine-1- carbonyl)phenyl)-5-(2,3- difluoro-4- methoxyphenyl)-1-methyl- 1H-imidazole-2- carboxamide formate | 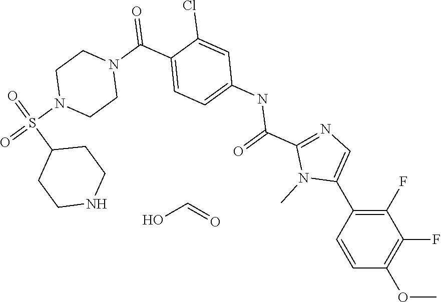 [SMILES: COc1ccc(-c2cnc(C(=O)Nc3ccc(C(=O)N4CCN(S(=O)(=O)C5CCNCC5)CC4)c(Cl)c3)n2C)c(F)c1F ; O=CO] | 683.2 | Intermediate 386 and tert- butyl 4- (chlorosulfonyl) piperidine-1- carboxylate |
| 217 | N-(4-(3-(2- aminoacetamido)azetidine- 1-carbonyl)-3- chlorophenyl)-5-(2,3- difluoro-4- methoxyphenyl)-1-methyl- 1H-imidazole-2- carboxamide formate | 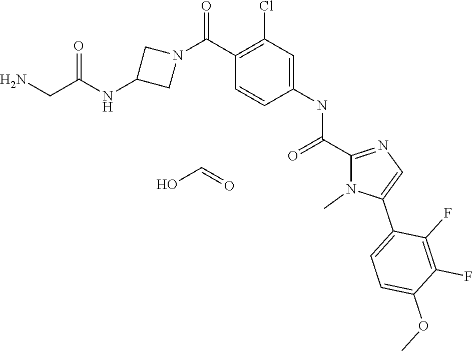 [SMILES: COc1ccc(-c2cnc(C(=O)Nc3ccc(C(=O)N4CC(NC(=O)CN)C4)c(Cl)c3)n2C)c(F)c1F ; O=CO] | 579.1 | Intermediate 387 and (tert- butoxycarbonyl) glycine |
| 218 | N-(4-(3-(3- (aminomethyl)cyclobutane- 1-carboxamido)azetidine- 1-carbonyl)-3- chlorophenyl)-5-(2,3- difluoro-4- methoxyphenyl)-1-methyl- 1H-imidazole-2- carboxamide 2,2,2- trifluoroacetate | 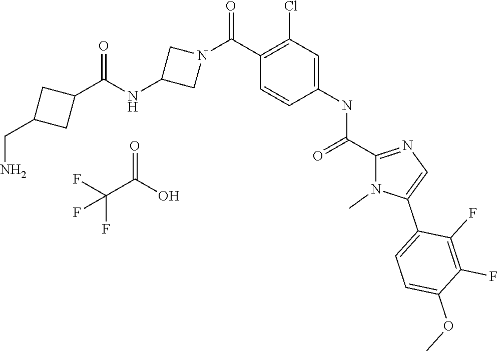 [SMILES: COc1ccc(-c2cnc(C(=O)Nc3ccc(C(=O)N4CC(NC(=O)C5CC(CN)C5)C4)c(Cl)c3)n2C)c(F)c1F ; O=C(O)C(F)(F)F] | 701.2 | Intermediate 387 and 3- (((tert- butoxycarbonyl) amino)methyl) cyclobutane- 1-carboxylic acid |
| 219 | N-(3-chloro-4-(4-(3- hydroxycyclobutane-1- carbonyl)piperazine-1- carbonyl)phenyl)-5-(2,3- difluoro-4- methoxyphenyl)-1-methyl- 1H-imidazole-2- carboxamide | 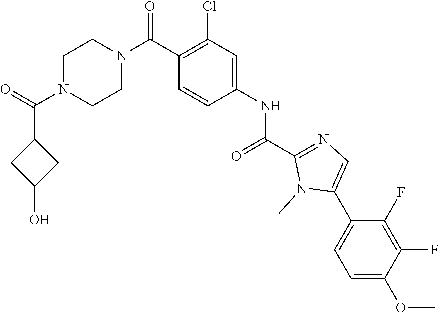 [SMILES: COc1ccc(-c2cnc(C(=O)Nc3ccc(C(=O)N4CCN(C(=O)C5CC(O)C5)CC4)c(Cl)c3)n2C)c(F)c1F] | 588.2 | Intermediate 386 and 3- hydroxycyclo- butane-1- carboxylic acid |
| 220 | N-(3-chloro-4-(4- (dimethylglycyl)piperazine- 1-carbonyl)phenyl)-5- (2,3-difluoro-4- (fluoromethoxy)phenyl)-1- methyl-1H-imidazole-2- carboxamide formate | 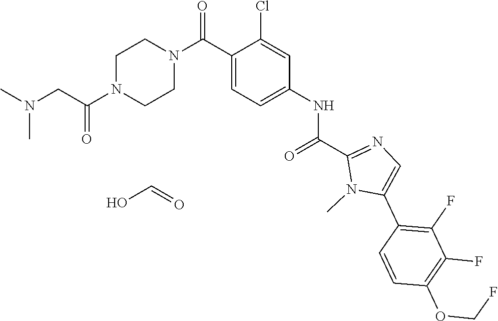 [SMILES: CN(C)CC(=O)N1CCN(C(=O)c2ccc(NC(=O)c3ncc(-c4ccc(OCF)c(F)c4F)n3C)cc2Cl)CC1 ; O=CO] | 639.2 | Intermediate 388 and dimethylglycine |
| 221 | N-(3-chloro-4-(4- ((2S,4R)-4- hydroxypyrrolidine-2- carbonyl)piperazine-1- carbonyl)phenyl)-5-(2,3- difluoro-4- (fluoromethoxy)phenyl)-1- methyl-1H-imidazole-2- carboxamide formate | 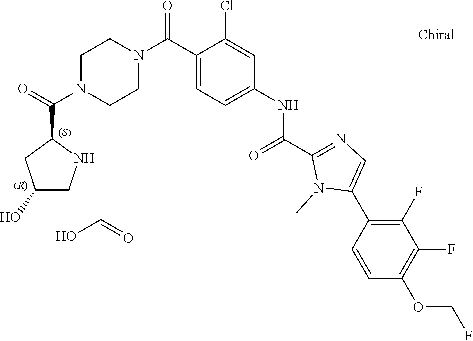 | 667.2 | Intermediate 388 and (2S,4R)-1- (tert- butoxycarbonyl)- 4- hydroxy- pyrrolidine- 2- carboxylic acid |
| 222 | N-(3-chloro-4-(4- (dimethylglycyl)piperazine- 1-carbonyl)phenyl)-5-(2- fluoro-4- (fluoromethoxy)phenyl)-1- methyl-1H-imidazole-2- carboxamide | 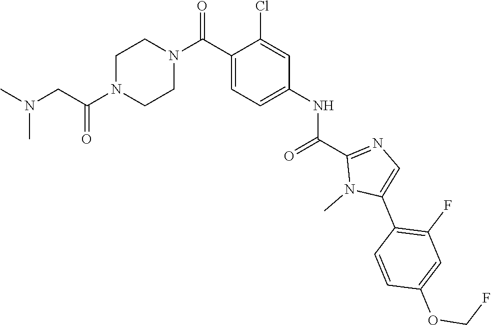 [SMILES: CN(C)CC(=O)N1CCN(C(=O)c2ccc(NC(=O)c3ncc(-c4ccc(OCF)cc4F)n3C)cc2Cl)CC1] | 575.1 | Intermediate 389 and dimethylglycine |
| 223 | N-(3-chloro-4-(4-(2-(4- hydroxypiperidin-4- yl)acetyl)piperazine-1- carbonyl)phenyl)-5-(4- (difluoromethoxy)-2,3- difluorophenyl)-1-methyl 1H-imidazole-2- carboxamide formate | 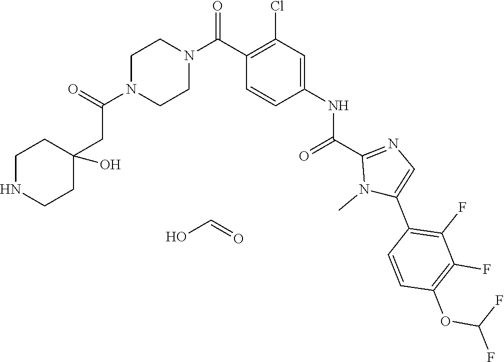 [SMILES: Cn1c(-c2ccc(OC(F)F)c(F)c2F)cnc1C(=O)Nc1ccc(C(=O)N2CCN(C(=O)CC3(O)CCNCC3)CC2)c(Cl)c1 ; O=CO] | 713.2 | Intermediate 390 and 2-(1- (tert- butoxycarbonyl)- 4- hydroxypiperidin- 4-yl)acetic acid |
| 224 | (S)-N-(3-chloro-4-(4-(2- (pyrrolidin-2- yl)acetyl)piperazine-1- carbonyl)phenyl)-5-(4- (difluoromethoxy)-2,3- difluorophenyl)-1-methyl- 1H-imidazole-2- carboxamide formate | 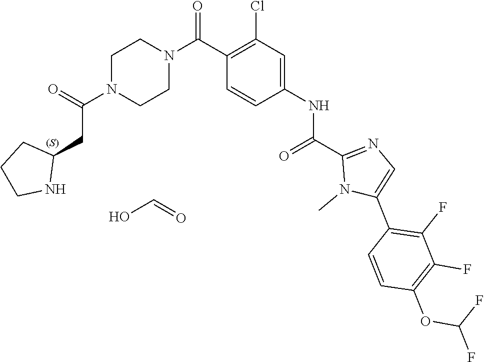 | 683.2 | Intermediate 390 and (S)-2- (1-(tert- butoxycarbonyl) pyrrolidin-2- yl)acetic acid |
| 225 | 5-[4-(cyanomethoxy)-2,3- difluoro-phenyl]-N-[4-[4- [(2S,4S)-4-hydroxy-4- methyl-pyrrolidine-2- carbonyl]piperazine-1- carbonyl]-3-methyl- phenyl]-1-methyl- imidazole-2-carboxamide formate | 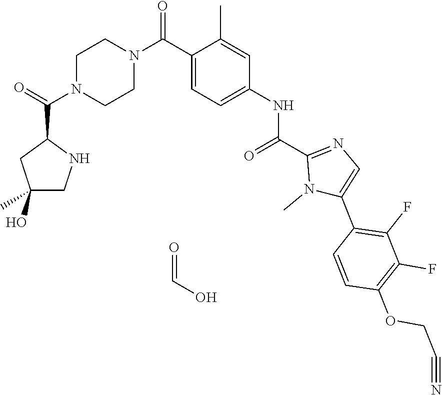 [SMILES: Cc1cc(NC(=O)c2ncc(-c3ccc(OCC#N)c(F)c3F)n2C)ccc1C(=O)N1CCN(C(=O)[C@@H]2C[C@](C)(O)CN2)CC1 ; O=CO] | 622.2 | Intermediate 5 and Intermediate 434 |
| 226 | 5-[4-(cyanomethoxy)-2,3- difluoro-phenyl]-N-[4-[4- [(2S,4S)-4-ethyl-4- hydroxy-pyrrolidine-2- carbonyl]piperazine-1- carbonyl]-3-methyl- phenyl]-1-methyl- imidazole-2-carboxamide formate | 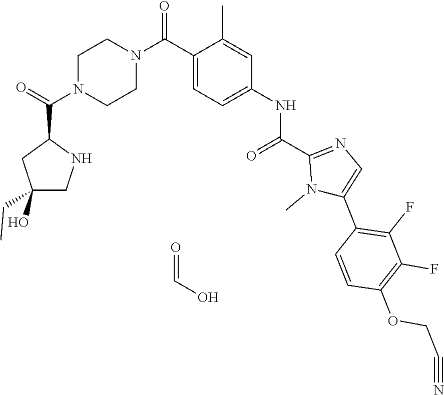 [SMILES: CC[C@@]1(O)CN[C@H](C(=O)N2CCN(C(=O)c3ccc(NC(=O)c4ncc(-c5ccc(OCC#N)c(F)c5F)n4C)cc3C)CC2)C1 ; O=CO] | 636.2 | Intermediate 5 and Intermediate 436 |
| 227 | N-(3-chloro-4-(4- ((3S,4R)-3- hydroxypiperidine-4- carbonyl)piperazine-1- carbonyl)phenyl)-5-(4- (cyanomethoxy)-2,3- difluorophenyl)-1-methyl 1H-imidazole-2- carboxamide (S)-2- hydroxypropanoate | 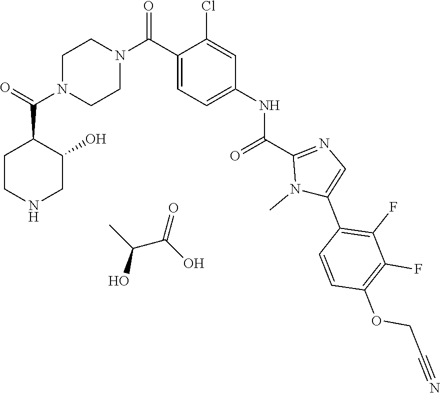 | 640.4 | Intermediate 438 and Intermediate 12 |
| 228 | N-[3-chloro-4-[4- [(3R,4R)-3- hydroxypiperidine-4- carbonyl]piperazine-1- carbonyl]phenyl]-5-(2,3- difluoro-4-methoxy- phenyl)-1-methyl- imidazole-2-carboxamide | 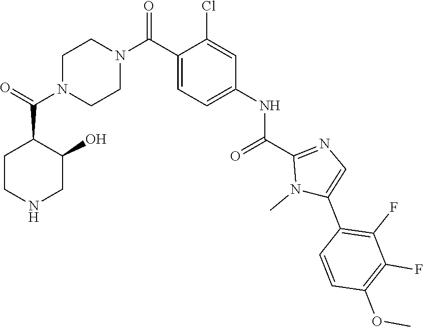 [SMILES: COc1ccc(-c2cnc(C(=O)Nc3ccc(C(=O)N4CCN(C(=O)[C@@H]5CCNC[C@@H]5O)CC4)c(Cl)c3)n2C)c(F)c1F] | 617.2 | Intermediate 9 and (3R,4R)- 1,4- Piperidinedi- carboxylic acid, 3-hydroxy-, 1- (1,1- dimethylethyl) ester |
| 229 | N-[3-chloro-4-[4-[(3S,4S)- 3-hydroxypiperidine-4- carbonyl]piperazine-1- carbonyl]phenyl]-5-(2,3- difluoro-4-methoxy- phenyl)-1-methyl- imidazole-2-carboxamide | 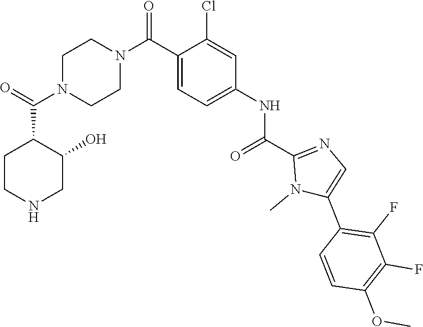 [SMILES: COc1ccc(-c2cnc(C(=O)Nc3ccc(C(=O)N4CCN(C(=O)[C@H]5CCNC[C@H]5O)CC4)c(Cl)c3)n2C)c(F)c1F] | 617.2 | Intermediate 9 and Intermediate 437 |
| 230 | N-[3-chloro-4-[4- [(3S,4R)-3- hydroxypiperidine-4- carbonyl]piperazine-1- carbonyl]phenyl]-5-(2,3- difluoro-4-methoxy- phenyl)-1-methyl- imidazole-2-carboxamide formate | 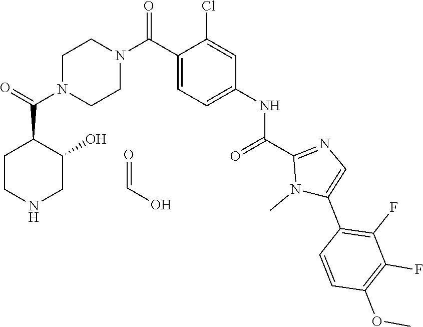 | 617.2 | Intermediate 9 and Intermediate 12 |
| 231 | N-[3-chloro-4-[4- [(3R,4S)-3- hydroxypiperidine-4- carbonyl]piperazine-1- carbonyl]phenyl]-5-(2,3- difluoro-4-methoxy- phenyl)-1-methyl- imidazole-2-carboxamide formate | 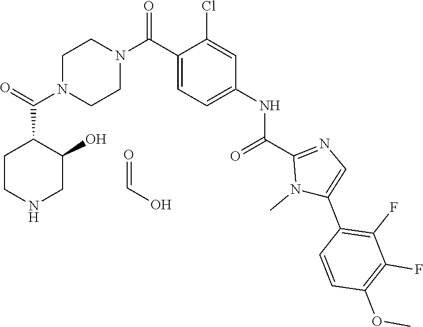 | 617.2 | Intermediate 9 and (3R,4S)- 1,4- Piperidinedi- carboxylic acid, 3-hydroxy-, 1- (1,1- dimethylethyl) ester |
| 232 | N-[3-chloro-4-[4-[(2S,4S)- 4-hydroxy-4-methyl- pyrrolidine-2- carbonyl]piperazine-1- carbonyl]phenyl]-5-(2,3- difluoro-4-methoxy- phenyl)-1-methyl- imidazole-2-carboxamide formate | 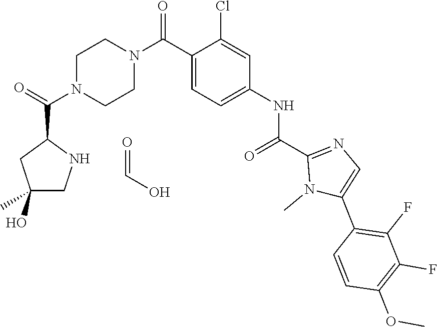 | 617.2 | Intermediate 9 and Intermediate 435 |
| 233 | N-[3-chloro-4-[4-[(2S,4S)- 4-ethyl-4-hydroxy- pyrrolidine-2- carbonyl]piperazine-1- carbonyl]phenyl]-5-(2,3- difluoro-4-methoxy- phenyl)-1-methyl- imidazole-2-carboxamide formate | 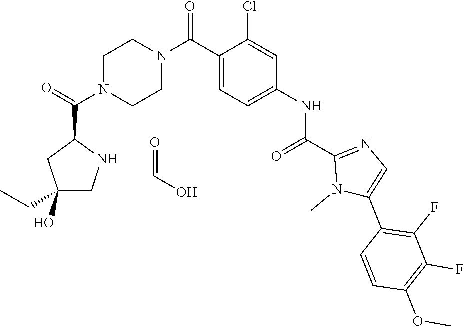 | 631.4 | Intermediate 9 and Intermediate 436 |
| 234 | 5-(2,3-difluoro-4- methoxy-phenyl)-N-[4-[4- [(3R,4R)-3- hydroxypiperidine-4- carbonyl]piperazine-1- carbonyl]-3-methyl- phenyl]-1-methyl- imidazole-2-carboxamide formate | 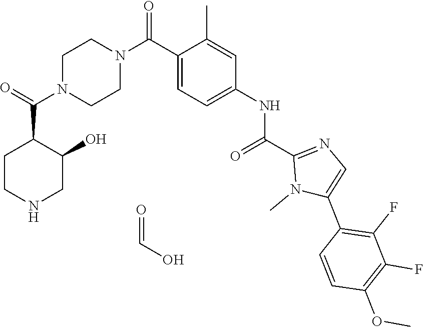 [SMILES: COc1ccc(-c2cnc(C(=O)Nc3ccc(C(=O)N4CCN(C(=O)[C@@H]5CCNC[C@@H]5O)CC4)c(C)c3)n2C)c(F)c1F ; O=CO] | 597.2 | Intermediate 10 and (3R,4R)-1,4- Piperidinedi- carboxylic acid, 3-hydroxy-, 1- (1,1- dimethylethyl) ester |

### Example Type II: 235

### N-[3-Chloro-4-[4-(4-piperidylsulfonylamino)piperidine-1-carbonyl]phenyl]-5-(2,3-difluoro-4-methoxy-phenyl)-1-methyl-imidazole-2-carboxamide formate

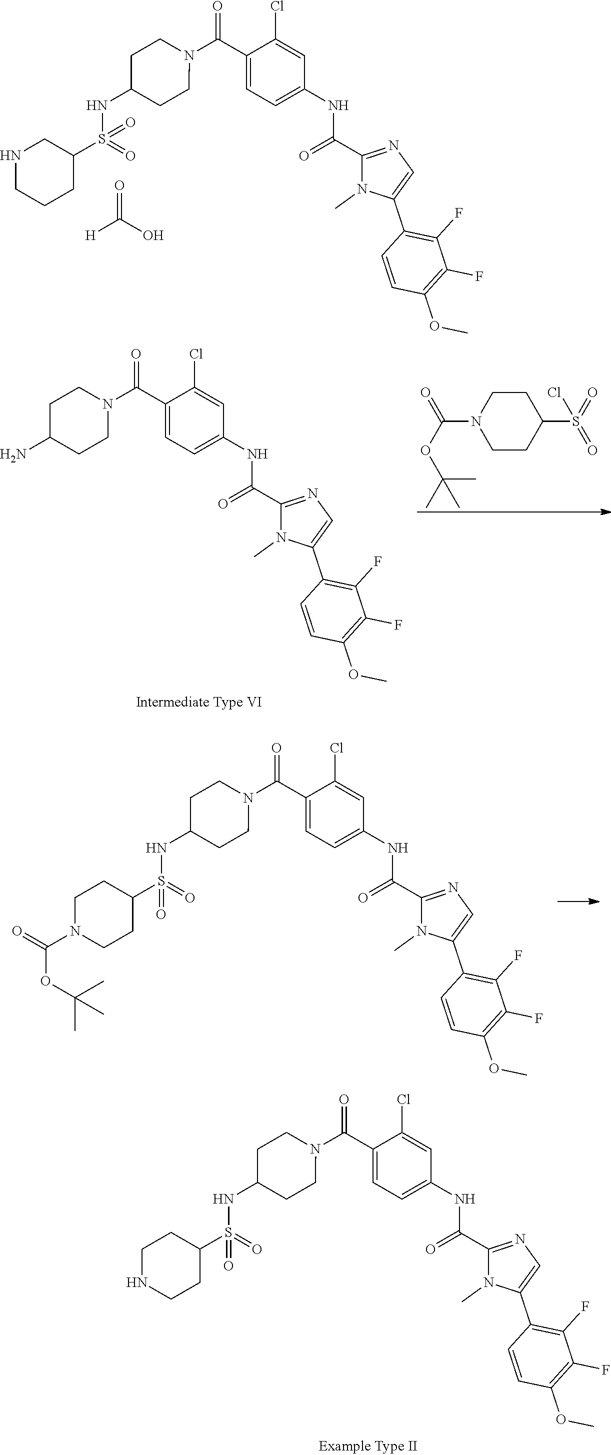

### Step 1: tert-butyl 4-[[1-[2-chloro-4-[[5-(2,3-difluoro-4-methoxy-phenyl)-1-methyl-imidazole-2-carbonyl]amino]benzoyl]-4-piperidyl]sulfamoyl]piperidine-1-carboxylate

A mixture of N-[4-(4-aminopiperidine-1-carbonyl)-3-chloro-phenyl]-5-(2,3-difluoro-4-methoxy-phenyl)-1-methyl-imidazole-2-carboxamide (intermediate 373, 300 mg, 595 μmol), tert-butyl 4-(chlorosulfonyl)piperidine-1-carboxylate (253 mg, 893 μmol) and Et₃N (120 mg, 166 μl, 1.19 mmol) in DCM (5 mL) was stirred overnight. Then the clear solution was washed with water, dried over anhydrous Na₂SO₄ and concentrated to give the title compound (300 mg) as a brown solid which was used into next step reaction without further purification. MS (ESI, m/z): 750.8 [M+H]⁺.

### Step 2: N-[3-chloro-4-[4-(4-piperidylsulfonylamino)piperidine-1-carbonyl]phenyl]-5-(2,3-difluoro-4-methoxy-phenyl)-1-methyl-imidazole-2-carboxamide; formic acid

tert-Butyl 4-(N-(1-(2-chloro-4-(5-(2,3-difluoro-4-methoxyphenyl)-1-methyl-1H-imidazole-2-carboxamido)benzoyl)piperidin-4-yl)sulfamoyl)piperidine-1-carboxylate (300 mg, 399 μmol) was dissolved in DCM (5 mL) and TFA (5 mL). The solution was stirred for 2 h. Under ice cooling, water (5 mL) was added followed by Et3N until pH 8-9. The water layer was extracted with DCM. The organic layer was concentrated to give the crude product which was purified by Prep-HPLC to give the title compound (126 mg). MS (ESI, m/z): 651.0 [M+H]⁺.

The following Type II and Type III Examples were prepared in analogy to example 235.

| | | | MS | |
|---|---|---|---|---|
| | | | ESI | |
| Ex. | Name | Structure | [M + H]⁺ | Starting Material |
| 236 | N-[4-[4-(2- aminoethylsulfonylami- no)piperidine-1- carbonyl]-3-chloro- phenyl]-5-(2,3- difluoro-4-methoxy- phenyl)-1-methyl- imidazole-2- carboxamide | 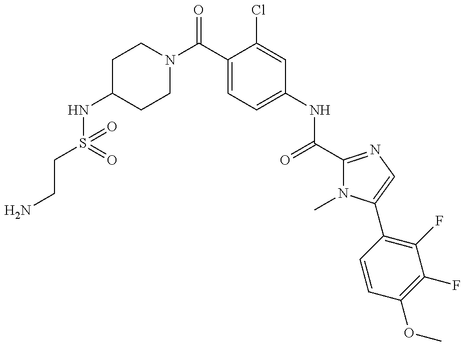 [SMILES: COc1ccc(-c2cnc(C(=O)Nc3ccc(C(=O)N4CCC(NS(=O)(=O)CCN)CC4)c(Cl)c3)n2C)c(F)c1F] | 611.0 | Intermediate 373 and tert-butyl (2- (chlorosulfonyl)eth- yl)carbamate |
| 237 | N-[3-chloro-4-[4- (pyrrolidin-3- ylsulfonylamino)piper- idine-1- carbonyl]phenyl]-5- (2,3-difluoro-4- methoxy-phenyl)-1- methyl-imidazole-2- carboxamide | 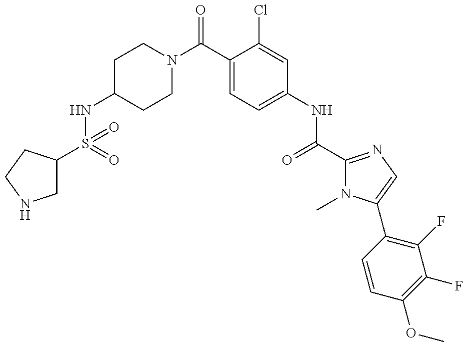 [SMILES: COc1ccc(-c2cnc(C(=O)Nc3ccc(C(=O)N4CCC(NS(=O)(=O)C5CCNC5)CC4)c(Cl)c3)n2C)c(F)c1F] | 637.0 | Intermediate 373 and tert-butyl 3- (chlorosulfonyl)pyr- rolidine-1- carboxylate |
| 238 | N-[3-chloro-4-[4- (methanesulfonamido) piperidine-1- carbonyl]phenyl]-5- (2,3-difluoro-4- methoxy-phenyl)-1- methyl-imidazole-2- carboxamide | 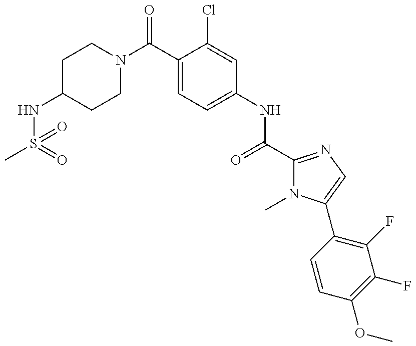 [SMILES: COc1ccc(-c2cnc(C(=O)Nc3ccc(C(=O)N4CCC(NS(C)(=O)=O)CC4)c(Cl)c3)n2C)c(F)c1F] | 582.1 | Intermediate 373 and methanesulfonyl chloride |
| 239 | N-[4-[4-(2- aminoethylsulfonyl)piper- razine-1-carbonyl]- 3-chloro-phenyl]-5- (2,3-difluoro-4- methoxy-phenyl)-1- methyl-imidazole-2- carboxamide | 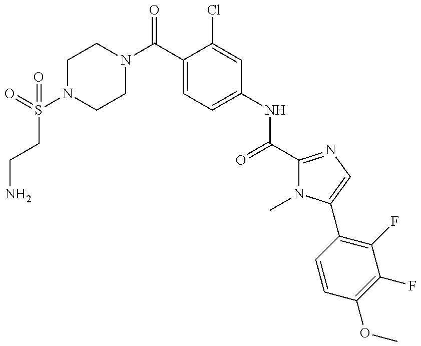 [SMILES: COc1ccc(-c2cnc(C(=O)Nc3ccc(C(=O)N4CCN(S(=O)(=O)CCN)CC4)c(Cl)c3)n2C)c(F)c1F] | 597.0 | Intermediate 374 and tert-butyl (2- (chlorosulfonyl)eth- yl)carbamate |
| 240 | N-[3-chloro-4-(4- methylsulfonylpiperazine- 1- carbonyl)phenyl]-5- (2,3-difluoro-4- methoxy-phenyl)-1- methyl-imidazole-2- carboxamide | 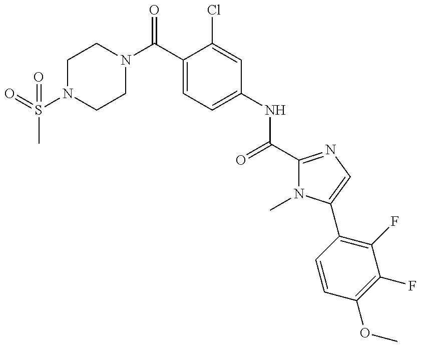 [SMILES: COc1ccc(-c2cnc(C(=O)Nc3ccc(C(=O)N4CCN(S(C)(=O)=O)CC4)c(Cl)c3)n2C)c(F)c1F] | 567.8 | Intermediate 374 and methanesulfonyl chloride |
| 241 | N-[3-chloro-4-(4- pyrrolidin-3- ylsulfonylpiperazine- 1-carbonyl)phenyl]-5- (2,3-difluoro-4- methoxy-phenyl)-1- methyl-imidazole-2- carboxamide | 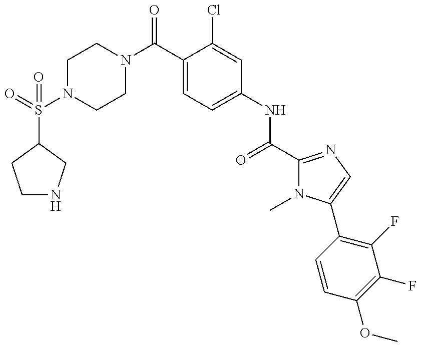 [SMILES: COc1ccc(-c2cnc(C(=O)Nc3ccc(C(=O)N4CCN(S(=O)(=O)C5CCNC5)CC4)c(Cl)c3)n2C)c(F)c1F] | 623.0 | Intermediate 374 and tert-butyl 3- (chlorosulfonyl)pyr- rolidine-1- carboxylate |
| 242 | N-[4-[4-[1-(2- aminoethylsulfonyl)pipe- ridine-4- carbonyl]piperazine- 1-carbonyl]-3-chloro- phenyl]-5-(2,3- difluoro-4-methoxy- phenyl)-1-methyl- imidazole-2- carboxamide | 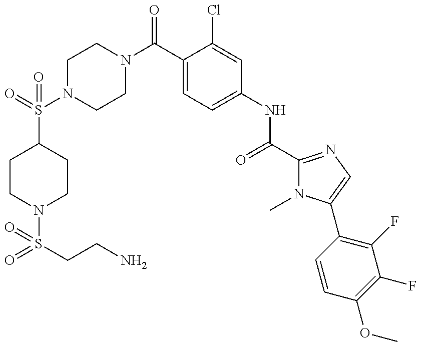 [SMILES: COc1ccc(-c2cnc(C(=O)Nc3ccc(C(=O)N4CCN(S(=O)(=O)C5CCN(S(=O)(=O)CCN)CC5)CC4)c(Cl)c3)n2C)c(F)c1F] | 708.0 | Intermediate 413 and tert-butyl (2- (chlorosulfonyl)eth- yl)carbamate |

### Example Type II: 243

### N-[3-Chloro-4-[4-[3-(methylamino)propanoyl]piperazine-1-carbonyl]phenyl]-5-(2,3-difluoro-4-methoxy-phenyl)-1-methyl-imidazole-2-carboxamide

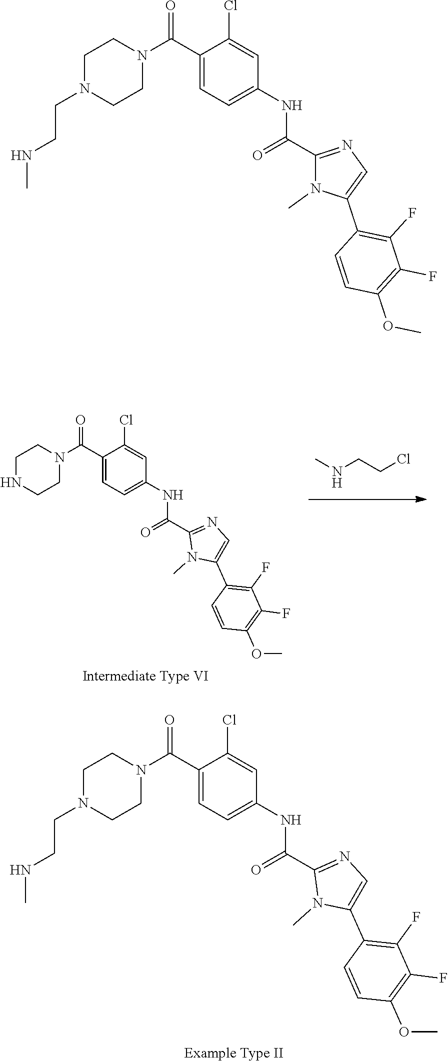

The mixture of N-(3-chloro-4-(piperazine-1-carbonyl)phenyl)-5-(2,3-difluoro-4-methoxyphenyl)-1-methyl-1H-imidazole-2-carboxamide (intermediate 374, 250 mg, 510 μmol), 2-chloro-N-methylethan-1-amine (57.3 mg, 612 μmol) and sodium iodide (76.5 mg, 510 μmol), K₂CO₃ (141 mg, 1.02 mmol) in DMF (2 mL) was stirred at 85° C. for 3 h. Then the mixture was poured into water. The water layer was extracted with DCM. The organic layer was washed with water and concentrated. The residue was purified by Prep-HPLC to give the title compound (42 mg). MS (ESI, m/z): 547.2 [M+H]⁺.

The following Type II Example was prepared in analogy to example 243.

| | | | ESI MS | |
|---|---|---|---|---|
| Ex. | Name | Structure | [M + H]⁺ | Starting Material |
| 244 | N-[4-[4-(2-amino-2- oxo-ethyl)piperazine-1- carbonyl]-3-chloro- phenyl]-5-(2,3-difluoro- 4-methoxy-phenyl)-1- methyl-imidazole-2- carboxamide | 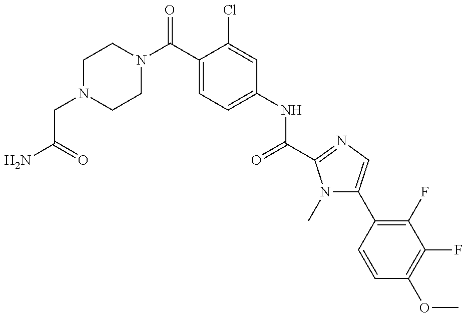 [SMILES: COc1ccc(-c2cnc(C(=O)Nc3ccc(C(=O)N4CCN(CC(N)=O)CC4)c(Cl)c3)n2C)c(F)c1F] | 547.1 | Intermediate 374 and 2-iodoacetamide |

### Example Type II: 245

### N-(Azetidin-3-ylmethyl)-1-[2-chloro-4-[[5-[4-(difluoromethoxy)-2,3-difluoro-phenyl]-1-methyl-imidazole-2-carbonyl]amino]benzoyl]piperidine-4-carboxamide trifluoroacetate

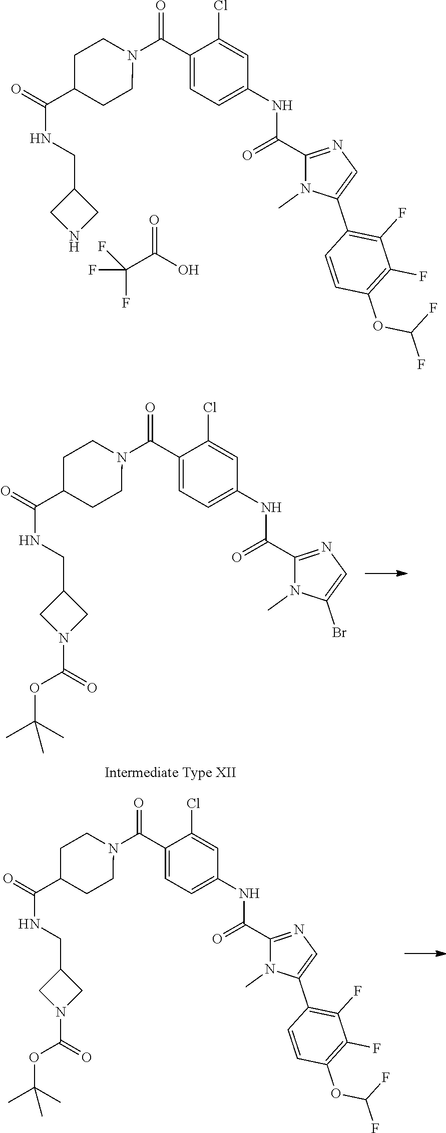

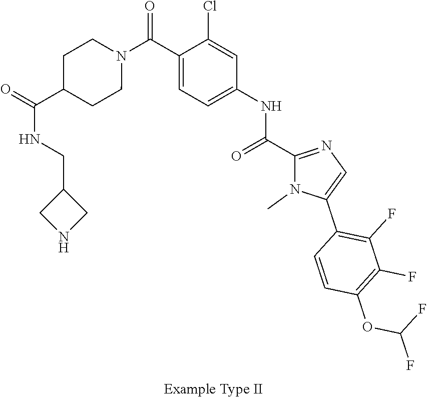 [SMILES: Cn1c(-c2ccc(OC(F)F)c(F)c2F)cnc1C(=O)Nc1ccc(C(=O)N2CCC(C(=O)NCC3CNC3)CC2)c(Cl)c1]

### Step 1: tert-butyl 3-[[[1-[2-chloro-4-[[5-[4-(difluoromethoxy)-2,3-difluoro-phenyl]-1-methyl-imidazole-2-carbonyl]amino]benzoyl]piperidine-4-carbonyl]amino]methyl]azetidine-1-carboxylate

A mixture of tert-butyl 3-((1-(4-(5-bromo-1-methyl-1H-imidazole-2-carboxamido)-2-chlorobenzoyl)piperidine-4-carboxamido)methyl)azetidine-1-carboxylate (intermediate 421, 530 mg, 831 μmol), 2-(4-(difluoromethoxy)-2,3-difluorophenyl)-4,4,5,5-tetramethyl-1,3,2-dioxaborolane (intermediate 315, 305 mg, 997 μmol), Na₂CO₃ (440 mg, 4.15 mmol) and 1,1′-bis(di-tert-butylphosphino)ferrocene palladium dichloride (54.1 mg, 83.1 μmol) in 1,4-dioxane (15 mL) and water (1.5 mL) was irradiated under microwave at 100° C. for 50 mins. Then the mixture was concentrated and the residue was purified by flash column to give the title compound (400 mg, 65.3% yield) as a black oil. MS (ESI, m/z): 737.8 [M+H]⁺.

### Step 2: N-(azetidin-3-ylmethyl)-1-[2-chloro-4-[[5-[4-(difluoromethoxy)-2,3-difluoro-phenyl]-1-methyl-imidazole-2-carbonyl]amino]benzoyl]piperidine-4-carboxamide; 2,2,2-trifluoroacetic acid

At room temperature, CF₃COOH (6 mL) was added to a solution of tert-butyl 3-[[[1-[2-chloro-4-[[5-[4-(difluoromethoxy)-2,3-difluoro-phenyl]-1-methyl-imidazole-2-carbonyl]amino]benzoyl]piperidine-4-carbonyl]amino]methyl]azetidine-1-carboxylate (400 mg, 543 μmol) in DCM (10 mL). The solution was stirred for 40 mins. Under ice cooling, NH₃.H₂O was added until pH 8-9 was reached. The solution was concentrated and the water layer was extracted with DCM. The organic layer was concentrated to give a crude product which was purified by Prep-HPLC to give the title compound (21 mg). MS (ESI, m/z): 637.3 [M+H]⁺.

The following Type II Examples were prepared in analogy to example 245.

| | | | ESI MS | |
|---|---|---|---|---|
| Ex. | Name | Structure | [M + H]⁺ | Starting Material |
| 246 | N-[4-[4-[3-(2- aminoethoxy)propanoyl] piperazine-1- carbonyl]-3-chloro- phenyl]-5-(2,3-difluoro- 4-methoxy-phenyl)-1- methyl-imidazole-2- carboxamide formate | 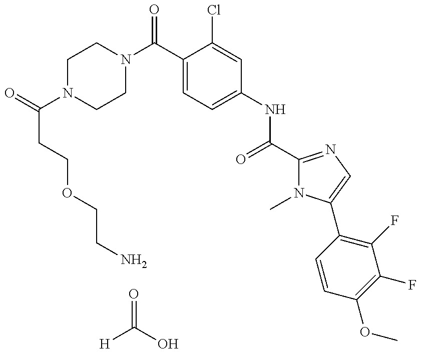 | 605.3 | Intermediate 422 and (2,3-difluoro-4- methoxyphenyl)boro- nic acid |
| 247 | 5-[3-chloro-2-fluoro-4- (fluoromethoxy)phenyl]- N-[3-chloro-4-[4- [(2S,4R)-4- hydroxypyrrolidine-2- carbonyl]piperazine-1- carbonyl]phenyl]-1- methyl-imidazole-2- carboxamide | 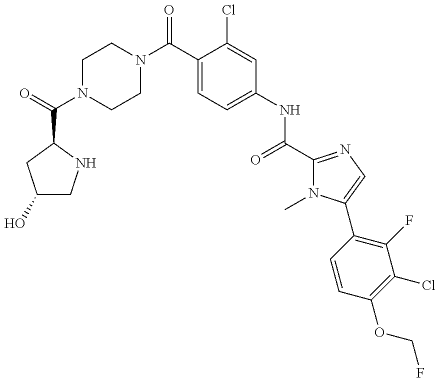 [SMILES: Cn1c(-c2ccc(OCF)c(Cl)c2F)cnc1C(=O)Nc1ccc(C(=O)N2CCN(C(=O)[C@@H]3C[C@@H](O)CN3)CC2)c(Cl)c1] | 637.1 | Intermediate 423 and intermediate 320 |
| 248 | 5-[3-chloro-2-fluoro-4- (fluoromethoxy)phenyl]- N-[4-[4-[(2S,4R)-4- hydroxypyrrolidine-2- carbonyl]piperazine-1- carbonyl]-3-methyl- phenyl]-1-methyl- imidazole-2- carboxamide | 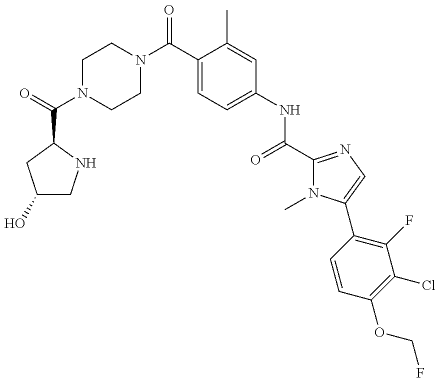 [SMILES: Cc1cc(NC(=O)c2ncc(-c3ccc(OCF)c(Cl)c3F)n2C)ccc1C(=O)N1CCN(C(=O)[C@@H]2C[C@@H](O)CN2)CC1] | 617.1 | Intermediate 424 and intermediate 320 |
| 249 | N-[3-chloro-4-[4- (piperidine-4- carbonyl)piperazine-1- carbonyl]phenyl]-5-[4- (difluoromethoxy)-2,3- difluoro-phenyl]-1- methyl-imidazole-2- carboxamide formate | 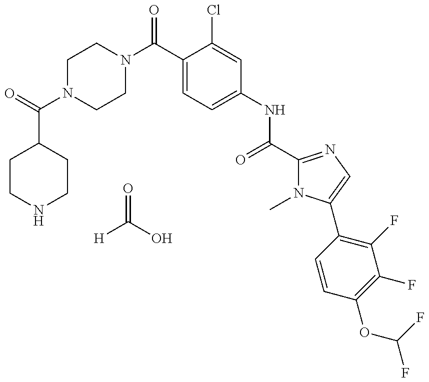 [SMILES: Cn1c(-c2ccc(OC(F)F)c(F)c2F)cnc1C(=O)Nc1ccc(C(=O)N2CCN(C(=O)C3CCNCC3)CC2)c(Cl)c1 ; O=CO] | 637.2 | Intermediate 426 and intermediate 315 |
| 250 | N-(azetidin-3-yl)-1-[2- chloro-4-[[5-(2,3- difluoro-4-methoxy- phenyl)-1-methyl- imidazole-2- carbonyl]amino]benzoyl] piperidine-4- carboxamide trifluoroacetate | 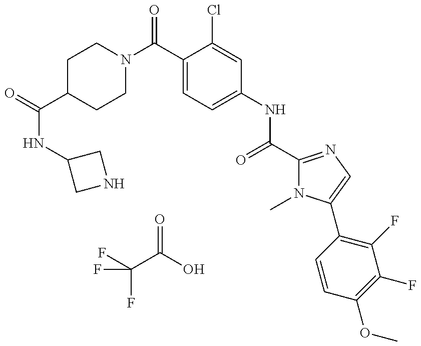 [SMILES: COc1ccc(-c2cnc(C(=O)Nc3ccc(C(=O)N4CCC(C(=O)NC5CNC5)CC4)c(Cl)c3)n2C)c(F)c1F ; O=C(O)C(F)(F)F] | 587.2 | Intermediate 425 and (2,3-difluoro-4- methoxyphenyl)boro- nic acid |
| 251 | 5-[2-chloro-4- (cyanomethoxy)-3- fluoro-phenyl]-N-[3- chloro-4-[4-(4- hydroxypiperidine-4- carbonyl)piperazine-1- carbonyl]phenyl]-1- methyl-imidazole-2- carboxamide formate | 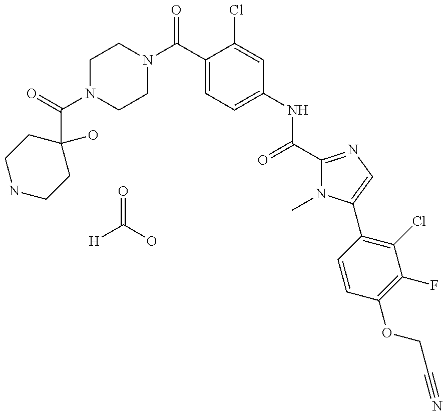 [SMILES: Cn1c(-c2ccc(OCC#N)c(F)c2Cl)cnc1C(=O)Nc1ccc(C(=O)N2CCN(C(=O)C3(O)CCNCC3)CC2)c(Cl)c1 ; O=CO] | 658.2 | Intermediate 427 and Intermediate 326 |
| 252 | 5-[2-chloro-4- (cyanomethoxy)-3- fluoro-phenyl]-N-[3- chloro-4-[4-(piperidine- 4-carbonyl)piperazine- 1-carbonyl]phenyl]-1- methyl-imidazole-2- carboxamide formate | 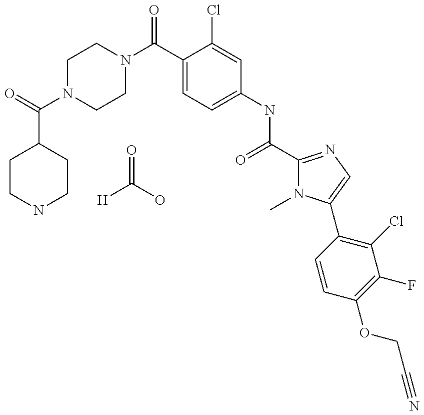 | 642.2 | Intermediate 426 and Intermediate 326 |
| 253 | 5-[2-chloro-4- (cyanomethoxy)-3- fluoro-phenyl]-N-[3- chloro-4-[4-[(3S)- pyrrolidine-3- carbonyl]piperazine-1- carbonyl]phenyl]-1- methyl-imidazole-2- carboxamide formate | 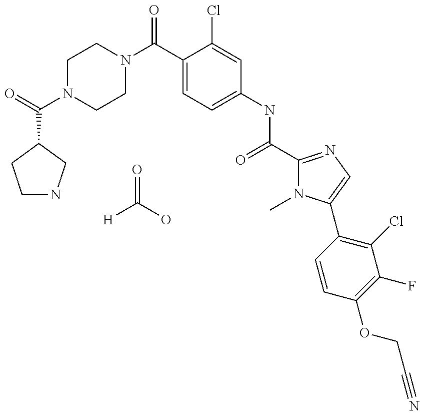 [SMILES: Cn1c(-c2ccc(OCC#N)c(F)c2Cl)cnc1C(=O)Nc1ccc(C(=O)N2CCN(C(=O)[C@H]3CCNC3)CC2)c(Cl)c1 ; O=CO] | 628.0 | Intermediate 428 and Intermediate 326 |
| 254 | 5-[2-chloro-4- (cyanomethoxy)-3- fluoro-phenyl]-N-[3- chloro-4-[4-[(3R)- pyrrolidine-3- carbonyl]piperazine-1- carbonyl]phenyl]-1- methyl-imidazole-2- carboxamide formate | 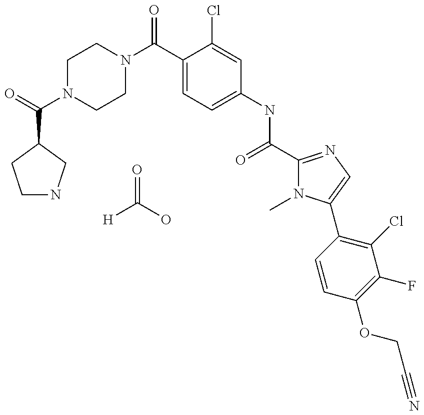 [SMILES: Cn1c(-c2ccc(OCC#N)c(F)c2Cl)cnc1C(=O)Nc1ccc(C(=O)N2CCN(C(=O)[C@@H]3CCNC3)CC2)c(Cl)c1 ; O=CO] | 628.2 | Intermediate 429 and Intermediate 326 |
| 255 | N-[3-chloro-4-[4- (piperidine-4- carbonyl)piperazine-1- carbonyl]phenyl]-5- [2,3-difluoro-4-(2- methoxyethoxy)phenyl]- 1-methyl-imidazole-2- carboxamide formate | 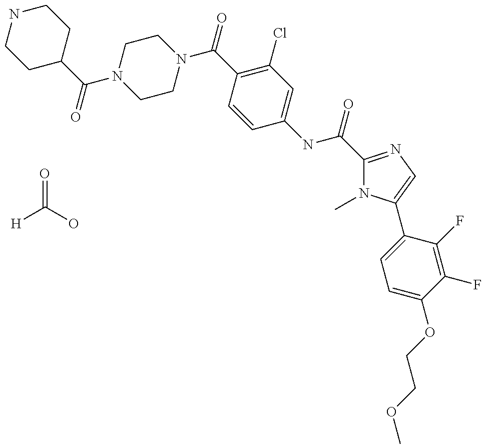 [SMILES: COCCOc1ccc(-c2cnc(C(=O)Nc3ccc(C(=O)N4CCN(C(=O)C5CCNCC5)CC4)c(Cl)c3)n2C)c(F)c1F ; O=CO] | 645.4 | Intermediate 426 and Intermediate 336 |
| 256 | N-[3-chloro-4-[4- (piperidine-4- carbonyl)piperazine-1- carbonyl]phenyl]-5-[4- (cyanomethyl)-2- fluoro-phenyl]-1- methyl-imidazole-2- carboxamide formate | 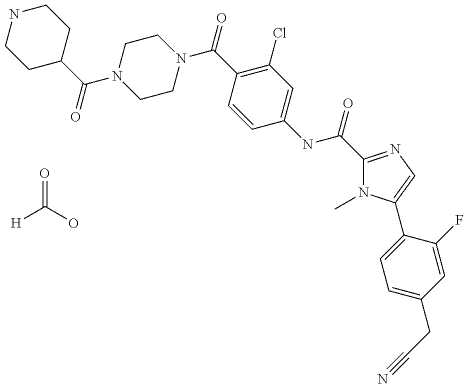 [SMILES: Cn1c(-c2ccc(CC#N)cc2F)cnc1C(=O)Nc1ccc(C(=O)N2CCN(C(=O)C3CCNCC3)CC2)c(Cl)c1 ; O=CO] | 592.2 | Intermediate 426 and Intermediate 329 |
| 257 | N-[3-chloro-4-[4- (piperidine-4- carbonyl)piperazine-1- carbonyl]phenyl]-5-(2- fluoro-3,4-dimethoxy- phenyl)-1-methyl- imidazole-2- carboxamide formate | 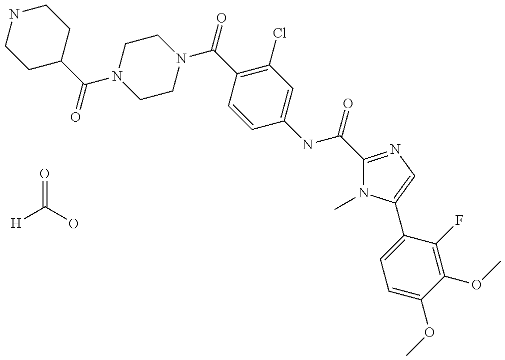 [SMILES: COc1ccc(-c2cnc(C(=O)Nc3ccc(C(=O)N4CCN(C(=O)C5CCNCC5)CC4)c(Cl)c3)n2C)c(F)c1OC ; O=CO] | 613.1 | Intermediate 426 and Intermediate 330 |
| 258 | N-[3-chloro-4-[4- (piperidine-4- carbonyl)piperazine-1- carbonyl]phenyl]-5- (3,4-difluoro-5- methoxy-phenyl)-1- methyl-imidazole-2- carboxamide formate | 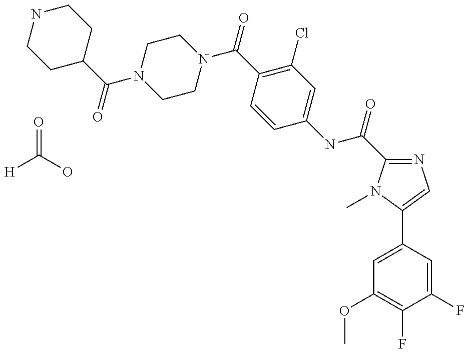 [SMILES: O=CO ; COc1cc(-c2cnc(C(=O)Nc3ccc(C(=O)N4CCN(C(=O)C5CCNCC5)CC4)c(Cl)c3)n2C)cc(F)c1F] | 601.2 | Intermediate 426 and Intermediate 337 |
| 259 | N-[3-chloro-4-[4-[2- [(3S)-pyrrolidin-3- yl]acetyl]piperazine-1- carbonyl]phenyl]-5-[3- fluoro-4- (fluoromethoxy)-2- methyl-phenyl]-1- methyl-imidazole-2- carboxamide formate | 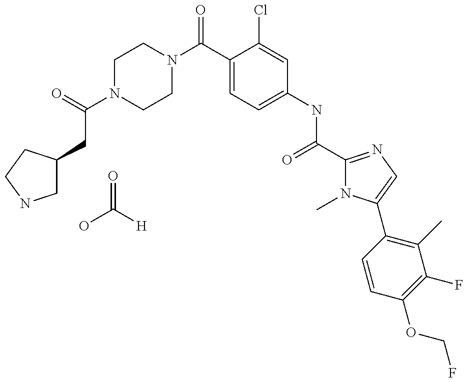 | 615.0 | Intermediate 430 and Intermediate 331 |
| 260 | N-[3-chloro-4-[4-[2- [(3S)-pyrrolidin-3- yl]acetyl]piperazine-1- carbonyl]phenyl]-5-(2- cyano-3-fluoro-4- methoxy-phenyl)-1- methyl-imidazole-2- carboxamide formate | 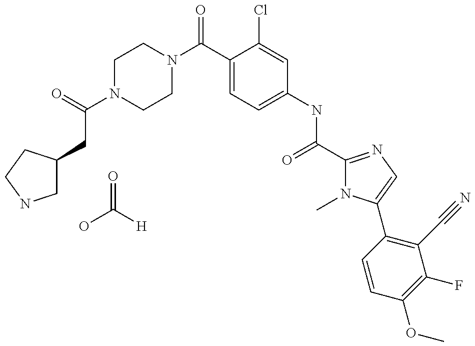 | 608.1 | Intermediate 430 and Intermediate 332 |
| 261 | N-[3-chloro-4-[4-[2- [(3S)-pyrrolidin-3- yl]acetyl]piperazine-1- carbonyl]phenyl]-5-[2- (difluoromethyl)-3- fluoro-4-methoxy- phenyl]-1-methyl- imidazole-2- carboxamide formate | 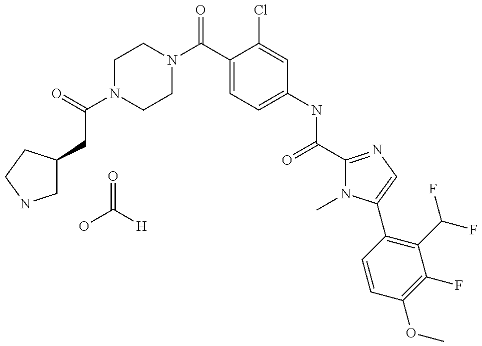 | 633.2 | Intermediate 430 and Intermediate 334 |
| 262 | N-[3-chloro-4-[4- (piperidine-4- carbonyl)piperazine-1- carbonyl]phenyl]-5-[4- (dimethylamino)-2,3- difluoro-phenyl]-1- methyl-imidazole-2- carboxamide formate | 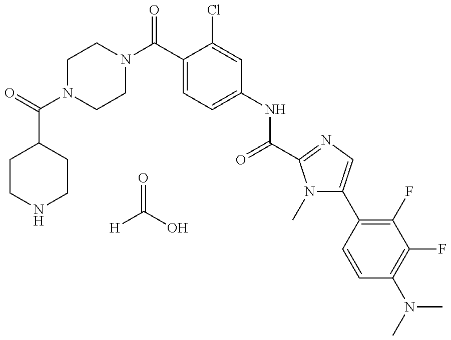 [SMILES: CN(C)c1ccc(-c2cnc(C(=O)Nc3ccc(C(=O)N4CCN(C(=O)C5CCNCC5)CC4)c(Cl)c3)n2C)c(F)c1F ; O=CO] | 614.2 | Intermediate 426 and Intermediate 323 |
| 263 | N-[3-chloro-4-[4- (piperidine-4- carbonyl)piperazine-1- carbonyl]phenyl]-5-[4- (dimethylcarbamoyl)- 2,3-difluoro-phenyl]-1- methyl-imidazole-2- carboxamide formate | 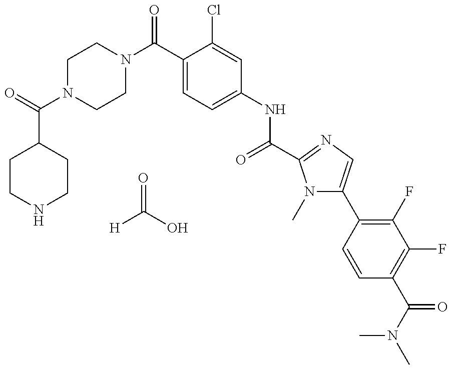 [SMILES: CN(C)C(=O)c1ccc(-c2cnc(C(=O)Nc3ccc(C(=O)N4CCN(C(=O)C5CCNCC5)CC4)c(Cl)c3)n2C)c(F)c1F ; O=CO] | 642.2 | Intermediate 426 and Intermediate 324 |
| 264 | N-(azetidin-3- ylmethyl)-1-[2-chloro- 4-[[5-[4- (cyanomethoxy)-2,3- difluoro-phenyl]-1- methyl-imidazole-2- carbonyl]amino]benzoyl] piperidine-4- carboxamide trifluoroacetate | 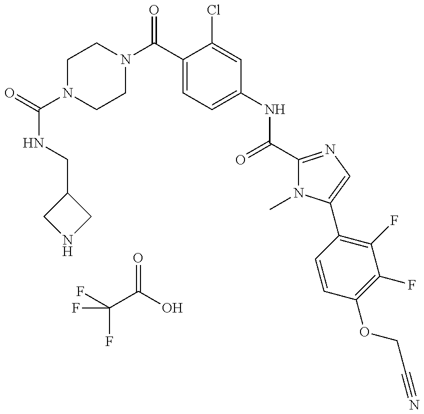 [SMILES: Cn1c(-c2ccc(OCC#N)c(F)c2F)cnc1C(=O)Nc1ccc(C(=O)N2CCN(C(=O)NCC3CNC3)CC2)c(Cl)c1 ; O=C(O)C(F)(F)F] | 626.6 | Intermediate 421 and Intermediate 327 |
| 265 | N-[3-chloro-4-[4- (piperidine-4- carbonyl)piperazine-1- carbonyl]phenyl]-5- (2,3-difluoro-4- methoxy-phenyl)-1- methyl-imidazole-2- carboxamide hydrochloride | 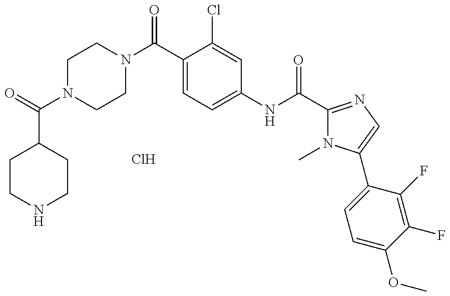 [SMILES: COc1ccc(-c2cnc(C(=O)Nc3ccc(C(=O)N4CCN(C(=O)C5CCNCC5)CC4)c(Cl)c3)n2C)c(F)c1F ; Cl] | 601.2 | Intermediate 266 and (2,3-difluoro-4- methoxyphenyl)boro- nic acid |
| 268 | 5-[3-chloro-4- (cyanomethoxy)-2- fluoro-phenyl]-N-[3- chloro-4-[4-(piperidine- 4-carbonyl)piperazine- 1-carbonyl]phenyl]-1- methyl-imidazole-2- carboxamide | 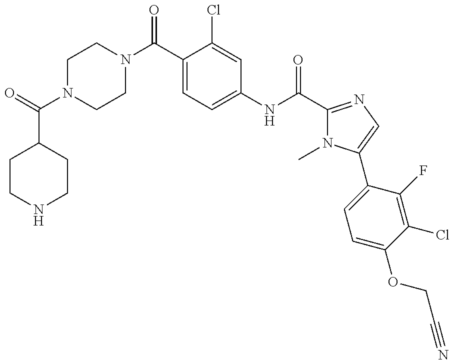 [SMILES: Cn1c(-c2ccc(OCC#N)c(Cl)c2F)cnc1C(=O)Nc1ccc(C(=O)N2CCN(C(=O)C3CCNCC3)CC2)c(Cl)c1] | 640.5 | Intermediate 266 and (3-chloro-4- (cyanomethoxy)-2- fluorophenyl)boronic acid |

### Example Type II: 269

### 5-[3-Chloro-4-(cyanomethoxy)phenyl]-N-[3-chloro-4-[4-[2-(dimethylamino)ethyl]piperazine-1-carbonyl]phenyl]-1-methyl-imidazole-2-carboxamide trifluoroacetate

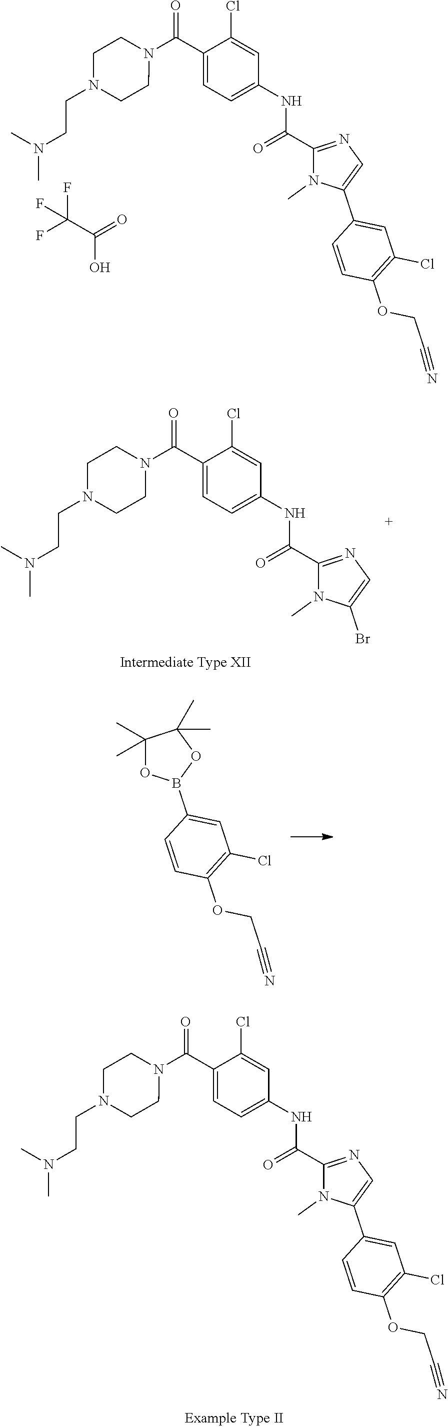

A mixture of 5-bromo-N-[3-chloro-4-[4-[2-(dimethylamino)ethyl]piperazine-1-carbonyl]phenyl]-1-methyl-imidazole-2-carboxamide (intermediate 431, 200 mg, 402 μmol), 2-[2-chloro-4-(4,4,5,5-tetramethyl-1,3,2-dioxaborolan-2-yl)phenoxy]acetonitrile.

(intermediate 325, 118 mg, 402 μmol), Na₂CO₃ (128 mg, 1.21 mmol) and 1,1′-bis(di-tert-butylphosphino)ferrocene palladium dichloride (26.2 mg, 40.2 μmol) in 1,4-dioxane (4 mL) and water (0.4 mL) was irradiated under microwave at 100° C. for 1 h. The mixture was filtered and concentrated. Water was added and the water layer was extracted with DCM. The organic layer was concentrated and the crude product was purified by Prep-HPLC to give the desired product (29 mg) as a light brown powder. MS (ESI, m/z): 584.3 [M+H]⁺.

The following Type II Examples were prepared in analogy to example 269.

| | | | ESI MS | |
|---|---|---|---|---|
| Ex. | Name | Structure | [M + H]⁺ | Starting Material |
| 270 | 5-[2-chloro-4- (cyanomethoxy)-3- fluoro-phenyl]-N-[3- chloro-4-[4-[2- (dimethylamino)ethyl] piperazine-1- carbonyl]phenyl]-1- methyl-imidazole-2- carboxamide | 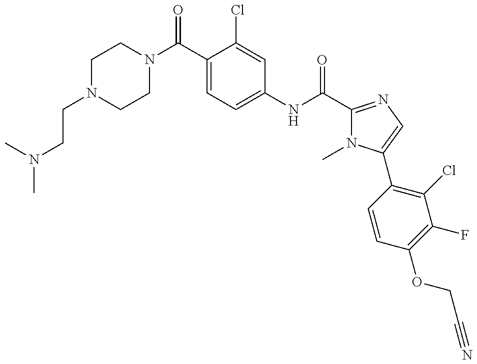 [SMILES: CN(C)CCN1CCN(C(=O)c2ccc(NC(=O)c3ncc(-c4ccc(OCC#N)c(F)c4Cl)n3C)cc2Cl)CC1] | 602.0 | Intermediate 431 and Intermediate 326 |
| 271 | N-[3-chloro-4-[4-[2- (dimethylamino)ethyl] piperazine-1- carbonyl]phenyl]-5- [2,3-difluoro-4-(2- pyridyloxy)phenyl]-1- methyl-imidazole-2- carboxamide trifluoroacetate | 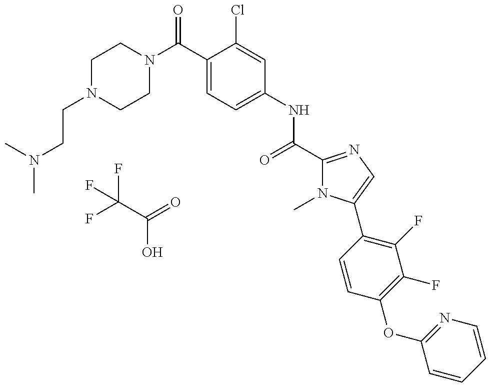 [SMILES: CN(C)CCN1CCN(C(=O)c2ccc(NC(=O)c3ncc(-c4ccc(Oc5ccccn5)c(F)c4F)n3C)cc2Cl)CC1 ; O=C(O)C(F)(F)F] | 624.4 | Intermediate 431 and Intermediate 338 |
| 272 | N-[3-chloro-4-[4-[2- (dimethylamino)ethyl] piperazine-1- carbonyl]phenyl]-5- [2,3-difluoro-4-(4- pyridyloxy)phenyl]-1- methyl-imidazole-2- carboxamide trifluoroacetate | 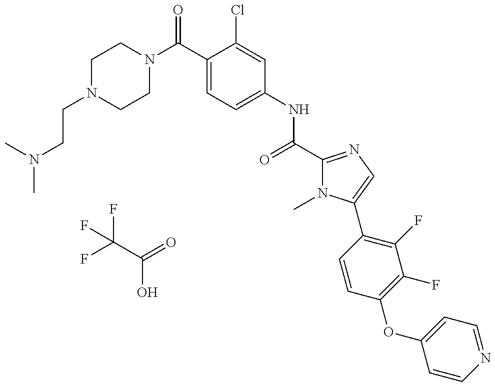 [SMILES: CN(C)CCN1CCN(C(=O)c2ccc(NC(=O)c3ncc(-c4ccc(Oc5ccncc5)c(F)c4F)n3C)cc2Cl)CC1 ; O=C(O)C(F)(F)F] | 624.4 | Intermediate 431 and Intermediate 339 |
| 273 | N-[3-chloro-4-[4-[2- (dimethylamino)ethyl] piperazine-1- carbonyl]phenyl]-5- (2,3-difluoro-4- pyrimidin-2-yloxy- phenyl)-1-methyl- imidazole-2- carboxamide trifluoroacetate | 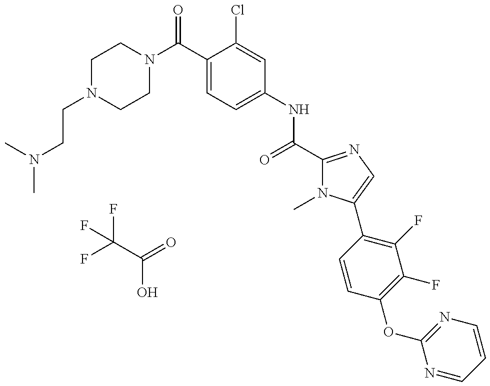 [SMILES: CN(C)CCN1CCN(C(=O)c2ccc(NC(=O)c3ncc(-c4ccc(Oc5ncccn5)c(F)c4F)n3C)cc2Cl)CC1 ; O=C(O)C(F)(F)F] | 625.4 | Intermediate 431 and Intermediate 340 |
| 274 | N-[3-chloro-4-[4-[2- (dimethylamino)ethyl] piperazine-1- carbonyl]phenyl]-5- (3-fluoro-4-methoxy- phenyl)-1-methyl- imidazole-2- carboxamide formate | 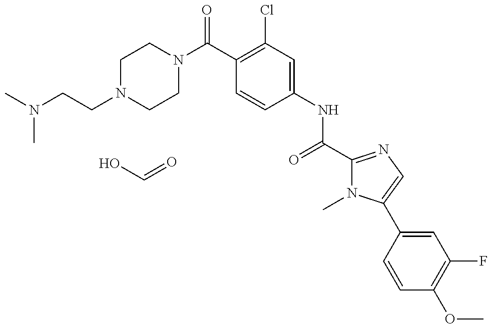 | 543.1 | Intermediate 432 and 3-fluoro-4- methoxyphenylbo- ronic acid |
| 275 | 5-[4- (difluoromethoxy)phen- yl]-N-[4-[4-[2- (dimethylamino)ethyl] piperazine-1- carbonyl]-3-ethyl- phenyl]-1-methyl- imidazole-2- carboxamide | 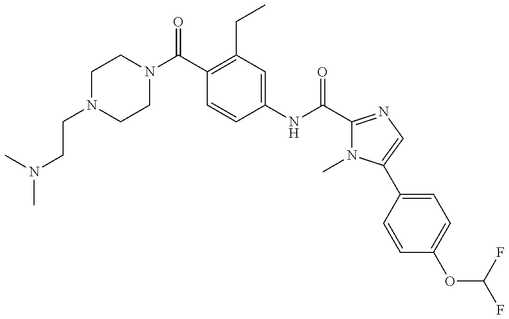 [SMILES: CCc1cc(NC(=O)c2ncc(-c3ccc(OC(F)F)cc3)n2C)ccc1C(=O)N1CCN(CCN(C)C)CC1] | 555.4 | Intermediate 445 and N,N- dimethyl-2- (piperazin-1- yl)ethan-1- amine |
| 276 | N-(4-(4-(2- (dimethylamino)ethyl) piperazine-1- carbonyl)-3- ethylphenyl)-5-(3- fluoro-4- isopropoxyphenyl)-1- methyl-1H-imidazole- 2-carboxamide | 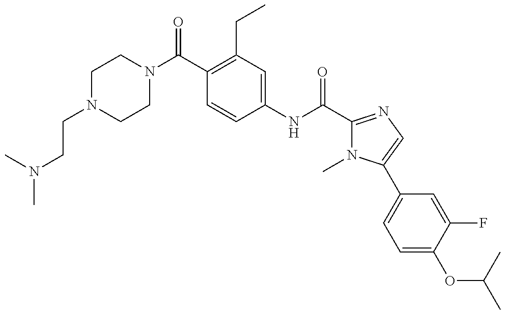 [SMILES: CCc1cc(NC(=O)c2ncc(-c3ccc(OC(C)C)c(F)c3)n2C)ccc1C(=O)N1CCN(CCN(C)C)CC1] | 565.4 | Intermediate 357 and N,N- dimethyl-2- (piperazin-1- yl)ethan-1- amine |
| 277 | 5-(2-chloro-4- methoxyphenyl)-N-(4- (4-(2- (dimethylamino)ethyl) piperazine-1- carbonyl)-3- ethylphenyl)-1- methyl-1H-imidazole- 2-carboxamide | 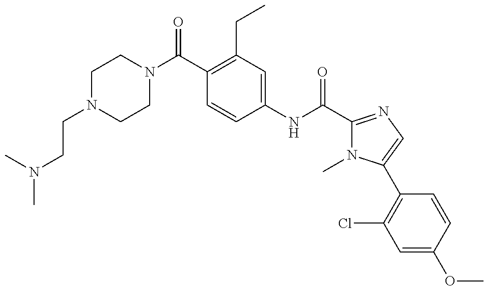 | 553.4 | Intermediate 359 and N,N- dimethyl-2- (piperazin-1- yl)ethan-1- amine |
| 278 | N-[3-chloro-4-[4-[2- (dimethylamino)ethyl] piperazine-1- carbonyl]phenyl]-5- (2,3-difluoro-4- methoxy-phenyl)-1- methyl-imidazole-2- carboxamide formate | 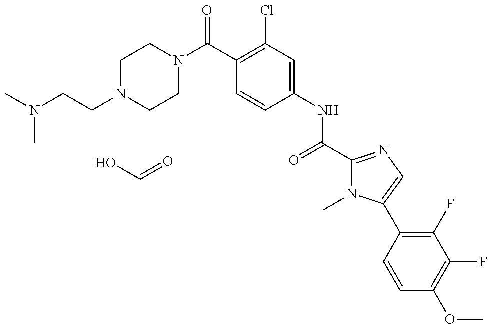 | 607.1 | Intermediate 431 and (2,3- difluoro-4- methoxyphenyl) boronic acid |

### Example Type II: 279

### N-(4-(4-(3-(3-Amino-3-oxopropoxy)propanoyl)piperazine-1-carbonyl)-3-chlorophenyl)-5-(2,3-difluoro-4-methoxyphenyl)-1-methyl-1H-imidazole-2-carboxamide

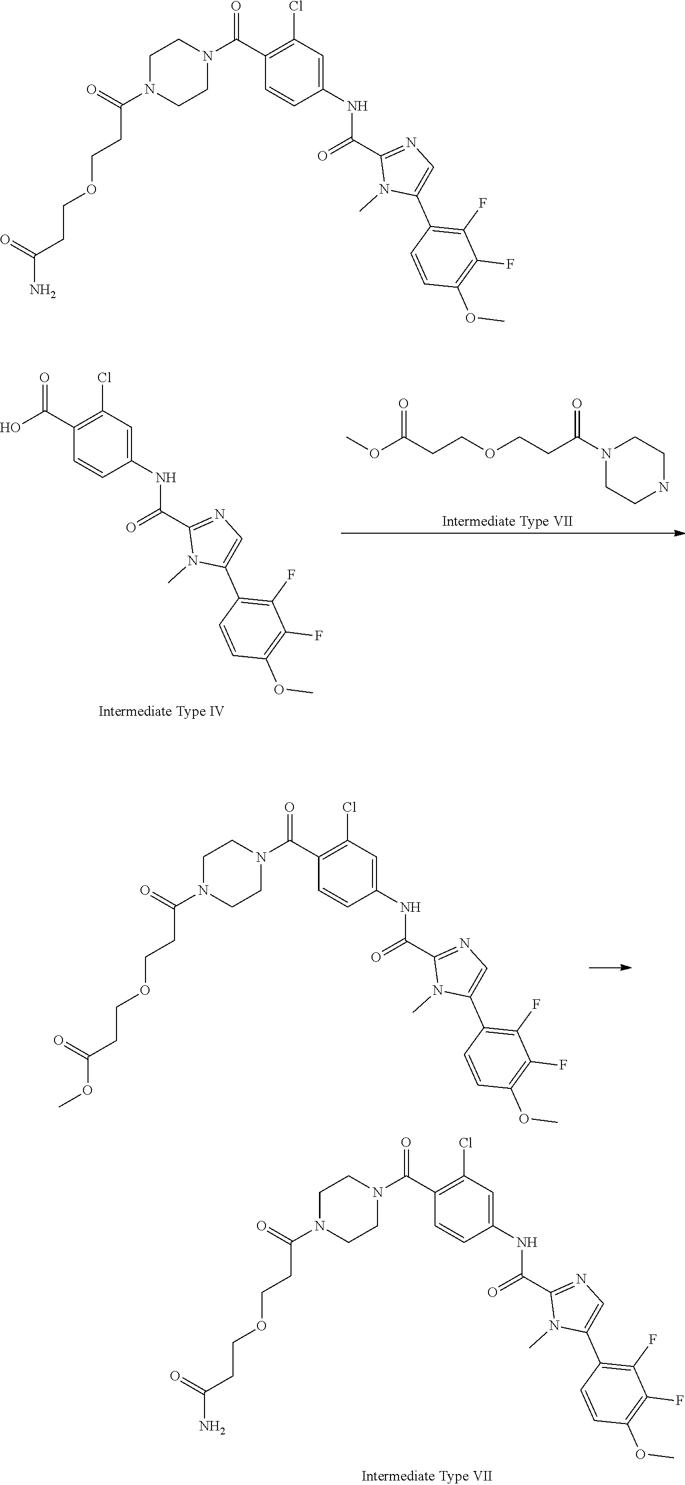

### Step 1: N-[3-chloro-4-[4-(3-methoxypropanoyl)piperazine-1-carbonyl]phenyl]-5-(2,3-difluoro-4-methoxy-phenyl)-1-methyl-imidazole-2-carboxamide

A mixture of 2-chloro-4-[[5-(2,3-difluoro-4-methoxy-phenyl)-1-methyl-imidazole-2-carbonyl]amino]benzoic acid (500.0 mg, 0.950 mmol), methyl 3-(3-oxo-3-piperazin-1-yl-propoxy)propanoate and 3-methoxy-1-(piperazin-1-yl)propan-1-one (467 mg), N,N-diisopropylethylamine (0.41 mL, 2.37 mmol) and 1-propylphosphonic anhydride solution, 50 wt. % in ethyl acetate (1207 mg, 1.9 mmol) in DMF (6 mL) was stirred at 25° C. for 16 h. The mixture was added to water (15 mL) and extracted with ethyl acetate (10 mL×3). The combined organic layers were washed with saturated aqueous NaHCO₃ solution (15 mL), dried over anhydrous Na₂SO₄ and concentrated under reduced pressure to afford the crude product (637 mg) as a brown gum. 200 mg of the crude was purified by prep-HPLC (Chromatographic column: Kromasil-C18 100×21.2 mm Sum; 5%-95% ACN in H₂O with 0.1% FA as eluent). The desired fractions were dried by lyophilization to afford final compound methyl 3-[3-[4-[2-chloro-4-[[5-(2,3-difluoro-4-methoxy-phenyl)-1-methyl-imidazole-2-carbonyl]amino]benzoyl]piperazin-1-yl]-3-oxo-propoxy]propanoate (22 mg) as a white solid. MS (ESI, m/z): 576.2 [M+H]⁺.

### Step 2: N-(4-(4-(3-(3-amino-3-oxopropoxy)propanoyl)piperazine-1-carbonyl)-3-chlorophenyl)-5-(2,3-difluoro-4-methoxyphenyl)-1-methyl-1H-imidazole-2-carboxamide

A mixture of methyl 3-[3-[4-[2-chloro-4-[[5-(2,3-difluoro-4-methoxy-phenyl)-1-methyl-imidazole-2-carbonyl]amino]benzoyl]piperazin-1-yl]-3-oxo-propoxy]propanoate (220 mg, 0.34 mmol), ammonia (5.0 mL, 0.340 mmol) was stirred at 40° C. for 12 h. The mixture was added to water (100 mL) and extracted with ethyl acetate (50 mL×3). The combined organic layers were dried over anhydrous Na₂SO₄ and concentrated under reduced pressure. The mixture was purified by prep-HPLC (Chromatographic column: Kromasil-C18 100×21.2 mm 5 um; 5%-95% ACN in H₂O with 0.1% FA as eluent) to afford the title compound (61.2 mg) as a white solid. MS (ESI, m/z): 633.2 [M+H]⁺.

The following Type II Example was prepared in analogy to 279.

| | | | ESI MS | |
|---|---|---|---|---|
| Ex. | Name | Structure | [M + H]⁺ | Starting Material |
| 280 | N-(4-(4-(3- carbamoylcyclobutane- 1-carbonyl)piperazine- 1-carbonyl)-3- chlorophenyl)-5-(2,3- difluoro-4- methoxyphenyl)-1- methyl-1H-imidazole- 2-carboxamide | 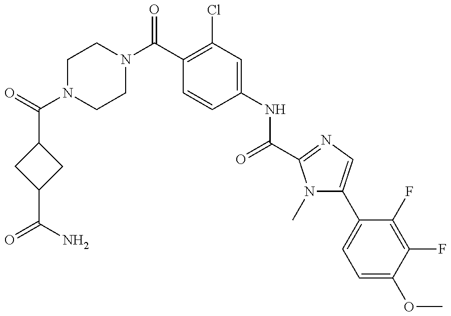 [SMILES: COc1ccc(-c2cnc(C(=O)Nc3ccc(C(=O)N4CCN(C(=O)C5CC(C(N)=O)C5)CC4)c(Cl)c3)n2C)c(F)c1F] | 615.2 | Intermediate 349 and 3- (methoxycarbonyl)cy- clobutane-1- carboxylic acid |

### Example Type III: 281

### N-(2-Aminoethyl)-4-(2-chloro-4-(5-(4-(cyanomethoxy)-2,3-difluorophenyl)-1-methyl-1H-imidazole-2-carboxamido)benzoyl)piperazine-1-carboxamide 2,2,2-trifluoroacetate

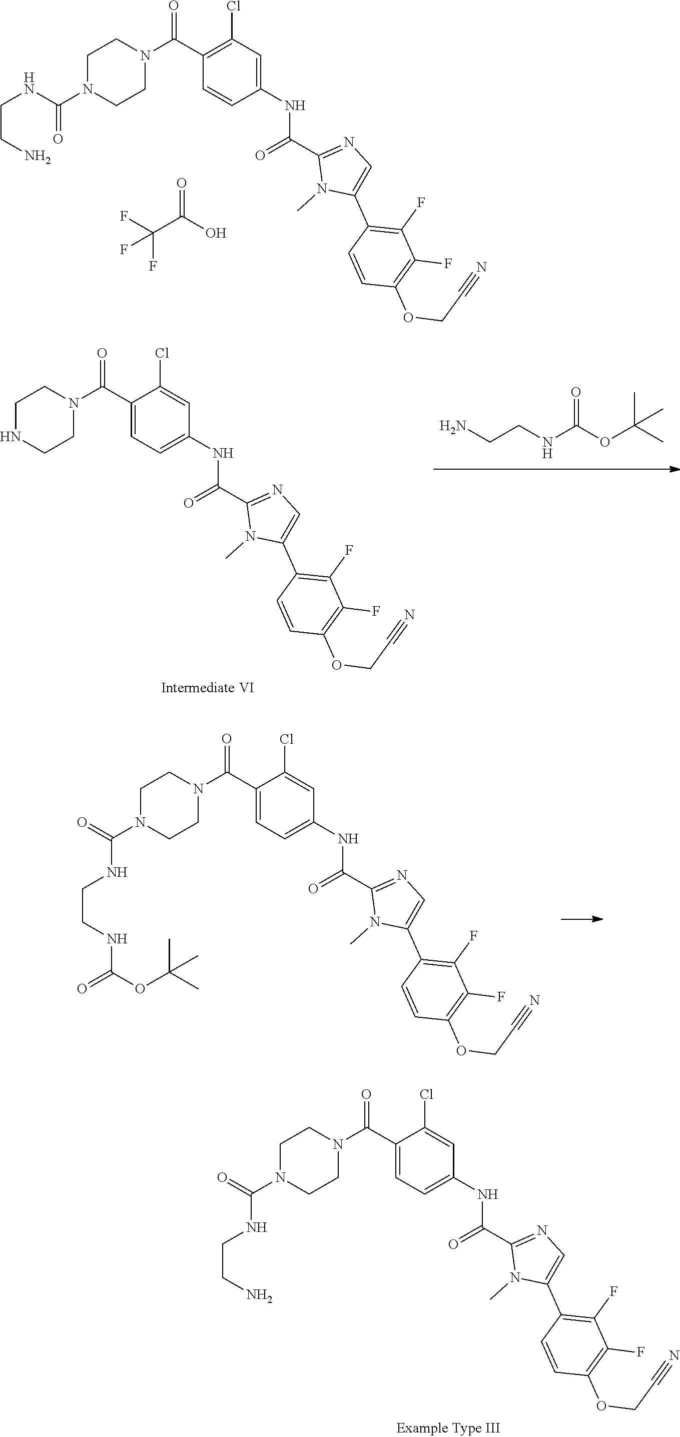 [SMILES: Cn1c(-c2ccc(OCC#N)c(F)c2F)cnc1C(=O)Nc1ccc(C(=O)N2CCN(C(=O)NCCN)CC2)c(Cl)c1 ; O=C(O)C(F)(F)F ; Cn1c(-c2ccc(OCC#N)c(F)c2F)cnc1C(=O)Nc1ccc(C(=O)N2CCNCC2)c(Cl)c1 ; CC(C)(C)OC(=O)NCCN ; Cn1c(-c2ccc(OCC#N)c(F)c2F)cnc1C(=O)Nc1ccc(C(=O)N2CCN(C(=O)NCCNC(=O)OC(C)(C)C)CC2)c(Cl)c1 ; Cn1c(-c2ccc(OCC#N)c(F)c2F)cnc1C(=O)Nc1ccc(C(=O)N2CCN(C(=O)NCCN)CC2)c(Cl)c1]

### Step 1:

### tert-butyl (2-(4-(2-chloro-4-(5-(4-(cyanomethoxy)-2,3-difluorophenyl)-1-methyl-1H-imidazole-2-carboxamido)benzoyl)piperazine-1-carboxamido)ethyl)carbamate

A solution of triethylamine (0.06 mL, 0.400 mmol), N-Boc-ethylenediamine (64.68 mg, 0.400 mmol) and triethylamine (0.06 mL, 0.400 mmol) in DMF (1.45 mL) was stirred at 25° C. for 1 h and a solution of N-[3-chloro-4-(piperazine-1-carbonyl)phenyl]-5-[4-(cyanomethoxy)-2,3-difluoro-phenyl]-1H-imidazole-2-carboxamide; 2,2,2-trifluoroacetic acid (124.13 mg, 0.200 mmol) in DMF (2 mL) was added. The reaction was quenched with water (20 mL) and the resulted solution was extracted with ethyl acetate (20 mL×3). The combined organic layers were dried over anhydrous Na₂SO₄ and concentrated in vacuo to give the title compound (150 mg) as a light brown solid. MS (ESI, m/z): 701.2 [M+H]⁺.

### Step 2:

### N-(2-aminoethyl)-4-(2-chloro-4-(5-(4-(cyanomethoxy)-2,3-difluorophenyl)-1-methyl-1H-imidazole-2-carboxamido)benzoyl)piperazine-1-carboxamide 2,2,2-trifluoroacetate

A solution of tert-butyl N-[2-[[4-[2-chloro-4-[[5-[4-(cyanomethoxy)-2,3-difluoro-phenyl]-1-methyl-imidazole-2-carbonyl]amino]benzoyl]piperazine-1-carbonyl]amino]ethyl]carbamate (150 mg, 0.110 mmol) and trifluoroacetic acid (1.0 mL, 12.98 mmol) in DCM (5 mL) was stirred at 25° C. for 1 h. After concentration in vacuo, the residue was purified by prep-HPLC (Chromatographic column: Kromasil-C18 100×21.2 mm Sum; 5%-95% ACN in H₂O with 0.1% TFA as eluent) to afford the title compound (17 mg) as a white solid. MS (ESI, m/z): 601.2 [M+H]⁺.

The following Type III Examples were prepared in analogy to 281.

| | | | ESI | |
|---|---|---|---|---|
| | | | MS | |
| | | | [M + | |
| Ex. | Name | Structure | H]⁺ | Starting Material |
| 7 | 4-[2-chloro-4-[[5-[4- (cyanomethoxy)-2,3- difluoro-phenyl]-1- methyl-imidazole-2- carbonyl]ami- no]benzoyl]- N-[(3R,4R)-4- hydroxypyrrolidin-3- yl]piperazine-1- carboxamide formate | 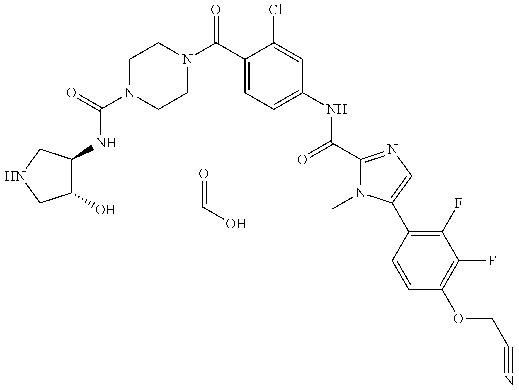 [SMILES: Cn1c(-c2ccc(OCC#N)c(F)c2F)cnc1C(=O)Nc1ccc(C(=O)N2CCN(C(=O)N[C@@H]3CNC[C@H]3O)CC2)c(Cl)c1 ; O=CO] | 643.3 | Intermediate 438 and tert-butyl (3R,4R)-3-amino-4- hydroxy- pyrrolidine-1- carboxylate |
| 282 | 4-[2-chloro-4-[[5-[4- (cyanomethoxy)-2,3- difluoro-phenyl]-1- methyl-imidazole-2- carbonyl]ami- no]benzoyl]- N-[(3S,4S)-4- hydroxypyrrolidin-3- yl]piperazine-1- carboxamide formate | 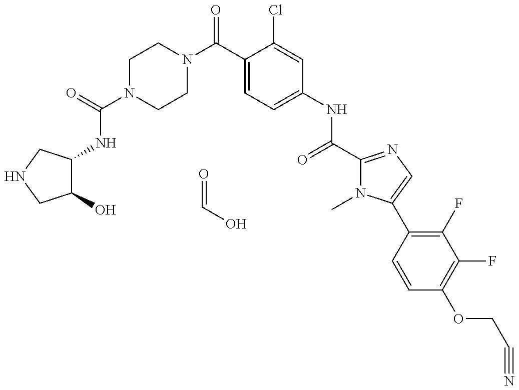 [SMILES: Cn1c(-c2ccc(OCC#N)c(F)c2F)cnc1C(=O)Nc1ccc(C(=O)N2CCN(C(=O)N[C@H]3CNC[C@@H]3O)CC2)c(Cl)c1 ; O=CO] | 643.3 | Intermediate 438 and tert-butyl trans- 3-amino-4-hydroxy- 1-pyrrolidine- carboxylate |
| 283 | 4-[2-chloro-4-[[5-(2,3- difluoro-4-methoxy- phenyl)-1-methyl- imidazole-2- carbonyl]ami- no]benzoyl]- N-[(3R,4R)-4- hydroxypyrrolidin-3- yl]piperazine-1- carboxamide formate | 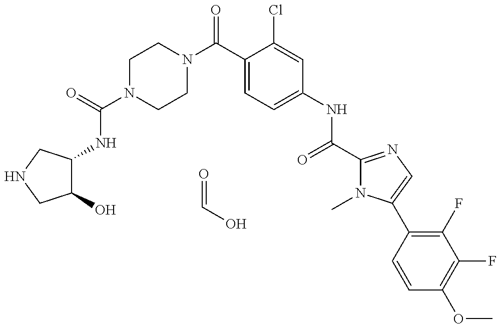 [SMILES: COc1ccc(-c2cnc(C(=O)Nc3ccc(C(=O)N4CCN(C(=O)N[C@H]5CNC[C@@H]5O)CC4)c(Cl)c3)n2C)c(F)c1F ; O=CO] | 618.2 | Intermediate 9 and tert-butyl (3R,4R)- 3-amino-4-hydroxy- pyrrolidine-1- carboxylate |
| 284 | 4-[2-chloro-4-[[5-(2,3- difluoro-4-methoxy- phenyl)-1-methyl- imidazole-2-carbonyl] amino]benzoyl]- N-[(3S,4S)-4- hydroxypyrrolidin-3- yl]piperazine-1- carboxamide formate | 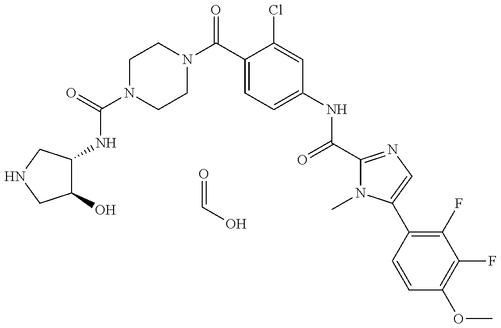 [SMILES: COc1ccc(-c2cnc(C(=O)Nc3ccc(C(=O)N4CCN(C(=O)N[C@H]5CNC[C@@H]5O)CC4)c(Cl)c3)n2C)c(F)c1F ; O=CO] | 618.2 | Intermediate 9 and tert-butyl trans-3- amino-4-hydroxy-1- pyrrolidine- carboxylate |
| 285 | 4-[4-[[5-(2,3-difluoro- 4-methoxy-phenyl)-1- methyl-imidazole-2- carbonyl]amino]-2- methyl-benzoyl]-N- [(3S,4S)-4- hydroxypyrrolidin-3- yl]piperazine-1- carboxamide formate | 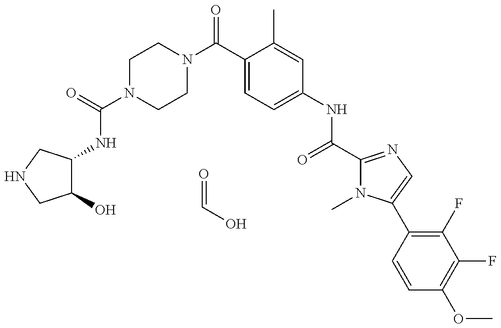 [SMILES: COc1ccc(-c2cnc(C(=O)Nc3ccc(C(=O)N4CCN(C(=O)N[C@H]5CNC[C@@H]5O)CC4)c(C)c3)n2C)c(F)c1F ; O=CO] | 598.2 | Intermediate 10 and tert-butyl trans-3- amino-4-hydroxy-1- pyrrolidine- carboxylate |
| 286 | 1-[4-[[5-(2,3-difluoro- 4-methoxy-phenyl)-1- methyl-imidazole-2- carbonyl]amino]-2- ethyl-benzoyl]-N-[3- (prop-2-ynylamino) propyl]piper- idine-4-carboxamide | 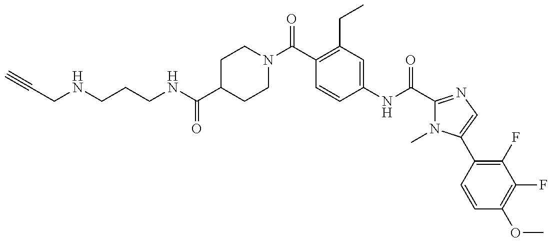 [SMILES: C#CCNCCCNC(=O)C1CCN(C(=O)c2ccc(NC(=O)c3ncc(-c4ccc(OC)c(F)c4F)n3C)cc2CC)CC1] | 621.5 | Intermediate 3 and tert-butyl (3- aminopropyl)(prop- 2-yn-1- yl)carbamate |
| 287 | N-[4-[4-[2-[(2-amino- 2-oxoethyl)amino] ethyl]piper- azine-1-carbonyl]-3- chloro-phenyl]-5-[4- (cyanomethoxy)-2,3- difluoro-phenyl]-1- methyl-imidazole-2- carboxamide formate | 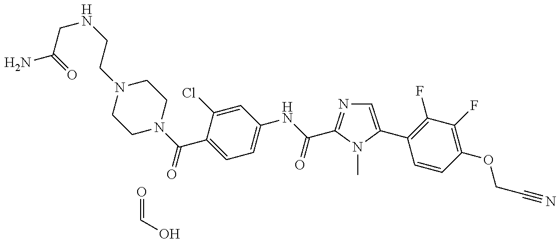 | 661.2 | Intermediate 438 and 2-(2-oxo- ethylamino)acet- amide |

### Example Type III: 288

### 2-[4-[4-[2-Chloro-4-[[5-(2,3-difluoro-4-methoxy-phenyl)-1-methyl-imidazole-2-carbonyl]amino]benzoyl]piperazine-1-carbonyl]-1-piperidyl]acetic acid trifluoroacetate

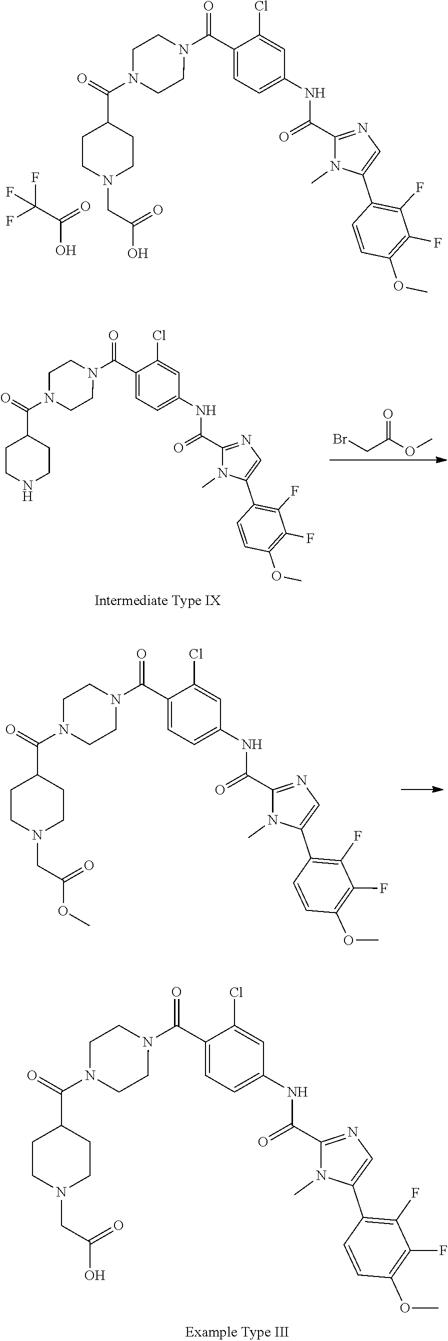

### Step 1: methyl 2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]acetate

A mixture of N-[3-chloro-4-[4-(piperidine-4-carbonyl)piperazine-1-carbonyl]phenyl]-5-(2,3-difluoro-4-methoxy-phenyl)-1-methyl-imidazole-2-carboxamide (intermediate 413, 400 mg, 666 μmol), methyl 2-bromoacetate (122 mg, 799 μmol) and Et₃N (337 mg, 464 μl, 3.33 mmol) in acetonitrile (10 mL) was stirred at 85° C. for 2 h and then concentrated. Water (5 mL) was added. The water layer was extracted with DCM. The organic layer was washed with water, dried over anhydrous Na₂SO₄ and concentrated. The residue (400 mg) was used in the next step reaction without further purification. MS (ESI, m/z): 673.3 [M+H]⁺.

### Step 2: 2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]acetic acid trifluoroacetate

To a solution of methyl 2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]acetate (400 mg, 594 μmol) in THF (24 mL), MeOH (1 mL) and water (12 mL) was added a solution of lithium hydroxide monohydrate (125 mg, 2.97 mmol). The mixture was stirred at room temperature overnight. Then the mixture was concentrated and was acidified by HCl until pH 3-4. The water layer was extracted with a 1:6 iPrOH:DCM mixture. The organic layer was concentrated and the residue was purified by Prep-HPLC to give the title compound (30 mg) as a white powder. MS (ESI, m/z): 659.3 [M+H]⁺.

The following Type III Example was prepared in analogy to example 288.

| | | | ESI MS | |
|---|---|---|---|---|
| Ex. | Name | Structure | [M + H]⁺ | Starting Material |
| 289 | N-[4-[4-[1-(2-amino-2- oxo-ethyl)piperidine-4- carbonyl]piperazine-1- carbonyl]-3-chloro- phenyl]-5-[4- (difluoromethxoy)-2,3- difluoro-phenyl]-1- methyl-imidazole-2- carboxamide | 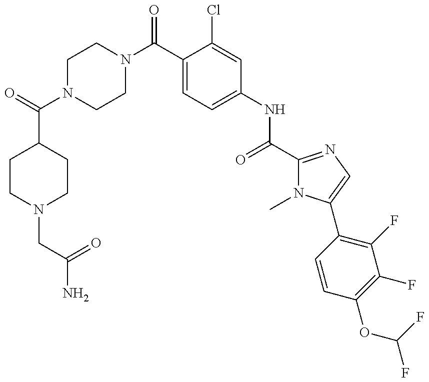 [SMILES: Cn1c(-c2ccc(OC(F)F)c(F)c2F)cnc1C(=O)Nc1ccc(C(=O)N2CCN(C(=O)C3CCN(CC(N)=O)CC3)CC2)c(Cl)c1] | 694.2 | Example 249 and 2- iodoacetamide |

### Example Type III: 290

### N-[4-[4-[1-(2-Aminoethyl)piperidine-4-carbonyl]piperazine-1-carbonyl]-3-chloro-phenyl]-5-(2,3-difluoro-4-methoxy-phenyl)-1-methyl-imidazole-2-carboxamide formate

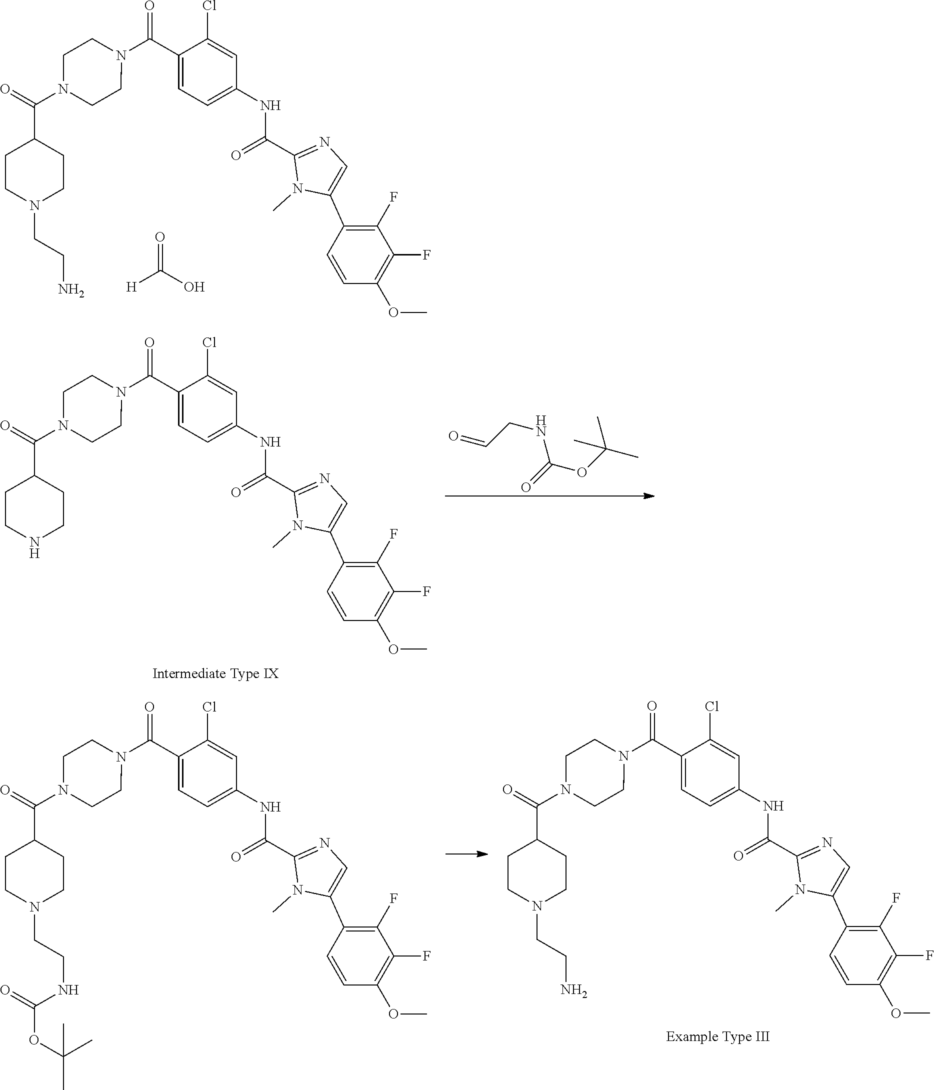 [SMILES: COc1ccc(-c2cnc(C(=O)Nc3ccc(C(=O)N4CCN(C(=O)C5CCN(CCN)CC5)CC4)c(Cl)c3)n2C)c(F)c1F ; O=CO ; COc1ccc(-c2cnc(C(=O)Nc3ccc(C(=O)N4CCN(C(=O)C5CCNCC5)CC4)c(Cl)c3)n2C)c(F)c1F ; CC(C)(C)OC(=O)NCC=O ; COc1ccc(-c2cnc(C(=O)Nc3ccc(C(=O)N4CCN(C(=O)C5CCN(CCNC(=O)OC(C)(C)C)CC5)CC4)c(Cl)c3)n2C)c(F)c1F ; COc1ccc(-c2cnc(C(=O)Nc3ccc(C(=O)N4CCN(C(=O)C5CCN(CCN)CC5)CC4)c(Cl)c3)n2C)c(F)c1F]

### Step 1: tert-butyl N-[2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]ethyl]carbamate

In a 100 mL round-bottomed flask, N-(3-chloro-4-(4-(piperidine-4-carbonyl)piperazine-1-carbonyl)phenyl)-5-(2,3-difluoro-4-methoxyphenyl)-1-methyl-1H-imidazole-2-carboxamide (55 mg, 91.5 μmol), tert-butyl (2-oxoethyl)carbamate (58.3 mg, 366 μmol) and sodium cyanoborohydride (28.8 mg, 458 μmol) were combined with MeOH (5 mL) to give a colorless solution. The reaction mixture was heated to 40° C. and stirred for 1 h. The crude reaction mixture was concentrated in vacuo. The reaction mixture was poured into 25 mL sat NaHCO₃ and extracted with EtOAc (3×25 mL). The organic layers were dried over Na₂SO₄ and concentrated in vacuo to afford tert-butyl (2-(4-(4-(2-chloro-4-(5-(2,3-difluoro-4-methoxyphenyl)-1-methyl-1H-imidazole-2-carboxamido)benzoyl)piperazine-1-carbonyl)piperidin-1-yl)ethyl)carbamate (68 mg, 8% yield). MS (ESI, m/z): 744.2 [M+H]⁺.

### Step 2: N-[4-[4-[1-(2-aminoethyl)piperidine-4-carbonyl]piperazine-1-carbonyl]-3-chloro-phenyl]-5-(2,3-difluoro-4-methoxy-phenyl)-1-methyl-imidazole-2-carboxamide formate

In a 100 mL round-bottomed flask, tert-butyl (2-(4-(4-(2-chloro-4-(5-(2,3-difluoro-4-methoxyphenyl)-1-methyl-1H-imidazole-2-carboxamido)benzoyl)piperazine-1-carbonyl)piperidin-1-yl)ethyl)carbamate (68 mg, 91.4 μmol) was combined with THF (3 mL) to give a colorless solution. HCl (1.14 mL, 4.57 mmol) in dioxane was added. the reaction was stirred at room temperature for 30 min, The crude reaction mixture was concentrated in vacuo. The crude material was purified by preparative HPLC to afford N-(4-(4-(1-(2-aminoethyl) piperidine-4-carbonyl)piperazine-1-carbonyl)-3-chlorophenyl)-5-(2,3-difluoro-4-methoxyphenyl)-1-methyl-1H-imidazole-2-carboxamide formate (19 mg, 29.5% yield). MS (ESI, m/z): 644.3 [M+H]⁺.

The following Type III Examples were prepared in analogy to example 290.

| | | | ESI MS | |
|---|---|---|---|---|
| Ex. | Name | Structure | [M + H]⁺ | Starting Material |
| 291 | N-[3-chloro-4-[4- (pyrrolidin-3- ylmethyl)piperazine- 1-carbonyl]phenyl]- 5-(2,3-difluoro-4- methoxy-phenyl)-1- methyl-imidazole-2- carboxamide formate | 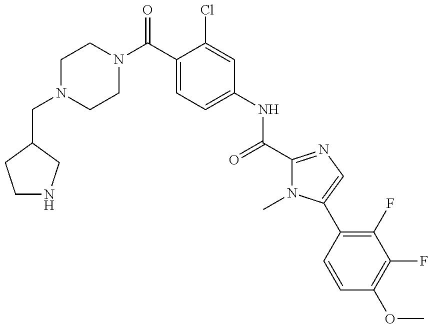 [SMILES: COc1ccc(-c2cnc(C(=O)Nc3ccc(C(=O)N4CCN(CC5CCNC5)CC4)c(Cl)c3)n2C)c(F)c1F] | 573.1 | Intermediate 374 and tert-butyl 3- formylpyrrolidine-1- carboxylate |
| 292 | N-[4-[4-[1-(2- aminoethyl)piperidine- 4- carbonyl]piperazine- 1-carbonyl]-3-chloro- phenyl]-5-[4- (difluoromethoxy)- 2,3-difluoro-phenyl]- 1-methyl-imidazole- 2-carboxamide trifluoroacetate | 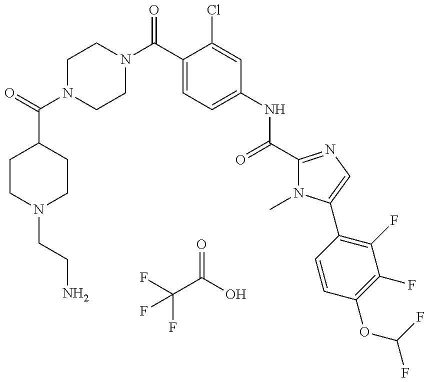 [SMILES: Cn1c(-c2ccc(OC(F)F)c(F)c2F)cnc1C(=O)Nc1ccc(C(=O)N2CCN(C(=O)C3CCN(CCN)CC3)CC2)c(Cl)c1 ; O=C(O)C(F)(F)F] | 680.2 | Example 249 and tert-butyl (2- oxoethyl)carbamate |

### Example Type III: 293

### N-[3-Chloro-4-[4-(2-pyrrolidin-1-ylacetyl)piperazine-1-carbonyl]phenyl]-5-(2,3-difluoro-4-methoxy-phenyl)-1-methyl-imidazole-2-carboxamide formate

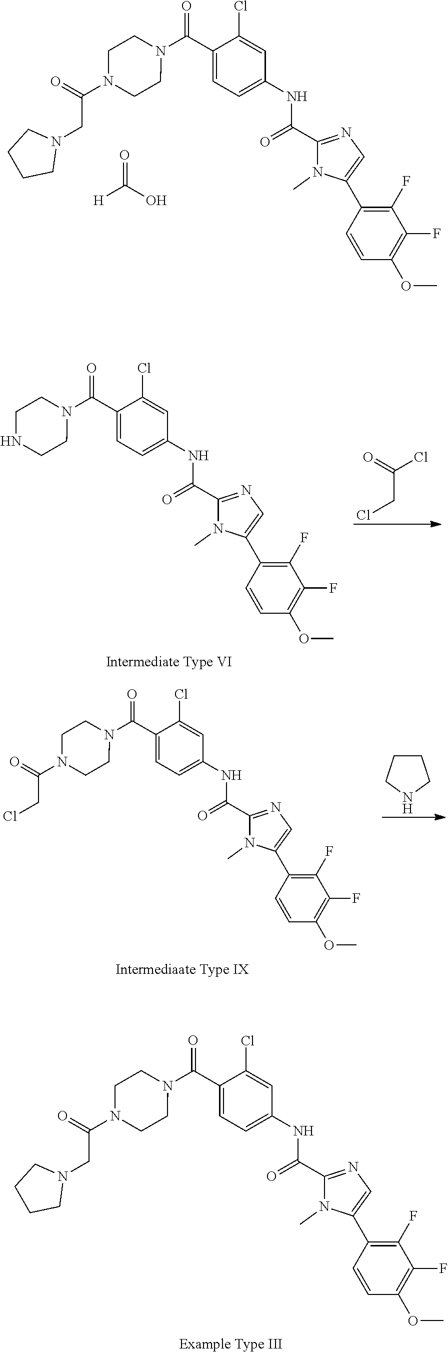

### Step 1 Intermediate 447:

### N-(3-chloro-4-(4-(2-chloroacetyl)piperazine-1-carbonyl)phenyl)-5-(2,3-difluoro-4-methoxyphenyl)-1-methyl-1H-imidazole-2-carboxamide

In a 100 mL round-bottomed flask, N-(3-chloro-4-(piperazine-1-carbonyl)phenyl)-5-(2,3-difluoro-4-methoxyphenyl)-1-methyl-1H-imidazole-2-carboxamide (500 mg, 1.02 mmol) and DIPEA (264 mg, 357 μl, 2.04 mmol) were combined with CH₂Cl₂ (20 mL) to give a light brown solution. 2-chloroacetyl chloride (138 mg, 1.22 mmol) was added. The reaction was stirred at room temperature for 20 min. The crude reaction mixture was concentrated in vacuo. The reaction mixture was poured into 50 mL H₂O and extracted with DCM (3×25 mL). The organic layers were combined, washed with sat NaCl (1×50 mL), The crude reaction mixture was concentrated in vacuo to afford N-(3-chloro-4-(4-(2-chloroacetyl)piperazine-1-carbonyl)phenyl)-5-(2,3-difluoro-4-methoxyphenyl)-1-methyl-1H-imidazole-2-carboxamide (530 mg, 936 μmol, 91.7% yield) which was used directly in the next step. (ESI, m/z): 566.0 [M+H]⁺.

### Step 2

### N-[3-chloro-4-[4-(2-pyrrolidin-1-ylacetyl)piperazine-1-carbonyl]phenyl]-5-(2,3-difluoro-4-methoxy-phenyl)-1-methyl-imidazole-2-carboxamide formate

To a 5 mL microwave vial were added N-(3-chloro-4-(4-(2-chloroacetyl)piperazine-1-carbonyl)phenyl)-5-(2,3-difluoro-4-methoxyphenyl)-1-methyl-1H-imidazole-2-carboxamide (75 mg, 132 μmol), pyrrolidine (47.1 mg, 662 μmol), DIEA (17.1 mg, 23.1 μl, 132 μmol) and sodium iodide (3.97 mg, 26.5 μmol) in acetonitrile (3 mL). The vial was capped and heated in the microwave at 80° C. for 30 min. The crude reaction mixture was concentrated in vacuo. The crude material was purified by preparative HPLC to afford N-[3-chloro-4-[4-(2-pyrrolidin-1-yl acetyl)piperazine-1-carbonyl]phenyl]-5-(2,3-difluoro-4-methoxy-phenyl)-1-methyl-imidazole-2-carboxamide formate (19 mg, 21.7% yield). (ESI, m/z): 601.3 [M+H]⁺.

The following Type III Examples were prepared in analogy to example example 293.

| | | | MS | |
|---|---|---|---|---|
| | | | ESI | Starting |
| Ex. | Name | Structure | [M + H]⁺ | Material |
| 294 | N-[3-chloro-4-[4-[2-(3- hydroxypyrrolidin-1- yl)acetyl]piperazine-1- carbonyl]phenyl]-5-(2,3- difluoro-4-methoxy- phenyl)- 1-methyl-imidazole-2- carboxamide formate | 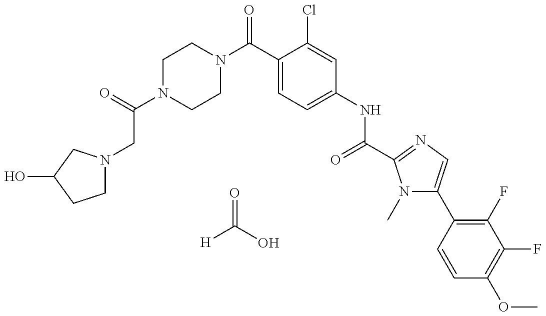 [SMILES: COc1ccc(-c2cnc(C(=O)Nc3ccc(C(=O)N4CCN(C(=O)CN5CCC(O)C5)CC4)c(Cl)c3)n2C)c(F)c1F ; O=CO] | 617.3 | Intermediate 447 and pyrrolidin-3-ol |
| 295 | N-[3-chloro-4-[4-[2-(3- hydroxyazetidin-1- yl)acetyl]piperazine-1- carbonyl]phenyl]-5-(2,3- difluoro-4-methoxy- phenyl)- 1-methyl-imidazole-2- carboxamide formate | 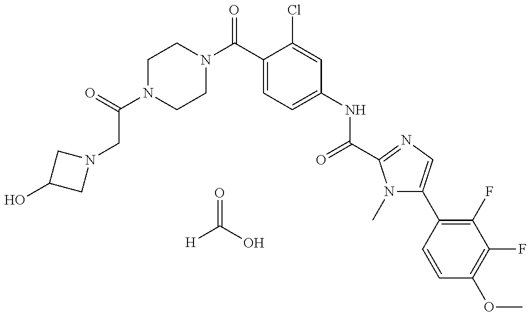 [SMILES: COc1ccc(-c2cnc(C(=O)Nc3ccc(C(=O)N4CCN(C(=O)CN5CC(O)C5)CC4)c(Cl)c3)n2C)c(F)c1F ; O=CO] | 603.2 | Intermediate 447 and azetidin-3-ol |

### Example Type III: 299

### N-[3-Chloro-4-[4-[3-[2-[[2-(dimethylamino)acetyl]amino]ethoxy]propanoyl]piperazine-1-carbonyl]phenyl]-5-(2,3-difluoro-4-methoxy-phenyl)-1-methyl-imidazole-2-carboxamide

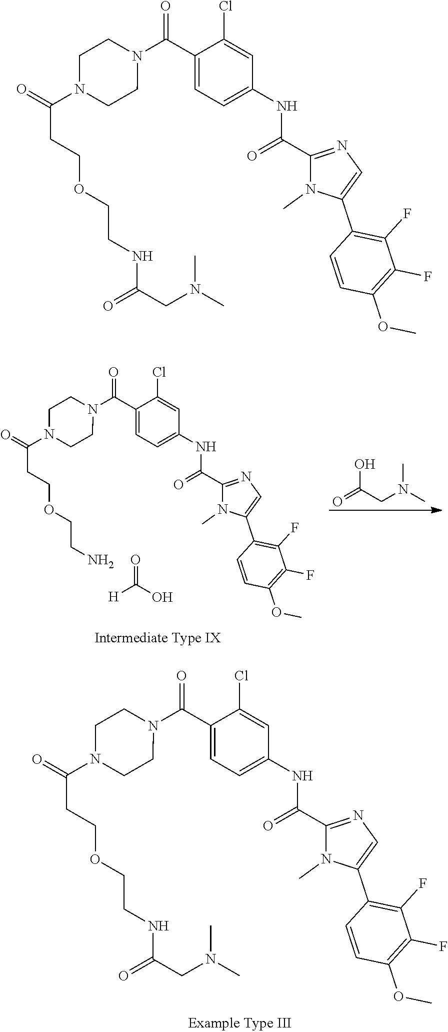

A mixture of N-[4-[4-[3-(2-aminoethoxy)propanoyl]piperazine-1-carbonyl]-3-chloro-phenyl]-5-(2,3-difluoro-4-methoxy-phenyl)-1-methyl-imidazole-2-carboxamide formate.

(246, 90 mg, 138 μmol), dimethylglycine (28.5 mg, 276 μmol), HATU (233 mg, 612 μmol) and DIPEA (158 mg, 214 μl, 1.22 mmol) in DMF (5 mL) was stirred overnight. The mixture was poured into water. The water layer was extracted with DCM. The organic layer was washed with water, dried and concentrated. The residue was purified by Prep-HPLC to give the title compound (29 mg). MS (ESI, m/z): 690.3 [M+H]⁺.

### Assay Procedures

### Antimicrobial Susceptibility Testing:

### 90% Growth Inhibitory Concentration (IC90) Determination

The in vitro antimicrobial activity of the compounds was determined according to the following procedure:

The assay used a 10-points Iso-Sensitest broth medium to measure quantitatively the in vitro activity of the compounds against Acinetobacter baumannii ATCC17978 or ATCC17961.

Stock compounds in DMSO were serially twofold diluted (e.g. range from 10 to 0.02 μM final concentration) in 384 wells microtiter plates and inoculated with 49 μl the bacterial suspension in Iso-Sensitest medium to have a final cell concentration of ˜5×10⁽⁵⁾ CFU/ml in a final volume/well of 50 ul/well. Microtiter plates were incubated at 35±2° C.

Bacterial cell growth was determined with the measurement of optical density at k=600 nm each 20 minutes over a time course of 16 h. Growth inhibition was calculated during the logarithmic growth of the bacterial cells with determination of the concentration inhibiting 50% (IC50) and 90% (IC90) of the growth.

Table 1 provides the 90% growth inhibitory concentrations (IC90) in micromoles per liter of the compounds of present invention obtained against the strain Acinetobacter baumannii ATCC17978 or ATCC17961.

Particular compounds of the present invention exhibit an IC90 (Acinetobacter baumannii ATCC17978 and/or ATCC17961)≤25 μmol/l.

More particular compounds of the present invention exhibit an IC90 (Acinetobacter baumannii ATCC17978 and/or ATCC17961)≤5 μmol/l.

Most particular compounds of the present invention exhibit an IC90 (Acinetobacter baumannii ATCC17978 and/or ATCC17961)≤1 μmol/l.

TABLE 1

| Example | |
|---|---|
| | ATCC |
| | 17978 |
| | IC90 |
| | [uM] |
| 16 | 0.30 |
| 20 | 0.07 |
| 27 | 5.70 |
| 73 | 0.06 |
| 74 | 0.10 |
| 75 | 0.10 |
| 76 | 0.21 |
| 77 | 0.17 |
| 78 | 0.12 |
| 79 | 0.21 |
| 80 | 0.07 |
| 81 | 0.07 |
| 104 | 0.07 |
| 105 | 0.07 |
| 106 | 0.10 |
| 107 | 0.08 |
| 113 | 0.08 |
| 114 | 0.11 |
| 115 | 0.14 |
| 116 | 0.13 |
| 117 | 0.15 |
| 196 | 0.07 |
| 197 | 0.07 |
| 198 | 0.03 |
| 199 | 0.04 |
| 200 | 0.05 |
| 201 | 0.05 |
| 202 | 0.09 |
| 203 | 0.09 |
| 204 | 0.04 |
| 205 | 0.06 |
| 206 | 0.10 |
| 207 | 0.05 |
| 269 | 0.07 |
| 270 | 0.05 |
| 274 | 0.29 |
| 275 | 1.52 |
| 276 | 4.02 |
| 277 | 2.3 |
| 278 | 0.08 |
| | ATCC |
| | 17961 |
| | IC90 |
| | [uM] |
| 7 | 0.07 |
| 11 | 0.07 |
| 13 | 0.11 |
| 14 | 0.13 |
| 17 | 0.03 |
| 18 | 0.07 |
| 19 | 0.06 |
| 21 | 0.1 |
| 22 | 0.04 |
| 23 | 0.06 |
| 24 | 0.07 |
| 25 | 0.06 |
| 26 | 0.03 |
| 28 | 0.17 |
| 29 | 0.19 |
| 30 | 0.17 |
| 31 | 0.07 |
| 32 | 0.08 |
| 33 | 0.09 |
| 34 | 0.07 |
| 35 | 0.06 |
| 36 | 0.13 |
| 37 | 0.17 |
| 38 | 0.06 |
| 39 | 0.07 |
| 40 | 0.07 |
| 41 | 0.14 |
| 42 | 0.17 |
| 43 | 0.09 |
| 44 | 0.03 |
| 45 | 0.08 |
| 46 | 0.07 |
| 47 | 0.04 |
| 48 | 0.04 |
| 49 | 0.15 |
| 50 | 0.05 |
| 51 | 0.03 |
| 52 | 0.13 |
| 53 | 0.05 |
| 54 | 0.04 |
| 55 | 0.05 |
| 56 | 0.07 |
| 57 | 0.06 |
| 58 | 0.05 |
| 59 | 0.05 |
| 60 | 0.05 |
| 61 | 0.06 |
| 62 | 0.06 |
| 63 | 0.09 |
| 64 | 0.06 |
| 65 | 0.05 |
| 66 | 0.14 |
| 67 | 3.30 |
| 68 | 2.10 |
| 69 | 0.12 |
| 70 | 0.14 |
| 71 | 0.47 |
| 72 | 0.05 |
| 82 | 0.07 |
| 83 | 0.17 |
| 84 | 0.09 |
| 85 | 0.15 |
| 86 | 0.1 |
| 87 | 0.12 |
| 88 | 0.05 |
| 89 | 0.05 |
| 90 | 0.04 |
| 91 | 0.04 |
| 92 | 0.06 |
| 93 | 0.04 |
| 94 | 0.04 |
| 95 | 0.05 |
| 96 | 0.04 |
| 97 | 0.26 |
| 99 | 0.48 |
| 100 | 0.34 |
| 101 | 0.05 |
| 102 | 0.09 |
| 103 | 0.06 |
| 108 | 0.04 |
| 109 | 0.15 |
| 110 | 0.18 |
| 111 | 0.3 |
| 112 | 0.23 |
| 118 | 0.14 |
| 119 | 0.12 |
| 120 | 0.06 |
| 121 | 0.04 |
| 122 | 0.09 |
| 123 | 0.04 |
| 124 | 0.04 |
| 125 | 0.11 |
| 126 | 0.11 |
| 127 | 0.1 |
| 128 | 0.27 |
| 129 | 0.04 |
| 130 | 0.26 |
| 131 | 0.09 |
| 132 | 0.07 |
| 133 | 0.17 |
| 134 | 0.1 |
| 135 | 0.03 |
| 136 | 0.03 |
| 137 | 0.04 |
| 138 | 0.03 |
| 139 | 0.04 |
| 140 | 0.05 |
| 141 | 0.06 |
| 142 | 0.04 |
| 143 | 0.03 |
| 144 | 0.06 |
| 145 | 0.04 |
| 146 | 0.04 |
| 147 | 0.04 |
| 148 | 0.04 |
| 149 | 0.03 |
| 150 | 0.08 |
| 151 | 0.04 |
| 152 | 0.05 |
| 153 | 0.06 |
| 154 | 0.07 |
| 155 | 0.04 |
| 156 | 0.03 |
| 157 | 0.03 |
| 158 | 0.04 |
| 159 | 0.07 |
| 160 | 0.04 |
| 161 | 0.03 |
| 162 | 0.03 |
| 163 | 0.04 |
| 164 | 0.07 |
| 165 | 0.05 |
| 166 | 0.05 |
| 167 | 0.05 |
| 168 | 0.15 |
| 169 | 0.03 |
| 170 | 0.06 |
| 171 | 0.05 |
| 172 | 0.05 |
| 173 | 0.05 |
| 174 | 0.07 |
| 175 | 0.05 |
| 176 | 0.08 |
| 177 | 0.05 |
| 178 | 0.07 |
| 179 | 0.05 |
| 180 | 0.06 |
| 181 | 0.13 |
| 182 | 0.07 |
| 183 | 0.08 |
| 184 | 0.02 |
| 185 | 0.06 |
| 186 | 0.1 |
| 187 | 0.11 |
| 188 | 0.14 |
| 189 | 0.14 |
| 190 | 0.18 |
| 191 | 0.23 |
| 192 | 0.46 |
| 193 | 2.59 |
| 194 | 0.05 |
| 195 | 0.09 |
| 208 | 0.05 |
| 209 | 0.08 |
| 210 | 0.06 |
| 211 | 0.05 |
| 212 | 0.85 |
| 213 | 0.1 |
| 214 | 0.07 |
| 215 | 0.8 |
| 216 | 0.12 |
| 217 | 0.09 |
| 218 | 0.09 |
| 219 | 0.11 |
| 220 | 0.04 |
| 221 | 0.05 |
| 222 | 0.09 |
| 223 | 0.05 |
| 224 | 0.03 |
| 225 | 0.16 |
| 226 | 0.13 |
| 227 | 0.09 |
| 228 | 0.18 |
| 229 | 0.09 |
| 230 | 0.1 |
| 231 | 0.16 |
| 232 | 0.09 |
| 233 | 0.13 |
| 234 | 0.18 |
| 235 | 0.06 |
| 236 | 0.06 |
| 237 | 0.05 |
| 238 | 0.13 |
| 239 | 0.15 |
| 240 | 0.32 |
| 241 | 0.15 |
| 242 | 0.09 |
| 243 | 0.11 |
| 244 | 0.1 |
| 245 | 0.03 |
| 246 | 0.1 |
| 247 | 0.03 |
| 248 | 0.05 |
| 249 | 0.03 |
| 250 | 0.08 |
| 251 | 0.05 |
| 252 | 0.06 |
| 253 | 0.05 |
| 254 | 0.04 |
| 255 | 0.59 |
| 256 | 8.70 |
| 257 | 0.23 |
| 258 | 1.12 |
| 259 | 0.05 |
| 260 | 0.25 |
| 261 | 0.08 |
| 262 | 0.07 |
| 263 | 6.06 |
| 264 | 0.09 |
| 265 | 0.07 |
| 268 | 0.07 |
| 271 | 0.64 |
| 272 | 0.88 |
| 273 | 1.30 |
| 279 | 0.15 |
| 280 | 0.1 |
| 281 | 0.06 |
| 282 | 0.06 |
| 283 | 0.04 |
| 284 | 0.08 |
| 285 | 0.1 |
| 286 | 0.18 |
| 287 | 0.14 |
| 288 | 0.47 |
| 289 | 0.07 |
| 290 | 0.05 |
| 291 | 0.05 |
| 292 | 0.04 |
| 293 | 0.03 |
| 294 | 0.04 |
| 295 | 0.11 |
| 297 | 0.12 |
| 299 | 0.15 |

### Example A

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Example C

A compound of formula (I) can be used in a manner known per se as the active ingredient for the production of an infusion solution of the following composition:

| Active ingredient | 100 mg |
|---|---|
| Lactic acid 90% | 100 mg |
| NaOH q.s. or HCl q.s. for adjustment to pH 4.0 | |
| Sodium chloride q.s. or glucose q.s. for | |
| adjustment of the osmolality to 290 mOsm/kg | |
| Water for injection (WFI) | ad 100 ml |

### Example D

A compound of formula (I) can be used in a manner known per se as the active ingredient for the production of an infusion solution of the following composition:

| | | |
|---|---|---|
| Active ingredient | 100 | mg |
| Hydroxypropyl-beta-cyclodextrin | 10 | g |

| NaOH q.s. or HCl q.s. for adjustment to pH 7.4 | |
|---|---|
| Sodium chloride q.s. or glucose q.s. for | |
| adjustment of the osmolality to 290 mOsm/kg | |
| Water for injection (WFI) | ad 100 ml |

1. A compound of formula (I) 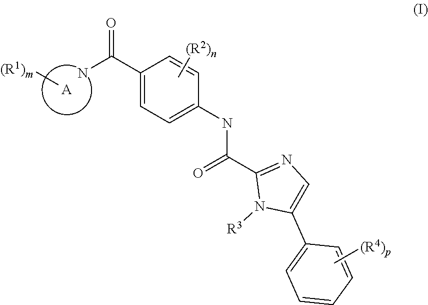
or a pharmaceutically acceptable salt thereof, wherein:
R¹ is, at each occurrence, independently selected from hydroxy, halogen, cyano, amino, C₁-C₆-alkoxy, halo-C₁-C₆-alkoxy, amino-C₁-C₆-alkoxy-, a group 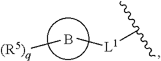
and a group C₁-C₆-alkyl-L²—; wherein C₁-C₆-alkyl is optionally substituted with 1-3 substituents selected from hydroxy, amino, halogen, cyano, C₁-C₆-alkyl-NH—, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy;
R² is, at each occurrence, independently selected from halogen, cyano, C₁-C₆-alkyl, C₁-C₆-alkoxy, halo-C₁-C₆-alkyl, and halo-C₁-C₆-alkoxy;
R³ is selected from hydrogen, C₁-C₆-alkyl, and halo-C₁-C₆-alkyl;
R⁴ is, at each occurrence, independently selected from halogen, C₁-C₆-alkyl, C₁-C₆-alkoxy, cyano, halo-C₁-C₆-alkyl, cyano-C₁-C₆-alkyl, (C₁-C₆-alkyl)₂N—, halo-C₁-C₆-alkoxy, cyano-C₁-C₆-alkoxy, C₁-C₆-alkoxy-C₁-C₆-alkoxy-, (C₁-C₆-alkyl)₂N—C(O)—, and 5- to 14-membered heteroaryloxy; and
R⁵ is, at each occurrence, independently selected from amino, hydroxy, C₁-C₆-alkyl, amino-C₁-C₆-alkyl-, hydroxy-C₁-C₆-alkyl-, halo-C₁-C₆-alkyl-, C₁-C₆-alkoxy, halo-C₁-C₆-alkoxy, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen (fluoro), cyano, C₁-C₆-aminoalkyl-S(O)₂—, and a group 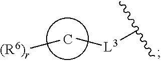
R⁶ is at each occurrence, independently selected from amino, hydroxy, C₁-C₆-alkyl, amino-C₁-C₆-alkyl-, hydroxy-C₁-C₆-alkyl-, halo-C₁-C₆-alkyl-, C₁-C₆-alkoxy, halo-C₁-C₆-alkoxy, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, cyano, and C₁-C₆-aminoalkyl-S(O)₂—;
A is 3- to 14-membered heterocyclyl;
B and C are independently selected from 3- to 14-membered heterocyclyl, C₃-C₁₀-cycloalkyl, 5- to 14-membered heteroaryl, and C₆-C₁₀-aryl;
L¹ and L³ are independently selected from a covalent bond, —O—, —NH—, —N(C₁-C₆-alkyl)-, —CH₂O—, —OCH₂—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—;
L² is selected from a covalent bond, carbonyl, —S(O)₂—, —NHC(O)—, —C(O)NH—, and —S(O)₂NH—;
m is 1, 2, 3, or 4;
n is 0, 1, 2, 3, or 4;
p is 0, 1, 2, 3, 4, or 5;
q is 0, 1, or 2;
r is 0 or 1; and
s is 0, 1, 2, 3, or 4.

2. The compound of formula (I) according to claim 1, or a pharmaceutically acceptable salt thereof, wherein:
m is 1; and
R¹ is selected from amino, amino-C₁-C₆-alkoxy-, a group 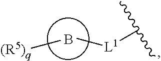
and a group C₁-C₆-alkyl-L²—; wherein:
C₁-C₆-alkyl is substituted with 1-2 substituents selected from hydroxy, amino, cyano, C₁-C₆-alkyl-NH—, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy;
L¹ is selected from —CH₂O—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—;
L² is selected from a covalent bond, carbonyl, —S(O)₂—, —NHC(O)—, —C(O)NH—, and —S(O)₂NH—;
q is 0, 1, or 2;
s is 0, 1, or 4;
B is selected from 3- to 14-membered heterocyclyl, C₃-C₁₀-cycloalkyl, and 5- to 14-membered heteroaryl; and
R⁵ is, at each occurrence, independently selected from amino, hydroxy, C₁-C₆-alkyl, amino-C₁-C₆-alkyl-, hydroxy-C₁-C₆-alkyl-, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, aminoalkyl-S(O)₂—, and a group 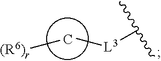
wherein:
L³ is a covalent bond or carbonyl;
r is 0 or 1;
C is C₃-C₁₀-cycloalkyl or 3- to 14-membered heterocyclyl; and
R⁶ is hydroxy.

3. The compound of formula (I) according to claim 1, or a pharmaceutically acceptable salt thereof, wherein:
m is 1; and
R¹ is a group 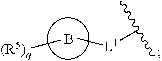
wherein:
L¹ is selected from carbonyl, —CH₂C(O)—, —CH₂NHC(O)—, and —NHC(O)—;
q is 0 or 1;
B is 3- to 14-membered heterocyclyl; and
R⁵ is selected from amino, hydroxy, and hydroxy-C₁-C₆-alkyl-.

4. The compound of formula (I) according to claim 1, or a pharmaceutically acceptable salt thereof, wherein:
m is 1; and
R¹ is a group 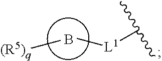
wherein:
L¹ is selected from carbonyl, —CH₂C(O)—, —CH₂NHC(O)—, and —NHC(O)—;
q is 0 or 1;
B is selected from azetidinyl, pyrrolidinyl, 3-azabicyclo[3.1.0]hexanyl, and piperidyl; and
R⁵ is selected from amino, hydroxy, and hydroxymethyl.

5. The compound of formula (I) according to any one of claims 1 to 4, or a pharmaceutically acceptable salt thereof, wherein n is 1 and R² is selected from halogen and C₁-C₆-alkyl.

6. The compound of formula (I) according to any one of claims 1 to 4, or a pharmaceutically acceptable salt thereof, wherein n is 1 and R² is selected from chloro and methyl.

7. The compound of formula (I) according to any one of claims 1 to 6, or a pharmaceutically acceptable salt thereof, wherein the compound of formula (I) is a compound of formula (II):

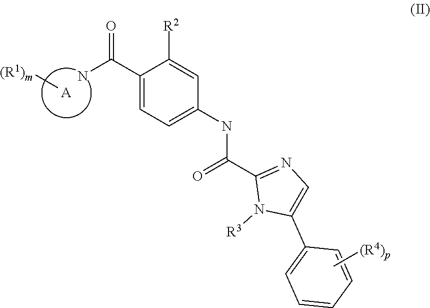

8. The compound of formula (I) according to any one of claims 1 to 7, or a pharmaceutically acceptable salt thereof, wherein R³ is C₁-C₆-alkyl.

9. The compound of formula (I) according to any one of claims 1 to 7, or a pharmaceutically acceptable salt thereof, wherein R³ is methyl.

10. The compound of formula (I) according to any one of claims 1 to 9, or a pharmaceutically acceptable salt thereof, wherein p is 1, 2, 3 or 4 and R⁴ is, at each occurrence, independently selected from halogen, C₁-C₆-alkyl, C₁-C₆-alkoxy, cyano, halo-C₁-C₆-alkyl, cyano-C₁-C₆-alkyl, (C₁-C₆-alkyl)₂N—, halo-C₁-C₆-alkoxy, cyano-C₁-C₆-alkoxy, C₁-C₆-alkoxy-C₁-C₆-alkoxy-, (C₁-C₆-alkyl)₂N—C(O)—, and heteroaryloxy.

11. The compound of formula (I) according to any one of claims 1 to 9, or a pharmaceutically acceptable salt thereof, wherein p is 2 or 3 and R⁴ is, at each occurrence, independently selected from halogen, C₁-C₆-alkoxy, halo-C₁-C₆-alkoxy, and cyano-C₁-C₆-alkoxy.

12. The compound of formula (I) according to any one of claims 1 to 9, or a pharmaceutically acceptable salt thereof, wherein p is 2 or 3 and R⁴ is, at each occurrence, independently selected from fluoro, chloro, methoxy, FCH₂O—, F₂CHO— and CNCH₂O—.

13. The compound of formula (I) according to any one of claims 1 to 9, or a pharmaceutically acceptable salt thereof, wherein the compound of formula (I) is a compound of formula (III): 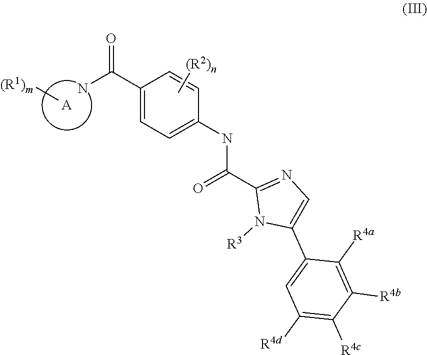
wherein:
R^{4a} is selected from hydrogen, halogen, C₁-C₆-alkyl, cyano, and halo-C₁-C₆-alkyl;
R^{4b} is selected from hydrogen, halogen, cyano, and C₁-C₆-alkoxy;
R^{4c} is selected from halogen, C₁-C₆-alkoxy, cyano-C₁-C₆-alkyl, cyano-C₁-C₆-alkoxy, (C₁-C₆-alkyl)₂N—, halo-C₁-C₆-alkoxy, C₁-C₆-alkoxy-C₁-C₆-alkoxy, (C₁-C₆-alkyl)₂N—C(O)—, and heteroaryloxy; and
R^{4d} is selected from hydrogen and halogen.

14. The compound of formula (III) according to claim 13, or a pharmaceutically acceptable salt thereof, wherein:
R^{4a} is halogen;
R^{4b} is selected from hydrogen and halogen;
R^{4c} is selected from C₁-C₆-alkoxy, cyano-C₁-C₆-alkoxy, and halo-C₁-C₆-alkoxy; and
R^{4d} is hydrogen.

15. The compound of formula (III) according to claim 13, or a pharmaceutically acceptable salt thereof, wherein:
R^{4a} is selected from fluoro and chloro;
R^{4b} is selected from hydrogen, fluoro and chloro;
R^{4c} is selected from methoxy, FCH₂O—, F₂CHO— and CNCH₂O—; and
R^{4d} is hydrogen.

16. The compound of formula (I) according to claim 1, or a pharmaceutically acceptable salt thereof, wherein:
m is 1; and
R¹ is selected from amino, amino-C₁-C₆-alkoxy-, a group 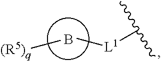
and a group C₁-C₆-alkyl-L²—; wherein:
C₁-C₆-alkyl is substituted with 1-2 substituents selected from hydroxy, amino, cyano, C₁-C₆-alkyl-NH—, (C₁-C₆-alkyl)₂N—, amino-C₁-C₆-alkoxy-C₁-C₆-alkoxy-, carbamoyl, carbamoyl-C₁-C₆-alkoxy-, carbamimidoyl, (C₁-C₆-alkyl)₂N—C₁-C₆-alkoxy-, (C₁-C₆-alkyl)₂N—C₁-C₆-alkyl-C(O)—NH—C₁-C₆-alkoxy-, C₂-C₆-alkynyl-NH—, carboxy, and C₁-C₆-alkoxy;
L¹ is selected from —CH₂O—, —(CH₂)ₛC(O)—, —CH₂NHC(O)—, —CH₂C(O)NH—, —CH₂—, —NHC(O)—, —S(O)₂—, —S(O)₂NH—, —C(O)NH(CH₂)₂—, and —NH—NHC(O)—;
L² is selected from a covalent bond, carbonyl, —S(O)₂—, —NHC(O)—, —C(O)NH—, and —S(O)₂NH—;
q is 0, 1, or 2;
s is 0, 1, or 4;
B is selected from 3- to 14-membered heterocyclyl, C₃-C₁₀-cycloalkyl, and 5- to 14-membered heteroaryl; and
R⁵ is, at each occurrence, independently selected from amino, hydroxy, C₁-C₆-alkyl, amino-C₁-C₆-alkyl-, hydroxy-C₁-C₆-alkyl-, (C₁-C₆-alkyl)₂N—, (C₁-C₆-alkyl)₂N—C₁-C₆-alkyl-, (C₁-C₆-alkyl)₂N—C₁-C₆-alkyl-C(O)—, oxo, carbamoyl, carbamoyl-C₁-C₆-alkyl, carboxy, carboxy-C₁-C₆-alkyl, halogen, aminoalkyl-S(O)₂—, and a group 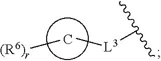
wherein:
L³ is a covalent bond or carbonyl;
r is 0 or 1;
C is C₃-C₁₀-cycloalkyl or 3- to 14-membered heterocyclyl;
R⁶ is hydroxy;
n is 1;
R² is selected from halogen and C₁-C₆-alkyl;
R³ is C₁-C₆-alkyl;
p is 1, 2, 3 or 4; and
R⁴ is, at each occurrence, independently selected from halogen, C₁-C₆-alkyl, C₁-C₆-alkoxy, cyano, halo-C₁-C₆-alkyl, cyano-C₁-C₆-alkyl, (C₁-C₆-alkyl)₂N—, halo-C₁-C₆-alkoxy, cyano-C₁-C₆-alkoxy, C₁-C₆-alkoxy-C₁-C₆-alkoxy-, (C₁-C₆-alkyl)₂N—C(O)—, and heteroaryloxy.

17. The compound of formula (I) according to claim 1, or a pharmaceutically acceptable salt thereof, wherein:
m is 1; and
R¹ is a group 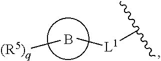
wherein:
L¹ is selected from carbonyl, —CH₂C(O)—, —CH₂NHC(O)—, and —NHC(O)—;
q is 0 or 1;
B is 3- to 14-membered heterocyclyl; and
R⁵ is selected from amino, hydroxy, and hydroxy-C₁-C₆-alkyl;
n is 1;
R² is selected from halogen and C₁-C₆-alkyl;
R³ is C₁-C₆-alkyl;
p is 2 or 3; and
R⁴ is, at each occurrence, independently selected from halogen, C₁-C₆-alkoxy, halo-C₁-C₆-alkoxy, and cyano-C₁-C₆-alkoxy.

18. The compound of formula (I) according to claim 1, or a pharmaceutically acceptable salt thereof, wherein:
m is 1; and
R¹ is a group 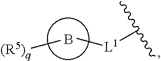
wherein:
L¹ is selected from carbonyl, —CH₂C(O)—, —CH₂NHC(O)—, and —NHC(O)—;
q is 0 or 1;
B is selected from azetidinyl, pyrrolidinyl, 3-azabicyclo[3.1.0]hexanyl, and piperidyl; and
R⁵ is selected from amino, hydroxy, and hydroxymethyl;
n is 1;
R² is selected from chloro and methyl;
R³ is methyl;
p is 2 or 3; and
R⁴ is, at each occurrence, independently selected from fluoro, chloro, methoxy, FCH₂O—, F₂CHO— and CNCH₂O—.

19. The compound of formula (I) according to claim 1, or a pharmaceutically acceptable salt thereof, wherein the compound of formula (I) is selected from:
N-[3-chloro-4-[4-[(3R)-pyrrolidine-3-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(3S)-pyrrolidine-3-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,4S)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-(piperidine-4-carbonyl)piperazine-1-carbonyl]phenyl]-5-[4-(cyanomethoxy)-2,3-difluoro-phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,3S)-3-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[3-(dimethylamino)propyl]piperazine-1-carbonyl]phenyl]-5-[4-(cyanomethoxy)-2,3-difluoro-phenyl]-1-methyl-imidazole-2-carboxamide;
1-[2-chloro-4-[[5-[4-(cyanomethoxy)-2,3-difluoro-phenyl]-1-methyl-imidazole-2-carbonyl]amino]benzoyl]-N-[2-(methylamino)ethyl]piperidine-4-carboxamide;
N-[4-[4-[(3R)-3-aminopyrrolidine-1-carbonyl]piperidine-1-carbonyl]-3-chloro-phenyl]-5-[4-(cyanomethoxy)-2,3-difluoro-phenyl]-1-methyl-imidazole-2-carboxamide;
N-[3-chloro-4-[4-(4-hydroxypiperidine-4-carbonyl)piperazine-1-carbonyl]phenyl]-5-[4-(cyanomethoxy)-2,3-difluoro-phenyl]-1-methyl-imidazole-2-carboxamide;
N-[4-[4-[2-(aminomethyl)morpholine-4-carbonyl]piperidine-1-carbonyl]-3-chloro-phenyl]-5-[4-(cyanomethoxy)-2,3-difluoro-phenyl]-1-methyl-imidazole-2-carboxamide;
N-[3-chloro-4-[4-[(3R)-3-(hydroxymethyl)piperazine-1-carbonyl]piperidine-1-carbonyl]phenyl]-5-[4-(cyanomethoxy)-2,3-difluoro-phenyl]-1-methyl-imidazole-2-carboxamide;
N-[3-chloro-4-[4-[2-(dimethylamino)ethyl]piperazine-1-carbonyl]phenyl]-5-(2,3-difluoro-4-methoxy-phenyl)-1-methyl-imidazole-2-carboxamide;
N-[4-[4-[(2S,4S)-4-aminopyrrolidine-2-carbonyl]piperazine-1-carbonyl]-3-chloro-phenyl]-5-[4-(cyanomethoxy)-2,3-difluoro-phenyl]-1-methyl-imidazole-2-carboxamide;
N-[3-chloro-4-[4-[(2S)-pyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[2-(dimethylamino)acetyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S)-piperidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(3S)-piperidine-3-carbonyl]piperazine-1-carbonyl]phenyl]-5-[4-(cyanomethoxy)-2,3-difluoro-phenyl]-1-methyl-imidazole-2-carboxamide;
N-[4-[4-(2-aminoethyl)piperazine-1-carbonyl]-3-chloro-phenyl]-5-[4-(cyanomethoxy)-2,3-difluoro-phenyl]-1-methyl-imidazole-2-carboxamide;
N-[3-chloro-4-[4-[2-(methylamino)acetyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2R)-pyrrolidine-2-carbonyl]piperazine-1-carbonyl]phenyl]-5-[4-(cyanomethoxy)-2,3-difluoro-phenyl]-1-methyl-imidazole-2-carboxamide;
N-(3-amino-2-hydroxy-propyl)-1-[2-chloro-4-[[5-[4-(cyanomethoxy)-2,3-difluoro-phenyl]-1-methyl-imidazole-2-carbonyl]amino]benzoyl]piperidine-4-carboxamide;
5-[4-(cyanomethoxy)-2,3-difluoro-phenyl]-1-methyl-N-[3-methyl-4-[4-[(3R)-pyrrolidine-3-carbonyl]piperazine-1-carbonyl]phenyl]imidazole-2-carboxamide;
N-[3-chloro-4-[4-[(3S)-3-(hydroxymethyl)piperazine-1-carbonyl]piperidine-1-carbonyl]phenyl]-5-[4-(cyanomethoxy)-2,3-difluoro-phenyl]-1-methyl-imidazole-2-carboxamide;
N-[3-chloro-4-[4-(1,1-dioxo-1,4-thiazinane-4-carbonyl)piperidine-1-carbonyl]phenyl]-5-[4-(cyanomethoxy)-2,3-difluoro-phenyl]-1-methyl-imidazole-2-carboxamide;
5-[4-(cyanomethoxy)-2,3-difluoro-phenyl]-1-methyl-N-[3-methyl-4-[4-[2-(methylamino)acetyl]piperazine-1-carbonyl]phenyl]imidazole-2-carboxamide;
N-[3-chloro-4-[4-(morpholine-4-carbonyl)piperidine-1-carbonyl]phenyl]-5-[4-(cyanomethoxy)-2,3-difluoro-phenyl]-1-methyl-imidazole-2-carboxamide;
5-[4-(cyanomethoxy)-2,3-difluoro-phenyl]-1-methyl-N-[3-methyl-4-[4-(morpholine-4-carbonyl)piperidine-1-carbonyl]phenyl]imidazole-2-carboxamide;
N-[3-chloro-4-[4-(2,6-diazaspiro[3.3]heptane-2-carbonyl)piperidine-1-carbonyl]phenyl]-5-[4-(cyanomethoxy)-2,3-difluoro-phenyl]-1-methyl-imidazole-2-carboxamide;
5-[4-(cyanomethoxy)-2,3-difluoro-phenyl]-N-[4-[4-(1,1-dioxo-1,4-thiazinane-4-carbonyl)piperidine-1-carbonyl]-3-methyl-phenyl]-1-methyl-imidazole-2-carboxamide;
N-[4-[4-[(2R)-2-(aminomethyl)morpholine-4-carbonyl]piperidine-1-carbonyl]-3-methyl-phenyl]-5-[4-(cyanomethoxy)-2,3-difluoro-phenyl]-1-methyl-imidazole-2-carboxamide;
N-[3-chloro-4-[4-(thiomorpholine-4-carbonyl)piperidine-1-carbonyl]phenyl]-5-[4-(cyanomethoxy)-2,3-difluoro-phenyl]-1-methyl-imidazole-2-carboxamide;
N-[4-[4-(3-aminoazetidine-1-carbonyl)piperidine-1-carbonyl]-3-methyl-phenyl]-5-[4-(cyanomethoxy)-2,3-difluoro-phenyl]-1-methyl-imidazole-2-carboxamide;
N-[4-[4-(aminomethyl)piperidine-1-carbonyl]-3-chloro-phenyl]-5-(2,3-difluoro-4-methoxy-phenyl)-1-methyl-imidazole-2-carboxamide;
N-[3-chloro-4-[4-(5-hydroxypiperidine-3-carbonyl)piperazine-1-carbonyl]phenyl]-5-[4-(cyanomethoxy)-2,3-difluoro-phenyl]-1-methyl-imidazole-2-carboxamide;
N-(2-aminoethyl)-4-[2-chloro-4-[[5-[4-(cyanomethoxy)-2,3-difluoro-phenyl]-1-methyl-imidazole-2-carbonyl]amino]benzoyl]piperazine-1-carboxamide;
N-[3-chloro-4-(4-piperazin-1-ylsulfonylpiperidine-1-carbonyl)phenyl]-5-(2,3-difluoro-4-methoxy-phenyl)-1-methyl-imidazole-2-carboxamide;
N-[3-chloro-4-[4-[2-[[(2S)-pyrrolidine-2-carbonyl]amino]ethyl]piperazine-1-carbonyl]phenyl]-5-[4-(cyanomethoxy)-2,3-difluoro-phenyl]-1-methyl-imidazole-2-carboxamide;
N-[4-[4-[(2S)-2-(aminomethyl)morpholine-4-carbonyl]piperidine-1-carbonyl]-3-methyl-phenyl]-5-[4-(cyanomethoxy)-2,3-difluoro-phenyl]-1-methyl-imidazole-2-carboxamide;
N-[3-chloro-4-[4-(4-piperidyl sulfonyl)piperazine-1-carbonyl]phenyl]-difluoro-4-methoxy-phenyl)-1-methyl-imidazole-2-carboxamide;
N-[4-[4-[2-[(2-amino-2-oxo-ethyl)amino]ethyl]piperazine-1-carbonyl]-3-chloro-phenyl]-5-[4-(cyanomethoxy)-2,3-difluoro-phenyl]-1-methyl-imidazole-2-carboxamide;
(3R)-1-[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]pyrrolidine-3-carboxylic acid;
1-[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]azetidine-3-carboxylic acid;
5-[3-chloro-4-(cyanomethoxy)-2-fluoro-phenyl]-N-[3-chloro-4-[4-(piperidine-4-carbonyl)piperazine-1-carbonyl]phenyl]-1-methyl-imidazole-2-carboxamide;
N-[3-chloro-4-[4-(piperidine-4-carbonyl)piperazine-1-carbonyl]phenyl]difluoro-4-methoxy-phenyl)-1-methyl-imidazole-2-carboxamide;
N-[3-chloro-4-[4-[2-(dimethylamino)ethyl]piperazine-1-carbonyl]phenyl]-5-(3-fluoro-4-methoxy-phenyl)-1-methyl-imidazole-2-carboxamide;
N-[3-chloro-4-[4-[rac-(1R,5S)-3-azabicyclo[3.1.0]hexane-6-carbonyl]piperazine-1-carbonyl]phenyl]-5-[2,3-difluoro-4-(fluoromethoxy)phenyl]-1-methyl-imidazole-2-carboxamide;
N-[4-[4-(2-aminoethyl)piperidine-1-carbonyl]-3-chloro-phenyl]-5-(2,3-difluoro-4-methoxy-phenyl)-1-methyl-imidazole-2-carboxamide;
N-[3-chloro-4-[4-[(3R)-pyrrolidine-3-carbonyl]piperazine-1-carbonyl]phenyl]-5-(2,3-difluoro-4-methoxy-phenyl)-1-methyl-imidazole-2-carboxamide;
N-[4-[4-(3-aminocyclobutanecarbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-[(3S)-pyrrolidine-3-carbonyl]piperazine-1-carbonyl]phenyl]-5-(2,3-difluoro-4-methoxy-phenyl)-1-methyl-imidazole-2-carboxamide;
N-[4-[4-(3-aminocyclobutanecarbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(3-hydroxypyrrolidine-3-carbonyl)piperazine-1-carbonyl]phenyl]-5-(2,3-difluoro-4-methoxy-phenyl)-1-methyl-imidazole-2-carboxamide;
N-[3-chloro-4-[4-[2-(4-piperidyl)acetyl]piperazine-1-carbonyl]phenyl]-difluoro-4-methoxy-phenyl)-1-methyl-imidazole-2-carboxamide;
N-[3-chloro-4-[4-(piperidine-4-carbonyl)piperazine-1-carbonyl]phenyl]-5-[2,3-difluoro-4-(fluoromethoxy)phenyl]-1-methyl-imidazole-2-carboxamide;
N-[3-chloro-4-[4-(piperidine-4-carbonyl)piperazine-1-carbonyl]phenyl]-5-[4-(difluoromethoxy)-3-fluoro-phenyl]-1-methyl-imidazole-2-carboxamide;
N-[4-[4-[2-(azetidin-3-yl)acetyl]piperazine-1-carbonyl]-3-chloro-phenyl]-difluoro-4-methoxy-phenyl)-1-methyl-imidazole-2-carboxamide;
5-[2-chloro-3-fluoro-4-(fluoromethoxy)phenyl]-N-[3-chloro-4-[4-[2-[(3S)-pyrrolidin-3-yl]acetyl]piperazine-1-carbonyl]phenyl]-1-methyl-imidazole-2-carboxamide;
1-[2-chloro-4-[[5-[2-chloro-3-fluoro-4-(fluoromethoxy)phenyl]-1-methyl-imidazole-2-carbonyl]amino]benzoyl]-N-[(3R,4R)-4-hydroxypyrrolidin-3-yl]piperidine-4-carboxamide;
5-[2-chloro-4-(difluoromethoxy)-3-fluoro-phenyl]-N-[3-chloro-4-[4-[2-[(3S)-pyrrolidin-3-yl]acetyl]piperazine-1-carbonyl]phenyl]-1-methyl-imidazole-2-carboxamide;
N-[3-chloro-4-[4-(4-hydroxypyrrolidine-3-carbonyl)piperazine-1-carbonyl]phenyl]-5-[2,3-difluoro-4-(fluoromethoxy)phenyl]-1-methyl-imidazole-2-carboxamide;
1-[2-chloro-4-[[5-[2,3-difluoro-4-(fluoromethoxy)phenyl]-1-methyl-imidazole-2-carbonyl]amino]benzoyl]-N-[[(3R)-pyrrolidin-3-yl]methyl]piperidine-4-carboxamide;
N-[4-[4-(2-aminoacetyl)piperazine-1-carbonyl]-3-chloro-phenyl]-5-(2,3-difluoro-4-methoxy-phenyl)-1-methyl-imidazole-2-carboxamide;
N-[4-[4-[(2S)-azetidine-2-carbonyl]piperazine-1-carbonyl]-3-chloro-phenyl]-5-(2,3-difluoro-4-methoxy-phenyl)-1-methyl-imidazole-2-carboxamide;
N-[3-chloro-4-[4-(2-pyrrolidin-1-ylacetyl)piperazine-1-carbonyl]phenyl]-difluoro-4-methoxy-phenyl)-1-methyl-imidazole-2-carboxamide;
N-[3-chloro-4-[4-(2-pyrrolidin-3-ylacetyl)piperazine-1-carbonyl]phenyl]-difluoro-4-methoxy-phenyl)-1-methyl-imidazole-2-carboxamide;
N-[3-chloro-4-[4-[2-[(2R)-pyrrolidin-2-yl]acetyl]piperazine-1-carbonyl]phenyl]-5-(2,3-difluoro-4-methoxy-phenyl)-1-methyl-imidazole-2-carboxamide;
N-[3-chloro-4-[4-[2-[(3R)-pyrrolidin-3-yl]acetyl]piperazine-1-carbonyl]phenyl]-5-(2,3-difluoro-4-methoxy-phenyl)-1-methyl-imidazole-2-carboxamide;
N-[3-chloro-4-[4-[2-[(2S)-pyrrolidin-2-yl]acetyl]piperazine-1-carbonyl]phenyl]-5-(2,3-difluoro-4-methoxy-phenyl)-1-methyl-imidazole-2-carboxamide;
N-[3-chloro-4-[4-[(3R)-pyrrolidine-3-carbonyl]piperazine-1-carbonyl]phenyl]-5-[2,3-difluoro-4-(fluoromethoxy)phenyl]-1-methyl-imidazole-2-carboxamide;
N-[3-chloro-4-[4-[2-(dimethylamino)acetyl]piperazine-1-carbonyl]phenyl]-5-[4-(difluoromethoxy)-2,3-difluoro-phenyl]-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[[(3R)-pyrrolidin-3-yl]methyl]piperidine-4-carboxamide;
1-[2-chloro-4-[[5-(2,3-difluoro-4-methoxy-phenyl)-1-methyl-imidazole-2-carbonyl]amino]benzoyl]-N-[[(3S)-pyrrolidin-3-yl]methyl]piperidine-4-carboxamide;
N-[3-chloro-4-[4-[2-(3-hydroxypyrrolidin-1-yl)acetyl]piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3-hydroxyazetidin-3-yl)methyl]piperidine-4-carboxamide;
N-[3-chloro-4-[4-[2-[(2S)-pyrrolidin-2-yl]acetyl]piperazine-1-carbonyl]phenyl]-5-[2,3-difluoro-4-(fluoromethoxy)phenyl]-1-methyl-imidazole-2-carboxamide;
N-[3-chloro-4-[4-[2-[(3S)-pyrrolidin-3-yl]acetyl]piperazine-1-carbonyl]phenyl]-5-[2,3-difluoro-4-(fluoromethoxy)phenyl]-1-methyl-imidazole-2-carboxamide;
N-[3-chloro-4-[4-[2-[(2S)-pyrrolidin-2-yl]acetyl]piperazine-1-carbonyl]phenyl]-5-[4-(difluoromethoxy)-2-fluoro-phenyl]-1-methyl-imidazole-2-carboxamide;
N-[3-chloro-4-[4-[2-(3-hydroxyazetidin-3-yl)acetyl]piperazine-1-carbonyl]phenyl]-5-[2,3-difluoro-4-(fluoromethoxy)phenyl]-1-methyl-imidazole-2-carboxamide;
N-[3-chloro-4-[4-[2-(4-hydroxy-4-piperidyl)acetyl]piperazine-1-carbonyl]phenyl]-5-(2,3-difluoro-4-methoxy-phenyl)-1-methyl-imidazole-2-carboxamide;
N-[3-chloro-4-[4-(4-hydroxypiperidine-4-carbonyl)piperazine-1-carbonyl]phenyl]-5-[2,3-difluoro-4-(fluoromethoxy)phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-5-[4-(difluoromethoxy)-2,3-difluoro-phenyl]-1-methyl-imidazole-2-carboxamide;
N-[4-[4-[1-(2-aminoethyl)piperidine-4-carbonyl]piperazine-1-carbonyl]-3-chloro-phenyl]-5-(2,3-difluoro-4-methoxy-phenyl)-1-methyl-imidazole-2-carboxamide;
N-[3-chloro-4-[4-[2-(4-hydroxy-4-piperidyl)acetyl]piperazine-1-carbonyl]phenyl]-5-[2,3-difluoro-4-(fluoromethoxy)phenyl]-1-methyl-imidazole-2-carboxamide;
N-[4-[4-(azetidine-3-carbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(pyrrolidin-3-ylmethyl)piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3S)-pyrrolidin-3-yl]piperidine-4-carboxamide;
1-[2-chloro-4-[[5-(2,3-difluoro-4-methoxy-phenyl)-1-methyl-imidazole-2-carbonyl]amino]benzoyl]-N-[rac-(1S,5R)-3-azabicyclo[3.1.0]hexan-6-yl]piperidine-4-carboxamide;
N-[3-chloro-4-[4-(piperidine-4-carbonyl)piperazine-1-carbonyl]phenyl]-5-[4-(dimethylamino)-2,3-difluoro-phenyl]-1-methyl-imidazole-2-carboxamide;
N-[3-chloro-4-[4-(4-hydroxypiperidine-4-carbonyl)piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3R,4R)-4-hydroxypyrrolidin-3-yl]piperidine-4-carboxamide;
5-(2-chloro-3-fluoro-4-methoxy-phenyl)-N-[3-chloro-4-[4-[2-[(3S)-pyrrolidin-3-yl]acetyl]piperazine-1-carbonyl]phenyl]-1-methyl-imidazole-2-carboxamide;
N-[3-chloro-4-[4-(piperidine-4-carbonyl)piperazine-1-carbonyl]phenyl]-1-methyl-5-(2,3,5-trifluoro-4-methoxy-phenyl)imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[rac-(1S,5R)-3-azabicyclo[3.1.0]hexan-6-yl]piperidine-4-carboxamide;
N-[4-[3-(2-aminoethoxy)azetidine-1-carbonyl]-3-chloro-phenyl]-5-(2,3-difluoro-4-methoxy-phenyl)-1-methyl-imidazole-2-carboxamide;
1-[2-chloro-4-[[5-[2-chloro-3-fluoro-4-(fluoromethoxy)phenyl]-1-methyl-imidazole-2-carbonyl]amino]benzoyl]-N-[[(3R)-pyrrolidin-3-yl]methyl]piperidine-4-carboxamide;
5-[2-chloro-4-(difluoromethoxy)-3-fluoro-phenyl]-N-[3-chloro-4-[4-(4-hydroxypiperidine-4-carbonyl)piperazine-1-carbonyl]phenyl]-1-methyl-imidazole-2-carboxamide;
N-[3-chloro-4-[4-(4-hydroxypiperidine-4-carbonyl)piperazine-1-carbonyl]phenyl]-5-(4-ethoxy-2,3-difluoro-phenyl)-1-methyl-imidazole-2-carboxamide;
N-[3-chloro-4-[4-(5-hydroxypiperidine-3-carbonyl)piperazine-1-carbonyl]phenyl]-5-[2,3-difluoro-4-(fluoromethoxy)phenyl]-1-methyl-imidazole-2-carboxamide;
5-[2-chloro-4-(difluoromethoxy)-3-fluoro-phenyl]-N-[3-chloro-4-[4-[2-(4-hydroxy-4-piperidyl)acetyl]piperazine-1-carbonyl]phenyl]-1-methyl-imidazole-2-carboxamide;
5-[2-chloro-3-fluoro-4-(fluoromethoxy)phenyl]-N-[3-chloro-4-[4-[2-(4-hydroxy-4-piperidyl)acetyl]piperazine-1-carbonyl]phenyl]-1-methyl-imidazole-2-carboxamide;
N-[4-[4-[1-(azetidine-3-carbonyl)piperidine-4-carbonyl]piperazine-1-carbonyl]-3-chloro-phenyl]-5-[4-(difluoromethoxy)-2,3-difluoro-phenyl]-1-methyl-imidazole-2-carboxamide;
N-[3-chloro-4-[4-[2-(dimethylamino)acetyl]piperazine-1-carbonyl]phenyl]-5-[4-(difluoromethoxy)-2-fluoro-phenyl]-1-methyl-imidazole-2-carboxamide;
N-[3-chloro-4-[4-[2-(dimethylamino)acetyl]piperazine-1-carbonyl]phenyl]-5-[4-(difluoromethoxy)-3-fluoro-phenyl]-1-methyl-imidazole-2-carboxamide;
N-(azetidin-2-ylmethyl)-1-[2-chloro-4-[[5-(2,3-difluoro-4-methoxy-phenyl)-1-methyl-imidazole-2-carbonyl]amino]benzoyl]piperidine-4-carboxamide;
N-[3-chloro-4-[4-[2-(3-hydroxyazetidin-3-yl)acetyl]piperazine-1-carbonyl]phenyl]-5-(2,3-difluoro-4-methoxy-phenyl)-1-methyl-imidazole-2-carboxamide;
N-[4-[4-[2-(azetidin-3-yl)acetyl]piperazine-1-carbonyl]-3-chloro-phenyl]-5-[4-(difluoromethoxy)-2-fluoro-phenyl]-1-methyl-imidazole-2-carboxamide;
N-[3-chloro-4-[4-[1-(4-hydroxypiperidine-4-carbonyl)piperidine-4-carbonyl]piperazine-1-carbonyl]phenyl]-5-[4-(difluoromethoxy)-2,3-difluoro-phenyl]-1-methyl-imidazole-2-carboxamide;
N-[3-chloro-4-[4-(piperidine-4-carbonyl)piperazine-1-carbonyl]phenyl]-5-[4-(difluoromethoxy)-2-fluoro-phenyl]-1-methyl-imidazole-2-carboxamide;
N-[3-chloro-4-[4-[2-[(2S)-pyrrolidin-2-yl]acetyl]piperazine-1-carbonyl]phenyl]-5-[4-(difluoromethoxy)-3-fluoro-phenyl]-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3R)-pyrrolidin-3-yl]piperidine-4-carboxamide;
1-[2-chloro-4-[[5-[2-chloro-3-fluoro-4-(fluoromethoxy)phenyl]-1-methyl-imidazole-2-carbonyl]amino]benzoyl]-N-[(3S)-pyrrolidin-3-yl]piperidine-4-carboxamide;
5-(2-chloro-3-fluoro-4-methoxy-phenyl)-N-[3-chloro-4-[4-[2-(4-hydroxy-4-piperidyl)acetyl]piperazine-1-carbonyl]phenyl]-1-methyl-imidazole-2-carboxamide;
N-[3-chloro-4-[4-[2-(4-hydroxy-4-piperidyl)acetyl]piperazine-1-carbonyl]phenyl]-5-(4-ethoxy-2,3-difluoro-phenyl)-1-methyl-imidazole-2-carboxamide;
N-[4-[3-[3-(aminomethyl)azetidine-1-carbonyl]azetidine-1-carbonyl]-3-chloro-phenyl]-5-(2,3-difluoro-4-methoxy-phenyl)-1-methyl-imidazole-2-carboxamide;
3-[[1-[2-chloro-4-[[5-(2,3-difluoro-4-methoxy-phenyl)-1-methyl-imidazole-2-carbonyl]amino]benzoyl]piperidine-4-carbonyl]amino]propanoic acid;
4-[[1-[2-chloro-4-[[5-(2,3-difluoro-4-methoxy-phenyl)-1-methyl-imidazole-2-carbonyl]amino]benzoyl]piperidine-4-carbonyl]amino]butanoic acid;
N-[3-chloro-4-[4-(piperidine-4-carbonyl)piperazine-1-carbonyl]phenyl]-5-[4-(dimethylcarbamoyl)-2,3-difluoro-phenyl]-1-methyl-imidazole-2-carboxamide;
N-[3-chloro-4-[4-[rac-(1R,5S)-3-azabicyclo[3.1.0]hexane-6-carbonyl]piperazine-1-carbonyl]phenyl]-5-(2,3-difluoro-4-methoxy-phenyl)-1-methyl-imidazole-2-carboxamide;
N-[4-[4-[3-(aminomethyl)cyclobutanecarbonyl]piperazine-1-carbonyl]-3-chloro-phenyl]-5-(2,3-difluoro-4-methoxy-phenyl)-1-methyl-imidazole-2-carboxamide;
N-[4-[4-(2-amino-2-oxo-ethyl)piperazine-1-carbonyl]-3-chloro-phenyl]-5-(2,3-difluoro-4-methoxy-phenyl)-1-methyl-imidazole-2-carboxamide;
N-[3-chloro-4-[rac-(3aR,6aS)-5-(piperidine-4-carbonyl)-1,3,3a,4,6,6a-hexahydropyrrolo[3,4-c]pyrrole-2-carbonyl]phenyl]-5-(2,3-difluoro-4-methoxy-phenyl)-1-methyl-imidazole-2-carboxamide;
N-[3-chloro-4-[4-[2-(3-hydroxyazetidin-1-yl)acetyl]piperazine-1-carbonyl]phenyl]-5-(2,3-difluoro-4-methoxy-phenyl)-1-methyl-imidazole-2-carboxamide;
N-[1-[2-chloro-4-[[5-(2,3-difluoro-4-methoxy-phenyl)-1-methyl-imidazole-2-carbonyl]amino]benzoyl]azetidin-3-yl]piperidine-4-carboxamide;
N-[3-chloro-4-[3-(piperazine-1-carbonyl)azetidine-1-carbonyl]phenyl]-5-(2,3-difluoro-4-methoxy-phenyl)-1-methyl-imidazole-2-carboxamide;
N-[3-chloro-4-[rac-(3aR,6aS)-5-[rac-(3R)-pyrrolidine-3-carbonyl]-1,3,3a,4,6,6a-hexahydropyrrolo[3,4-c]pyrrole-2-carbonyl]phenyl]-5-(2,3-difluoro-4-methoxy-phenyl)-1-methyl-imidazole-2-carboxamide;
N-[3-chloro-4-[3-[[2-(dimethylamino)acetyl]amino]azetidine-1-carbonyl]phenyl]-5-(2,3-difluoro-4-methoxy-phenyl)-1-methyl-imidazole-2-carboxamide;
N-[3-chloro-4-[3-[rac-(3aR,6aS)-2,3,3a,4,6,6a-hexahydro-1H-pyrrolo[3,4-c]pyrrole-5-carbonyl]azetidine-1-carbonyl]phenyl]-5-(2,3-difluoro-4-methoxy-phenyl)-1-methyl-imidazole-2-carboxamide;
N-[3-chloro-4-[rac-(3aS,6aR)-2-[2-(dimethylamino)acetyl]-1,3,3a,4,6,6a-hexahydropyrrolo[3,4-c]pyrrole-5-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-1-methyl-5-(2,3,4-trifluorophenyl)imidazole-2-carboxamide;
N-[3-chloro-4-[4-(piperidine-4-carbonyl)piperazine-1-carbonyl]phenyl]-5-(3-cyano-4-methoxy-phenyl)-1-methyl-imidazole-2-carboxamide;
N-[3-chloro-4-[3-[rac-(3aR,6aR)-2,3,3a,4,6,6a-hexahydro-1H-pyrrolo[3,4-b]pyrrole-5-carbonyl]pyrrolidine-1-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-(3-cyano-2,4-difluoro-phenyl)-1-methyl-imidazole-2-carboxamide;
N-[3-chloro-4-[4-[2-[(3S)-pyrrolidin-3-yl]acetyl]piperazine-1-carbonyl]phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-[(3S,4S)-4-hydroxypyrrolidin-3-yl]piperidine-4-carboxamide;
N-[3-chloro-4-[4-[[2-(dimethylamino)acetyl]amino]piperidine-1-carbonyl]phenyl]-5-(2,3-difluoro-4-methoxy-phenyl)-1-methyl-imidazole-2-carboxamide;
1-[4-[[5-(2,3-difluoro-4-methoxy-phenyl)-1-methyl-imidazole-2-carbonyl]amino]-2-ethyl-benzoyl]-N-[3-(prop-2-ynyl amino)propyl]piperidine-4-carboxamide;
5-(2,3-difluoro-4-methoxy-phenyl)-N-[4-[4-[4-[3-(dimethyl amino)propyl]piperazine-1-carbonyl]piperidine-1-carbonyl]-3-ethyl-phenyl]-1-methyl-imidazole-2-carboxamide;
N-(2-aminoethyl)-1-[4-[[5-(2,3-difluoro-4-methoxy-phenyl)-1-methyl-imidazole-2-carbonyl]amino]-2-ethyl-benzoyl]piperidine-4-carboxamide;
1-[4-[[5-(2,3-difluoro-4-methoxy-phenyl)-1-methyl-imidazole-2-carbonyl]amino]-2-ethyl-benzoyl]-N-[2-[2-(dimethylamino)ethoxy]ethyl]piperidine-4-carboxamide;
5-[4-(difluoromethoxy)phenyl]-N-[4-[4-[2-(dimethylamino)ethyl]piperazine-1-carbonyl]-3-ethyl-phenyl]-1-methyl-imidazole-2-carboxamide;
5-(2-chloro-4-methoxy-phenyl)-N-[4-[4-[2-(dimethyl amino)ethyl]piperazine-1-carbonyl]-3-ethyl-phenyl]-1-methyl-imidazole-2-carboxamide;
N-[4-[4-[2-(dimethyl amino)ethyl]piperazine-1-carbonyl]-3-ethyl-phenyl]-5-(3-fluoro-4-isopropoxy-phenyl)-1-methyl-imidazole-2-carboxamide;
N-[4-[4-[(3-cyclobutyl-1,2,4-oxadiazol-5-yl)methyl]piperidine-1-carbonyl]-3-ethyl-phenyl]-5-(2,3-difluoro-4-methoxy-phenyl)-1-methyl-imidazole-2-carboxamide;
4-[2-chloro-4-[[5-[4-(cyanomethoxy)-2,3-difluoro-phenyl]-1-methyl-imidazole-2-carbonyl]amino]benzoyl]-N-[(3S,4S)-4-hydroxypyrrolidin-3-yl]piperazine-1-carboxamide;
4-[2-chloro-4-[[5-[4-(cyanomethoxy)-2,3-difluoro-phenyl]-1-methyl-imidazole-2-carbonyl]amino]benzoyl]-N-[(3R,4R)-4-hydroxypyrrolidin-3-yl]piperazine-1-carboxamide;
4-[2-chloro-4-[[5-(2,3-difluoro-4-methoxy-phenyl)-1-methyl-imidazole-2-carbonyl]amino]benzoyl]-N-[(3S,4S)-4-hydroxypyrrolidin-3-yl]piperazine-1-carboxamide;
N-[3-chloro-4-[4-[(3S,4S)-3-hydroxypiperidine-4-carbonyl]piperazine-1-carbonyl]phenyl]-5-(2,3-difluoro-4-methoxy-phenyl)-1-methyl-imidazole-2-carboxamide;
N-[3-chloro-4-[4-[(2S,4S)-4-hydroxy-4-methyl-pyrrolidine-2-carbonyl]piperazine-1-carbonyl]phenyl]-difluoro-4-methoxy-phenyl)-1-methyl-imidazole-2-carboxamide;
N-[3-chloro-4-[4-[(3S,4R)-3-hydroxypiperidine-4-carbonyl]piperazine-1-carbonyl]phenyl]-difluoro-4-methoxy-phenyl)-1-methyl-imidazole-2-carboxamide;
4-[4-[[5-(2,3-difluoro-4-methoxy-phenyl)-1-methyl-imidazole-2-carbonyl]amino]-2-methyl-benzoyl]-N-[(3S,4S)-4-hydroxypyrrolidin-3-yl]piperazine-1-carboxamide;
N-[3-chloro-4-[4-[(2S,4S)-4-ethyl-4-hydroxy-pyrrolidine-2-carbonyl]piperazine-1-carbonyl]phenyl]-difluoro-4-methoxy-phenyl)-1-methyl-imidazole-2-carboxamide;
5-[4-(cyanomethoxy)-2,3-difluoro-phenyl]-N-[4-[4-[(2S,4S)-4-ethyl-4-hydroxy-pyrrolidine-2-carbonyl]piperazine-1-carbonyl]-3-methyl-phenyl]-1-methyl-imidazole-2-carboxamide;
5-[4-(cyanomethoxy)-2,3-difluoro-phenyl]-N-[4-[4-[(2S,4S)-4-hydroxy-4-methyl-pyrrolidine-2-carbonyl]piperazine-1-carbonyl]-3-methyl-phenyl]-1-methyl-imidazole-2-carboxamide;
N-[3-chloro-4-[4-[(3R,4S)-3-hydroxypiperidine-4-carbonyl]piperazine-1-carbonyl]phenyl]-difluoro-4-methoxy-phenyl)-1-methyl-imidazole-2-carboxamide;
5-(2,3-difluoro-4-methoxy-phenyl)-N-[4-[4-[(3R,4R)-3-hydroxypiperidine-4-carbonyl]piperazine-1-carbonyl]-3-methyl-phenyl]-1-methyl-imidazole-2-carboxamide;
N-[3-chloro-4-[4-[(3R,4R)-3-hydroxypiperidine-4-carbonyl]piperazine-1-carbonyl]phenyl]-difluoro-4-methoxy-phenyl)-1-methyl-imidazole-2-carboxamide;
4-[2-chloro-4-[[5-(2,3-difluoro-4-methoxy-phenyl)-1-methyl-imidazole-2-carbonyl]amino]benzoyl]-N-[(3R,4R)-4-hydroxypyrrolidin-3-yl]piperazine-1-carboxamide;
N-[3-chloro-4-(piperazine-1-carbonyl)phenyl]-5-[4-(cyanomethoxy)-2,3-difluoro-phenyl]-1-methyl-imidazole-2-carboxamide;
N-[3-chloro-4-[4-[(3S,4R)-3-hydroxypiperidine-4-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-(4-guanidinobutanoyl)piperazine-1-carbonyl]phenyl]-5-(2,3-difluoro-4-methoxy-phenyl)-1-methyl-imidazole-2-carboxamide;
N-[4-[4-(3-aminopropanoyl)piperazine-1-carbonyl]-3-chloro-phenyl]-5-(2,3-difluoro-4-methoxy-phenyl)-1-methyl-imidazole-2-carboxamide;
N-[4-[4-(5-aminopentanoyl)piperazine-1-carbonyl]-3-chloro-phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-[4-(difluoromethoxy)-2,3-difluoro-phenyl]-1-methyl-imidazole-2-carboxamide;
N-[3-chloro-4-[4-(3-cyanopropanoyl)piperazine-1-carbonyl]phenyl]-5-(2,3-difluoro-4-methoxy-phenyl)-1-methyl-imidazole-2-carboxamide;
N-[4-[4-(3-aminopropanoylamino)piperidine-1-carbonyl]-3-chloro-phenyl]-5-(2,3-difluoro-4-methoxy-phenyl)-1-methyl-imidazole-2-carboxamide;
N-(azetidin-3-yl)-1-[2-chloro-4-[[5-(2,3-difluoro-4-methoxy-phenyl)-1-methyl-imidazole-2-carbonyl]amino]benzoyl]piperidine-4-carboxamide;
N-[4-[4-[(1S,3R)-3-aminocyclopentanecarbonyl]piperazine-1-carbonyl]-3-chloro-phenyl]-5-(2,3-difluoro-4-methoxy-phenyl)-1-methyl-imidazole-2-carboxamide;
N-[4-[4-(2-aminoethylsulfonylamino)piperidine-1-carbonyl]-3-chloro-phenyl]-5-(2,3-difluoro-4-methoxy-phenyl)-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-(4-piperidyl)piperidine-4-carboxamide;
1-[2-chloro-4-[[5-(2,3-difluoro-4-methoxy-phenyl)-1-methyl-imidazole-2-carbonyl]amino]benzoyl]-N-(4-pyridylmethyl)piperidine-4-carboxamide;
5-[2-chloro-4-(cyanomethoxy)-3-fluoro-phenyl]-N-[3-chloro-4-[4-[(3R)-pyrrolidine-3-carbonyl]piperazine-1-carbonyl]phenyl]-1-methyl-imidazole-2-carboxamide;
5-[2-chloro-4-(cyanomethoxy)-3-fluoro-phenyl]-N-[3-chloro-4-[4-[(3S)-pyrrolidine-3-carbonyl]piperazine-1-carbonyl]phenyl]-1-methyl-imidazole-2-carboxamide;
1-[2-chloro-4-[[5-[2,3-difluoro-4-(fluoromethoxy)phenyl]-1-methyl-imidazole-2-carbonyl]amino]benzoyl]-N-[(3S,4R)-4-fluoropyrrolidin-3-yl]piperidine-4-carboxamide;
5-[3-chloro-2-fluoro-4-(fluoromethoxy)phenyl]-N-[3-chloro-4-[4-[(2S,4R)-4-hydroxypyrrolidine-2-carbonyl]piperazine-1-carbonyl]phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3S,4R)-4-fluoropyrrolidin-3-yl]piperidine-4-carboxamide;
1-[2-chloro-4-[[5-[4-(difluoromethoxy)-2,3-difluoro-phenyl]-1-methyl-imidazole-2-carbonyl]amino]benzoyl]-N-[[(3S)-pyrrolidin-3-yl]methyl]piperidine-4-carboxamide;
N-[4-[4-(2-aminoethoxy)piperidine-1-carbonyl]-3-chloro-phenyl]-5-(2,3-difluoro-4-methoxy-phenyl)-1-methyl-imidazole-2-carboxamide;
N-[4-[4-(azetidin-3-ylmethoxy)piperidine-1-carbonyl]-3-chloro-phenyl]-5-(2,3-difluoro-4-methoxy-phenyl)-1-methyl-imidazole-2-carboxamide;
N-[4-[4-(4-aminobutanoyl)piperazine-1-carbonyl]-3-chloro-phenyl]-5-(2,3-difluoro-4-methoxy-phenyl)-1-methyl-imidazole-2-carboxamide;
N-[3-chloro-4-[4-[2-(dimethyl amino)acetyl]piperazine-1-carbonyl]phenyl]-5-(2,3-difluoro-4-methoxy-phenyl)-1-methyl-imidazole-2-carboxamide;
N-[(2S)-2-aminopropyl]-1-[2-chloro-4-[[5-(2,3-difluoro-4-methoxy-phenyl)-1-methyl-imidazole-2-carbonyl]amino]benzoyl]piperidine-4-carboxamide;
N-[4-[4-[1-(2-aminoethyl)piperidine-4-carbonyl]piperazine-1-carbonyl]-3-chloro-phenyl]-5-[4-(difluoromethoxy)-2,3-difluoro-phenyl]-1-methyl-imidazole-2-carboxamide;
N-[3-chloro-4-[4-[1-[2-(dimethylamino)acetyl]piperidine-4-carbonyl]piperazine-1-carbonyl]phenyl]-5-(2,3-difluoro-4-methoxy-phenyl)-1-methyl-imidazole-2-carboxamide;
5-(2-chloro-3-fluoro-4-methoxy-phenyl)-N-[4-[4-(4-hydroxypiperidine-4-carbonyl)piperazine-1-carbonyl]-3-methyl-phenyl]-1-methyl-imidazole-2-carboxamide;
N-[4-[4-(4-aminopiperidine-1-carbonyl)piperidine-1-carbonyl]-3-chloro-phenyl]-5-(2,3-difluoro-4-methoxy-phenyl)-1-methyl-imidazole-2-carboxamide;
N-[3-chloro-4-[4-[2-[(3S)-pyrrolidin-3-yl]acetyl]piperazine-1-carbonyl]phenyl]-5-[2-(difluoromethyl)-3-fluoro-4-methoxy-phenyl]-1-methyl-imidazole-2-carboxamide;
N-[3-chloro-4-[4-(pyrrolidin-3-yl sulfonyl amino)piperidine-1-carbonyl]phenyl]-5-(2,3-difluoro-4-methoxy-phenyl)-1-methyl-imidazole-2-carboxamide;
N-[4-[4-[(3S)-3-aminopyrrolidine-1-carbonyl]piperidine-1-carbonyl]-3-chloro-phenyl]-5-[4-(difluoromethoxy)-2,3-difluoro-phenyl]-1-methyl-imidazole-2-carboxamide;
5-[2-chloro-4-(cyanomethoxy)-3-fluoro-phenyl]-N-[3-chloro-4-[4-(piperidine-4-carbonyl)piperazine-1-carbonyl]phenyl]-1-methyl-imidazole-2-carboxamide;
N-[4-[4-[(3R)-3-aminopyrrolidine-1-carbonyl]piperidine-1-carbonyl]-3-chloro-phenyl]-5-[4-(difluoromethoxy)-2,3-difluoro-phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[[(3R)-pyrrolidin-3-yl]methyl]piperidine-4-carboxamide;
N-[3-chloro-4-[4-(4-piperidyl sulfonylamino)piperidine-1-carbonyl]phenyl]-5-(2,3-difluoro-4-methoxy-phenyl)-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3S,4S)-4-hydroxypyrrolidin-3-yl]piperidine-4-carboxamide;
N-[3-chloro-4-[4-[3-(dimethylamino)azetidine-1-carbonyl]piperidine-1-carbonyl]phenyl]-5-[4-(difluoromethoxy)-2,3-difluoro-phenyl]-1-methyl-imidazole-2-carboxamide;
5-[2-chloro-4-(cyanomethoxy)-3-fluoro-phenyl]-N-[3-chloro-4-[4-(4-hydroxypiperidine-4-carbonyl)piperazine-1-carbonyl]phenyl]-1-methyl-imidazole-2-carboxamide;
N-[4-[4-(3-carbamoylpyrrolidine-1-carbonyl)piperidine-1-carbonyl]-3-chloro-phenyl]-5-[4-(difluoromethoxy)-2,3-difluoro-phenyl]-1-methyl-imidazole-2-carboxamide;
N-(azetidin-3-ylmethyl)-1-[2-chloro-4-[[5-(2,3-difluoro-4-methoxy-phenyl)-1-methyl-imidazole-2-carbonyl]amino]benzoyl]piperidine-4-carboxamide;
N-[4-[4-[1-(2-amino-2-oxo-ethyl)piperidine-4-carbonyl]piperazine-1-carbonyl]-3-chloro-phenyl]-5-[4-(difluoromethoxy)-2,3-difluoro-phenyl]-1-methyl-imidazole-2-carboxamide;
N-[4-[4-[1-(2-aminoethyl sulfonyl)piperidine-4-carbonyl]piperazine-1-carbonyl]-3-chloro-phenyl]-5-(2,3-difluoro-4-methoxy-phenyl)-1-methyl-imidazole-2-carboxamide;
N-[3-chloro-4-[4-[(2S,4S)-4-hydroxypyrrolidine-2-carbonyl]piperazine-1-carbonyl]phenyl]-5-(2,3-difluoro-4-methoxy-phenyl)-1-methyl-imidazole-2-carboxamide;
N-(azetidin-3-ylmethyl)-1-[2-chloro-4-[[5-[4-(difluoromethoxy)-2,3-difluoro-phenyl]-1-methyl-imidazole-2-carbonyl]amino]benzoyl]piperidine-4-carboxamide;
N-[3-chloro-4-(4-methylsulfonylpiperazine-1-carbonyl)phenyl]-5-(2,3-difluoro-4-methoxy-phenyl)-1-methyl-imidazole-2-carboxamide;
N-[3-chloro-4-[4-(methanesulfonamido)piperidine-1-carbonyl]phenyl]-5-(2,3-difluoro-4-methoxy-phenyl)-1-methyl-imidazole-2-carboxamide;
N-[4-[4-(2-aminoethyl sul fonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(2-oxoimidazolidine-4-carbonyl)piperazine-1-carbonyl]phenyl]-5-(2,3-difluoro-4-methoxy-phenyl)-1-methyl-imidazole-2-carboxamide;
N-[4-[4-[3-(2-aminoethyl)azetidine-1-carbonyl]piperidine-1-carbonyl]-3-chloro-phenyl]-5-(2,3-difluoro-4-methoxy-phenyl)-1-methyl-imidazole-2-carboxamide;
N-[3-chloro-4-[4-[(2-pyrrolidin-3-ylacetyl)amino]piperidine-1-carbonyl]phenyl]-5-(2,3-difluoro-4-methoxy-phenyl)-1-methyl-imidazole-2-carboxamide;
N-[3-chloro-4-[4-[2-[(3S)-pyrrolidin-3-yl]acetyl]piperazine-1-carbonyl]phenyl]-5-[3-fluoro-4-(fluoromethoxy)-2-methyl-phenyl]-1-methyl-imidazole-2-carboxamide;
5-[3-chloro-2-fluoro-4-(fluoromethoxy)phenyl]-N-[4-[4-[(2S,4R)-4-hydroxypyrrolidine-2-carbonyl]piperazine-1-carbonyl]-3-methyl-phenyl]-1-methyl-imidazole-2-carboxamide;
N-[4-[4-[(3S,4S)-3-amino-4-fluoro-pyrrolidine-1-carbonyl]piperidine-1-carbonyl]-3-chloro-phenyl]-5-(2,3-difluoro-4-methoxy-phenyl)-1-methyl-imidazole-2-carboxamide;
N-[3-chloro-4-(4-pyrrolidin-3-yl sulfonylpiperazine-1-carbonyl)phenyl]-5-(2,3-difluoro-4-methoxy-phenyl)-1-methyl-imidazole-2-carboxamide;
N-[4-[4-(3-aminobicyclo[1.1.1]pentane-1-carbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-[[(1-methyl-4-piperidyl)amino]carbamoyl]piperidine-1-carbonyl]phenyl]-5-[2,3-difluoro-4-(fluoromethoxy)phenyl]-1-methyl-imidazole-2-carboxamide;
N-[3-chloro-4-[4-[2-[(3S)-pyrrolidin-3-yl]acetyl]piperazine-1-carbonyl]phenyl]-5-(2-cyano-3-fluoro-4-methoxy-phenyl)-1-methyl-imidazole-2-carboxamide;
N-[3-chloro-4-[4-[2-(5-oxopyrrolidin-3-yl)acetyl]piperazine-1-carbonyl]phenyl]-5-(2,3-difluoro-4-methoxy-phenyl)-1-methyl-imidazole-2-carboxamide;
N-[3-chloro-4-[4-[3-(dimethylamino)propanoylamino]piperidine-1-carbonyl]phenyl]-5-(2,3-difluoro-4-methoxy-phenyl)-1-methyl-imidazole-2-carboxamide;
5-[2-chloro-4-(cyanomethoxy)-3-fluoro-phenyl]-N-[3-chloro-4-[4-[2-(dimethyl amino)ethyl]piperazine-1-carbonyl]phenyl]-1-methyl-imidazole-2-carboxamide;
5-[3-chloro-4-(cyanomethoxy)phenyl]-N-[3-chloro-4-[4-[2-(dimethyl amino)ethyl]piperazine-1-carbonyl]phenyl]-1-methyl-imidazole-2-carboxamide;
1-[2-chloro-4-[[5-(2,3-difluoro-4-methoxy-phenyl)-1-methyl-imidazole-2-carbonyl]amino]benzoyl]-N-(4-piperidylmethyl)piperidine-4-carboxamide;
N-[4-[4-[3-(aminomethyl)azetidine-1-carbonyl]piperidine-1-carbonyl]-3-chloro-phenyl]-5-(2,3-difluoro-4-methoxy-phenyl)-1-methyl-imidazole-2-carboxamide;
N-[3-chloro-4-[4-[2-(methylamino)acetyl]piperazine-1-carbonyl]phenyl]-5-(2,3-difluoro-4-methoxy-phenyl)-1-methyl-imidazole-2-carboxamide;
N-[4-[4-(3-aminoazetidine-1-carbonyl)piperidine-1-carbonyl]-3-chloro-phenyl]-5-(2,3-difluoro-4-methoxy-phenyl)-1-methyl-imidazole-2-carboxamide;
N-[3-chloro-4-[4-[[(2S,4R)-4-hydroxypyrrolidine-2-carbonyl]amino]piperidine-1-carbonyl]phenyl]-5-(2,3-difluoro-4-methoxy-phenyl)-1-methyl-imidazole-2-carboxamide;
N-[4-[4-[3-[2-(2-aminoethoxy)ethoxy]propanoyl]piperazine-1-carbonyl]-3-chloro-phenyl]-5-(2,3-difluoro-4-methoxy-phenyl)-1-methyl-imidazole-2-carboxamide;
N-[3-chloro-4-[4-[2-(5-oxopyrrolidin-2-yl)acetyl]piperazine-1-carbonyl]phenyl]-5-(2,3-difluoro-4-methoxy-phenyl)-1-methyl-imidazole-2-carboxamide;
N-[3-chloro-4-[4-(5-oxopyrrolidine-2-carbonyl)piperazine-1-carbonyl]phenyl]-5-(2,3-difluoro-4-methoxy-phenyl)-1-methyl-imidazole-2-carboxamide;
5-(2-chloro-3-fluoro-4-methoxy-phenyl)-N-[3-chloro-4-[4-[(2S,4S)-4-hydroxypyrrolidine-2-carbonyl]piperazine-1-carbonyl]phenyl]-1-methyl-imidazole-2-carboxamide;
N-[4-(4-aminopiperidine-1-carbonyl)-3-chloro-phenyl]-5-(2,3-difluoro-4-methoxy-phenyl)-1-methyl-imidazole-2-carboxamide;
N-[3-chloro-4-[4-[2-(methylamino)ethyl]piperazine-1-carbonyl]phenyl]-5-(2,3-difluoro-4-methoxy-phenyl)-1-methyl-imidazole-2-carboxamide;
N-[3-chloro-4-[4-(6-oxopiperidine-3-carbonyl)piperazine-1-carbonyl]phenyl]-5-(2,3-difluoro-4-methoxy-phenyl)-1-methyl-imidazole-2-carboxamide;
N-[4-[4-(2-azaspiro[3.3]heptane-6-carbonyl)piperazine-1-carbonyl]-3-methyl-phenyl]-5-(2-chloro-3-fluoro-4-methoxy-phenyl)-1-methyl-imidazole-2-carboxamide;
5-(2-chloro-3-fluoro-4-methoxy-phenyl)-N-[3-chloro-4-[4-[(3R)-3-(hydroxymethyl)piperazine-1-carbonyl]piperidine-1-carbonyl]phenyl]-1-methyl-imidazole-2-carboxamide;
5-(2-chloro-3-fluoro-4-methoxy-phenyl)-N-[3-chloro-4-[4-[(2R,4R)-4-hydroxypyrrolidine-2-carbonyl]piperazine-1-carbonyl]phenyl]-1-methyl-imidazole-2-carboxamide;
N-[3-chloro-4-[6-[(3R)-3-(hydroxymethyl)piperazine-1-carbonyl]-2-azaspiro[3.3]heptane-2-carbonyl]phenyl]-5-(2,3-difluoro-4-methoxy-phenyl)-1-methyl-imidazole-2-carboxamide;
N-[3-chloro-4-[4-[3-[2-[[2-(dimethylamino)acetyl]amino]ethoxy]propanoyl]piperazine-1-carbonyl]phenyl]-5-(2,3-difluoro-4-methoxy-phenyl)-1-methyl-imidazole-2-carboxamide;
5-(2-chloro-3-fluoro-4-methoxy-phenyl)-1-methyl-N-[3-methyl-4-[4-(piperidine-4-carbonyl)piperazine-1-carbonyl]phenyl]imidazole-2-carboxamide;
N-[3-chloro-4-[3-[[rac-(3R)-pyrrolidine-3-carbonyl]amino]pyrrolidine-1-carbonyl]phenyl]-difluoro-4-methoxy-phenyl)-1-methyl-imidazole-2-carboxamide;
N-[3-chloro-4-(2,6-diazaspiro[3.3]heptane-2-carbonyl)phenyl]-5-(2,3-difluoro-4-methoxy-phenyl)-1-methyl-imidazole-2-carboxamide;
N-[(1S)-2-amino-1-methyl-ethyl]-1-[2-chloro-4-[[5-(2,3-difluoro-4-methoxy-phenyl)-1-methyl-imidazole-2-carbonyl]amino]benzoyl]piperidine-4-carboxamide;
N-[3-chloro-4-[4-[(3R)-3-(hydroxymethyl)piperazine-1-carbonyl]piperidine-1-carbonyl]phenyl]-difluoro-4-methoxy-phenyl)-1-methyl-imidazole-2-carboxamide;
N-[1-[2-chloro-4-[[5-(2,3-difluoro-4-methoxy-phenyl)-1-methyl-imidazole-2-carbonyl]amino]benzoyl]pyrrolidin-3-yl]piperidine-4-carboxamide;
N-[3-chloro-4-[4-[(3S)-3-(hydroxymethyl)piperazine-1-carbonyl]piperidine-1-carbonyl]phenyl]-difluoro-4-methoxy-phenyl)-1-methyl-imidazole-2-carboxamide;
N-[3-chloro-4-[2-[(2S,4R)-4-hydroxypyrrolidine-2-carbonyl]-2,6-diazaspiro[3.3]heptane-6-carbonyl]phenyl]-5-(2,3-difluoro-4-methoxy-phenyl)-1-methyl-imidazole-2-carboxamide;
N-[3-chloro-4-[4-(piperidine-4-carbonyl)piperazine-1-carbonyl]phenyl]-5-(2-fluoro-3,4-dimethoxy-phenyl)-1-methyl-imidazole-2-carboxamide;
N-[4-[4-[5-[(3aS,4 S,6aR)-2-oxo-1,3,3a,4,6,6a-hexahydrothieno[3,4-d]imidazol-4-yl]pentanoyl]piperazine-1-carbonyl]-3-chloro-phenyl]-5-(2,3-difluoro-4-methoxy-phenyl)-1-methyl-imidazole-2-carboxamide;
N-[3-chloro-4-[4-[2-(2,5-dioxoimidazolidin-4-yl)acetyl]piperazine-1-carbonyl]phenyl]-difluoro-4-methoxy-phenyl)-1-methyl-imidazole-2-carboxamide;
N-[4-[3-[3-(2-aminoethoxy)propanoylamino]pyrrolidine-1-carbonyl]-3-chloro-phenyl]-5-(2,3-difluoro-4-methoxy-phenyl)-1-methyl-imidazole-2-carboxamide;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]acetic acid;
N-[3-chloro-4-[4-(piperidine-4-carbonyl)piperazine-1-carbonyl]phenyl]-5-[2,3-difluoro-4-(2-methoxyethoxy)phenyl]-1-methyl-imidazole-2-carboxamide;
N-[3-chloro-4-[4-[2-(dimethylamino)ethyl]piperazine-1-carbonyl]phenyl]-5-[2,3-difluoro-4-(2-pyridyloxy)phenyl]-1-methyl-imidazole-2-carboxamide;
N-[3-chloro-4-[4-[2-(dimethylamino)ethyl]piperazine-1-carbonyl]phenyl]-5-[2,3-difluoro-4-(4-pyridyloxy)phenyl]-1-methyl-imidazole-2-carboxamide;
N-[3-chloro-4-[4-(piperidine-4-carbonyl)piperazine-1-carbonyl]phenyl]-5-(3,4-difluoro-5-methoxy-phenyl)-1-methyl-imidazole-2-carboxamide;
N-[3-chloro-4-[4-[2-(dimethylamino)ethyl]piperazine-1-carbonyl]phenyl]-5-(2,3-difluoro-4-pyrimidin-2-yloxy-phenyl)-1-methyl-imidazole-2-carboxamide;
N-[3-chloro-4-[4-(piperidine-4-carbonyl)piperazine-1-carbonyl]phenyl]-5-[4-(cyanomethyl)-2-fluoro-phenyl]-1-methyl-imidazole-2-carboxamide;
N-[4-[4-(4-hydroxypiperidine-4-carbonyl)piperazine-1-carbonyl]-3-methyl-phenyl]-9-methoxy-6,7-dihydro-5H-imidazo[5,1-a][2]benzazepine-3-carboxamide;
N-[3-chloro-4-[4-[(2S,4R)-4-hydroxypyrrolidine-2-carbonyl]piperazine-1-carbonyl]phenyl]-5-(2,3-difluoro-4-methoxy-phenyl)-1-methyl-imidazole-2-carboxamide;
N-(azetidin-3-ylmethyl)-1-[2-chloro-4-[[5-[4-(cyanomethoxy)-2,3-difluoro-phenyl]-1-methyl-imidazole-2-carbonyl]amino]benzoyl]piperidine-4-carboxamide;
5-(2-chloro-3-fluoro-4-methoxy-phenyl)-N-[3-chloro-4-[4-(4-hydroxypiperidine-4-carbonyl)piperazine-1-carbonyl]phenyl]-1-methyl-imidazole-2-carboxamide;
N-[4-[4-[3-(2-aminoethoxy)propanoyl]piperazine-1-carbonyl]-3-chloro-phenyl]-5-(2,3-difluoro-4-methoxy-phenyl)-1-methyl-imidazole-2-carboxamide;
N-[4-[4-[1-(2-amino-2-oxo-ethyl)piperidine-4-carbonyl]piperazine-1-carbonyl]-3-chloro-phenyl]-5-[2,3-difluoro-4-(fluoromethoxy)phenyl]-1-methyl-imidazole-2-carboxamide;
N-[4-[4-[(3R)-3-aminopyrrolidine-1-carbonyl]piperazine-1-carbonyl]-3-chloro-phenyl]-5-[2,3-difluoro-4-(fluoromethoxy)phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3S)-pyrrolidin-3-yl]piperidine-4-carboxamide;
N-[3-chloro-4-[4-[2-[(2S)-pyrrolidin-2-yl]acetyl]piperazine-1-carbonyl]phenyl]-5-[4-(difluoromethoxy)-2,3-difluoro-phenyl]-1-methyl-imidazole-2-carboxamide;
N-[3-chloro-4-[4-[2-(dimethylamino)acetyl]piperazine-1-carbonyl]phenyl]-5-[2,3-difluoro-4-(fluoromethoxy)phenyl]-1-methyl-imidazole-2-carboxamide;
N-[3-chloro-4-[4-(piperidine-4-carbonyl)piperazine-1-carbonyl]phenyl]-5-[2-fluoro-4-(fluoromethoxy)phenyl-1-methyl-imidazole-2-carboxamide;
N-[3-chloro-4-[4-[(2S,4R)-4-hydroxypyrrolidine-2-carbonyl]piperazine-1-carbonyl]phenyl]-difluoro-4-(fluoromethoxy)phenyl]-1-methyl-imidazole-2-carboxamide;
N-(2-aminoethyl)-1-[2-chloro-4-[[5-[2,3-difluoro-4-(fluoromethoxy)phenyl]-1-methyl-imidazole-2-carbonyl]amino]benzoyl]piperidine-4-carboxamide;
N-(2-aminoethyl)-1-[2-chloro-4-[[5-[4-(difluoromethoxy)-2,3-difluoro-phenyl]-1-methyl-imidazole-2-carbonyl]amino]benzoyl]piperidine-4-carboxamide;
1-[2-chloro-4-[[5-[2,3-difluoro-4-(fluoromethoxy)phenyl]-1-methyl-imidazole-2-carbonyl]amino]benzoyl]-N-(4-piperidyl)piperidine-4-carboxamide;
1-[2-chloro-4-[[5-[4-(difluoromethoxy)-2,3-difluoro-phenyl]-1-methyl-imidazole-2-carbonyl]amino]benzoyl]-N-[(3R)-pyrrolidin-3-yl]piperidine-4-carboxamide;
1-[2-chloro-4-[[5-[4-(difluoromethoxy)-2,3-difluoro-phenyl]-1-methyl-imidazole-2-carbonyl]amino]benzoyl]-N-(4-piperidyl)piperidine-4-carboxamide;
1-[2-chloro-4-[[5-[4-(difluoromethoxy)-2,3-difluoro-phenyl]-1-methyl-imidazole-2-carbonyl]amino]benzoyl]-N-[(3S,4S)-4-hydroxypyrrolidin-3-yl]piperidine-4-carboxamide;
N-[3-chloro-4-[4-[2-(4-hydroxy-4-piperidyl)acetyl]piperazine-1-carbonyl]phenyl]-5-[4-(difluoromethoxy)-2,3-difluoro-phenyl]-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3R,4R)-4-hydroxypyrrolidin-3-yl]piperidine-4-carboxamide;
N-[3-chloro-4-[4-[2-(dimethylamino)acetyl]piperazine-1-carbonyl]phenyl]-5-[2-fluoro-4-(fluoromethoxy)phenyl]-1-methyl-imidazole-2-carboxamide;
1-[2-chloro-4-[[5-[2,3-difluoro-4-(fluoromethoxy)phenyl]-1-methyl-imidazole-2-carbonyl]amino]benzoyl]-N-[(3R)-pyrrolidin-3-yl]piperidine-4-carboxamide;
N-[4-[3-[[3-(aminomethyl)cyclobutanecarbonyl]amino]azetidine-1-carbonyl]-3-chloro-phenyl]-5-(2,3-difluoro-4-methoxy-phenyl)-1-methyl-imidazole-2-carboxamide;
N-[4-[3-[(2-aminoacetyl)amino]azetidine-1-carbonyl]-3-chloro-phenyl]-5-(2,3-difluoro-4-methoxy-phenyl)-1-methyl-imidazole-2-carboxamide;
N-[4-[4-(3-carbamoylcyclobutanecarbonyl)piperazine-1-carbonyl]-3-chloro-phenyl]-5-(2,3-difluoro-4-methoxy-phenyl)-1-methyl-imidazole-2-carboxamide;
N-[3-chloro-4-[4-(3-hydroxycyclobutanecarbonyl)piperazine-1-carbonyl]phenyl]-5-(2,3-difluoro-4-methoxy-phenyl)-1-methyl-imidazole-2-carboxamide;
N-[3-chloro-4-[4-(3-methoxypropanoyl)piperazine-1-carbonyl]phenyl]-5-(2,3-difluoro-4-methoxy-phenyl)-1-methyl-imidazole-2-carboxamide;
N-[4-[4-[3-(3-amino-3-oxo-propoxy)propanoyl]piperazine-1-carbonyl]-3-chloro-phenyl]-5-(2,3-difluoro-4-methoxy-phenyl)-1-methyl-imidazole-2-carboxamide; and
N-[4-[4-(5-amino-5-oxo-pentanoyl)piperazine-1-carbonyl]-3-chloro-phenyl]-5-(2,3-difluoro-4-methoxy-phenyl)-1-methyl-imidazole-2-carboxamide.

20. The compound of formula (I) according to claim 1, or a pharmaceutically acceptable salt thereof, wherein the compound of formula (I) is selected from:
N-[3-chloro-4-[4-[rac-(1R,5S)-3-azabicyclo[3.1.0]hexane-6-carbonyl]piperazine-1-carbonyl]phenyl]-5-(2,3-difluoro-4-methoxy-phenyl)-1-methyl-imidazole-2-carboxamide;
5-[3-chloro-2-fluoro-4-(fluoromethoxy)phenyl]-N-[3-chloro-4-[4-[(2S,4R)-4-hydroxypyrrolidine-2-carbonyl]piperazine-1-carbonyl]phenyl]-1-methyl-imidazole-2-carboxamide;
N-[3-chloro-4-[4-(piperidine-4-carbonyl)piperazine-1-carbonyl]phenyl]-5-[4-(difluoromethoxy)-2,3-difluoro-phenyl]-1-methyl-imidazole-2-carboxamide;
N-(azetidin-3-ylmethyl)-1-[2-chloro-4-[[5-[4-(difluoromethoxy)-2,3-difluoro-phenyl]-1-methyl-imidazole-2-carbonyl]amino]benzoyl]piperidine-4-carboxamide;
N-[3-chloro-4-[4-(2-pyrrolidin-3-ylacetyl)piperazine-1-carbonyl]phenyl]-5-(2,3-difluoro-4-methoxy-phenyl)-1-methyl-imidazole-2-carboxamide;
N-[4-[4-[2-(azetidin-3-yl)acetyl]piperazine-1-carbonyl]-3-chloro-phenyl]-5-(2,3-difluoro-4-methoxy-phenyl)-1-methyl-imidazole-2-carboxamide;
1-[2-chloro-4-[[5-[4-(difluoromethoxy)-2,3-difluoro-phenyl]-1-methyl-imidazole-2-carbonyl]amino]benzoyl]-N-[(3R,4R)-4-hydroxypyrrolidin-3-yl]piperidine-4-carboxamide;
4-[2-chloro-4-[[5-(2,3-difluoro-4-methoxy-phenyl)-1-methyl-imidazole-2-carbonyl]amino]benzoyl]-N-[(3R,4R)-4-hydroxypyrrolidin-3-yl]piperazine-1-carboxamide;
1-[2-chloro-4-[[5-[4-(difluoromethoxy)-2,3-difluoro-phenyl]-1-methyl-imidazole-2-carbonyl]amino]benzoyl]-N-[(3S,4S)-4-hydroxypyrrolidin-3-yl]piperidine-4-carboxamide;
N-[3-chloro-4-[4-[2-[(3S)-pyrrolidin-3-yl]acetyl]piperazine-1-carbonyl]phenyl]-5-(2,3-difluoro-4-methoxy-phenyl)-1-methyl-imidazole-2-carboxamide;
5-[3-chloro-2-fluoro-4-(fluoromethoxy)phenyl]-N-[4-[4-[(2S,4R)-4-hydroxypyrrolidine-2-carbonyl]piperazine-1-carbonyl]-3-methyl-phenyl]-1-methyl-imidazole-2-carboxamide;
N-[3-chloro-4-[4-[(2S,4S)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,3S)-3-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
4-[2-chloro-4-[[5-[4-(cyanomethoxy)-2,3-difluoro-phenyl]-1-methyl-imidazole-2-carbonyl]amino]benzoyl]-N-[(3S,4S)-4-hydroxypyrrolidin-3-yl]piperazine-1-carboxamide;
N-[3-chloro-4-[4-(4-hydroxypiperidine-4-carbonyl)piperazine-1-carbonyl]phenyl]-5-[4-(cyanomethoxy)-2,3-difluoro-phenyl]-1-methyl-imidazole-2-carboxamide;
N-[3-chloro-4-[4-(piperidine-4-carbonyl)piperazine-1-carbonyl]phenyl]-5-[4-(cyanomethoxy)-2,3-difluoro-phenyl]-1-methyl-imidazole-2-carboxamide;
N-[4-[4-[2-(azetidin-3-yl)acetyl]piperazine-1-carbonyl]-3-chloro-phenyl]-5-[4-(difluoromethoxy)-2-fluoro-phenyl]-1-methyl-imidazole-2-carboxamide;
N-(azetidin-3-ylmethyl)-1-[2-chloro-4-[[5-(2,3-difluoro-4-methoxy-phenyl)-1-methyl-imidazole-2-carbonyl]amino]benzoyl]piperidine-4-carboxamide;
N-[3-chloro-4-[4-[2-[(2S)-pyrrolidin-2-yl]acetyl]piperazine-1-carbonyl]phenyl]-5-(2,3-difluoro-4-methoxy-phenyl)-1-methyl-imidazole-2-carboxamide;
N-[3-chloro-4-[4-[(3R)-3-(hydroxymethyl)piperazine-1-carbonyl]piperidine-1-carbonyl]phenyl]-5-[4-(cyanomethoxy)-2,3-difluoro-phenyl]-1-methyl-imidazole-2-carboxamide;
4-[2-chloro-4-[[5-[4-(cyanomethoxy)-2,3-difluoro-phenyl]-1-methyl-imidazole-2-carbonyl]amino]benzoyl]-N-[(3R,4R)-4-hydroxypyrrolidin-3-yl]piperazine-1-carboxamide;
N-[4-[4-[(2S,4S)-4-aminopyrrolidine-2-carbonyl]piperazine-1-carbonyl]-3-chloro-phenyl]-5-[4-(cyanomethoxy)-2,3-difluoro-phenyl]-1-methyl-imidazole-2-carboxamide;
4-[2-chloro-4-[[5-(2,3-difluoro-4-methoxy-phenyl)-1-methyl-imidazole-2-carbonyl]amino]benzoyl]-N-[(3S,4S)-4-hydroxypyrrolidin-3-yl]piperazine-1-carboxamide;
N-[3-chloro-4-[4-[(3S,4R)-3-hydroxypiperidine-4-carbonyl]piperazine-1-carbonyl]phenyl]-5-[4-(cyanomethoxy)-2,3-difluoro-phenyl]-1-methyl-imidazole-2-carboxamide;
N-(azetidin-3-ylmethyl)-1-[2-chloro-4-[[5-[4-(cyanomethoxy)-2,3-difluoro-phenyl]-1-methyl-imidazole-2-carbonyl]amino]benzoyl]piperidine-4-carboxamide;
N-[3-chloro-4-[4-[(2S,4R)-4-hydroxypyrrolidine-2-carbonyl]piperazine-1-carbonyl]phenyl]-5-[4-(cyanomethoxy)-2,3-difluoro-phenyl]-1-methyl-imidazole-2-carboxamide;
N-[3-chloro-4-[4-[(2S,4R)-4-hydroxypyrrolidine-2-carbonyl]piperazine-1-carbonyl]phenyl]-difluoro-4-methoxy-phenyl)-1-methyl-imidazole-2-carboxamide;
N-[3-chloro-4-[4-[(3S,4R)-3-hydroxypiperidine-4-carbonyl]piperazine-1-carbonyl]phenyl]-difluoro-4-methoxy-phenyl)-1-methyl-imidazole-2-carboxamide;
4-[4-[[5-(2,3-difluoro-4-methoxy-phenyl)-1-methyl-imidazole-2-carbonyl]amino]-2-methyl-benzoyl]-N-[(3S,4S)-4-hydroxypyrrolidin-3-yl]piperazine-1-carboxamide; and
5-(2-chloro-3-fluoro-4-methoxy-phenyl)-N-[4-[4-(4-hydroxypiperidine-4-carbonyl)piperazine-1-carbonyl]-3-methyl-phenyl]-1-methyl-imidazole-2-carboxamide.

21. A process of manufacturing the compounds of formula (I) according to any one of claims 1 to 20, wherein said process is as described in any one of Schemes 1 to 5 herein.

22. A compound of formula (I) according to any one of claims 1 to 20, when manufactured according to the process of claim 21.

23. A compound of formula (I) according to any one of claims 1 to 20 and 22, or a pharmaceutically acceptable salt thereof, for use as therapeutically active substance.

24. A pharmaceutical composition comprising a compound of formula (I) according to any one of claims 1 to 20 and 22, or a pharmaceutically acceptable salt thereof, and a therapeutically inert carrier.

25. A compound of formula (I) according to any of claims 1 to 20 and 22, or a pharmaceutically acceptable salt thereof, for use as antibiotic.

26. A compound of formula (I) according to any of claims 1 to 20 and 22, or a pharmaceutically acceptable salt thereof, for use in the treatment or prevention of nosocomial infections and resulting diseases.

27. A compound of formula (I) according to any of claims 1 to 20 and 22, or a pharmaceutically acceptable salt thereof, for use in the treatment or prevention of infections and resulting diseases caused by Gram-negative bacteria.

28. The compound for use according to claim 27, wherein said Gram-negative bacteria are selected from Klebsiella pneumoniae, Acinetobacter baumannii, Pseudomonas aeruginosa, Enterobacter species and E. coli.

29. The compound for use according to claim 28, wherein said Gram-negative bacteria are Acinetobacter baumannii.

30. A compound of formula (I) according to any of claims 1 to 20 and 22, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

31. A method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) according to any of claims 1 to 20 and 22, or a pharmaceutically acceptable salt thereof, to a mammal.

32. Use of a compound of formula (I) according to any of claims 1 to 20 and 22, or a pharmaceutically acceptable salt thereof, as an antibiotic.

33. Use of a compound of formula (I) according to any of claims 1 to 20 and 22, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

34. The use of a compound of formula (I) according to any of claims 1 to 20 and 22, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

35. The invention as described hereinbefore.
